(19) United States
(12) Patent Application Publication
(10) Pub. No.: US 2021/0087206 A1
(43) Pub. Date: Mar. 25, 2021

(54) MACROCYCLIC KINASE INHIBITORS AND THEIR USE

(71) Applicant: TURNING POINT THERAPEUTICS, INC., San Diego, CA (US)

(72) Inventors: Evan W. ROGERS, San Diego, CA (US); Jane UNG, San Diego, CA (US); Han ZHANG, San Diego, CA (US); Jeffrey WHITTEN, San Diego, CA (US); Dayong ZHAI, San Diego, CA (US); Wei DENG, San Diego, CA (US); Jingrong Jean CUI, San Diego, CA (US)

(21) Appl. No.: 16/772,014

(22) PCT Filed: Dec. 18, 2018
(86) PCT No.: PCT/US2018/066159

§ 371 Date: Jun. 11, 2020

Related U.S. Application Data
(60) Provisional application No. 62/607,529, filed on Dec. 19, 2017.
(60) Provisional application No. 62/779,151, filed on Dec. 13, 2018.

(51) Int. Cl. C07D 498/22; A61P 35/02

(52) U.S. Cl. CPC C07D 498/22; A61P 35/02

(57) ABSTRACT

The present disclosure relates to certain chiral diaryl macrocyclic derivatives, pharmaceutical compositions containing them, and methods of using them to treat cancer, pain, neurological diseases, autoimmune diseases, and inflammation.

### CROSS-REFERENCE TO RELATED APPLICATIONS

This application claims priority under 35 U.S.C. § 119(e) to U.S. Provisional Application Ser. No. 62/607,529 filed on Dec. 19, 2017 and U.S. Provisional Application Ser. No. 62/779,151 filed on Dec. 13, 2018, the entire disclosures of which are incorporated herein by reference.

### TECHNICAL FIELD

The present disclosure relates to certain chiral diaryl macrocyclic derivatives, pharmaceutical compositions containing them, and methods of using them to treat cancer, pain, neurological diseases, autoimmune diseases, and inflammation.

### BACKGROUND

Protein kinases are key regulators for cell growth, proliferation and survival. Genetic and epigenetic alterations accumulate in cancer cells leading to abnormal activation of signal transduction pathways which drive malignant processes. Manning, G. et al., Science 2002, 298, 1912-1934. Pharmacological inhibition of these signaling pathways presents promising intervention opportunities for targeted cancer therapies. Sawyers, C., Nature 2004, 432, 294-297.

ALK, along with leukocyte tyrosine kinase (LTK), is grouped within the insulin receptor (IR) superfamily of receptor tyrosine kinases. ALK is mainly expressed in the central and peripheral nervous systems suggesting a potential role in normal development and function of the nervous system. Pulford, K. et al., Cell Mol. Life Sci. 2004, 61, 2939. ALK was first discovered as a fusion protein, NPM (nucleophosmin)-ALK, encoded by a fusion gene arising from the t(2;5)(p23;q35) chromosomal translocation in anaplastic large cell lymphoma (ALCL) cell lines. Morris, S. W. et al., Science 1994, 263, 1281. More than twenty distinct ALK translocation partners have been discovered in many cancers, including ALCL (60-90% incidence), inflammatory myofibroblastic tumors (IMT, 50-60%), non-small cell lung carcinomas (NSCLC, 3-7%), colorectal cancers (CRC, 0-2.4%), breast cancers (0-2.4%), and other carcinomas. Grande, E. et al., Mol. Cancer Ther. 2011, 10, 569-579. The ALK-fusion proteins are located in the cytoplasm, and the fusion partners with ALK play a role in dimerization or oligomerization of the fusion proteins through a coil-coil interaction to generate constitutive activation of ALK kinase function. Bischof, D. et al., Mol. Cell Biol., 1997, 17, 2312-2325. EML4-ALK, which comprises portions of the echinoderm microtubule associated protein-like 4 (EML4) gene and the ALK gene, was first discovered in NSCLC, is highly oncogenic, and was shown to cause lung adenocarcinoma in transgenic mice. Soda, M. et al., Nature 2007, 448, 561-566. Oncogenic point mutations of ALK in both familial and sporadic cases of neuroblastoma. Moss6, Y. P. et al., Nature 2008, 455, 930-935. ALK is an attractive molecular target for cancer therapeutic intervention because of the important roles in haematopoietic, solid, and mesenchymal tumors. Grande, supra.

The tropomyosin-related receptor tyrosine kinases (Trks) are the high-affinity receptor for neurotrophins (NTs), a nerve growth factor (NGF) family of proteins. Members of the Trk family are highly expressed in cells of neural origin. Activation of Trks (TrkA, TrkB, and TrkC) by their preferred neurotrophins (NGF to TrkA, brain-derived neurotrophic factor [BDNF] and NT4/5 to TrkB, and NT3 to TrkC) mediates the survival and differentiation of neurons during development. The NT/Trk signaling pathway functions as an endogenous system that protects neurons after biochemical insults, transient ischemia, or physical injury. Thiele, C. J. et al., Clin. Cancer Res. 2009, 15, 5962-5967. However, Trk was originally cloned as an oncogene fused with the tropomyosin gene in the extracellular domain. The activating mutations caused by chromosomal rearrangements or mutations in NTRK1 (TrkA) has been identified in papillary and medullary thyroid carcinoma, and recently in non-small cell lung cancer. Pierotti, M. A. et al., Cancer Lett. 2006, 232, 90-98; Vaishnavi, A. et al., Nat. Med. 2013, 19, 1469-1472. Because Trks play important roles in pain sensation as well as tumor cell growth and survival signaling, inhibitors of Trk receptor kinases may provide benefits as treatments for pain and cancer.

The Janus family of kinases (JAKs) includes JAK1, JAK2, JAK3 and TYK2, and are cytoplastic tyrosine kinases required for the physiologic signaling of cytokines and growth factors. Quintas-Cardama, A. et al., Nat. Rev. Drug Discov. 2011, 10(2), 127-40; Pesu, M. et al., Immunol. Rev. 2008, 223, 132-142; Murray, P. J., J. Immunol. 2007, 178(5), 2623-2329. JAKs activate by ligand-induced oligomerization, resulting in the activation of a downstream transcriptional signaling pathway called STAT (signal transducers and activators of transcription). The phosphorylated STATs dimerize and translocate into the nucleus to drive the expression of specific genes involved in proliferation, apoptosis, differentiation, which are essential for hematopoiesis, inflammation and immune response. Murray, supra.

Mouse knockout studies have implicated the primary roles of JAK-STAT signaling with some overlap between them. JAK1 plays a critical role in the signaling of various proinflammatory cytokines such as IL-1, IL-4, IL-6, and tumor necrosis factor alpha (TNFα). Muller, M. et al., Nature 1993, 366(6451), 129-135. JAK2 functions for hematopoietic growth factors signaling such as Epo, IL-3, IL-5, GM-CSF, thrombopoietin growth hormone, and prolactin-mediated signaling. Neubauer, H. et al., Cell 1998 93(3), 397-409. JAK3 plays a role in mediating immune responses, and TYK2 associates with JAK2 or JAK3 to transduce signaling of cytokines, such as IL-12. Nosaka, T. et al., Science 1995, 270(5237), 800-802; Vainchenker, W. et al., Semin. Cell. Dev. Biol. 2008, 19(4), 385-393.

Aberrant regulation of JAK/STAT pathways has been implicated in multiple human pathological diseases, including cancer (JAK2) and rheumatoid arthritis (JAK1, JAK3). A gain-of-function mutation of JAK2 (JAK2V617F) has been discovered with high frequency in MPN patients. Levine, R. L. et al., Cancer Cell 2005, 7(4), 387-397; Kralovics, R. et al., N. Engl. J. Med. 2005, 253(17), 1779-1790; James, C. et al., Nature 2005, 434(7037), 1144-1148; Baxter, E. J. et al. Lancet 2005, 365(9464), 1054-1061. The mutation in the JH2 pseudokinase domain of JAK2 leads to constitutively kinase activity. Cells containing JAK2V617F mutation acquire cytokine-independent growth ability and often become a tumor, providing a strong rational for the development of JAK inhibitors as target therapy.

Multiple JAK inhibitors in clinical trial showed significant benefit in splenomegaly and disease-related constitutional symptoms for the myelofibrosis patients, including the first FDA-approved JAK2 inhibitor ruxolitinib in 2011. Quintas-Cardama, supra; Sonbol, M. B. et al., Ther. Adv. Hematol. 2013, 4(1), 15-35; LaFave, L. M. et al., Trends Pharmacol. Sci. 2012, 33(11), 574-582. The recently collected clinical data related to ruxolitinib treatment indicated that JAK inhibitors work on both JAK2 wild-type and JAK2 mutated cases. Verstovsek, S. et al., N. Engl. J. Med. 2012, 366(9), 799-807; Quintas-Cardama, A. et al., Blood 2010, 115(15), 3109-3117. The discovery of selective inhibitors of JAK2 vs JAK1/3 remains an unsolved challenge. In addition, hyperactivation of the JAK2/signal transducers and activators of transcription 3 (JAK2/STAT3) is responsible for abnormal dendritic cell differentiation leading to abnormal dendritic cell differentiation and accumulation of immunosuppressive myeloid cells in cancer (Nefedova, Y. et al., Cancer Res 2005; 65(20): 9525-35). In Pten-null senescent tumors, activation of the Jak2/Stat3 pathway establishes an immunosuppressive tumor microenvironment that contributes to tumor growth and chemoresistance (Toso, A. et al., Cell Reports 2014, 9, 75-89). Therefore, pharmacologic inhibition of the JAK2/STAT3 pathway can be an important new therapeutic strategy to enhance antitumor activity via the regulation of antitumor immunity.

ROS1 kinase is a receptor tyrosine kinase with an unknown ligand. The normal functions of human ROS1 kinase have not been fully understood. However, it has been reported that ROS1 kinase undergoes genetic rearrangements to create constitutively active fusion proteins in a variety of human cancers including glioblastoma, non-small cell lung cancer (NSCLC), cholangiocarcinoma, ovarian cancer, gastric adenocarcinoma, colorectal cancer, inflammatory myofibroblastic tumor, angiosarcoma, and epithelioid hemangioendothelioma (Davies, K. D. et al., Clin Cancer Res 2013, 19 (15): 4040-4045). Targeting ROS1 fusion proteins with crizotinib has demonstrated promising clinical efficacy in NSCLC patients whose tumors are positive for ROS1 genetic abnormalities (Shaw, A. T. et al., N Engl J Med. 2014, 371(21):1963-1971). Acquired resistant mutations have been observed in crizotinib treatment patients (Awad, M. M. et al., N Engl J Med. 2013, 368(25):2396-2401). It is urgent to develop the second generation of ROS1 inhibitors for overcoming crizotinib ROS1 resistance.

Crizotinib (PF-02341066) is a tyrosine kinase drug targeting MET/ALK/ROS1/RON with moderate activity against TRKs and AXL. Cui, J. J. et al., J. Med. Chem. 2011, 54, 6342-6363. It was approved to treat certain patients with late-stage (locally advanced or metastatic) NSCLC that expresses the abnormal ALK fusion gene identified by a companion diagnostic test (Vysis ALK Break Apart FISH Probe Kit). Similar to imatinib and other kinase inhibitor drugs, resistance invariably develops after a certain time of treatment with crizotinib. The resistance mechanisms include ALK gene amplification, secondary ALK mutations, and aberrant activation of other kinases including KIT and EGFR. Katayama, R. et al., Sci. Transl. Med. 2012, 4, 120ra17. Based on the clinical success of second-generation ABL inhibitors for the treatment of imatinib resistance in CML patients, a second generation of ALK inhibitors is emerging. These drugs target the treatment of crizotinib-refractory or resistant NSCLC patient with more potent inhibition against both wild and mutant ALK proteins. Gridelli, C. et al., Cancer Treat Rev. 2014, 40, 300-306.

There remains a need for small molecule inhibitors of these multiple protein or tyrosine kinase targets with desirable pharmaceutical properties. Certain chiral diaryl macrocyclic compounds have been found in the context of this disclosure to have this advantageous activity profile.

### SUMMARY

In one aspect, the disclosure relates to a compound of the formula I

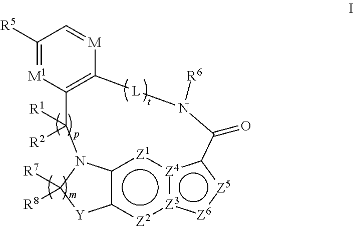

wherein

L is independently —C(R¹)(R²)— or X;

X is —O—, —S—, —S(O)—, or —S(O)₂—;

each R¹ and R² is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

M is CR³ or N;

M¹ is CR⁴;

each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅-C₈ cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

Y is O, S, NR⁸, or CR⁷R⁸;

each R⁷ and R⁸ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is optionally substituted by a halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e};

each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl;

each of Z¹, Z², Z³, Z⁴, Z⁵, and Z⁶ is independently N, NH, C or CH;

m is 0, 1, 2, or 3;

p is 1, 2, 3, or 4; and

t is 1, 2, 3, 4, or 5;

or a pharmaceutically acceptable salt thereof.

In another aspect, the disclosure relates to a compound of the formula I

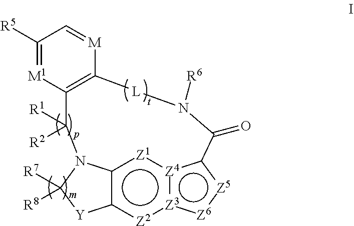

wherein

L is independently —C(R¹)(R²)— or X, with the proviso that when t is 1, then L is —C(R¹)(R²)—;

X is —O—, —S—, —S(O)—, or —S(O)₂—;

each R¹ and R² is independently H, deuterium, halogen, C₁—C alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

M is CR³ or N;

M¹ is CR⁴;

each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring atoms to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅-C₈ cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

Y is O, S, NR⁸, or CR⁷R⁸;

each R⁷ and R⁸ is independently H, deuterium, halogen, —CN, —OR^{e}, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₂-C₆ alkenyl, C₂-C₆ alkynyl, C₃-C₆ cycloalkyl, 4- to 6-membered heterocycloalkyl, 3- to 7-membered heterocycloalkyl, C₆-C₁₀ aryl, exocyclic ethylene group, or mono- or bicyclic heteroaryl is optionally substituted by a halogen, —N₃, —CN, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e};

each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl;

each of Z¹, Z², Z³, Z⁴, Z⁵, and Z⁶ is independently N, NH, C or CH;

m is 0, 1, 2, or 3;

p is 1, 2, 3, or 4; and

t is 1, 2, 3, 4, or 5;

or a pharmaceutically acceptable salt thereof

In another aspect, the disclosure relates to a compound or a pharmaceutically acceptable salt thereof, having the formula II having the formula II

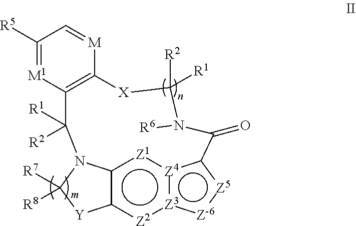

wherein

M is CR³ or N;

M¹ is CR⁴;

X is O, S, S(O), or S(O)₂;

each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸;

each R⁷ and R⁸ is independently H, deuterium, halogen, or C₁-C₆ alkyl, wherein each hydrogen atom in C₁-C₆ alkyl is optionally substituted by a halogen, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl;

each of Z¹, Z², Z³, Z⁴, Z⁵, and Z⁶ is independently N, NH, C or CH;

m is 0, 1, 2, or 3; and

n is 2 or 3.

In another aspect, the disclosure relates to a compound or a pharmaceutically acceptable salt thereof, having the formula II

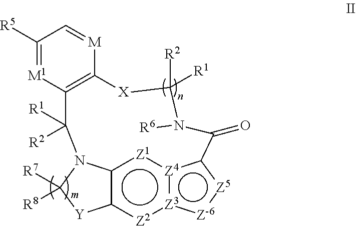

wherein

M is CR³ or N;

M¹ is CR⁴;

X is O, S, S(O), or S(O)₂;

each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸;

each R⁷ and R⁸ is independently H, deuterium, halogen, —CN, —OR^{e}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group is optionally substituted by a halogen, —N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl;

each of Z¹, Z², Z³, Z⁴, Z⁵, and Z⁶ is independently N, NH, C or CH;

m is 0, 1, 2, or 3; and

n is 2, 3, or 4.

In another aspect, the disclosure relates to a compound selected from the group consisting of

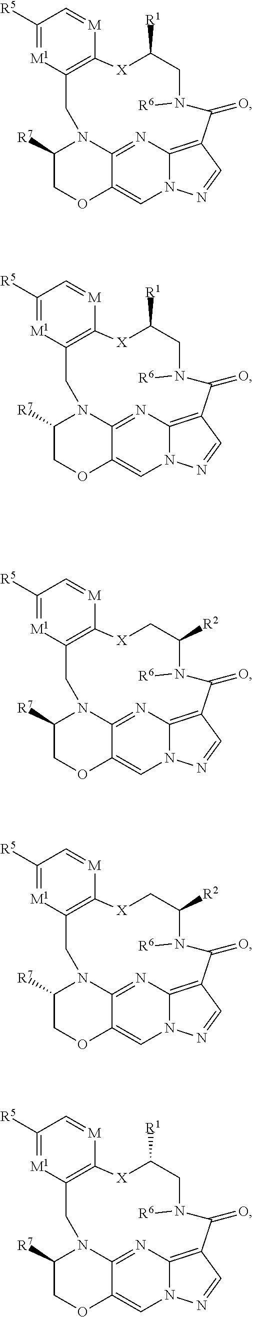

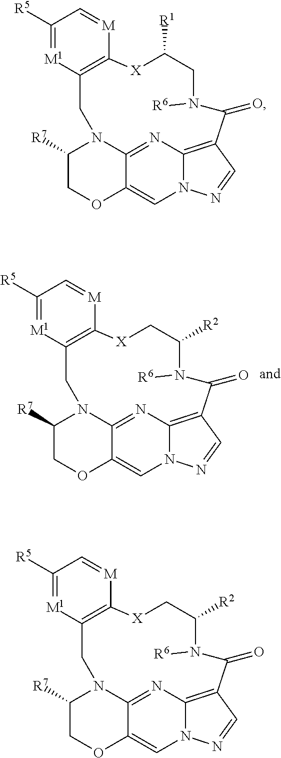

wherein

M is CR³ or N;

M¹ is CR⁴;

X is O, S, S(O), or S(O)₂;

R¹ and R² are each independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR^{B}, or CR⁷R⁸; and

each R⁷ and R⁸ is independently H, deuterium, halogen, or C₁-C₆ alkyl, wherein each hydrogen atom in C₁-C₆ alkyl is optionally substituted by a halogen, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl.

In another aspect, the disclosure relates to a compound selected from the group consisting of

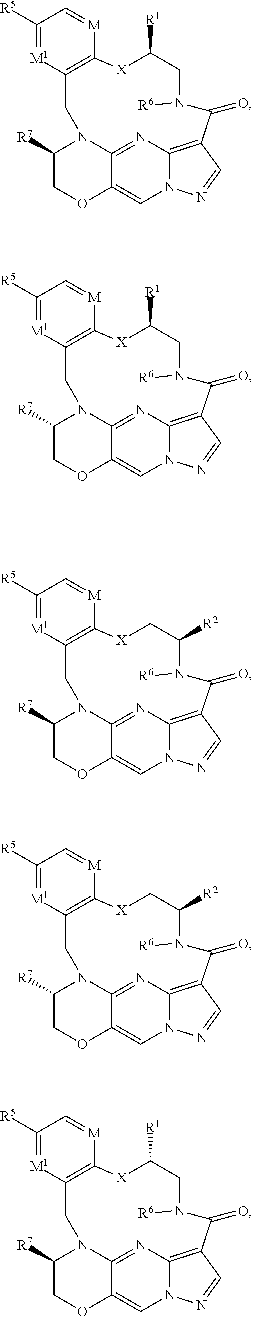

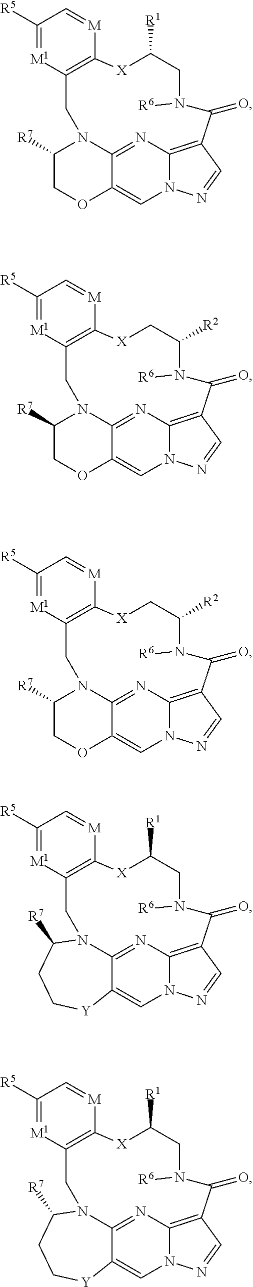

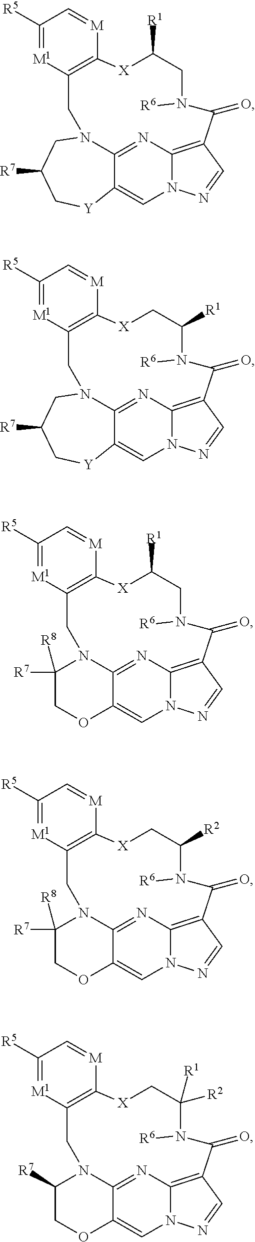

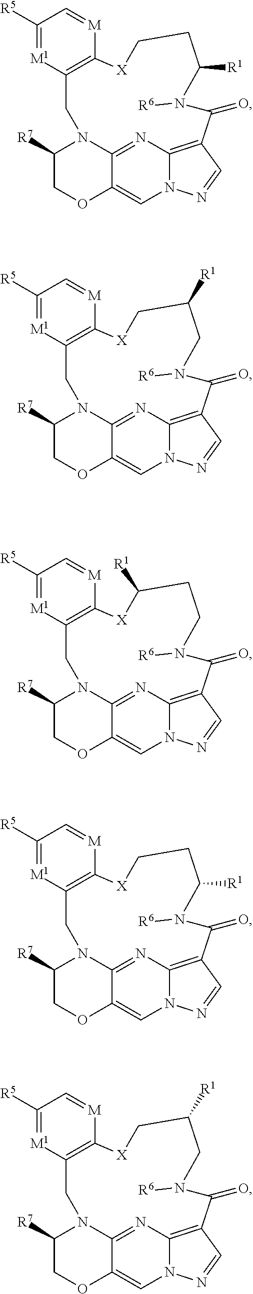

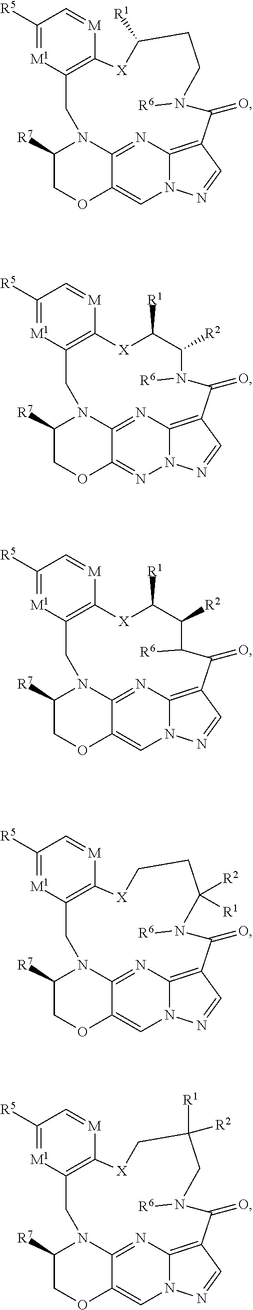

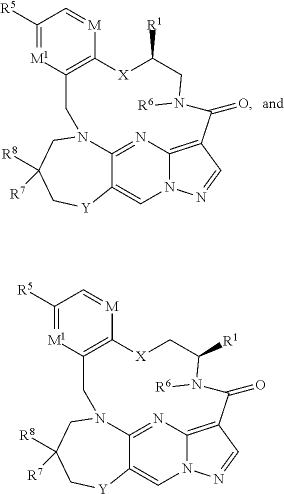

wherein

M is CR³ or N;

M¹ is CR⁴;

X is O, S, S(O), or S(O)₂;

R¹ and R² are each independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁—C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR^{B}, or CR⁷R⁸; and

each R⁷ and R⁸ is independently H, deuterium, halogen, —CN, —OR^{c}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group, or mono- or bicyclic heteroaryl wherein each hydrogen atom in C₁-C₆ alkyl is optionally substituted by a halogen, N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁—C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I

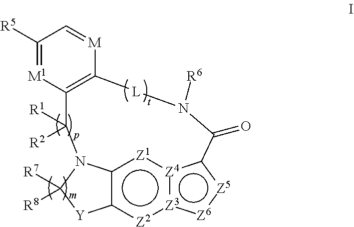

wherein

L is independently —C(R¹)(R²)— or X;

X is —O—, —S—, —S(O)—, or —S(O)₂—;

each R¹ and R² is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂R^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

M is CR³ or N;

M¹ is CR⁴;

each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅-C₈ cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

Y is O, S, NR^{B}, or CR⁷R⁸;

each R⁷ and R⁸ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is optionally substituted by a halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e};

each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl;

each of Z¹, Z², Z³, Z⁴, Z⁵, and Z⁶ is independently N, NH, C or CH;

m is 0, 1, 2, or 3;

p is 1, 2, 3, or 4; and

t is 1, 2, 3, 4, or 5;

or a pharmaceutically acceptable salt thereof.

1a. A compound of the formula I

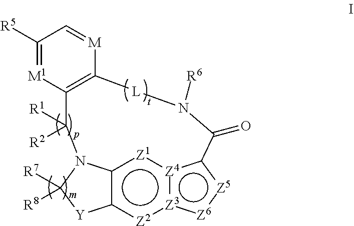

wherein

L is independently —C(R¹)(R²)— or X, with the proviso that when t is 1, then L is —C(R¹)(R²)—;

X is —O—, —S—, —S(O)—, or —S(O)₂—;

each R¹ and R² is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂R^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

M is CR³ or N;

M¹ is CR⁴;

each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{e}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring atoms to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅-C₈ cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

Y is O, S, NR⁸, or CR⁷R⁸;

each R⁷ and R⁸ is independently H, deuterium, halogen, —CN, —OR^{e}, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₂-C₆ alkenyl, C₂-C₆ alkynyl, C₃-C₆ cycloalkyl, 4- to 6-membered heterocycloalkyl, 3- to 7-membered heterocycloalkyl, C₆-C₁₀ aryl, exocyclic ethylene group, or mono- or bicyclic heteroaryl is optionally substituted by a halogen, —N₃, —CN, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e};

each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl;

each of Z¹, Z², Z³, Z⁴, Z⁵, and Z⁶ is independently N, NH, C or CH;

m is 0, 1, 2, or 3;

p is 1, 2, 3, or 4; and

t is 1, 2, 3, 4, or 5;

or a pharmaceutically acceptable salt thereof.

2. The compound of clause 1 or 1a, or a pharmaceutically acceptable salt thereof, wherein p is 1.

3. The compound of any of the preceding clauses, or a pharmaceutically acceptable salt thereof, wherein t is 3.

3. The compound of any of the preceding clauses, or a pharmaceutically acceptable salt thereof, wherein t is 3 or 4.

4. The compound of clause 1 or 1a, or a pharmaceutically acceptable salt thereof, having the formula II

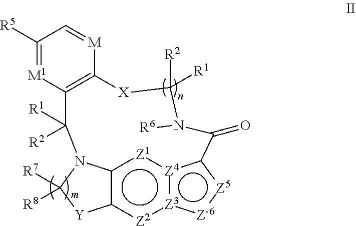

wherein

M is CR³ or N;

M¹ is CR⁴;

X is O, S, S(O), or S(O)₂;

each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸;

each R⁷ and R⁸ is independently H, deuterium, halogen, or C₁-C₆ alkyl, wherein each hydrogen atom in C₁-C₆ alkyl is optionally substituted by a halogen, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl;

each of Z¹, Z², Z³, Z⁴, Z⁵, and Z⁶ is independently N, NH, C or CH;

m is 0, 1, 2, or 3; and

n is 2 or 3.

4a. The compound of clause 1, or a pharmaceutically acceptable salt thereof, having the formula II

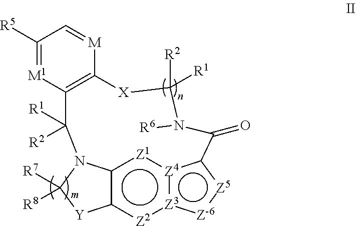

wherein

M is CR³ or N;

M¹ is CR⁴;

X is O, S, S(O), or S(O)₂;

each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}, or R¹ and R² taken together with the carbon to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸;

each R⁷ and R⁸ is independently H, deuterium, halogen, —CN, —OR^{c}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group is optionally substituted by a halogen, —N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl;

each of Z¹, Z², Z³, Z⁴, Z⁵, and Z⁶ is independently N, NH, C or CH;

m is 0, 1, 2, or 3; and

n is 2, 3, or 4.

5. The compound of any of the preceding clauses, having the formula III

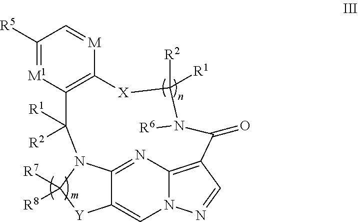

or a pharmaceutically acceptable salt thereof.

6. The compound of clause 4, 4a, or 5, or a pharmaceutically acceptable salt thereof, wherein n is 2.

7. The compound of clause 4, 4a, 5 or 6, or a pharmaceutically acceptable salt thereof, wherein m is 2.

8. The compound of any one of the preceding clauses, having the formula IV

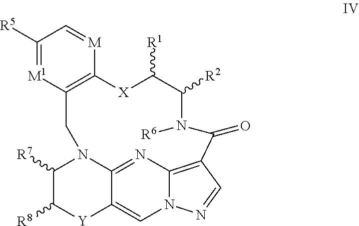

or a pharmaceutically acceptable salt thereof.

8a. The compound of any one of the preceding clauses, having the formula IV

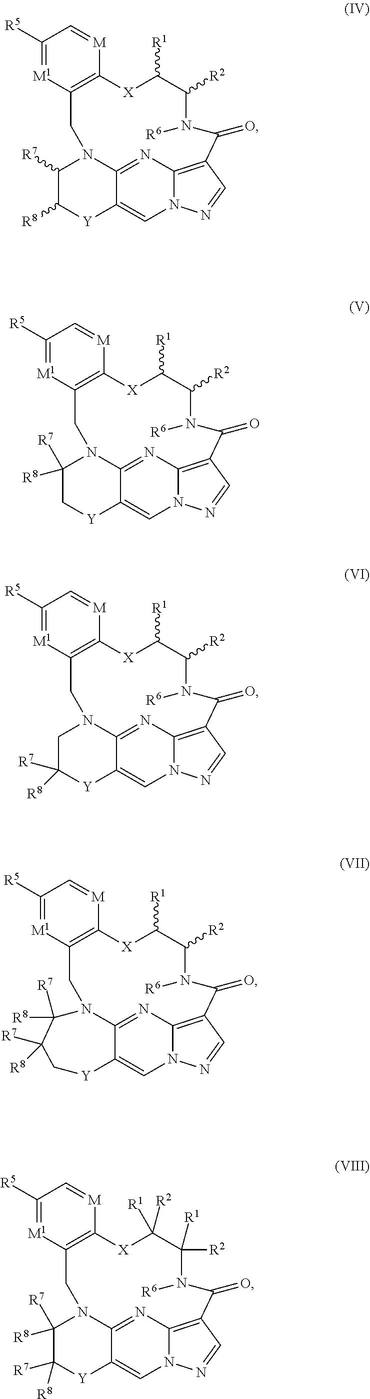

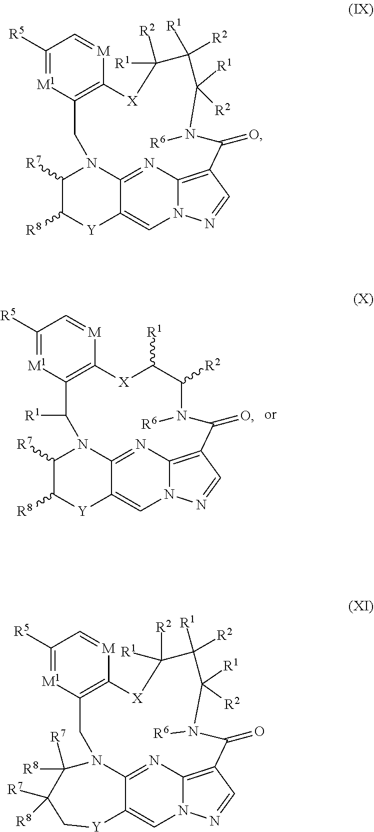

or a pharmaceutically acceptable salt thereof.

9. The compound of any one of the preceding clauses, or a pharmaceutically acceptable salt thereof, wherein Y is O.

10. The compound of any one of the preceding clauses, or a pharmaceutically acceptable salt thereof, wherein M is CR³.

11. The compound of any one of the preceding clauses, or a pharmaceutically acceptable salt thereof, wherein R³ is H, deuterium, C₁-C₆ alkyl or halogen.

12. The compound of any one of the preceding clauses, or a pharmaceutically acceptable salt thereof, wherein R³ is H or F.

13. The compound of any one of clauses 1 to 9, or a pharmaceutically acceptable salt thereof, wherein M is N.

14. The compound of any one of the preceding clauses, or a pharmaceutically acceptable salt thereof, wherein M¹ is CR⁴.

15. The compound of any one of the preceding clauses, or a pharmaceutically acceptable salt thereof, wherein R⁴ is H, deuterium, —CN, C₁-C₆ alkyl or halogen.

16. The compound of any one of the preceding clauses, or a pharmaceutically acceptable salt thereof, wherein R⁴ is H or Cl.

17. The compound of any one of the preceding clauses, or a pharmaceutically acceptable salt thereof, wherein R⁵ is F.

18. The compound of any one of the preceding clauses, or a pharmaceutically acceptable salt thereof, wherein R⁸ is H.

19. The compound of any one of the preceding clauses, or a pharmaceutically acceptable salt thereof, wherein R² is H.

20. The compound of any one of the preceding clauses, or a pharmaceutically acceptable salt thereof, wherein R¹ is H.

20a. The compound of any one of the preceding clauses, or a pharmaceutically acceptable salt thereof, wherein each R¹ and R² is independently H, or R¹ and R² taken together with the carbon to which they are attached form a C₃-C₆ cycloalkyl.

21. The compound of any one of clauses 1 to 19, or a pharmaceutically acceptable salt thereof, wherein R¹ is C₁-C₆ alkyl.

21a. The compound of any one of clauses 1 to 19, or a pharmaceutically acceptable salt thereof, wherein on one carbon atom, R¹ and R² taken together with the carbon to which they are attached form a C₃-C₆ cycloalkyl, and any other R¹ and R² when present is H.

22. The compound of any one of clauses 1 to 19, or a pharmaceutically acceptable salt thereof, wherein R¹ is H and R² is C₁-C₆ alkyl.

22a. The compound of any one of clauses 1 to 19, or a pharmaceutically acceptable salt thereof, wherein at least one of R¹ is H and at least one of R² is C₁-C₆ alkyl.

23. The compound of any one of the preceding clauses, or a pharmaceutically acceptable salt thereof, wherein R⁷ is H or C₁-C₆ alkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl.

24. The compound of any one of the preceding clauses, wherein R⁷ is H or C₁-C₆ alkyl, wherein each hydrogen atom in C₁-C₆ alkyl is independently optionally substituted by deuterium, —OH, or —OC₁-C₆ alkyl.

25. The compound of clause 1 or 1a, selected from the group consisting of

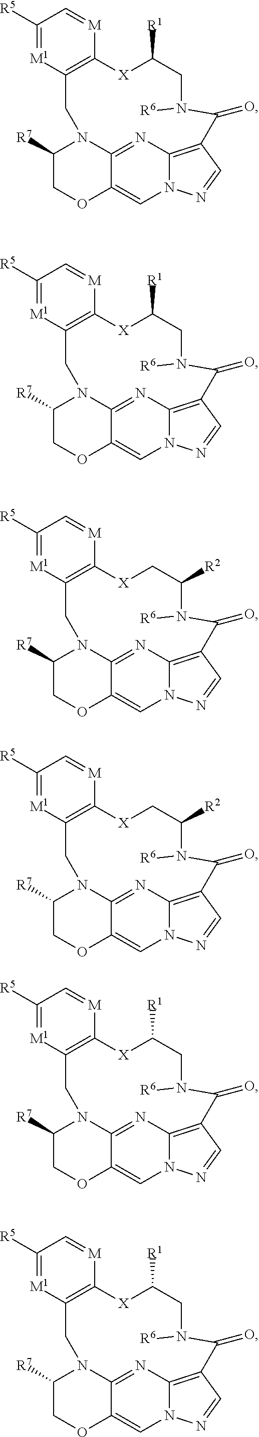

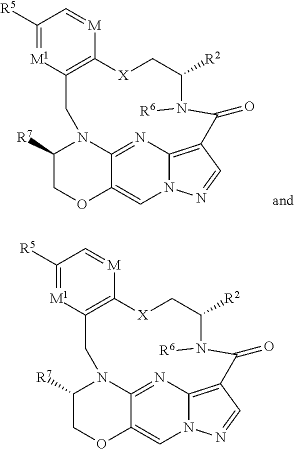

wherein

M is CR³ or N;

M¹ is CR⁴;

X is O, S, S(O), or S(O)₂;

R¹ and R² are each independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸; and

each R⁷ and R⁸ is independently H, deuterium, halogen, or C₁-C₆ alkyl, wherein each hydrogen atom in C₁-C₆ alkyl is optionally substituted by a halogen, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl.

25a. The compound of clause 1, selected from the group consisting of

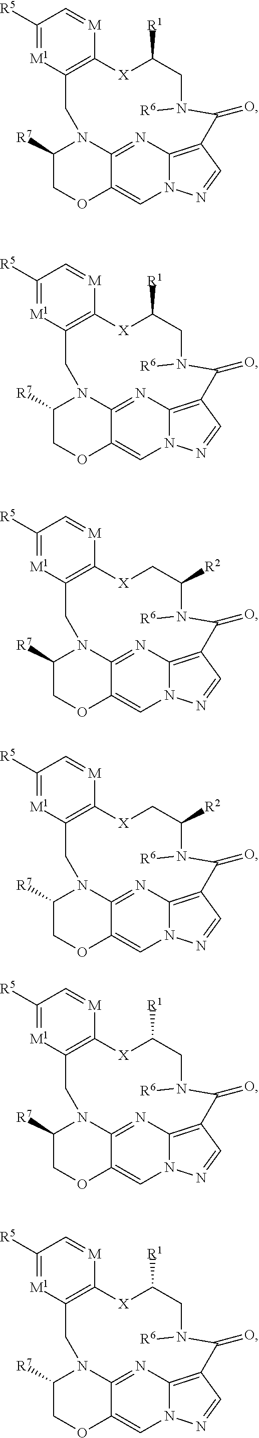

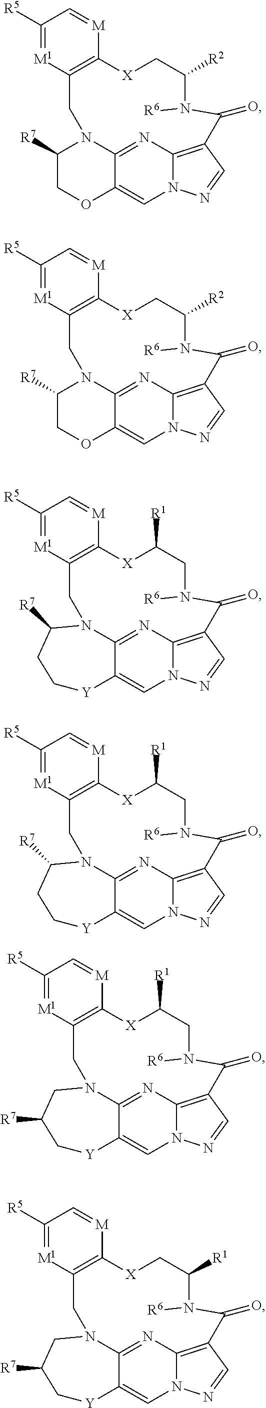

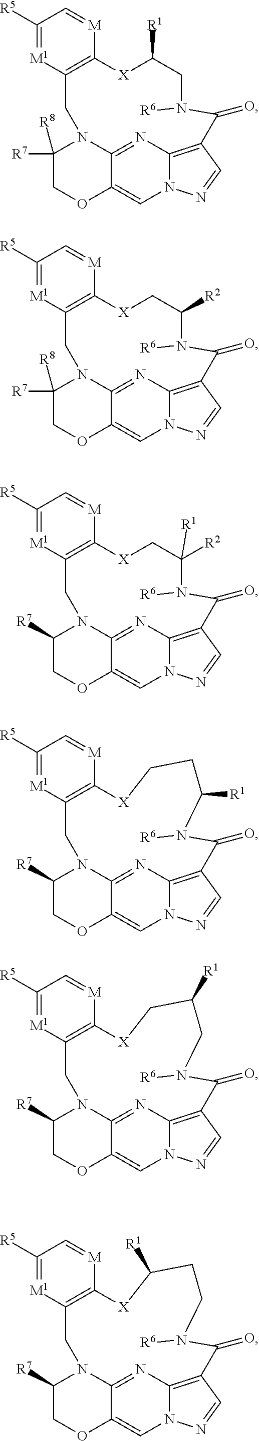

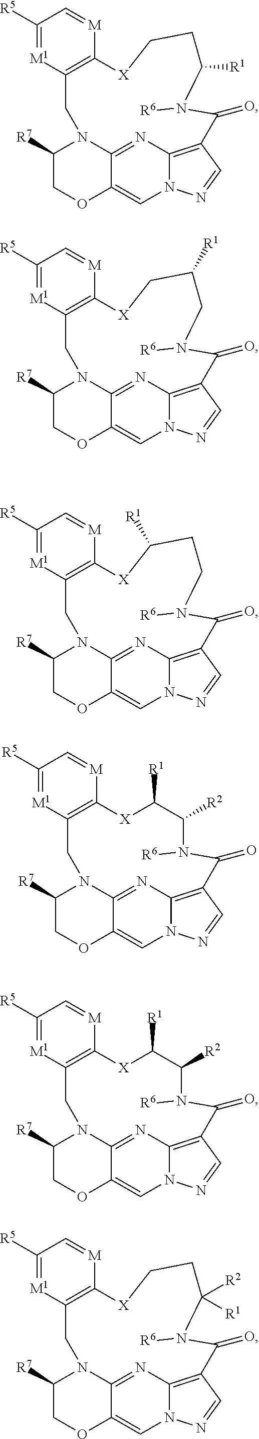

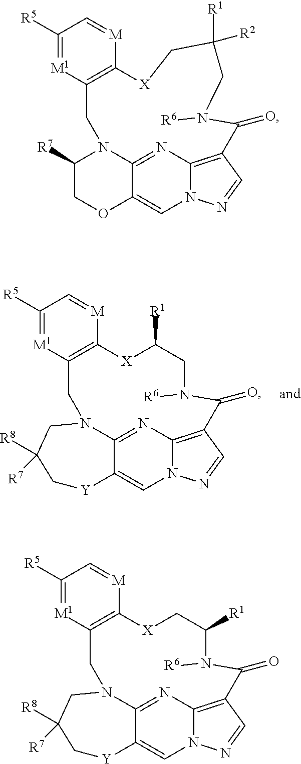

wherein

M is CR³ or N;

M¹ is CR⁴;

X is O, S, S(O), or S(O)₂;

R¹ and R² are each independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;

R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;

R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;

Y is O, S, NR⁸, or CR⁷R⁸; and

each R⁷ and R⁸ is independently H, deuterium, halogen, —CN, —OR^{c}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group, or mono- or bicyclic heteroaryl wherein each hydrogen atom in C₁-C₆ alkyl is optionally substituted by a halogen, N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁- C₆ alkyl.

26. The compound of clause 25, or a pharmaceutically acceptable salt thereof, wherein M is CR³.

27. The compound of clause 25 or 26, or a pharmaceutically acceptable salt thereof, wherein R³ is H, deuterium, C₁-C₆ alkyl or halogen.

28. The compound of any one of clauses 25 to 27, or a pharmaceutically acceptable salt thereof, wherein R³ is H or F.

29. The compound of clause 25, or a pharmaceutically acceptable salt thereof, wherein M is N.

30. The compound of any one of clauses 25 to 29, or a pharmaceutically acceptable salt thereof, wherein M¹ is CR⁴.

31. The compound of any one of clauses 25 to 30, or a pharmaceutically acceptable salt thereof, wherein R⁴ is H, deuterium, C₁-C₆ alkyl or halogen.

32. The compound of any one of clauses 25 to 31, or a pharmaceutically acceptable salt thereof, wherein R⁴ is H or Cl.

33. The compound of any one of clauses 25 to 32, or a pharmaceutically acceptable salt thereof, wherein R⁵ is F.

34. The compound of any one of clauses 25 to 33, or a pharmaceutically acceptable salt thereof, wherein R² is H.

34a. The compound of any one of clauses 25 to 33, or a pharmaceutically acceptable salt thereof, wherein each R¹ and R² is independently H, or R¹ and R² taken together with the carbon to which they are attached form a C₃-C₆ cycloalkyl.

35. The compound of any one of clauses 25 to 34, or a pharmaceutically acceptable salt thereof, wherein R¹ is C₁-C₆ alkyl.

35a. The compound of any one of clauses 25 to 34, or a pharmaceutically acceptable salt thereof, wherein, on one carbon atom, R¹ and R² taken together with the carbon to which they are attached form a C₃-C₆ cycloalkyl, and any other R¹ and R² when present is H.

36. The compound of any one of clauses 25 to 33 or 35, or a pharmaceutically acceptable salt thereof, wherein R² is C₁-C₆ alkyl.

36a. The compound of any one of clauses 25 to 33, or a pharmaceutically acceptable salt thereof, wherein at least one of R¹ is H and at least one of R² is C₁-C₆ alkyl.

37. The compound of any one of clauses 25 to 36, or a pharmaceutically acceptable salt thereof, wherein R⁷ is H or C₁-C₆ alkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl.

38. The compound of any one of clauses 25 to 37, wherein R⁷ is H or C₁-C₆ alkyl, wherein each hydrogen atom in C₁-C₆ alkyl is independently optionally substituted by deuterium, —OH, or —OC₁-C₆ alkyl.

39. The compound of clause 1 or 1a, selected from the group consisting of

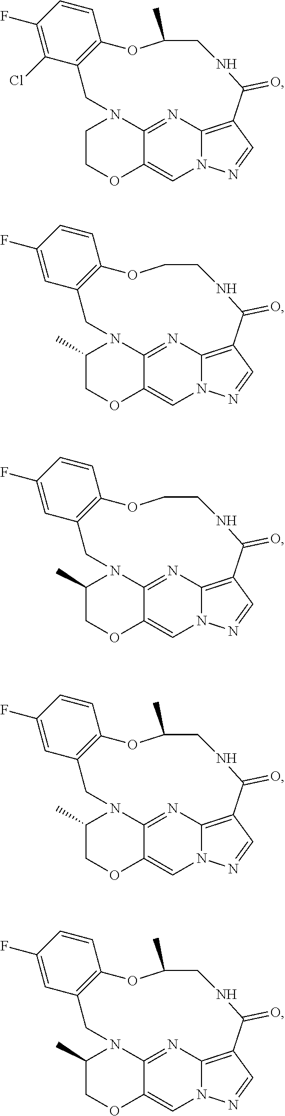 [SMILES: C[C@H]1CNC(=O)c2cnn3cc4c(nc23)N(CCO4)Cc2c(ccc(F)c2Cl)O1 ; C[C@H]1COc2cn3ncc4c3nc2N1Cc1cc(F)ccc1OCCNC4=O ; C[C@@H]1COc2cn3ncc4c3nc2N1Cc1cc(F)ccc1OCCNC4=O ; C[C@H]1CNC(=O)c2cnn3cc4c(nc23)N(Cc2cc(F)ccc2O1)[C@@H](C)CO4 ; C[C@@H]1COc2cn3ncc4c3nc2N1Cc1cc(F)ccc1O[C@@H](C)CNC4=O]

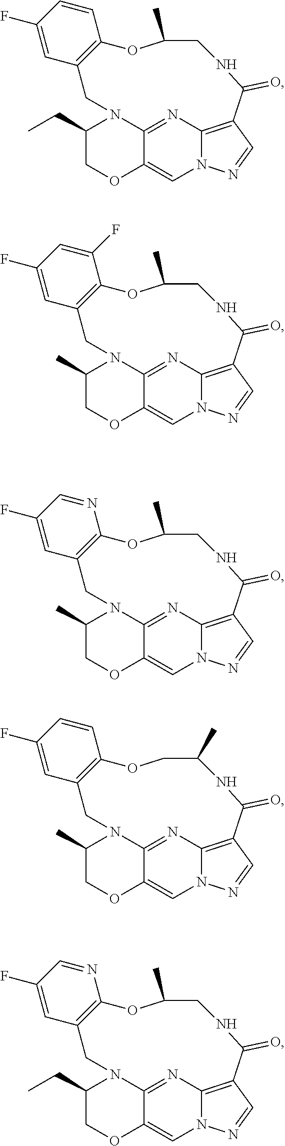 [SMILES: CC[C@@H]1COc2cn3ncc4c3nc2N1Cc1cc(F)ccc1O[C@@H](C)CNC4=O ; C[C@@H]1COc2cn3ncc4c3nc2N1Cc1cc(F)cc(F)c1O[C@@H](C)CNC4=O ; C[C@@H]1COc2cn3ncc4c3nc2N1Cc1cc(F)cnc1O[C@@H](C)CNC4=O ; C[C@@H]1COc2ccc(F)cc2CN2c3nc4c(cnn4cc3OC[C@H]2C)C(=O)N1 ; CC[C@@H]1COc2cn3ncc4c3nc2N1Cc1cc(F)cnc1O[C@@H](C)CNC4=O]

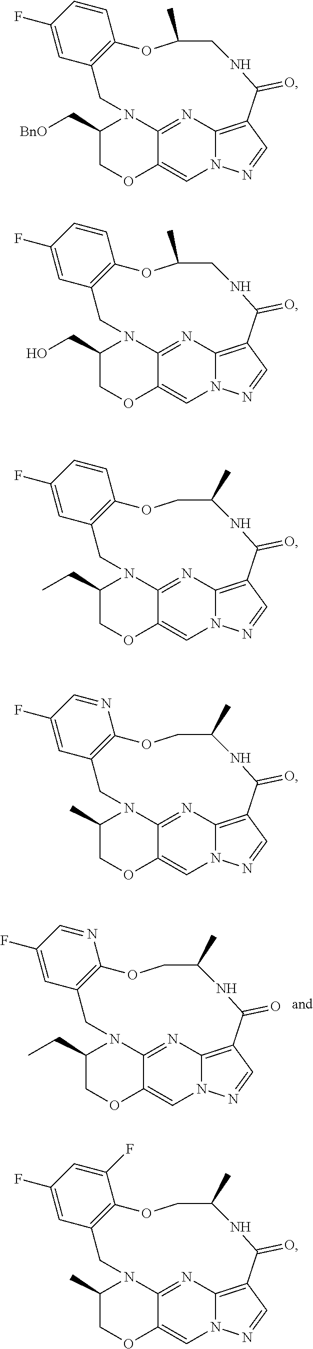

or a pharmaceutically acceptable salt thereof.

40. The compound of clause 1 or 1a, selected from the group consisting of

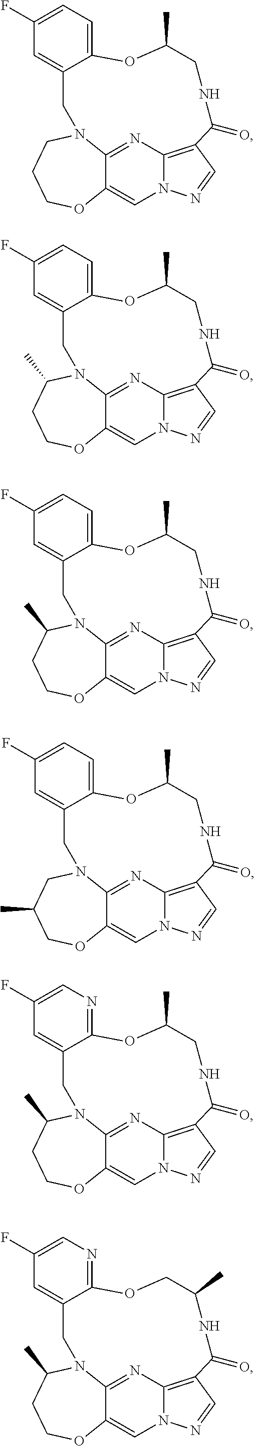

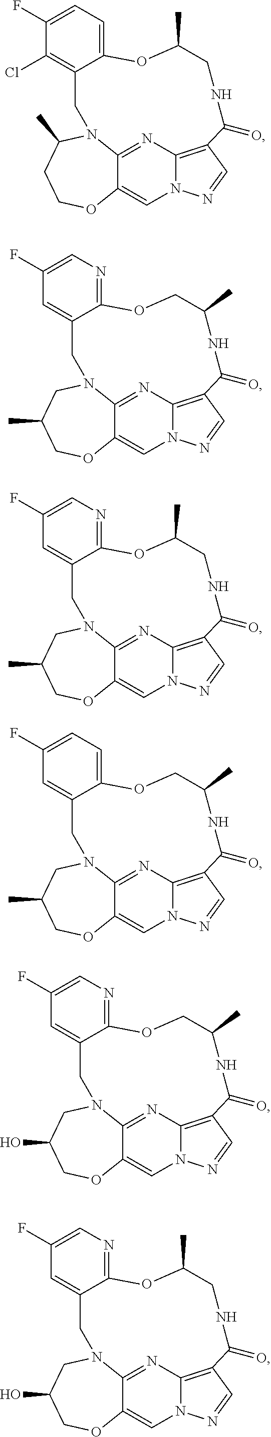

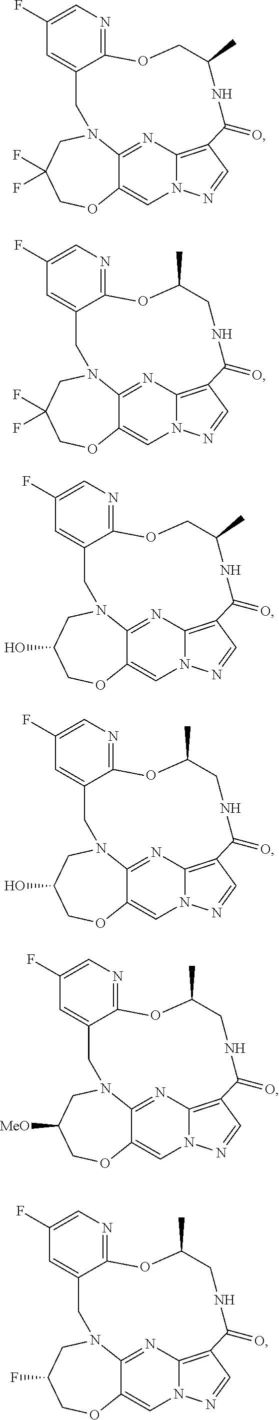

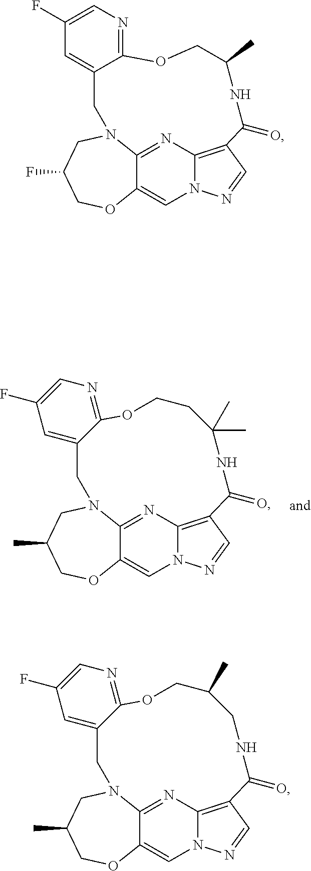 [SMILES: C[C@@H]1COc2ncc(F)cc2CN2C[C@@H](F)COc3cn4ncc(c4nc32)C(=O)N1 ; C[C@@H]1COc2cn3ncc4c3nc2N(Cc2cc(F)cnc2OCCC(C)(C)NC4=O)C1 ; C[C@@H]1COc2cn3ncc4c3nc2N(Cc2cc(F)cnc2OC[C@@H](C)CNC4=O)C1]

or a pharmaceutically acceptable salt thereof.

41. The compound of clause 1 or 1a, selected from the group consisting of

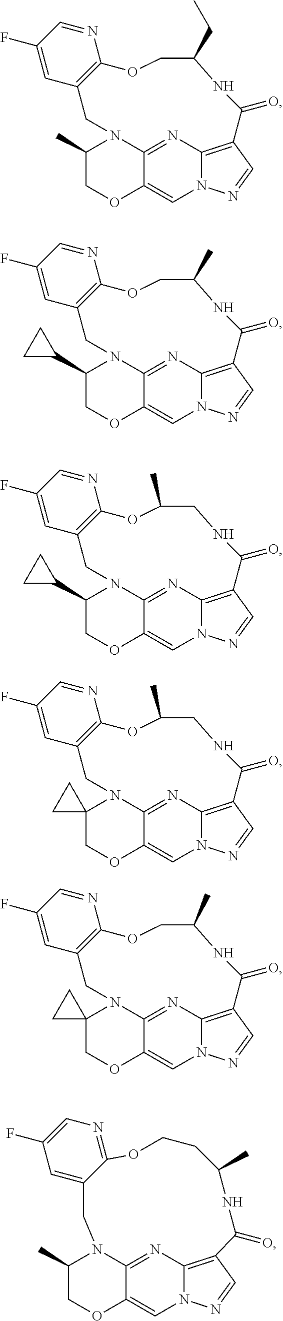

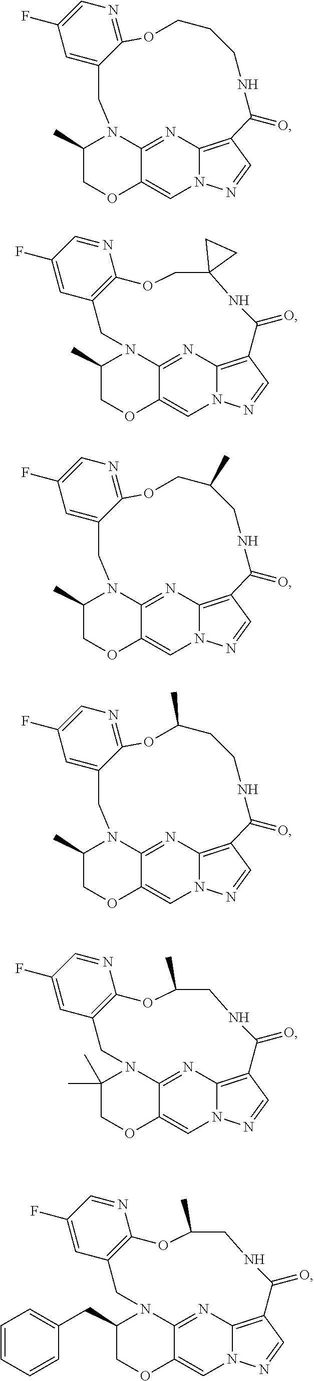

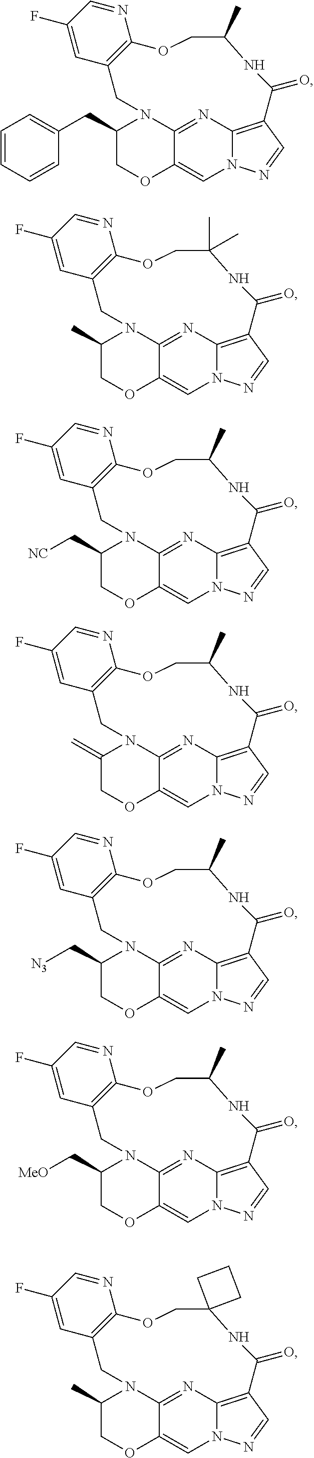

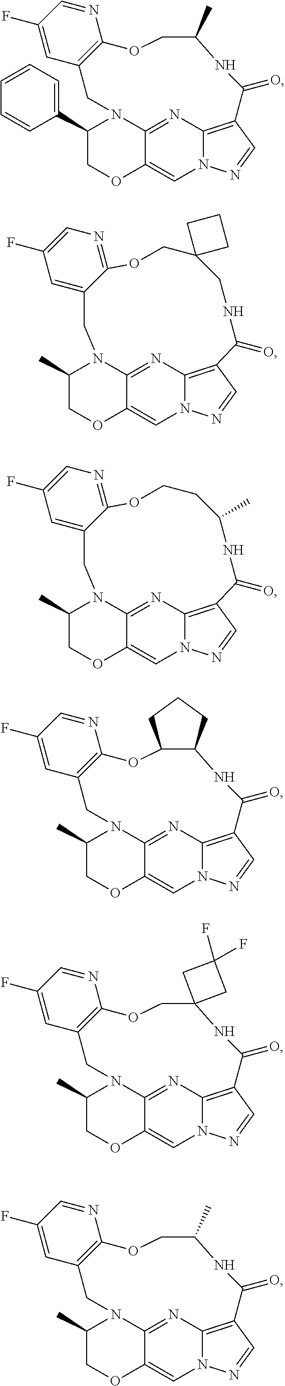

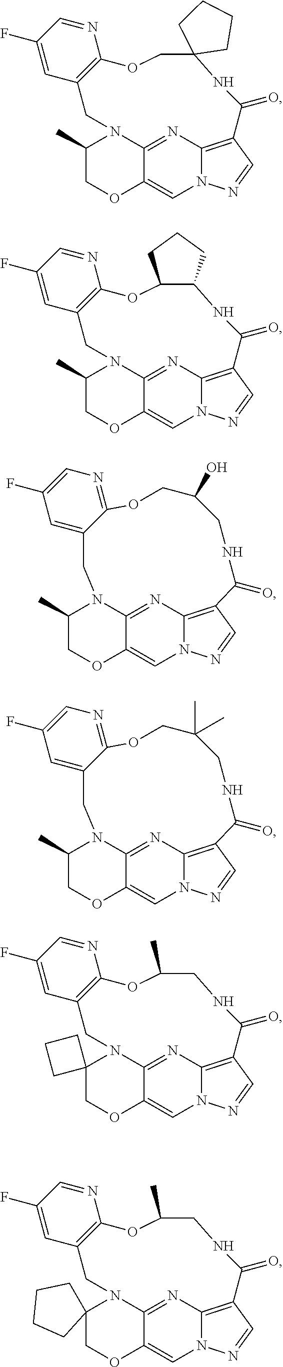

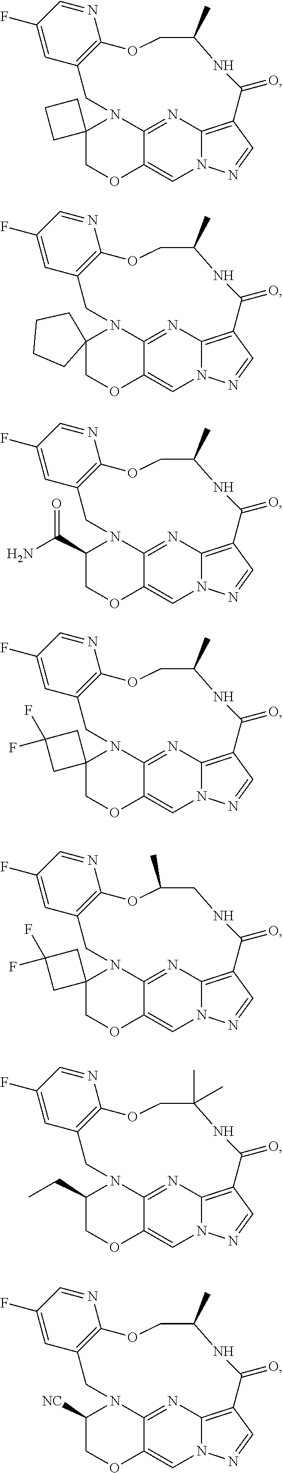

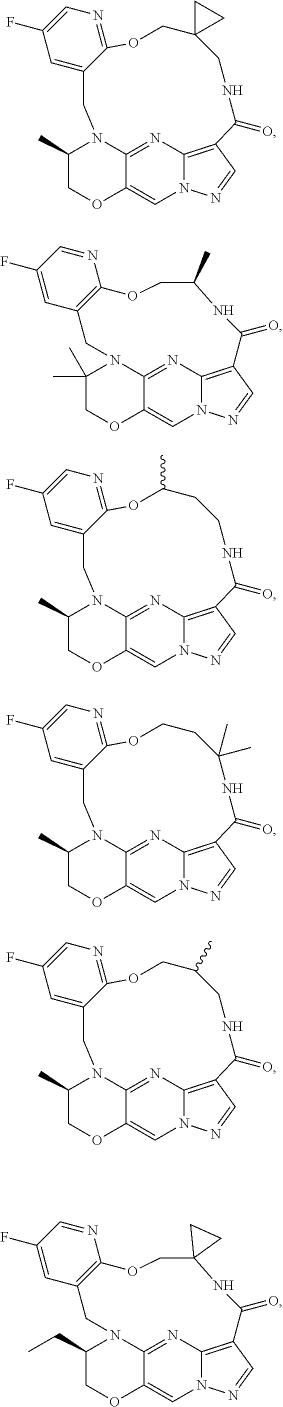

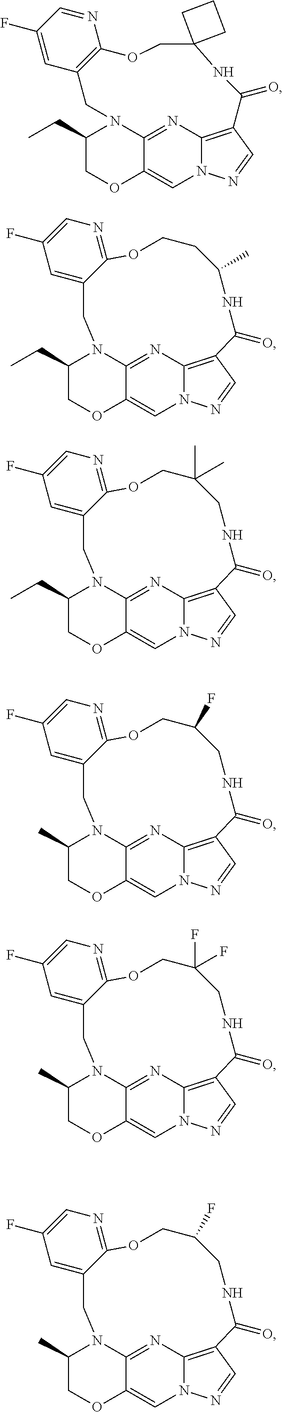

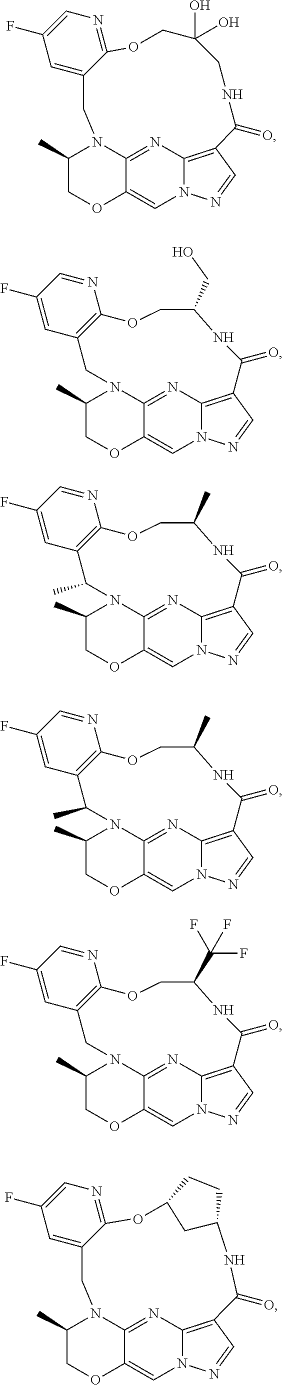

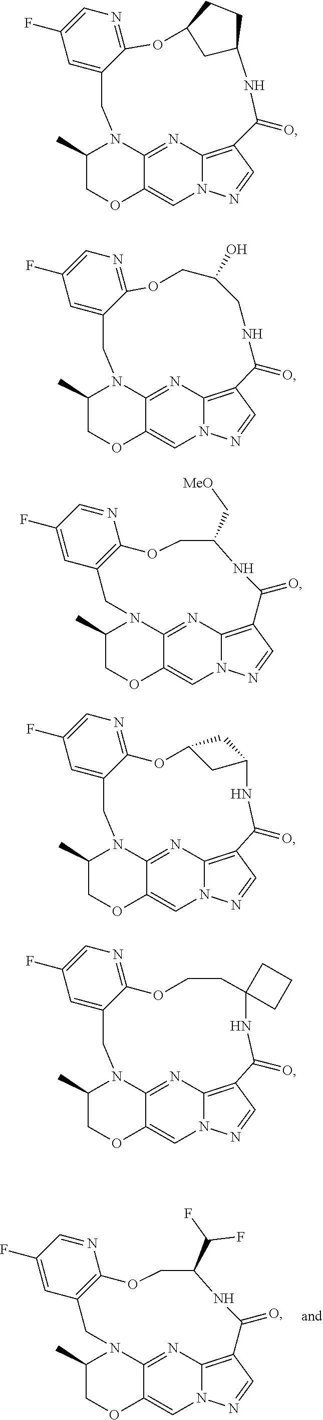

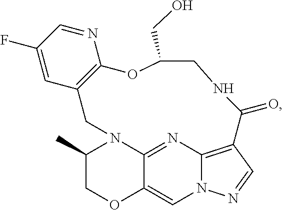 [SMILES: C[C@@H]1COc2cn3ncc4c3nc2N1Cc1cc(F)cnc1O[C@H](CO)CNC4=O]

or a pharmaceutically acceptable salt thereof.

42. A pharmaceutical composition comprising a compound of any one of the preceding clauses, or a pharmaceutically acceptable salt thereof, and optionally at least one diluent, carrier or excipient.

43. A method of treating cancer, pain, neurological diseases, autoimmune diseases, or inflammation comprising administering to a subject in need of such treatment an effective amount of at least one compound of any one of clauses 1 to 41, or a pharmaceutically acceptable salt thereof.

44. Use of a compound of any one of clauses 1 to 41, or a pharmaceutically acceptable salt thereof, in the preparation of a medicament for the treatment of cancer.

45. Use of a compound of any one of clauses 1 to 41, or a pharmaceutically acceptable salt thereof, for treating cancer.

46. A method of inhibiting protein or tyrosine kinases selected from one or more of ALK, ROS1, TRK, JAK, and FGFRs, comprising contacting a cell comprising one or more of such kinases with an effective amount of at least one compound of any one of clauses 1 to 41, or a pharmaceutically acceptable salt thereof, and/or with at least one pharmaceutical composition of the disclosure, wherein the contacting is in vitro, ex vivo, or in vivo.

47. A compound of any one of clauses 1 to 41, for use in treating cancer in a patient.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments, alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthylenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, including an all-carbon 5-member/6-member or 6-member/6-member fused bicyclic ring, or a multicyclic fused ring (a “fused” ring system means that each ring in the system shares an adjacent pair of carbon atoms with each other ring in the system) group, or a carbocyclic ring that is fused to another group such as a heterocyclic, such as ring 5- or 6-membered cycloalkyl fused to a 5- to 7-membered heterocyclic ring, where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₉, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like. Illustrative examples of cycloalkyl groups shown in graphical representations include the following entities, in the form of properly bonded moieties:

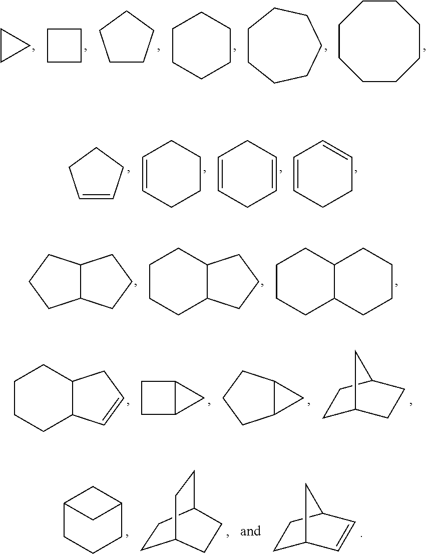

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. A heterocycloalkyl group may be fused to another group such as another heterocycloalkyl, or a heteroaryl group. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, 3-, 4-, 5- or 6-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like. Illustrative examples of heterocycloalkyl groups shown in graphical representations include the following entities, in the form of properly bonded moieties:

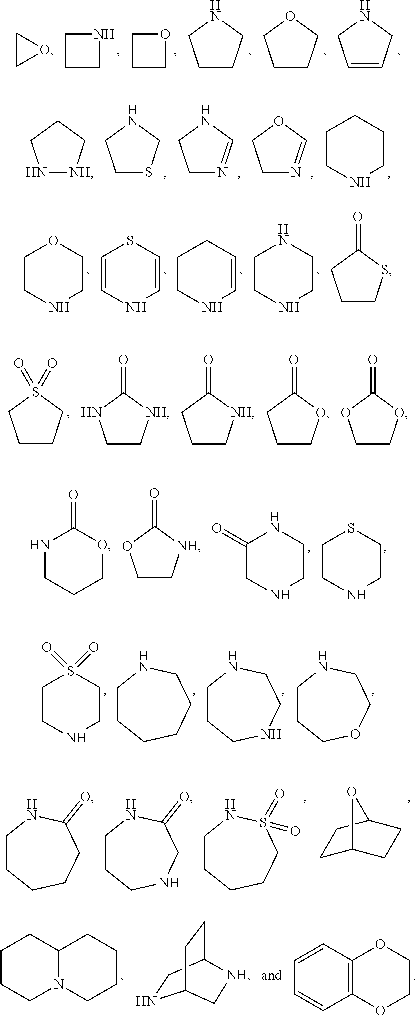

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like. Illustrative examples of heteroaryl groups shown in graphical representations, include the following entities, in the form of properly bonded moieties:

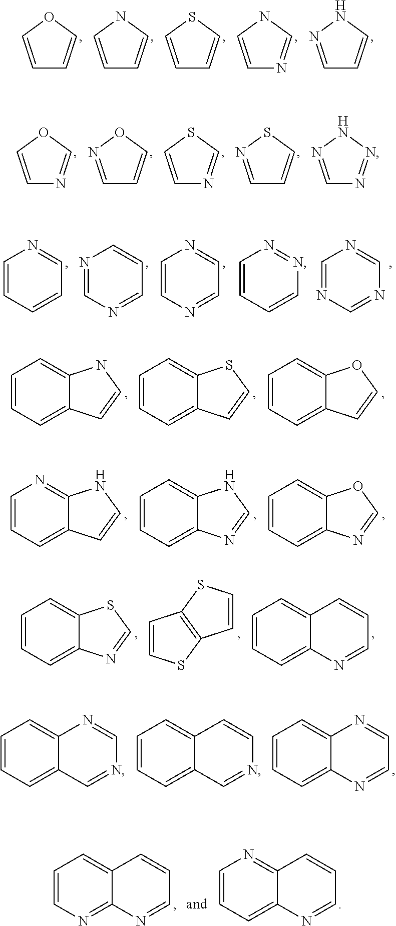

As used herein, “hydroxy” or ““hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “cyano” refers to a —CN group.

The term “oxo” represents a carbonyl oxygen. For example, a cyclopentyl substituted with oxo is cyclopentanone.

As used herein, “bond” refers to a covalent bond.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by C₁-C₆ alkyl” means that an alkyl may be but need not be present on any of the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl by replacement of a hydrogen atom for each alkyl group, and the description includes situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is substituted with an alkyl group and situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the phrase “taken together with the carbon to which they are attached” or “taken together with the carbon atom to which they are attached” means that two substituents (e.g. R⁷ and R⁸) attached to the same carbon atom form the groups that are defined by the claim, such as C₃-C₆ cycloalkyl or a 4- to 6-membered heterocycloalkyl. In particular, the phrase “taken together with the carbon to which they are attached” means that when, for example, R⁷ and R⁸, and the carbon atom to which they are attached form a C₃-C₆ cycloalkyl, then the formed ring will be attached at the same carbon atom. For example, the phrase “R⁷ and R⁸ taken together with the carbon to which they are attached form a C₃-C₆ cycloalkyl” used in connection with the embodiments described herein includes the compounds represented as follows:

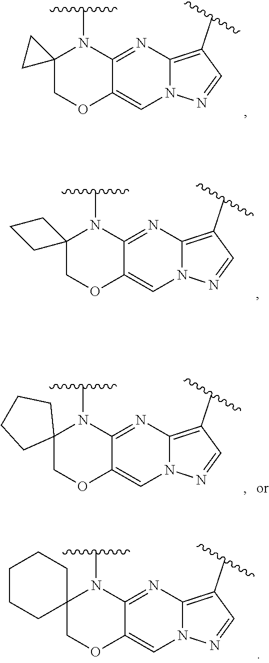 [SMILES: *c1cnn2cc3c(nc12)N(*)C1(CC1)CO3 ; *c1cnn2cc3c(nc12)N(*)C1(CCC1)CO3 ; *c1cnn2cc3c(nc12)N(*)C1(CCCC1)CO3 ; *c1cnn2cc3c(nc12)N(*)C1(CCCCC1)CO3]

where the above spirocyclic rings can be optionally substituted as defined in the given embodiment.

As used herein, the phrase “taken together with the carbons to which they are attached” or “taken together with the carbon atoms to which they are attached” means that two substituents (e.g. R¹ and R²) attached to different carbon atoms form the groups that are defined by the claim, such as C₃-C₆ cycloalkyl or a 4- to 6-membered heterocycloalkyl. In particular, the phrase “taken together with the carbons to which they are attached form a” means that when, for example, R¹ and R², and the carbon atoms, which are not the same carbon atom, to which they are attached form a C₃-C₆ cycloalkyl, then the formed ring will be attached at different carbon atoms. For example, the phrase “R¹ and R² taken together with the carbons to which they are attached form a C₃-C₆ cycloalkyl” used in connection with the embodiments described herein includes the compounds represented as follows:

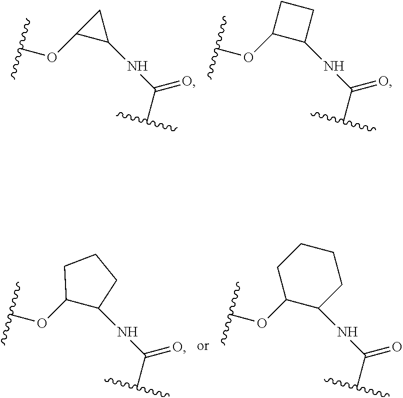

where the above fused rings can be optionally substituted as defined in the given embodiment.

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula I-XI that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula I-XI, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I-XI). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula I-XI, and uses of such metabolites in the methods of the disclosure. A “pharmaceutically active metabolite” means a pharmacologically active product of metabolism in the body of a compound of Formula I-XI, or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”. For example, in some exemplary embodiments, certain compounds provided herein can be described by the formula

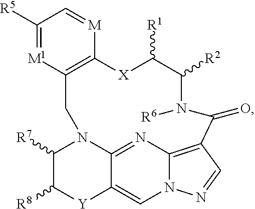

which formula will be understood to encompass compounds having both stereochemical configurations at the relevant carbon atom, specifically in this example

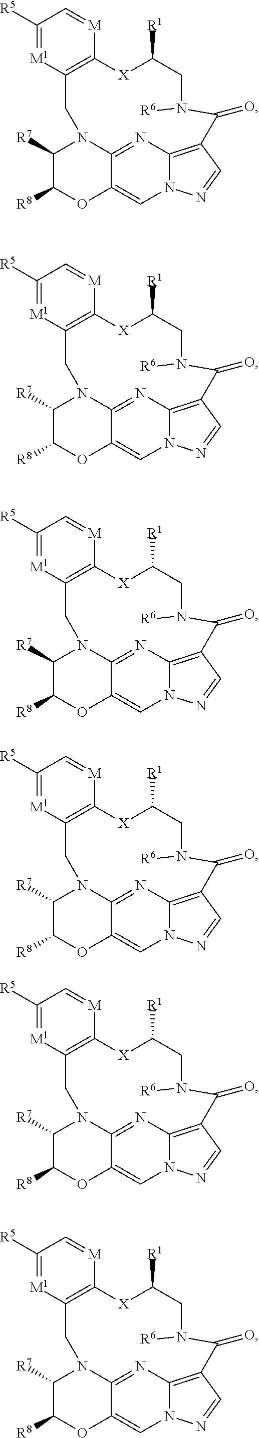

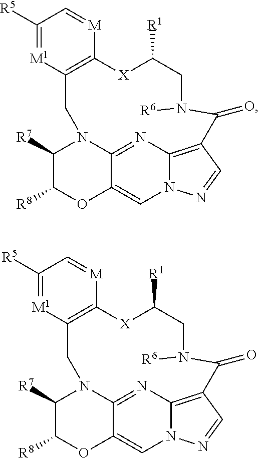

and other stereochemical combinations.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

### Representative Embodiments

In some embodiments, compounds described herein comprise a moiety of the formula

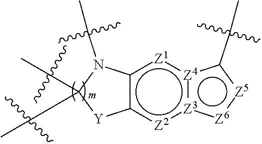

wherein Z¹-Z⁶, Y and m are defined as described herein, and the substituents on the non-aromatic ring marked by a bond and ˜ correspond to R⁷ and R⁸ as described herein. In other embodiments, compounds described herein comprise a moiety of the formula

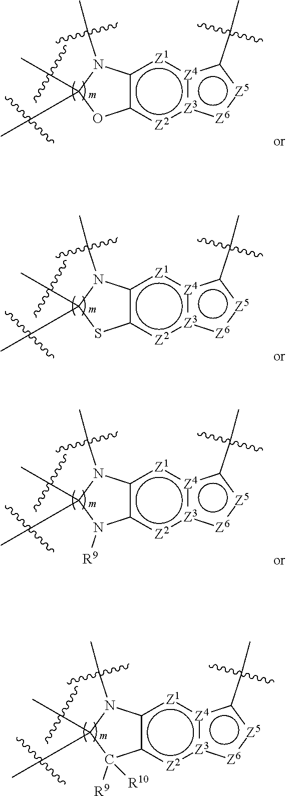

wherein Z¹-Z⁶, m, and R⁹ are otherwise defined as described herein, and the substituents on the non-aromatic ring marked by a bond and ˜ correspond to R⁷ and R⁸ as described herein. In still other embodiments, compounds described herein comprise a moiety of the formula

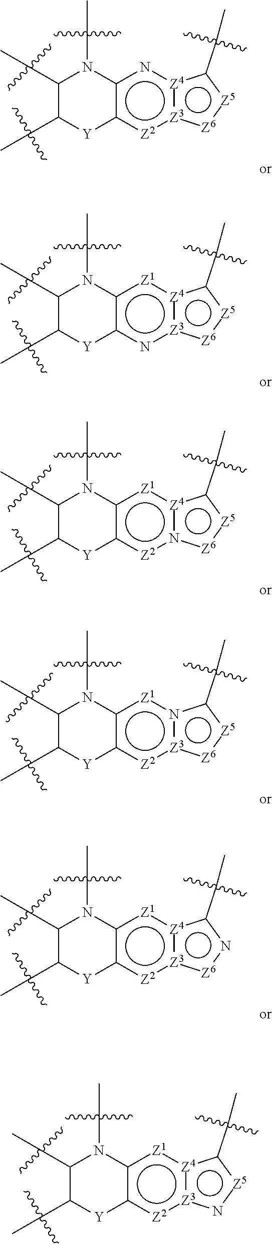

wherein Z¹-Z⁶ and Y are otherwise defined as described herein, and the substituents on the non-aromatic ring marked by a bond and ˜ correspond to R⁷ and/or R⁸ as described herein. In still other embodiments, compounds described herein comprise a moiety of the formula

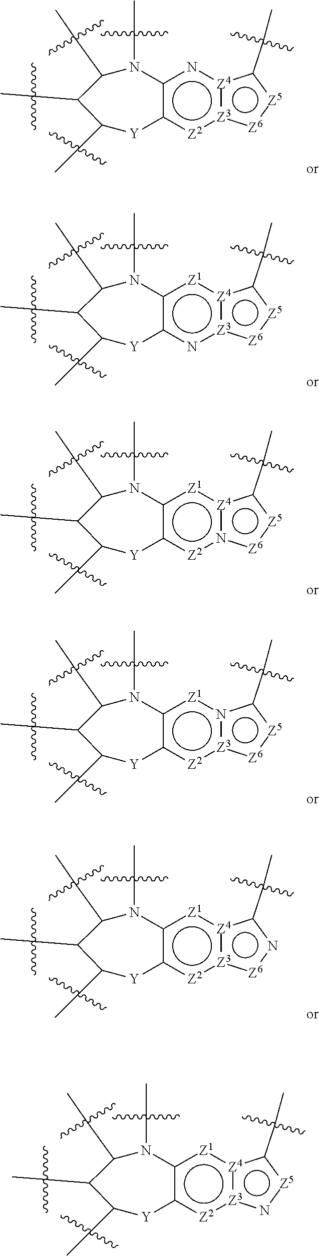

wherein Z¹-Z⁶ and Y are otherwise defined as described herein, and the substituents on the non-aromatic ring marked by a bond and ˜ correspond to R⁷ and/or R⁸ as described herein. In still other embodiments, compounds described herein comprise a moiety of the formula

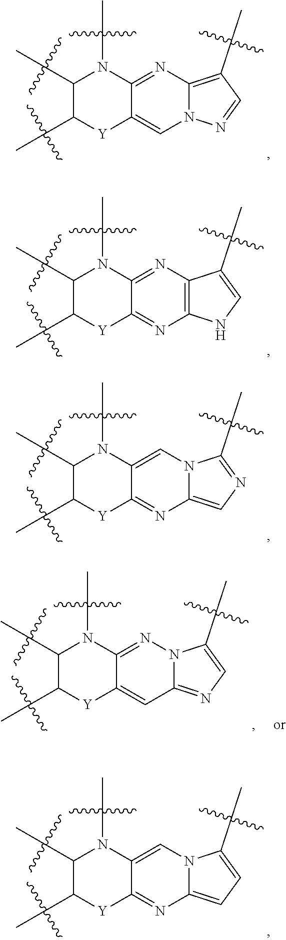

wherein Y is otherwise defined as described herein, and the substituents on the non-aromatic ring marked by a bond and ˜ correspond to R⁷ and/or R⁸ as described herein In still other embodiments, compounds described herein comprise a moiety of the formula

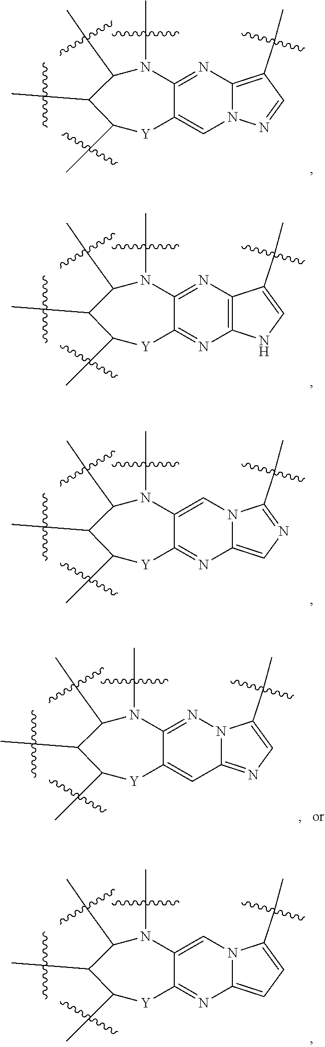

wherein Y is otherwise defined as described herein, and the substituents on the non-aromatic ring marked by a bond and ˜ correspond to R⁷ and/or R⁸ as described herein. In still other embodiments, compounds described herein comprise a moiety of the formula

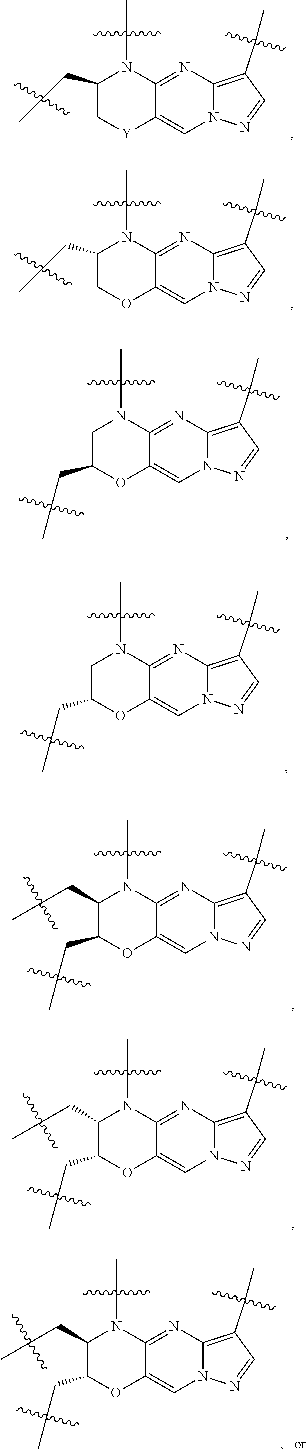

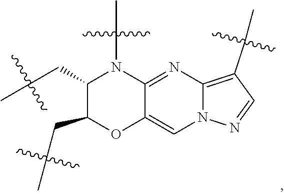 [SMILES: *C[C@@H]1Oc2cn3ncc(*)c3nc2N(*)[C@H]1C*]

wherein the substituents on the non-aromatic ring marked by a bond and ˜ correspond to R⁷ and/or R⁸ as described herein.

In some embodiments, each of Z¹, Z², Z³, Z⁴, Z⁵, and Z⁶ is independently N, NH, C or CH. In some embodiments, Z¹, Z³ and Z⁶ are N, Z² and Z⁵ are CH, and Z⁴ is C. In some embodiments, Z¹, Z³ and Z⁶ are N, Z² and Z⁵ are CH, Z⁴ is C, and Y is O. In some embodiments, Z¹, Z² and Z⁶ are N, Z⁵ is CH, and Z³ and Z⁴ are C. In some embodiments, Z¹, Z² and Z⁶ are N, Z⁵ is CH, Z³ and Z⁴ are C, and Y is O. In some embodiments, Z², Z⁴ and Z⁵ are N, Z¹ and Z⁶ are CH, and Z³ is C. In some embodiments, Z², Z⁴ and Z⁵ are N, Z¹ and Z⁶ are CH, Z³ is C and Y is O. In some embodiments, Z¹, Z⁴ and Z⁶ are N, Z² and Z⁵ are CH, and Z³ is C. In some embodiments, Z¹, Z⁴ and Z⁶ are N, Z² and Z⁵ are CH, Z³ is C, and Y is O. In some embodiments, Z² and Z⁴ are N, Z¹, Z⁵ and Z⁶ are CH, and Z³ is C. In some embodiments, Z² and Z⁴ are N, Z¹, Z⁵ and Z⁶ are CH, Z³ is C, and Y is O.

In some embodiments, L is —C(R¹)(R²)—. In some embodiments, L is X. In some embodiments, when t is 1, L is —C(R¹)(R²)—.

In some embodiments, X is —O—. In some embodiments, X is —S—. In some embodiments, X is —S(O)—. In some embodiments, X is —S(O)₂—. In some embodiments, when t is 1, L is not X. In some embodiments, when t is 2, 2, or 4, the L attached directly to the amide nitrogen in the macrocycle is not X.

In some embodiments, each R¹ and R² is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂R^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}; wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl.

In some embodiments, each R¹ and R² is independently H, or R¹ and R² taken together with the carbon to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each R¹ and R² is independently H, or R¹ and R² taken together with the carbon to which they are attached form a C₃-C₆ cycloalkyl. In some embodiments, R¹ and R² taken together with the carbon to which they are attached on one carbon atom of L form a C₃-C₆ cycloalkyl, and any other R¹ and R² on L is H. In some embodiments, R¹ is H. In some embodiments, R¹ is C₁-C₆ alkyl. In some embodiments, R² is H. In some embodiments, R¹ is H and R² is C₁-C₆ alkyl.

In some embodiments, M is CR³. In some embodiments, M is N. In some embodiments, M¹ is CR⁴.

In some embodiments, each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅-C₈ cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃. In some embodiments, R³ is H, deuterium, C₁-C₆ alkyl or halogen. In some embodiments, R³ is H or F. In some embodiments, R⁴ is H, deuterium, —CN, C₁-C₆ alkyl or halogen. In some embodiments, R⁴ is H or Cl. In some embodiments, R⁵ is F.

In some embodiments, R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl.

In some embodiments, Y is O, S, NR^{B}, or CR⁷R⁸. In some embodiments, Y is O. In some embodiments, Y is S. In some embodiments, Y is NR⁸. In some embodiments, Y is CR⁷R⁸.

In some embodiments, each R⁷ and R⁸ is independently H, deuterium, halogen, —CN, —OR^{e}, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₂-C₆ alkenyl, C₂-C₆ alkynyl, C₃-C₆ cycloalkyl, 4- to 6-membered heterocycloalkyl, 3- to 7-membered heterocycloalkyl, C₆-C₁₀ aryl, exocyclic ethylene group, or mono- or bicyclic heteroaryl is optionally substituted by a halogen, —N₃, —CN, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e}

In some embodiments, each R⁷ and R⁸ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is optionally substituted by a halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e}.

In some embodiments, each R⁷ and R⁸ is independently H, deuterium, halogen, or C₁-C₆ alkyl, wherein each hydrogen atom in C₁-C₆ alkyl is optionally substituted by a halogen, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl. In some embodiments, R⁸ is H.

In some embodiments, R⁷ is H or C₁-C₆ alkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl. In some embodiments, R⁷ is H or C₁-C₆ alkyl, wherein each hydrogen atom in C₁-C₆ alkyl is independently optionally substituted by deuterium, —OH, or —OC₁-C₆ alkyl.

In some embodiments, each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl.

In some embodiments, m is 0, 1, 2, or 3. In some embodiments, m is 2. In some embodiments, m is 3. In some embodiments, m is 2 or 3.

In some embodiments, p is 1, 2, 3, or 4. In some embodiments, p is 1.

In some embodiments, t is 1, 2, 3, 4, or 5. In some embodiments, t is 3. In some embodiments, t is 4. In some embodiments, t is 3 or 4.

In some embodiments, n, if present, is 2 or 3. In some embodiments, n is 2. In some embodiments, n is 3. In some embodiments, n is 2 or 3.

The following represent illustrative embodiments of compounds of the Formula I-XI:

| Cpd | Structure | Name |
|---|---|---|
| 1 | 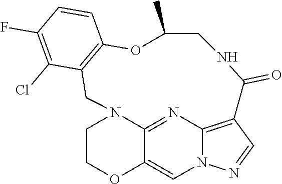 [SMILES: C[C@H]1CNC(=O)c2cnn3cc4c(nc23)N(CCO4)Cc2c(ccc(F)c2Cl)O1] | (12S)-7-chloro-8-fluoro-12- methyl-3,4,13,14-tetrahydro-6H- 18,1-(metheno)[1,4]oxazino[3,4- i]pyrazolo[4,3-f][1,4,8,10]benzo- xatriazacyclotridecin-15(12H)-one |
| 2 | 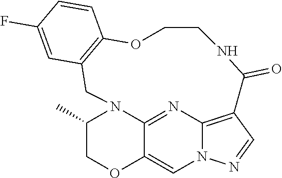 [SMILES: C[C@H]1COc2cn3ncc4c3nc2N1Cc1cc(F)ccc1OCCNC4=O] | (4S)-8-fluoro-4-methyl-3,4,13,14- tetrahydro-6H-18,1-(metheno)- [1,4]oxazino[3,4-i]pyrazolo[4,3-f] [1,4,8,10]benzoxatriazacyclo- tridecin-15(12H)-one |
| 3 | 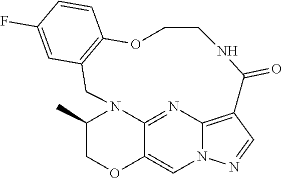 [SMILES: C[C@@H]1COc2cn3ncc4c3nc2N1Cc1cc(F)ccc1OCCNC4=O] | (4R,12S)-8-fluoro-4,12-dimethyl- 3,4,13,14-tetrahydro-6H-18,1- (metheno)[1,4]oxazino[3,4-i]- pyrazolo[4,3-f][1,4,8,10]benzo- xatriazacyclotridecin-15(12H)-one |
| 4 | 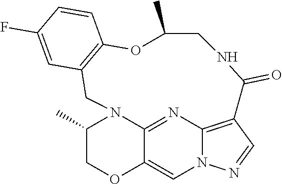 [SMILES: C[C@H]1CNC(=O)c2cnn3cc4c(nc23)N(Cc2cc(F)ccc2O1)[C@@H](C)CO4] | (4S,12S)-8-fluoro-4,12-dimethyl- 3,4,13,14-tetrahydro-6H-18,1- (metheno)[1,4]oxazino[3,4-i]- pyrazolo[4,3-f][1,4,8,10]benzo- xatriazacyclotridecin-15(12H)-one |
| 5 | 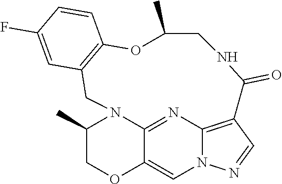 [SMILES: C[C@@H]1COc2cn3ncc4c3nc2N1Cc1cc(F)ccc1O[C@@H](C)CNC4=O] | (4R,12S)-8-fluoro-4,12-dimethyl- 3,4,13,14-tetrahydro-6H-18,1- (metheno)[1,4]oxazino[3,4-i]- pyrazolo[4,3-f][1,4,8,10]benzo- xatriazacyclotridecin-15(12H)-one |
| 6 | 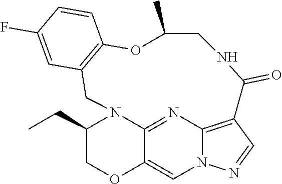 [SMILES: CC[C@@H]1COc2cn3ncc4c3nc2N1Cc1cc(F)ccc1O[C@@H](C)CNC4=O] | (4R,12S)-4-ethyl-8-fluoro-12- methyl-3,4,13,14-tetrahydro-6H- 18,1-(metheno)[1,4]oxazino[3,4- i]pyrazolo[4,3-f][1,4,8,10]benzo- xatriazacyclotridecin-15(12H)-one |
| 7 | 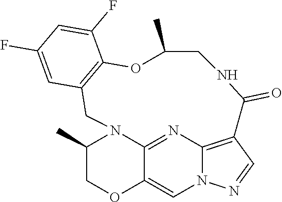 [SMILES: C[C@@H]1COc2cn3ncc4c3nc2N1Cc1cc(F)cc(F)c1O[C@@H](C)CNC4=O] | (4R,12S)-8,10-difluoro-4,12- dimethyl-3,4,13,14-tetrahydro-6H- 18,1-(metheno)[1,4]oxazino[3,4- i]pyrazolo[4,3-f][1,4,8,10]benzo- xatriazacyclotridecin-15(12H)-one |
| 8 | 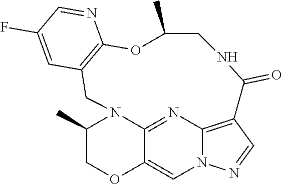 [SMILES: C[C@@H]1COc2cn3ncc4c3nc2N1Cc1cc(F)cnc1O[C@@H](C)CNC4=O] | (4R,12S)-8-fluoro-4,12-dimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 9 | 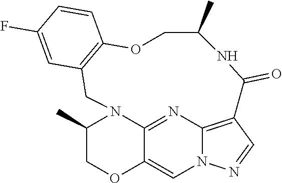 [SMILES: C[C@@H]1COc2ccc(F)cc2CN2c3nc4c(cnn4cc3OC[C@H]2C)C(=O)N1] | (4R,13R)-8-fluoro-4,13-dimethyl- 3,4,13,14-tetrahydro-6H-18,1- (metheno)[1,4]oxazino[3,4-i]- pyrazolo[4,3-f][1,4,8,10]benzo- xatriazacyclotridecin-15(12H)-one |
| 10 | 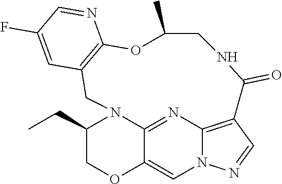 [SMILES: CC[C@@H]1COc2cn3ncc4c3nc2N1Cc1cc(F)cnc1O[C@@H](C)CNC4=O] | (4R,12S)-4-ethyl-8-fluoro-12- methyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 11 | 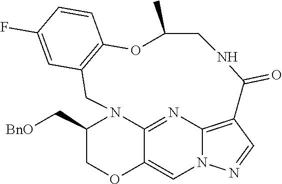 [SMILES: C[C@H]1CNC(=O)c2cnn3cc4c(nc23)N(Cc2cc(F)ccc2O1)[C@H](COCc1ccccc1)CO4] | (4R,12S)-4-[(benzyloxy)methyl)- 8-fluoro-12-methyl-3,4,13,14- tetrahydro-6H-18,1-(metheno)- [1,4]oxazino[3,4-i]pyrazolo[4,3- f][1,4,8,10]benzoxatriazacyclo- tridecin-15(12H)-one |
| 12 | 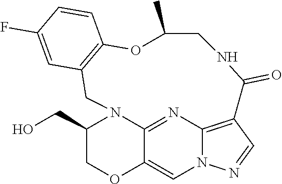 [SMILES: C[C@H]1CNC(=O)c2cnn3cc4c(nc23)N(Cc2cc(F)ccc2O1)[C@H](CO)CO4] | (4R,12S)-8-fluoro-4-(hydroxy- methyl)-12-methyl-3,4,13,14- tetrahydro-6H-18,1-(metheno)- [1,4]oxazino[3,4-i]pyrazolo[4,3- f][1,4,8,10]benzoxatriazacyclo- tridecin-15(12H)-one |
| 13 | 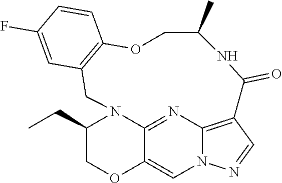 [SMILES: CC[C@@H]1COc2cn3ncc4c3nc2N1Cc1cc(F)ccc1OC[C@@H](C)NC4=O] | (4R,13R)-4-ethyl-8-fluoro-13- methyl-3,4,13,14-tetrahydro-6H- 18,1-(metheno)[1,4]oxazino[3,4- i]pyrazolo[4,3-f][1,4,8,10]benzo- xatriazacyclotridecin-15(12H)-one |
| 14 | 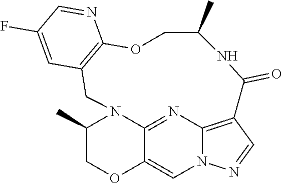 [SMILES: C[C@@H]1COc2ncc(F)cc2CN2c3nc4c(cnn4cc3OC[C@H]2C)C(=O)N1] | (4R,13R)-8-fluoro-4,13-dimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 15 | 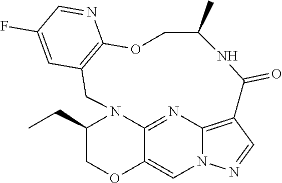 [SMILES: CC[C@@H]1COc2cn3ncc4c3nc2N1Cc1cc(F)cnc1OC[C@@H](C)NC4=O] | (4R,13R)-4-ethyl-8-fluoro-13- methyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 16 | 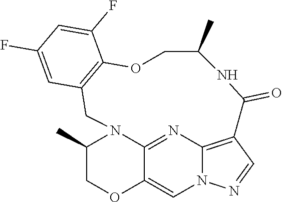 [SMILES: C[C@@H]1COc2c(F)cc(F)cc2CN2c3nc4c(cnn4cc3OC[C@H]2C)C(=O)N1] | (4R,13R)-8,10-difluoro-4,13- dimethyl-3,4,13,14-tetrahydro- 6H-18,1-(metheno)[1,4]oxazino- [3,4-i]pyrazolo[4,3-f][1,4,8,10]- benzoxatriazacyclotridecin- 15(12H)-one |
| 17 | 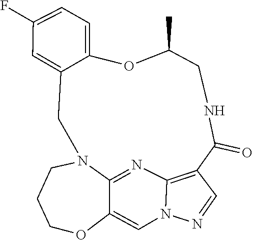 [SMILES: C[C@H]1CNC(=O)c2cnn3cc4c(nc23)N(CCCO4)Cc2cc(F)ccc2O1] | (13S)-9-fluoro-13-methyl- 4,5,14,15-tetrahydro-3H,7H- 19,1-(metheno)[1,4]oxazepino- [3,4-i]pyrazolo[4,3-f][1,4,8,10]- benzoxatriazacyclotridecin- 16(13H)-one |
| 18 | 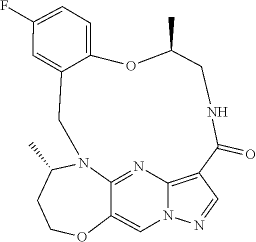 [SMILES: C[C@H]1CNC(=O)c2cnn3cc4c(nc23)N(Cc2cc(F)ccc2O1)[C@@H](C)CCO4] | (5S,13S)-9-fluoro-5,13-dimethyl- 4,5,14,15-tetrahydro-3H,7H-19,1- (metheno)[1,4]oxazepino[3,4-i]- pyrazolo[4,3-f][1,4,8,10]benzo- xatriaxacyclotridecin-16(13H)-one |
| 19 | 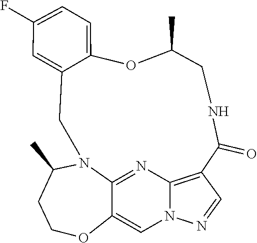 [SMILES: C[C@@H]1CCOc2cn3ncc4c3nc2N1Cc1cc(F)ccc1O[C@@H](C)CNC4=O] | (5R,13S)-9-fluoro-5,13-dimethyl- 4,5,14,15-tetrahydro-3H,7H-19,1- (metheno)[1,4]oxazepino[3,4-i]- pyrazolo[4,3-f][1,4,8,10]benzo- xatriazacyclotridecin-16(13H)-one |
| 20 | 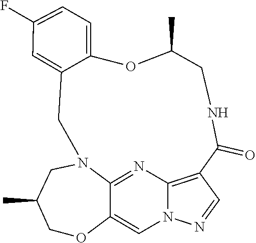 [SMILES: C[C@@H]1COc2cn3ncc4c3nc2N(Cc2cc(F)ccc2O[C@@H](C)CNC4=O)C1] | (4S,13S)-9-fluoro-4,13-dimethyl- 4,5,14,15-tetrahydro-3H,7H-19,1- (metheno)[1,4]oxazepino[3,4-i]- pyrazolo[4,3-f][1,4,8,10]benzo- xatriazacyclotridecin-16(13H)-one |
| 21 | 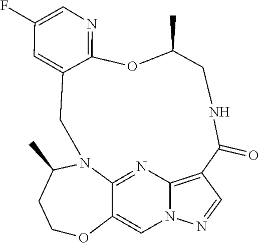 [SMILES: C[C@@H]1CCOc2cn3ncc4c3nc2N1Cc1cc(F)cnc1O[C@@H](C)CNC4=O] | (5R,13S)-9-fluoro-5,13-dimethyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 22 | 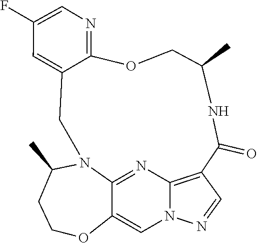 [SMILES: C[C@@H]1COc2ncc(F)cc2CN2c3nc4c(cnn4cc3OCC[C@H]2C)C(=O)N1] | (5R,14R)-9-fluoro-5,14-dimethyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 23 | 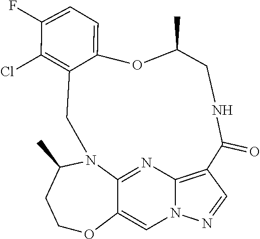 [SMILES: C[C@@H]1CCOc2cn3ncc4c3nc2N1Cc1c(ccc(F)c1Cl)O[C@@H](C)CNC4=O] | (5R,13S)-8-chloro-9-fluoro-5,13- dimethyl-4,5,14,15-tetrahydro- 3H,7H-19,1-(metheno)[1,4]- oxazepino[3,4-i]pyrazolo[4,3-f] [1,4,8,10]benzoxatriazacyclo- tridecin-16(13H)-one |
| 24 | 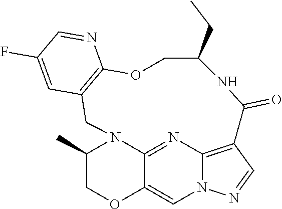 [SMILES: CC[C@@H]1COc2ncc(F)cc2CN2c3nc4c(cnn4cc3OC[C@H]2C)C(=O)N1] | (4R,13R)-13-ethyl-8-fluoro-4- methyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 25 | 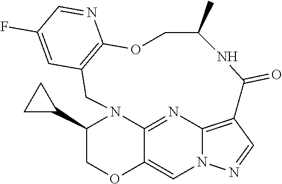 [SMILES: C[C@@H]1COc2ncc(F)cc2CN2c3nc4c(cnn4cc3OC[C@H]2C2CC2)C(=O)N1] | (4R,13R)-4-cyclopropyl-8-fluoro- 13-methyl-3,4,13,14-tetrahydro- 6H-18,1-(metheno)[1,4]oxazino- [3,4-i]pyrazolo[4,3-f]pyrido[3,2-l] [1,4,8,10]oxatriazacyclotridecin- 15(12H)-one |
| 26 | 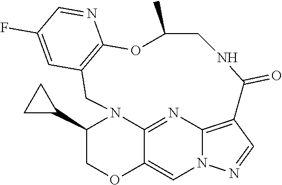 [SMILES: C[C@H]1CNC(=O)c2cnn3cc4c(nc23)N(Cc2cc(F)cnc2O1)[C@H](C1CC1)CO4] | (4R,12S)-4-cyclopropyl-8-fluoro- 12-methyl-3,4,13,14-tetrahydro- 6H-18,1-(metheno)[1,4]oxazino- [3,4-i]pyrazolo[4,3-f]pyrido- [3,2-l]-[1,4,8,10]oxatriazacyclo- tridecin-15(12H)-one |
| 27 | 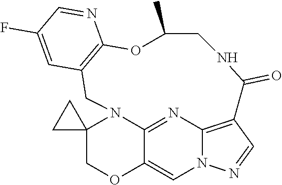 [SMILES: C[C@H]1CNC(=O)c2cnn3cc4c(nc23)N(Cc2cc(F)cnc2O1)C1(CC1)CO4] | (12′S)-8′-fluoro-12′-methyl- 3′H,6′H,12′H,13′H,14′H,15′H- spiro[cyclopropane-1,4′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 28 | 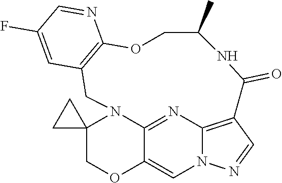 [SMILES: C[C@@H]1COc2ncc(F)cc2CN2c3nc4c(cnn4cc3OCC23CC3)C(=O)N1] | (13′R)-8′-fluoro-13′-methyl- 3′H,6′H,12′H,14′H,15′H-spiro- [cyclopropane-1,4′-[2,11]dioxa- [5,10,14,17,18,19]hexaaza[18,1]- (metheno)[1,4]oxazino[3,4-i]- pyrazolo[4,3-f]pyrido[3,2-l] [1,4,8,10]oxatriazacyclotridecin]- 15′-one |
| 29 | 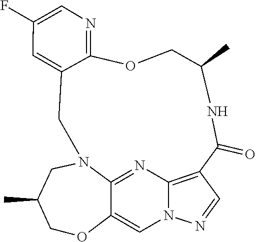 [SMILES: C[C@@H]1COc2cn3ncc4c3nc2N(Cc2cc(F)cnc2OC[C@@H](C)NC4=O)C1] | (4S,14R)-9-fluoro-4,14-dimethyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 30 | 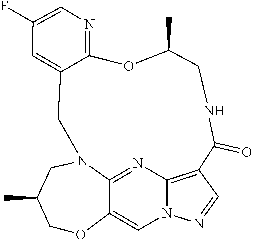 [SMILES: C[C@@H]1COc2cn3ncc4c3nc2N(Cc2cc(F)cnc2O[C@@H](C)CNC4=O)C1] | (4S,13S)-9-fluoro-4,13-dimethyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 31 | 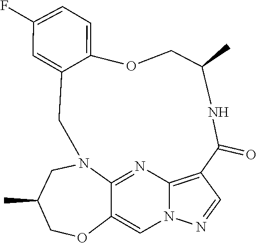 [SMILES: C[C@@H]1COc2cn3ncc4c3nc2N(Cc2cc(F)ccc2OC[C@@H](C)NC4=O)C1] | (4S,14R)-9-fluoro-4,14-dimethyl- 4,5,14,15-tetrahydro-3H,7H-19,1- (metheno)[1,4]oxazepino[3,4-i]- pyrazolo[4,3-f][1,4,8,10]benzo- xatriazacyclotridecin-16(13H)-one |
| 32 | 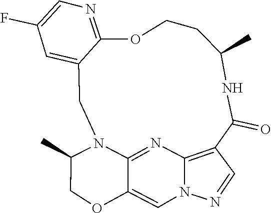 [SMILES: C[C@@H]1CCOc2ncc(F)cc2CN2c3nc4c(cnn4cc3OC[C@H]2C)C(=O)N1] | (6R,16R)-12-fluoro-6,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 33 | 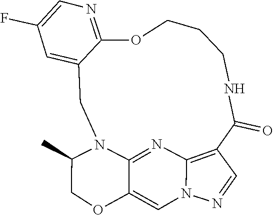 [SMILES: C[C@@H]1COc2cn3ncc4c3nc2N1Cc1cc(F)cnc1OCCCNC4=O] | (16R)-12-fluoro-16-methyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 34 | 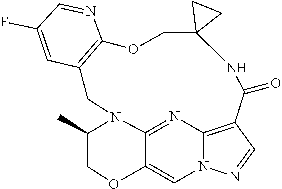 [SMILES: C[C@@H]1COc2cn3ncc4c3nc2N1Cc1cc(F)cnc1OCC1(CC1)NC4=O] | (4′R)-8′-fluoro-4′-methyl- 3′H,4′H,6′H,12′H,14′H,15′H- spiro[cyclopropane-1,13′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 35 | 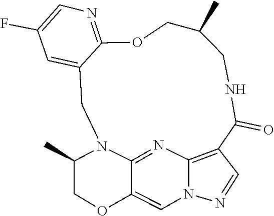 [SMILES: C[C@H]1CNC(=O)c2cnn3cc4c(nc23)N(Cc2cc(F)cnc2OC1)[C@H](C)CO4] | (7S,16R)-12-fluoro-7,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetra- decin-4-one |
| 36 | 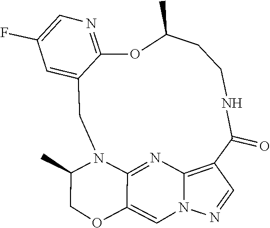 [SMILES: C[C@@H]1COc2cn3ncc4c3nc2N1Cc1cc(F)cnc1O[C@@H](C)CCNC4=O] | (8S,16R)-12-fluoro-8,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetra- decin-4-one |
| 37 | 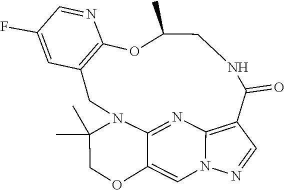 [SMILES: C[C@H]1CNC(=O)c2cnn3cc4c(nc23)N(Cc2cc(F)cnc2O1)C(C)(C)CO4] | (12S)-8-fluoro-4,4,12-trimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 38 | 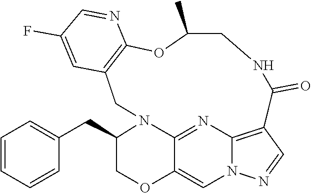 [SMILES: C[C@H]1CNC(=O)c2cnn3cc4c(nc23)N(Cc2cc(F)cnc2O1)[C@H](Cc1ccccc1)CO4] | (4R,12S)-4-benzyl-8-fluoro-12- methyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 39 | 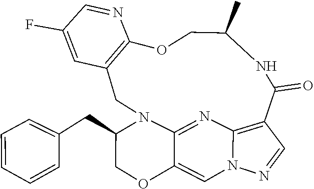 [SMILES: C[C@@H]1COc2ncc(F)cc2CN2c3nc4c(cnn4cc3OC[C@H]2Cc2ccccc2)C(=O)N1] | (4R,13R)-4-benzyl-8-fluoro-13- methyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 40 | 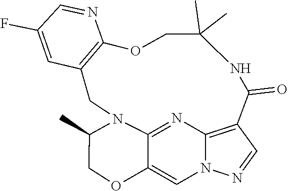 [SMILES: C[C@@H]1COc2cn3ncc4c3nc2N1Cc1cc(F)cnc1OCC(C)(C)NC4=O] | (4R)-8-fluoro-4,13,13-trimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 41 | 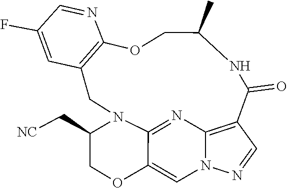 [SMILES: C[C@@H]1COc2ncc(F)cc2CN2c3nc4c(cnn4cc3OC[C@H]2CC#N)C(=O)N1] | [(4R,13R)-8-fluoro-13-methyl- 15-oxo-3,4,12,13,14,15-hexa- hydro-6H-18,1-(metheno)[1,4]- oxazino[3,4-i]pyrazolo[4,3-f]- pyrido[3,2-l][1,4,8,10]oxatriaza- cyclotridecin-4-yl]acetonitrile |
| 42 | 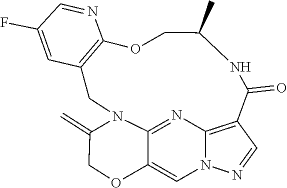 [SMILES: C=C1COc2cn3ncc4c3nc2N1Cc1cc(F)cnc1OC[C@@H](C)NC4=O] | (13R)-8-fluoro-13-methyl-4- methylidene-3,4,13,14-tetrahydro- 6H-18,1-(metheno)[1,4]oxazino- [3,4-i]pyrazolo[4,3-f]pyrido[3,2- l][1,4,8,10]oxatriazacyclotridecin- 15(12H)-one |
| 43 | 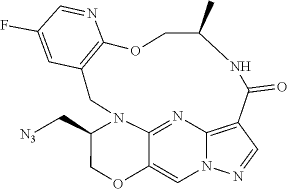 [SMILES: C[C@@H]1COc2ncc(F)cc2CN2c3nc4c(cnn4cc3OC[C@H]2CN=[N+]=[N-])C(=O)N1] | (4R,13R)-4-(azidomethyl)-8- fluoro-13-methyl-3,4,13,14- tetrahydro-6H-18,1-(metheno)- [1,4]oxazino[3,4-i]pyrazolo[4,3- f]pyrido[3,2-l][1,4,8,10]oxatriaza- cyclotridecin-15(12H)-one |
| 44 | 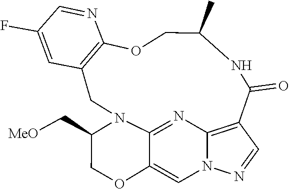 [SMILES: COC[C@@H]1COc2cn3ncc4c3nc2N1Cc1cc(F)cnc1OC[C@@H](C)NC4=O] | (4R,13R)-8-fluoro-4-(methoxy- methyl)-13-methyl-3,4,13,14- tetrahydro-6H-18,1-(metheno)- [1,4]oxazino[3,4-i]pyrazolo[4,3- f]pyrido[3,2-l][1,4,8,10]oxatriaza- cyclotridecin-15(12H)-one |
| 45 | 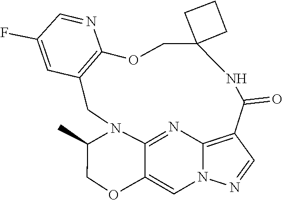 [SMILES: C[C@@H]1COc2cn3ncc4c3nc2N1Cc1cc(F)cnc1OCC1(CCC1)NC4=O] | (4′R)-8′-fluoro-4′-methyl- 3′H,4′H,6′H,12′H,14′H,15′H- spiro[cyclobutane-1,13′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 46 | 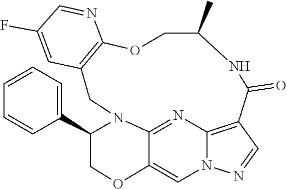 [SMILES: C[C@@H]1COc2ncc(F)cc2CN2c3nc4c(cnn4cc3OC[C@H]2c2ccccc2)C(=O)N1] | (4R,13R)-8-fluoro-13-methyl-4- phenyl-3,4,13,14-tetrahydro-6H- 18,1-(metheno)[1,4]oxazino[3,4- i]pyrazolo[4,3-f]pyrido[3,2-l] [1,4,8,10]oxatriazacyclotridecin- 15(12H)-one |
| 47 | 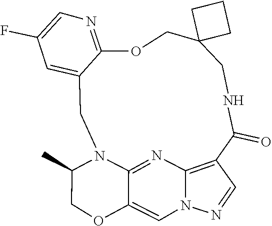 [SMILES: C[C@@H]1COc2cn3ncc4c3nc2N1Cc1cc(F)cnc1OCC1(CCC1)CNC4=O] | (16′R)-12′-fluoro-16′-methyl- 4′H,5′H,6′H,8′H,14′H,16′H,17′H- spiro[cyclobutane-1,7′-[9,18]- dioxa[1,2,5,10,15,20]hexaaza- [1,19](metheno)[1,4]oxazino- [4,3-e]pyrazolo[3,4-h]pyrido- [2,3-b][1,5,7,11]oxatriazacyclo- tetradecin]-4′-one |
| 48 | 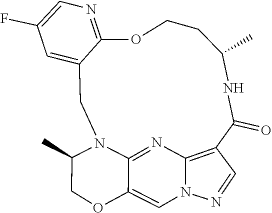 [SMILES: C[C@@H]1COc2cn3ncc4c3nc2N1Cc1cc(F)cnc1OCC[C@H](C)NC4=O] | (6S,16R)-12-fluoro-6,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetra- decin-4-one |
| 49 | 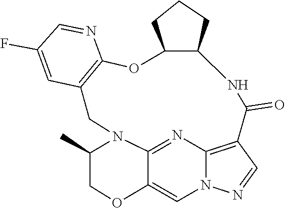 [SMILES: C[C@@H]1COc2cn3ncc4c3nc2N1Cc1cc(F)cnc1O[C@H]1CCC[C@H]1NC4=O] | (8R,15aS,18aR)-12-fluoro-8- methyl-7,8,15a,16,17,18,18a, 19-octahydro-10H,20H-3,5- (metheno)cyclopenta[b][1,4]- oxazino[3,4-i]pyrazolo[4,3-f]- pyrido[3,2-l][1,4,8,10]- oxatriazacyclotridecin-20-one |
| 50 | 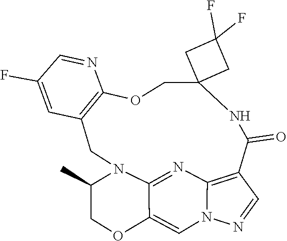 [SMILES: C[C@@H]1COc2cn3ncc4c3nc2N1Cc1cc(F)cnc1OCC1(CC(F)(F)C1)NC4=O] | (4′R)-3,3,8′-trifIuoro-4′-methyl- 3′H,4′H,6′H,12′H,14′H,15′H- spiro[cyclobutane-1,13′-[2,11]- dioxa[5,10,14,17,18,19]hexaaza- [18,1](metheno)[1,4]oxazino[3,4- i][4,3-f]pyrido[3,2-l][1,4,8,10]- oxatriazacyclotridecin]-15′-one |
| 51 | 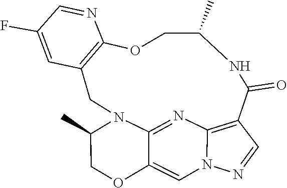 [SMILES: C[C@@H]1COc2cn3ncc4c3nc2N1Cc1cc(F)cnc1OC[C@H](C)NC4=O] | (4R,13S)-8-fluoro-4,13-dimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 52 | 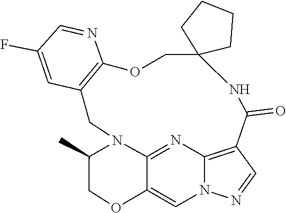 [SMILES: C[C@@H]1COc2cn3ncc4c3nc2N1Cc1cc(F)cnc1OCC1(CCCC1)NC4=O] | (4'R)-8'-fluoro-4'-methyl- 3'H,4'H,6'H,12'H,14'H,15'H- spiro[cyclopentane-1,13'-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15’-one |
| 53 | 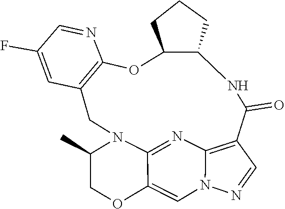 [SMILES: C[C@@H]1COc2cn3ncc4c3nc2N1Cc1cc(F)cnc1O[C@H]1CCC[C@@H]1NC4=O] | (8R,15aS,18aS)-12-fluoro-8- methyl-7,8,15a,16,17,18,18a, 19-octahydro-10H,20H-3,5- (metheno)cyclopenta[b][1,4]- oxazino[3,4-i]pyrazolo[4,3-f]- pyrido[3,2-l][1,4,8,10]oxatriaza- cyclotridecin-20-one |
| 54 | 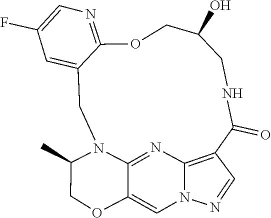 [SMILES: C[C@@H]1COc2cn3ncc4c3nc2N1Cc1cc(F)cnc1OC[C@@H](O)CNC4=O] | (7S,16R)-12-fluoro-7-hydroxy- 16-methyl-5,6,7,8,16,17-hexa- hydro-4H,14H-1,19-(metheno)- [1,4]oxazino[4,3-e]pyrazolo- [3,4-h]pyrido[2,3-b][1,5,7,11]- oxatriazacyclotetradecin-4-one |
| 55 | 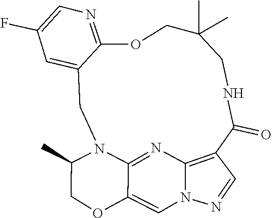 [SMILES: C[C@@H]1COc2cn3ncc4c3nc2N1Cc1cc(F)cnc1OCC(C)(C)CNC4=O] | (16R)-12-fluoro-7,7,16-trimethyl- 5,6.7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 56 | 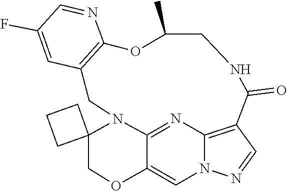 [SMILES: C[C@H]1CNC(=O)c2cnn3cc4c(nc23)N(Cc2cc(F)cnc2O1)C1(CCC1)CO4] | (12′S)-8′-fluoro-12′-methyl- 3′H,6′H,12′H,13′H,14′H,15′H- spiro[cyclobutane-1,4′-[2,11]- dioxa[5,10,14,17,18,19]- hexaaza[18,1](metheno)[1,4]- oxazino[3,4-i]pyrazolo[4,3-f]- pyrido[3,2-l][1,4,8,10]oxatriaza- cyclotridecin]-15′-one |
| 57 | 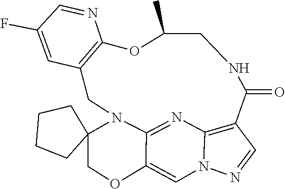 [SMILES: C[C@H]1CNC(=O)c2cnn3cc4c(nc23)N(Cc2cc(F)cnc2O1)C1(CCCC1)CO4] | (12′S)-8′-fluoro-12′-methyl- 3′H,6′H,12′H,13′H,14′H,15′H- spiro[cyclopentane-1,4′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 58 | 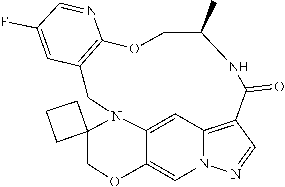 [SMILES: C[C@@H]1COc2ncc(F)cc2CN2c3cc4c(cnn4cc3OCC23CCC3)C(=O)N1] | (13′R)-8′-fluoro-13′-methyl- 3′H,6′H,12′H,13′H,14′H,15′H- spiro[cyclobutane-1,4′-[2,11]- dioxa[5,10,14,17,18,19]hexaaza- [18,1](metheno)[1,4]oxazino- [3,4-i]pyrazolo[4,3-f]pyrido- [3,2-l][1,4,8,10]oxatriazacyclo- tridecin]-15′-one |
| 59 | 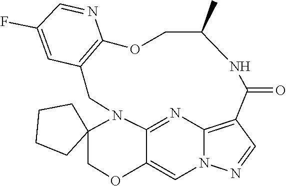 [SMILES: C[C@@H]1COc2ncc(F)cc2CN2c3nc4c(cnn4cc3OCC23CCCC3)C(=O)N1] | (13′R)-8′-fluoro-13′-methyl- 3′H,6′H,12′H,13′H,14′H,15′H- spiro[cyclopentane-1,4′-[2,11]- dioxa[5,10,14,17,18,19]hexaaza- [18,1](metheno)[1,4]oxazino- [3,4-i]pyrazolo[4,3-f]pyrido- [3,2-l][1,4,8,10]oxatriazacyclo- tridecin]-15′-one |
| 60 | 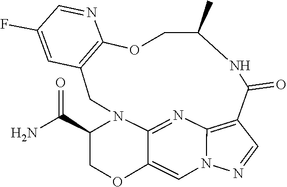 [SMILES: C[C@@H]1COc2ncc(F)cc2CN2c3nc4c(cnn4cc3OC[C@H]2C(N)=O)C(=O)N1] | (4S,13R)-8-fluoro-13-methyl-15- oxo-3,4,12,13,14,15-hexahydro- 6H-18,1-(metheno)[1,4]oxazino- [3,4-i]pyrazolo[4,3-f]pyrido[3,2-l] [1,4,8,10]oxatriazacyclotridecine- 4-carboxamide |
| 61 | 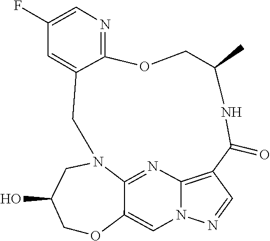 [SMILES: C[C@@H]1COc2ncc(F)cc2CN2C[C@H](O)COc3cn4ncc(c4nc32)C(=O)N1] | (4S,14R)-9-fluoro-4-hydroxy-14- methyl-4,5,14,15-tetrahydro- 3H,7H-19,1-(methano)[1,4]- oxazepino[3,4-i]pyrazolo[4,3-f]- pyrido[3,2-l][1,4,8,10]oxatriaza- cyclotridecin-16(13H)-one |
| 62 | 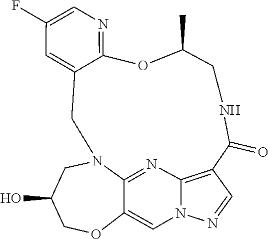 [SMILES: C[C@H]1CNC(=O)c2cnn3cc4c(nc23)N(Cc2cc(F)cnc2O1)C[C@H](O)CO4] | (4S,13S)-9-fluoro-4-hydroxy-13- methyl-4,5,14,15-tetrahydro- 3H,7H-19,1-(metheno)[1,4]- oxazepino[3,4-i]pyrazolo[4,3-f]- pyrido[3,2-l][1,4,8,10]oxatria- cyclotridecin-16(13H)-one |
| 63 | 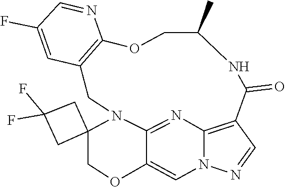 [SMILES: C[C@@H]1COc2ncc(F)cc2CN2c3nc4c(cnn4cc3OCC23CC(F)(F)C3)C(=O)N1] | (13′R)-3,3,8′-trifluoro-13′-methyl- 3′H,6′H,12′H,13′H,14′H,15′H- spiro[cyclobutane-1,4′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 64 | 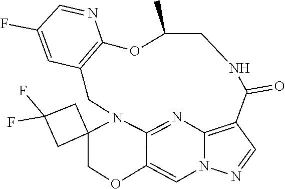 [SMILES: C[C@H]1CNC(=O)c2cnn3cc4c(nc23)N(Cc2cc(F)cnc2O1)C1(CO4)CC(F)(F)C1] | (12′S)-3,3,8′-trifluoro-12′-methyl- 3′H,6′H,12′H,13′H,14′H,15′H- spiro[cyclobutane-1,4′-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 65 | 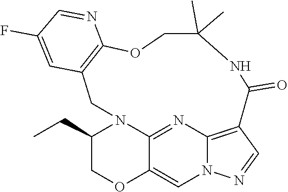 [SMILES: CC[C@@H]1COc2cn3ncc4c3nc2N1Cc1cc(F)cnc1OCC(C)(C)NC4=O] | (4R)-4-ethyl-8-fluoro-13,13- dimethyl-3,4,13,14-tetrahydro- 6H-18,1-(metheno)[1,4]oxazino- [3,4-i]pyrazolo[4,3-f]pyrido- [3,2-l][1,4,8,10]oxatriazacyclo- tridecin-15(12H)-one |
| 66 | 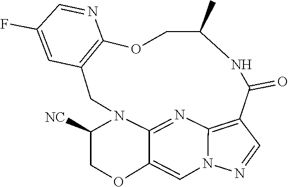 [SMILES: C[C@@H]1COc2ncc(F)cc2CN2c3nc4c(cnn4cc3OC[C@H]2C#N)C(=O)N1] | (4R,13R)-8-fluoro-13-methyl-15- oxo-3,4,12,13,14,15-hexahydro- 6H-18,1-(metheno)[1,4]oxazino- [3,4-i]pyrazolo[4,3-f]pyrido- [3,2-l][1,4,8,10]oxatriazacyclo- tridecine-4-carbonitrile |
| 67 | 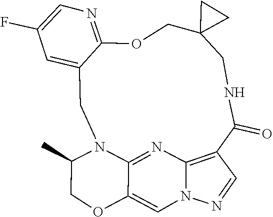 [SMILES: C[C@@H]1COc2cn3ncc4c3nc2N1Cc1cc(F)cnc1OCC1(CC1)CNC4=O] | (16′R)-12′-fluoro-16′-methyl- 4′H,5′H,6′H,8′H,14′H,16′H,17′H- spiro[cyclopropane-1,7′-[9,18]- dioxa[1,2,5,10,15,20]hexaaza- [1,19](metheno)[1,4]oxazino[4,3- e]pyrazolo[3,4-h]pyrido[2,3-b] [1,5,7,11]oxatriazacyclotetradecin]- 4′-one |
| 68 | 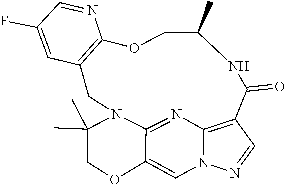 [SMILES: C[C@@H]1COc2ncc(F)cc2CN2c3nc4c(cnn4cc3OCC2(C)C)C(=O)N1] | (13R)-8-fluoro-4,4,13-trimethyl- 3,4,13,14-tetrahydro-6H-18,1- (metheno)[1,4]oxazino[3,4-i]- pyrazolo[4,3-f]pyrido[3,2-l] [1,4,8,10]oxatriazacyclotridecin- 15(12H)-one |
| 69 | 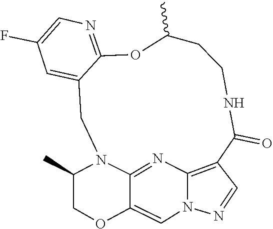 [SMILES: CC1CCNC(=O)c2cnn3cc4c(nc23)N(Cc2cc(F)cnc2O1)[C@H](C)CO4] | (16R)-12-fluoro-8,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 70 | 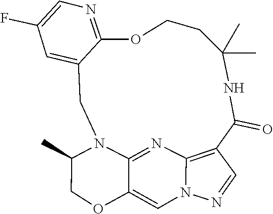 [SMILES: C[C@@H]1COc2cn3ncc4c3nc2N1Cc1cc(F)cnc1OCCC(C)(C)NC4=O] | (16R)-12-fluoro-6,6,16-trimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 71 | 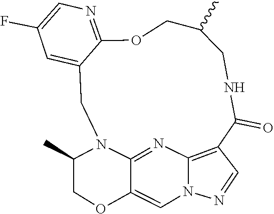 [SMILES: CC1CNC(=O)c2cnn3cc4c(nc23)N(Cc2cc(F)cnc2OC1)[C@H](C)CO4] | (16R)-12-fluoro-7,16-dimethyl- 5,6,7,8,16,17-hexahydro-4H,14H- 1,19-(metheno)[1,4]oxazino[4,3- e]pyrazolo[3,4-h]pyrido[2,3-b] [1,5,7,11]oxatriazacyclotetradecin- 4-one |
| 72 | 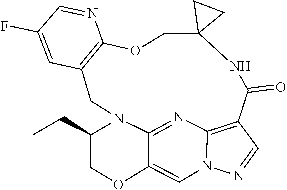 [SMILES: CC[C@@H]1COc2cn3ncc4c3nc2N1Cc1cc(F)cnc1OCC1(CC1)NC4=O] | (4′R)-4′-ethyl-8′-fluoro- 3′H,4′H,6′H,12′H,14′H,15′H- spiro[cyclopropane-1,13′-[2,11] dioxa[5,10,14,17,18,19]hexaaza- [18,1](metheno)[1,4]oxazino- [3,4-i]pyrazolo[4,3-f]pyrido[3,2- l][1,4,8,10]oxatriazacyclotridecin]- 15′-one |
| 73 | 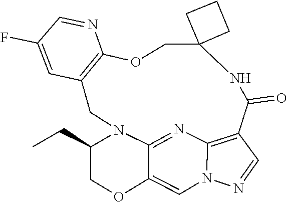 [SMILES: CC[C@@H]1COc2cn3ncc4c3nc2N1Cc1cc(F)cnc1OCC1(CCC1)NC4=O] | (4′R)-4′-ethyl-8′-fluoro- 3′H,4′H,6′H,12′H,14′H,15′H- spiro[cyclobutane-1,13’-[2,11]- dioxa[5,10,14,17,18,19]hexaaza- [18,1](metheno)[1,4]oxazino[3,4- i]pyrazolo[4,3-f]pyrido[3,2-l] [1,4,8,10]oxatriazacyclotridecin]- 15′-one |
| 74 | 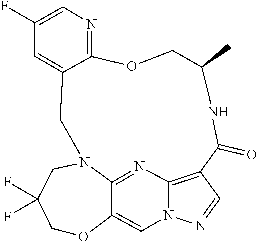 [SMILES: C[C@@H]1COc2ncc(F)cc2CN2CC(F)(F)COc3cn4ncc(c4nc32)C(=O)N1] | (14R)-4,4,9-trifluoro-14-methyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 75 | 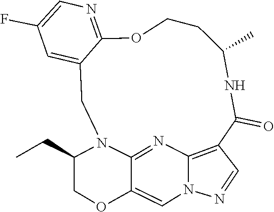 [SMILES: CC[C@@H]1COc2cn3ncc4c3nc2N1Cc1cc(F)cnc1OCC[C@H](C)NC4=O] | (6S,16R)-16-ethyl-12-fluoro-6- methyl-5,6,7,8,16,17-hexahydro- 4H,14H-1,19-(metheno)[1,4]- oxazino[4,3-e]pyrazolo[3,4-h]- pyrido[2,3-b][1,5,7,11]oxatriaza- cyclotetradecin-4-one |
| 76 | 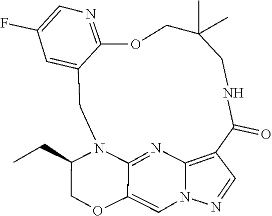 [SMILES: CC[C@@H]1COc2cn3ncc4c3nc2N1Cc1cc(F)cnc1OCC(C)(C)CNC4=O] | (16R)-16-ethyl-12-fluoro-7,7- dimethyl-5,6,7,8,16,17-hexahydro- 4H,14H-1,19-(metheno)[1,4]- oxazino[4,3-e]pyrazolo[3,4-h]- pyrido[2,3-b][1,5,7,11]oxatriaza- cyclotetradecin-4-one |
| 77 | 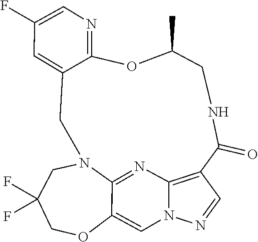 [SMILES: C[C@H]1CNC(=O)c2cnn3cc4c(nc23)N(Cc2cc(F)cnc2O1)CC(F)(F)CO4] | (13S)-4,4,9-trifluoro-13-methyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 78 | 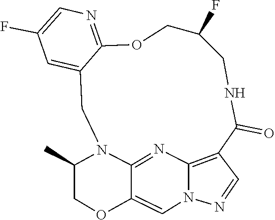 [SMILES: C[C@@H]1COc2cn3ncc4c3nc2N1Cc1cc(F)cnc1OC[C@@H](F)CNC4=O] | (7S,16R)-7,12-difluoro-16-methyl- 5,6,7,8,16,17-hexahydro-4H,14H,- 1,19-(metheno)[1,4]oxazino[4,3- e]pyrazolo[3,4-h]pyrido[2,3-b] [1,5,7,11]oxatriazacyclotetradecin- 4-one |
| 79 | 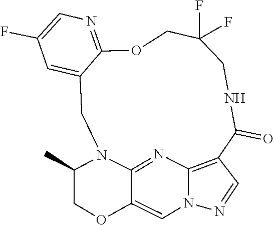 [SMILES: C[C@@H]1COc2cn3ncc4c3nc2N1Cc1cc(F)cnc1OCC(F)(F)CNC4=O] | (16R)-7,7,12-trifluoro-16-methyl- 5,6,7,8,16,17-hexahydro-4H,14H- 1,19-(metheno)[1,4]oxazino[4,3- e]pyrazolo[3,4-h]pyrido[2,3- b][1,5,7,11]oxatriazacyclotetra- decin-4-one |
| 80 | 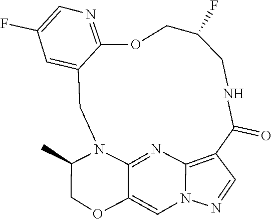 [SMILES: C[C@@H]1COc2cn3ncc4c3nc2N1Cc1cc(F)cnc1OC[C@H](F)CNC4=O] | (7R,16R)-7,12-difluoro-16- methyl-5,6,7,8,16,17-hexahydro- 4H,14H-1,19-(metheno)[1,4]- oxazino[4,3-e]pyrazolo[3,4- h]pyrido[2,3-b][1,5,7,11]oxa- triazacyclotetradecin-4-one |
| 81 | 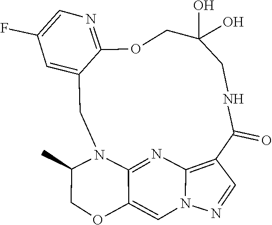 [SMILES: C[C@@H]1COc2cn3ncc4c3nc2N1Cc1cc(F)cnc1OCC(O)(O)CNC4=O] | (16R)-12-fluoro-7,7-dihydroxy- 16-methyl-5,6,7,8,16,17-hexa- hydro-4H,14H-1,19-(metheno)- [1,4]oxazino[4,3-e]pyrazolo[3,4- h]pyrido[2,3-b][1,5,7,11]oxa- triazacyclotetradecin-4-one |
| 82 | 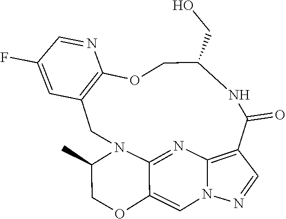 [SMILES: C[C@@H]1COc2cn3ncc4c3nc2N1Cc1cc(F)cnc1OC[C@H](CO)NC4=O] | (4R,13S)-8-fluoro-13-(hydroxy- methyl)-4-methyl-3,4,13,14- tetrahydro-6H-18,1-(metheno)- [1,4]oxazino[3,4-i]pyrazolo[4,3- f]pyrido[3,2-l][1,4,8,10]oxatriaza- cyclotridecin-15(12H)-one |
| 83 | 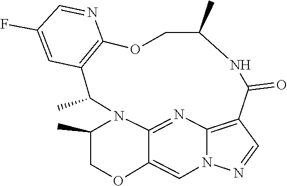 [SMILES: C[C@@H]1COc2ncc(F)cc2[C@@H](C)N2c3nc4c(cnn4cc3OC[C@H]2C)C(=O)N1] | (4R,6R,13R)-8-fluoro-4,6,13- trimethyl-3,4,13,14-tetrahydro- 6H-18,1-(metheno)[1,4]oxazino- [3,4-i]pyrazolo[4,3-f]pyrido[3,2- l][1,4,8,10]oxatriazacyclotridecin- 15(12H)-one |
| 84 | 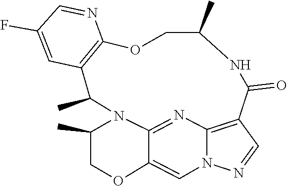 [SMILES: C[C@@H]1COc2ncc(F)cc2[C@H](C)N2c3nc4c(cnn4cc3OC[C@H]2C)C(=O)N1] | (4R,6S,13R)-8-fluoro-4,6,13- trimethyl-3,4,13,14-tetrahydro- 6H-18,1-(metheno)[1,4]oxazino- [3,4-i]pyrazolo[4,3-f]pyrido(3,2- l][1,4,8,10]oxatriazacyclotridecin- 15(12H)-one |
| 85 | 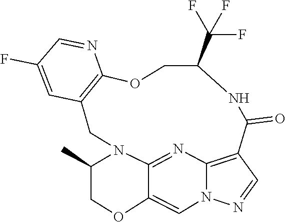 [SMILES: C[C@@H]1COc2cn3ncc4c3nc2N1Cc1cc(F)cnc1OC[C@@H](C(F)(F)F)NC4=O] | (4R,13S)-8-fluoro-4-methyl-13- (trifluoromethyl)-3,4,13,14- tetrahydro-6H-18,1-(metheno)- [1,4]oxazino[3,4-i]pyrazolo[4,3- f]pyridol[3,2-l][1,4,8,10]oxatriaza- cyclotridecin-15(12H)-one |
| 86 | 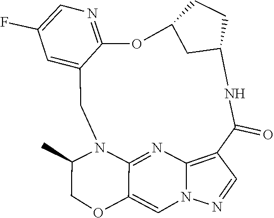 [SMILES: C[C@@H]1COc2cn3ncc4c3nc2N1Cc1cc(F)cnc1O[C@@H]1CC[C@@H](C1)NC4=O] | (6S,9R,17R)-13-fluoro-17-methyl- 6,7,8,9,17,18-hexahydro-15H- 6,9-methano-1,20-(metheno)[1,4]- oxazino[4,3-e]pyrazolo[3,4-h]- pyrido[2,3-b][1,5,7,11]oxatriaza- cyclopentadecin-4(5H)-one |
| 87 | 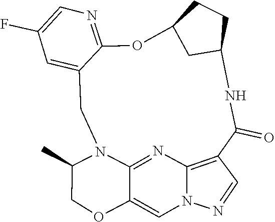 [SMILES: C[C@@H]1COc2cn3ncc4c3nc2N1Cc1cc(F)cnc1O[C@H]1CC[C@H](C1)NC4=O] | (6R,9S,17R)-13-fluoro-17-methyl- 6,7,8,9,17,18-hexahydro-15H-6,9- methano-1,20-(metheno)[1,4]- oxazino[4,3-e]pyrazolo[3,4-h]- pyrido[2,3-b][1,5,7,11]oxatriaza- cyclopentadecin-4(5H)-one |
| 88 | 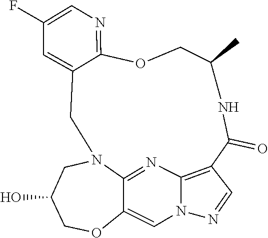 [SMILES: C[C@@H]1COc2ncc(F)cc2CN2C[C@@H](O)COc3cn4ncc(c4nc32)C(=O)N1] | (4R,14R)-9-fluoro-4-hydroxy-14- methyl-4,5,14,15-tetrahydro- 3H,7H-19,1-(metheno)[1,4]- oxazepino[3,4-i]pyrazolo[4,3- f]pyrido[3,2-l][1,4,8,10]oxatriaza- cyclotridecin-16(13H)-one |
| 89 | 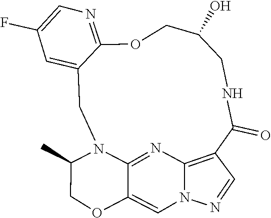 [SMILES: C[C@@H]1COc2cn3ncc4c3nc2N1Cc1cc(F)cnc1OC[C@H](O)CNC4=O] | (7R,16R)-12-fluoro-7-hydroxy- 16-methyl-5,6,7,8,16,17-hexa- hydro-4H,14H-1,19-(metheno)- [1,4]oxazino[4,3-e]pyrazolo[3,4- h]pyrido[2,3-b][1,5,7,11]oxatriaza- cyclotetradecin-4-one |
| 90 | 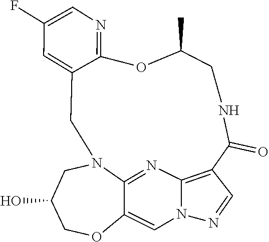 [SMILES: C[C@H]1CNC(=O)c2cnn3cc4c(nc23)N(Cc2cc(F)cnc2O1)C[C@@H](O)CO4] | (4R,13S)-9-fluoro-4-hydroxy-13- methyl-4,5,14,15-tetrahydro- 3H,7H-19,1-(metheno)[1,4]- oxazepino[3,4-i]pyrazolo[4,3- f]pyrido[3,2-l][1,4,8,10]oxatriaza- cyclotridecin-16(13H)-one |
| 91 | 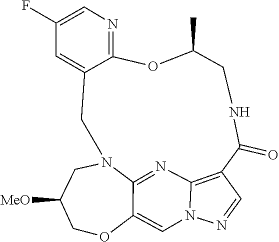 [SMILES: CO[C@@H]1COc2cn3ncc4c3nc2N(Cc2cc(F)cnc2O[C@@H](C)CNC4=O)C1] | (4S,13S)-9-fluoro-4-methoxy-13- methyl-4,5,14,15-tetrahydro- 3H,7H-19,1-(metheno)[1,4]- oxazepino[3,4-i]pyrazolo[4,3- f]pyrido[3,2-l][1,4,8,10]oxatriaza- cyclotridecin-16(13H)-one |
| 92 | 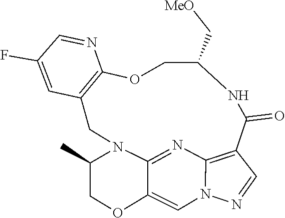 [SMILES: COC[C@H]1COc2ncc(F)cc2CN2c3nc4c(cnn4cc3OC[C@H]2C)C(=O)N1] | (4R,13S)-8-fluoro-13-(methoxy- methyl)-4-methyl-3,4,13,14- tetrahydro-6H-18,1-(metheno)- [1,4]oxazino[3,4-i]pyrazolo[4,3- f]pyrido[3,2-l][1,4,8,10]oxatriaza- cyclotridecin-15(12H)-one |
| 93 | 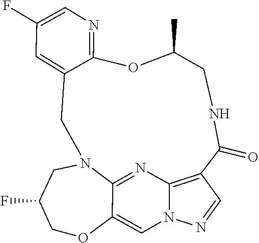 [SMILES: C[C@H]1CNC(=O)c2cnn3cc4c(nc23)N(Cc2cc(F)cnc2O1)C[C@@H](F)CO4] | (4R,13S)-4,9-difluoro-13-methyl- 4,5,14,15-tetrahydro-3H,7H-19,1- (metheno)[1,4]oxazepino[3,4-i]- pyrazolo[4,3-f]pyrido[3,2-l] [1,4,8,10]oxatriazacyclotridecin- 16(13H)-one |
| 94 | 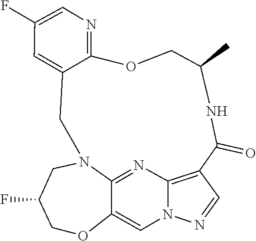 [SMILES: C[C@@H]1COc2ncc(F)cc2CN2C[C@@H](F)COc3cn4ncc(c4nc32)C(=O)N1] | (4R,14R)-4,9-difluoro-14-methyl- 4,5,14,15-tetrahydro-3H,7H-19,1- (metheno)[1,4]oxazepino[3,4- i]pyrazolo[4,3-f]pyrido[3,2-l] [1,4,8,10]oxatriazacyclotridecin- 16(13H)-one |
| 95 | 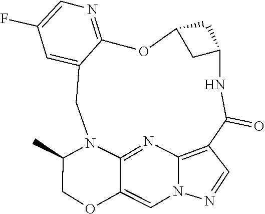 [SMILES: C[C@@H]1COc2cn3ncc4c3nc2N1Cc1cc(F)cnc1O[C@H]1C[C@H](C1)NC4=O] | (6S,8s,16R)-12-fluoro-16-methyl- 5,6,7,8,16,17-hexahydro-4H,14H- 6,8-methano-1,19-(metheno)[1,4]- oxazino[4,3-e]pyrazolo[3,4-h]- pyrido[2,3-b][1,5,7,11]oxatriaza- cyclotetradecin-4-one |
| 96 | 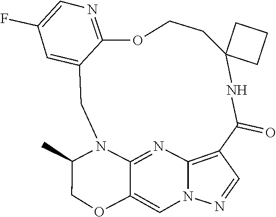 [SMILES: C[C@@H]1COc2cn3ncc4c3nc2N1Cc1cc(F)cnc1OCCC1(CCC1)NC4=O] | (16′R)-12′-fluoro-16′-methyl- 4′H,5′H,7′H,8′H,14′H,16′H,17′H- spiro[cyclobutane-1,6′-[9,18]- dioxa[1,2,5,10,15,20]hexaaza- [1,19](metheno)[1,4]oxazino- [4,3-e]pyrazolo[3,4-h]pyrido[2,3- b][1,5,7,11]oxatriazacyclotetra- decin]-4′-one |
| 97 | 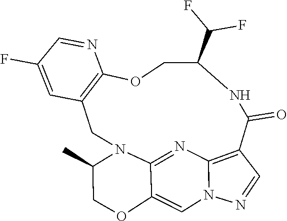 [SMILES: C[C@@H]1COc2cn3ncc4c3nc2N1Cc1cc(F)cnc1OC[C@@H](C(F)F)NC4=O] | (4R,13S)-13-(difluoromethyl)-8- fluoro-4-methyl-3,4,13,14-tetra- hydro-6H-18,1-(metheno)[1,4]- oxazino[3,4-i]pyrazolo[4,3- f]pyrido[3,2-l][1,4,8,10]oxatriaza- cyclotridecin-15(12H)-one |
| 98 | 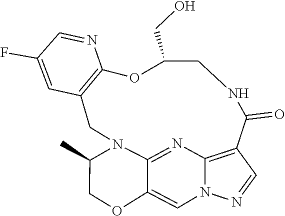 [SMILES: C[C@@H]1COc2cn3ncc4c3nc2N1Cc1cc(F)cnc1O[C@H](CO)CNC4=O] | (4R,12S)-8-fluoro-12-(hydroxy- methyl)-4-methyl-3,4,13,14- tetrahydro-6H-18,1-(metheno)- [1,4]oxazino[3,4-i]pyrazolo[4,3- f]pyrido[3,2-l][1,4,8,10]oxatriaza- cyclotridecin-15(12H)-one |
| 99 | 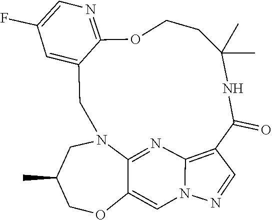 [SMILES: C[C@@H]1COc2cn3ncc4c3nc2N(Cc2cc(F)cnc2OCCC(C)(C)NC4=O)C1] | (17S)-12-fluoro-6,6,17- trimethyl-5,6,7,8,17,18-hexa- hydro-4H,14H,16H-1,20- (metheno)[1,4]oxazepino- [4,3-e]pyrazolo[3,4-h]pyrido- [2,3-b][1,5,7,11]oxatriaza- cyclotetradecin-4-one |
| 100 | 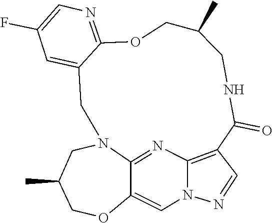 [SMILES: C[C@@H]1COc2cn3ncc4c3nc2N(Cc2cc(F)cnc2OC[C@@H](C)CNC4=O)C1] | (7S,17S)-12-fluoro-7,17- dimethyl-5,6,7,8,17,18- hexahydro-4H,14H,16H-1,20- (metheno)[1,4]oxazepino- [4,3-e]pyrazolo[3,4-h]pyrido- [2,3-b][1,5,7,11]oxatriaza- cyclotetradecin-4-one |

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or a spills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. Cancer includes, for example, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the invention specifically target tyrosine receptor kinases, in particular ALK, ROS1, TRK, JAK, and FGFRs. Thus, these compounds and pharmaceutical compositions can be used to prevent, reverse, slow, or inhibit the activity of one or more of these kinases. In preferred embodiments, methods of treatment target cancer. In other embodiments, methods are for treating lung cancer or non-small cell lung cancer.

In the inhibitory methods of the invention, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to upregulation of ALK, ROS1, TRK, JAK, and FGFRs.

In treatment methods according to the invention, an “effective amount” means an amount or dose sufficient to generally bring about the desired therapeutic benefit in subjects needing such treatment. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as EGFR inhibitors (e.g., erlotinib, gefitinib), Raf inhibitors (e.g., vemurafenib), VEGFR inhibitors (e.g., sunitinib), ALK inhibitors (e.g., crizotinib) standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the invention may additionally comprise one or more of such active agents, and methods of treatment may additionally comprise administering an effective amount of one or more of such active agents.

Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

Abbreviations The examples described herein use materials,
including but not limited to, those described by the following
abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| μm | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |

### General Method A

### Preparation of Ethyl 2-(((2-((tert-butyldimethylsilyl)oxy)ethyl)amino)methyl)-3-chloro-4-fluorophenol (A-1)

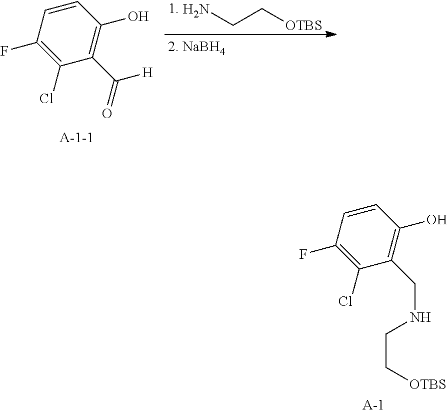

Step 1. A solution of A-1-1 (250 mg, 1.4 mmol, 1 eq.) and 2-((tert-butyldimethylsilyl)oxy)ethanamine (401 mg, 2.3 mmol, 1.6) in methanol (4.8 mL) was stirred for 1 hour at 65° C. The reaction mixture was cooled to room temperature and NaBH₄ (81 mg, 1.5 mmol, 1.5 eq.) was added, the reaction mixture was stirred at 25° C. for 1 hr. The mixture was quenched with water (15 mL) and stirred for 5 min. The mixture was extracted with DCM (3×15 mL), dried with Na₂SO₄ and concentrated under reduced pressure. Flash chromatography (ISCO system, silica (40 g), 0-30% ethyl acetate in hexane) provided A-1 (447 mg, 93% yield).

Compound A-2 was prepared according to General Method A using 5-fluoro-2-hydroxybenzaldehyde and (S)-2-aminopropan-1-ol.

Compound A-3 was prepared according to General Method A using 5-fluoro-2-hydroxybenzaldehyde and (R)-2-aminopropan-1-ol.

### General Method B

### Preparation of Tert-Butyl ((S)-2-(4-fluoro-2-((((S)-1-hydroxypropan-2-yl)amino)methyl)phenoxy)propyl)carbamate (A-4)

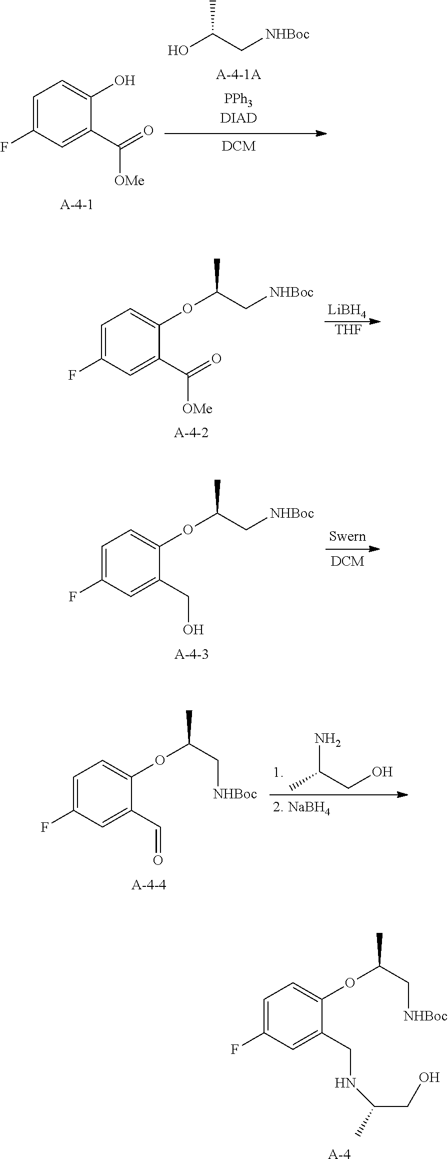

Step 1. To an azeotrope dried mixture of A-4-1 (0.9615 g, 5.65 mmol) and A-4-1A (1.19 g, 6.78 mmol) in DCM (3.62 mL) was added PPh3 (2.22 g, 8.48 mmol) The mixture was stirred until everything completely dissolved. Added in DIAD (1.83 g, 9.04 mmol, 1.78 mL) very slowly with mixing at 0° C. The reaction was warmed to 25° C. and stirred for 16 hr. Added DCM (5 mL) and 2M NaOH solution (20 mL), stirred vigorously for 4 hours. The mixture was extracted with DCM (3×15 mL), dried with Na2SO4 and concentrated under reduced pressure. Flash chromatography (ISCO system, silica 12 g, 0-30% ethyl acetate in hexane) provided A-4-2 (1.35 g, 73%).

Step 2. To a solution of A-4-2 (1.35 g, 4.13 mmol) in THF (8.27 mL) at 0° C. was added lithium borohydride (720.51 mg, 33.08 mmol) in small batches and the mixture was stirred for 1 hr and was removed from the cold bath. The mixture was stirred at ambient temperature for 20 hr, diluted with water (5 mL) and extracted with ethyl acetate (3×5 mL). The combined organic phase was washed with brine and dried over sodium sulfate. Flash column chromatography (ISCO, Silica 24 g, ethyl acetate in hexanes) afforded A-4-3 (1.08 g, 3.60 mmol, 87.09% yield).

Step 3. DMSO (422.82 mg, 5.41 mmol, 384.38 L) in DCM (6 mL) was added dropwise at −78° C. to oxalyl chloride (686.85 mg, 5.41 mmol, 464.09 uL) in DCM (6 mL). Stirred for 20 minutes and A-4-3 (1.08 g, 3.61 mmol) in DCM (6 mL) was added dropwise at −78° C. and stirred for 20 min followed by addition of TEA (1.83 g, 18.04 mmol, 2.51 mL). Stirred as temperature increased to ambient temperature over 18 hr. The reaction was quenched with water (10 mL) and layers were separated. The aqueous layer was extracted twice more with DCM (2×10 mL). The combined organic layer was washed with brine and dried over sodium sulfate. Flash chromatography (ISCO, 24 g Silica Gold, 0-30% ethyl acetate in hexanes) afforded A-4-4 (460.2 mg, 1.55 mmol, 42.90% yield).

Step 4. A solution of (S)-2-aminopropan-1-ol (56.84 mg, 756.76 μmol) and A-4-4 (150.00 mg, 504.51 μmol) in dry MeOH (2.50 mL) was heated to 65° C. for 1 hr. The reaction was cooled to room temperature and NaBH₄ (28.63 mg, 756.76 μmol) was added. The mixture was stirred for 30 min then quenched with water (3 mL) and stirred for 5 min. The mixture was extracted with DCM (3×5 mL), dried with Na₂SO₄ and concentrated under reduced pressure. Flash chromatography (ISCO system, silica (12 g), 70-100% ethyl acetate in hexane) provided A-4 (140.70 mg, 394.75 μmol, 78.24% yield).

Compound A-5 was prepared according to General Method B using (R)-2-aminopropan-1-ol in step 4.

Compound A-6 was prepared according to General Method B using (R)-2-aminobutan-1-ol in step 4.

Compound A-7 was prepared according to General Method A using 3,5-difluoro-2-hydroxybenzaldehyde and (R)-2-aminopropan-1-ol.

Compound A-8 was prepared according to General Method A using 5-fluoro-2-methoxynicotinaldehyde and (R)-2-aminopropan-1-ol.

Compound A-9 was prepared according to General Method B using (R)-tert-butyl (1-hydroxypropan-2-yl)carbamate in step 1 and (R)-2-aminopropan-1-ol in step 4.

Compound A-10 was prepared according to General Method A using 5-fluoro-2-methoxynicotinaldehyde and (R)-2-aminobutan-1-ol.

Compound A-11 was prepared according to General Method B using (S)-2-amino-3-(benzyloxy)propan-1-ol in step 4.

Compound A-12 was prepared according to General Method B using (R)-tert-butyl (1-hydroxypropan-2-yl)carbamate in step 1 and (R)-2-aminobutan-1-ol in step 4.

### General Method C

### Preparation of Tert-Butyl ((S)-2-(2,4-difluoro-6-((((R)-1-hydroxypropan-2-yl)amino)methyl)phenoxy)propyl)carbamate (A-13)

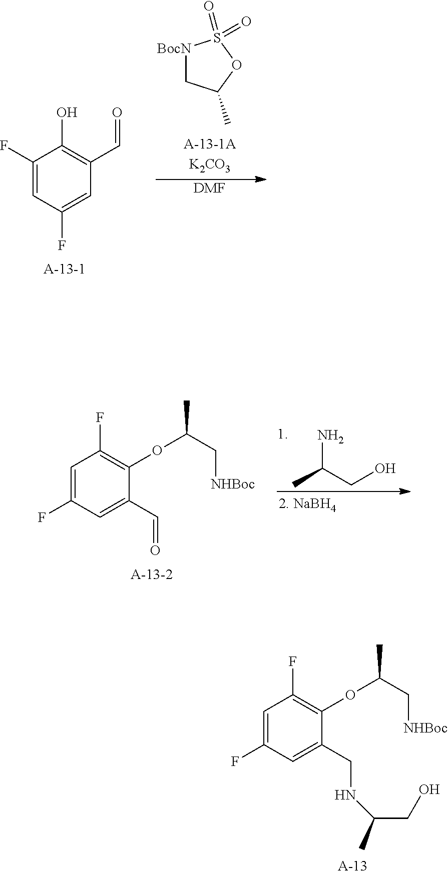

Step 1. Added K₂CO₃ (330.00 mg, 2.39 mmol) to A-13-1 (151 mg, 955.08 μmol) and A-13-1A (283.27 mg, 1.19 mmol) in DMF (4.78 mL) and heated to 50° C. with stirring for 1 hr. Cooled reaction and diluted with DCM (3 mL), filtered through a syringe filter and concentrated under reduced pressure. Flash chromatography (ISCO system, silica (12 g), 0-30% ethyl acetate in hexane) provide A-13-2 (301 mg, 954 μmol, 99% yield).

Step 4. A solution of (R)-2-aminopropan-1-ol (143 mg, 1.9 mmol) and A-13-2 (301 mg, 954 μmol) in dry MeOH (4.78 mL) was heated to 65° C. for 1 hr. The reaction was cooled to −10° C. and NaBH₄ (72 mg, 1.9 mmol) was added. The mixture was stirred for 30 min while warming up then quenched with water (15 mL) and stirred for 5 min. The mixture was extracted with DCM (3×15 mL), dried with Na₂SO₄ and concentrated under reduced pressure. Flash chromatography (ISCO system, silica (12 g), 25-100% ethyl acetate in hexane) provided A-13 (286 mg, 764 μmol, 80% yield).

Compound A-14 through A-17 were prepared according to General Method C.

Compound A-18 was prepared according to General Method A using 5-fluoro-2-methoxynicotinaldehyde and (R)-3-aminopentan-1-ol.

Compound A-19 was prepared according to General Method C.

Compound A-20 and A-21 were prepared according to General Method A.

| | | MS |
|---|---|---|
| | | [M + H] |
| Compd# | Structure | m/z |
| A-1 | 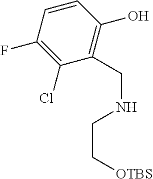 [SMILES: CC(C)(C)[Si](C)(C)OCCNCc1c(O)ccc(F)c1Cl] | 334.1 |
| A-2 | 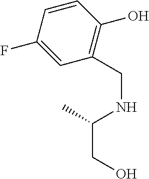 | 200.1 |
| A-3 | 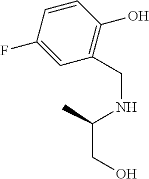 | 200.1 |
| A-4 | 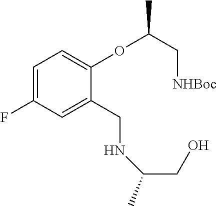 [SMILES: C[C@@H](CO)NCc1cc(F)ccc1O[C@@H](C)CNC(=O)OC(C)(C)C] | 357.2 |
| A-5 | 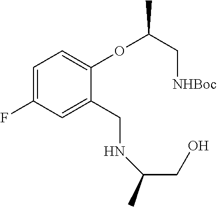 [SMILES: C[C@H](CO)NCc1cc(F)ccc1O[C@@H](C)CNC(=O)OC(C)(C)C] | 357.2 |
| A-6 | 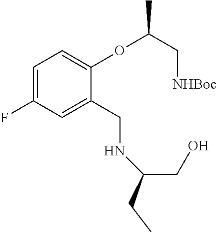 [SMILES: CC[C@H](CO)NCc1cc(F)ccc1O[C@@H](C)CNC(=O)OC(C)(C)C] | 371.2 |
| A-7 | 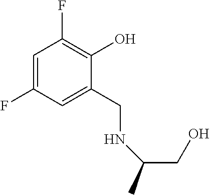 | 218.1 |
| A-8 | 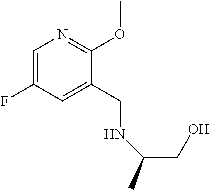 | 215.1 |
| A-9 | 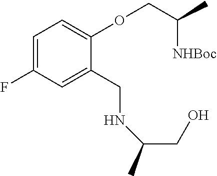 [SMILES: C[C@H](CO)NCc1cc(F)ccc1OC[C@@H](C)NC(=O)OC(C)(C)C] | 357.1 |
| A-10 | 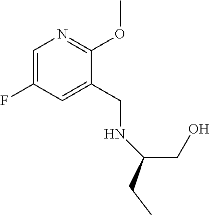 [SMILES: CC[C@H](CO)NCc1cc(F)cnc1OC] | 229.1 |
| A-11 | 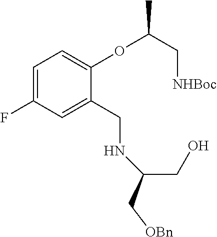 [SMILES: C[C@@H](CNC(=O)OC(C)(C)C)Oc1ccc(F)cc1CN[C@@H](CO)COCc1ccccc1] | 463.2 |
| A-12 | 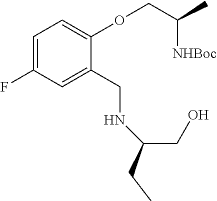 [SMILES: CC[C@H](CO)NCc1cc(F)ccc1OC[C@@H](C)NC(=O)OC(C)(C)C] | 371.1 |
| A-13 | 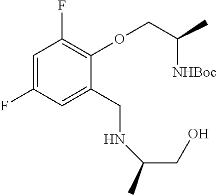 [SMILES: C[C@H](CO)NCc1cc(F)cc(F)c1OC[C@@H](C)NC(=O)OC(C)(C)C] | 375.1 |
| A-14 | 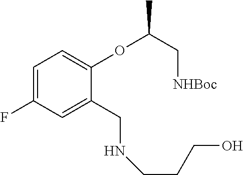 [SMILES: C[C@@H](CNC(=O)OC(C)(C)C)Oc1ccc(F)cc1CNCCCO] | 357.2 |
| A-15 | 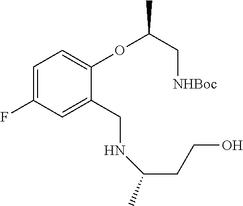 [SMILES: C[C@@H](CCO)NCc1cc(F)ccc1O[C@@H](C)CNC(=O)OC(C)(C)C] | 371.2 |
| A-16 | 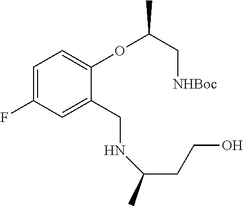 [SMILES: C[C@H](CCO)NCc1cc(F)ccc1O[C@@H](C)CNC(=O)OC(C)(C)C] | 371.2 |
| A-17 | 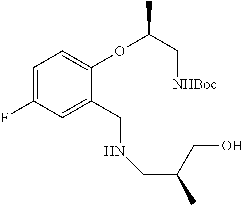 [SMILES: C[C@H](CO)CNCc1cc(F)ccc1O[C@@H](C)CNC(=O)OC(C)(C)C] | 371.2 |
| A-18 | 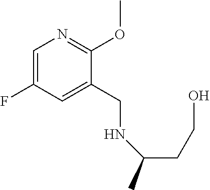 [SMILES: COc1ncc(F)cc1CN[C@H](C)CCO] | 229.0 |
| A-19 | 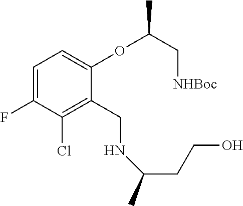 [SMILES: C[C@H](CCO)NCc1c(O[C@@H](C)CNC(=O)OC(C)(C)C)ccc(F)c1Cl] | 405.2 |
| A-20 | 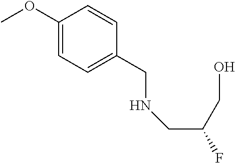 | 214.0 |
| A-21 | 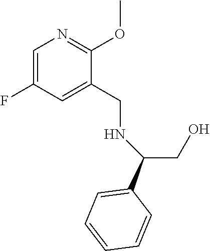 [SMILES: COc1ncc(F)cc1CN[C@@H](CO)c1ccccc1] | |
| A-22 | 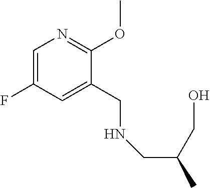 [SMILES: COc1ncc(F)cc1CNC[C@H](C)CO] | 229.1 |

### General Method D

### Preparation of Ethyl 6-bromo-5-chloropyrazolo[1,5-a]pyrimidine-3-carboxylate (B-1)

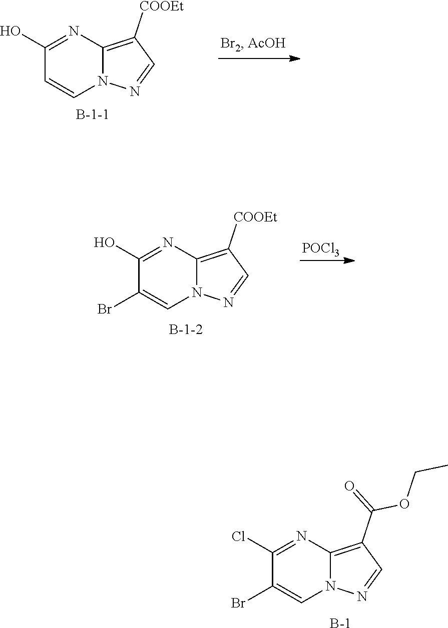

Step 1. To a solution of B-1-1 (10.00 g, 47.80 mmol, 1.00 eq.) in acetic acid (100.00 mL) was added bromine (7.64 g, 47.80 mmol, 2.46 mL, 1.00 eq.). The mixture was stirred at 180° C. for 6 hours. TLC (petroleum ether/ethyl acetate=1/1) showed the starting material was consumed completely and one new spot was found. The mixture was quenched by water (30 mL). The mixture was filtered and the cake was concentrated to give B-1-2 (10.00 g, 34.71 mmol, 72.62% yield) as a white solid: ¹H NMR (400 MHz, DMSO-d6) δ:12.34 (br. s., 1H), 9.25 (s, 1H), 8.15 (s, 1H), 4.28 (q, J=7.2 Hz, 2H), 1.29 (t, J=7.2 Hz, 3H).

Step 2. To a solution of B-1-2 (6.00 g, 20.97 mmol, 1.00 eq.) in phosphorus oxychloride (60.00 mL). The mixture was stirred at 120° C. for 16 hours. TLC (Petroleum ether/Ethyl acetate=3/1) indicated the starting material was consumed completely and one new spot was found. The reaction mixture was filtered and concentrated under reduced pressure to give a residue. The residue was purified by column chromatography (SiO₂, petroleum ether/ethyl acetate=10/1 to 1/1) to give B-1(2.50 g, 8.21 mmol, 39.15% yield) as a white solid; ¹H NMR (400 MHz, CDCl₃) δ: 8.94 (s, 1H), 8.54 (s, 1H), 4.43 (q, J=7.2 Hz, 2H), 1.42 (t, J=7.2 Hz, 3H).

### General Method E

### Preparation of (12S)-7-chloro-8-fluoro-12-methyl-3,4,13,14-tetrahydro-6H-18,1-(metheno)[1,4]oxazino[3,4-i]pyrazolo[4,3-f][1,4,8,10]benzoxatriazacyclotridecin-15(12H)-one (1)

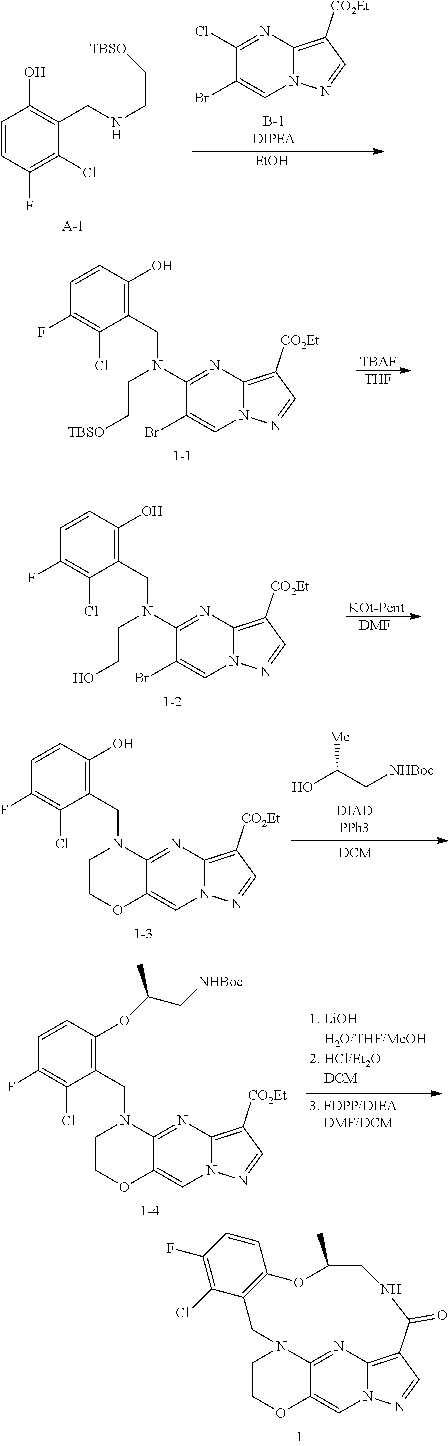

Step 1. To a solution of B-1 (125 mg, 410 μmol) and A-1 (137 mg, 410 μmol) in EtOH (2.05 mL) was added Hunig's base (212 mg, 1.6 mmol, 287 μL). The mixture was heated to 65° C. for 45 min. The reaction cooled and concentrated under reduced pressure. Flash chromatography (ISCO system, silica (24 g), 5-30% ethyl acetate in hexane) provided 1-1 (211 mg, 351 μmol, 85% yield).

Step 2. To a solution of 1-1 (211 mg, 351 μmol) in THF (10.00 mL) was added TBAF (642 mg, 2.46 mmol). The reaction mixture was stirred for 3 hr. The reaction was quenched by addition of saturated NH₄Cl solution (10 mL). The mixture was extracted with DCM (3×15 mL), dried with Na₂SO₄ and concentrated under reduced pressure. Flash chromatography (ISCO system, silica (24 g), 2.5-5% methanol in dichloromethane) provided 1-2 (159 mg, 327 μmol, 93% yield).

Step 3. To as solution of 1-2 (159 mg, 327 μmol) in DMF (6.56 mL) was added KOt-Pent (1.7 M, 771 μL) in toluene. The reaction was heated to 50° C. for 45 min. The reaction was cooled to −20° C. and quenched with saturated NH₄Cl sol (5 mL) then extracted with DCM (3×10 mL). Combined extracts were dried with Na₂SO₄ and concentrated under reduced pressure. Flash chromatography (ISCO system, silica (24 g), 0-60% ethyl acetate in hexane) provided 1-3 (17.8 mg, 43 μmol, 13% yield).

Step 4. To a mixture of 1-3 (17.8 mg, 43 μmol) and (R)-tert-butyl (2-hydroxypropyl)carbamate (11 mg, 63 μmol) and PPh₃ (17 mg, 65.6 μmol) dissolved in DCM (400 μL) was added DIAD (13.7 mg, 67.8 μmol, 13.3 μL) very slowly with mixing. The reaction was warmed to 35° C. and stirred for 2 hr. Flash chromatography (ISCO system, silica (12 g), 0-50% ethyl acetate in hexane) provided 1-4 (14.3 mg, 25 μmol, 57% yield).

Step 5. To a solution of 1-4 (14.3 mg, 25 μmol) in MeOH (3 mL) and THF (1 mL) at ambient temperature was added aqueous LiGH solution (2.0 M, 0.75 mL). The mixture was heated at 70° C. for 4 hours, cooled to −20° C. then quenched with aqueous HCl solution (2.0 M) to acidic. The mixture was extracted with DCM (3×5 mL), dried with Na₂SO₄, concentrated under reduced pressure, and dried under high vacuum. The crude material was dissolved in DCM (4 mL) followed by addition of HCl in 1,4-dioxane (4 M, 3 mL). The mixture was stirred ambient temperature for 30 min, concentrated under reduced pressure, and dried under high vacuum. The crude material was dissolved in in DMF (2.0 mL) and DCM (4.0 mL) and Hunig's base (33 mg, 0.25 mmol, 44 μL) then FDPP (19.5 mg, 50.7 μmol) was added in one portion. The reaction was stirred for 1 hour then quenched with 2 M Na₂CO₃ solution (5 mL). The mixture was stirred for 5 min then extracted with DCM (4×10 mL). Combined extracts were dried with Na₂SO₄ and concentrated under reduced pressure. Flash chromatography (ISCO system, silica (12 g), 0-7.5% methanol in dichloromethane) provided 1 (9.3 mg, 22 μmol, 87% yield).

### General Method F

### Preparation of (4S)-8-fluoro-4-methyl-3,4,13,14-tetrahydro-6H-18,1-(metheno)[1,4]oxazino[3,4-i]pyrazolo[4,3-f][1,4,8,10]benzoxatriazacyclotridecin-15(12H)-one (2)

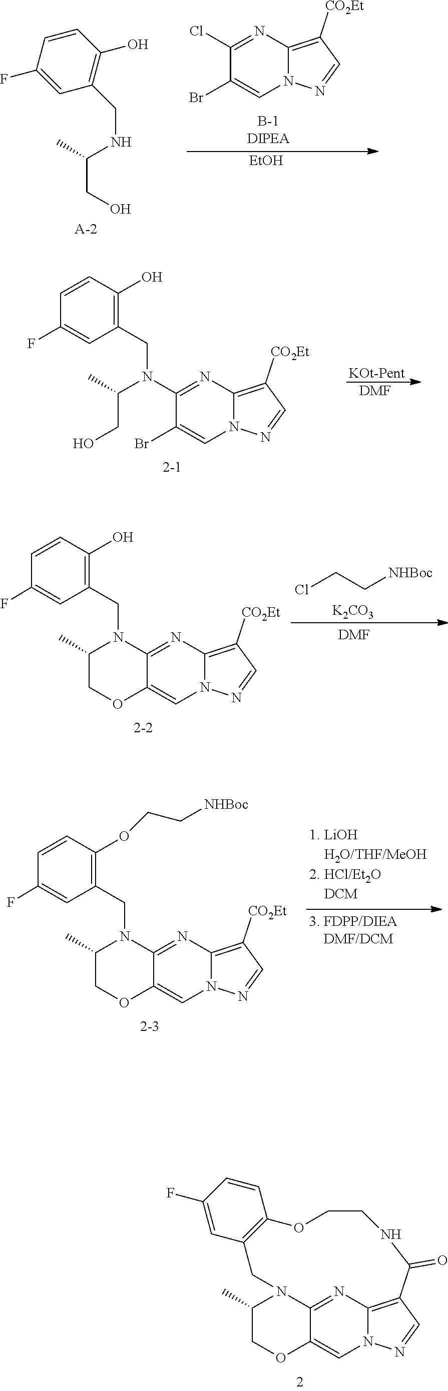

Step 1. To a solution of B-1 (99 mg, 326 μmol) and A-2 (65 mg, 326 μmol) in EtOH (1.6 mL) was added Hunig's base (210 mg, 1.6 mmol, 285 μL). The mixture was heated to 50° C. for 45 min. The reaction cooled and concentrated under reduced pressure. Flash chromatography (ISCO system, silica (24 g), 2.5-7.5% methanol in dichloromethane) provided 2-1 (136.6 mg, 292 μmol, 89% yield).

Step 2. To a solution of 2-1 (136.6 mg, 292 μmol) in DMF (10 mL) was added KOt-Pent (1.7 M, 430 μL) in toluene. The reaction stirred at room temperature for 1.5 hours. The reaction was cooled to −20° C. and quenched with saturated NH₄Cl sol (5 mL) then extracted with DCM (3×10 mL). Combined extracts were dried with Na₂SO₄ and concentrated under reduced pressure. Flash chromatography (ISCO system, silica (24 g), 0-60% ethyl acetate in hexane) provided 2-2 (6.3 mg, 16 μmol, 5% yield).

Step 3. To a solution of to 2-2 (6.3 mg, 16 μmol) and tert-butyl (2-chloroethyl)carbamate (16 mg, 89 μmol, 15 uL) in DMF (500 uL) was added K₂CO₃ (15 mg, 108 μmol). The mixture was heated to 80° C. with stirring for 4 hr. The reaction was cooled and diluted with DCM (3 mL), filtered through a syringe filter and concentrated under reduced pressure. Flash chromatography (ISCO system, silica (12 g), 0-60% ethyl acetate in hexane) provided 2-3 (4.8 mg, 9 μmol, 55% yield).

Step 4. This step was performed in a manner similar to that of step 5 in General Method E to give compound 2 in 98% yield.

Compound 3 was prepared according to General Method F using A-3 in step 1.

### General Method G

### Preparation of (4S,12S)-8-fluoro-4,12-dimethyl-3,4,13,14-tetrahydro-6H-18,1-(metheno)[1,4]oxazino[3,4-i]pyrazolo[4,3-f][1,4,8,10]benzoxatriazacyclotridecin-15(12H)-one (4)

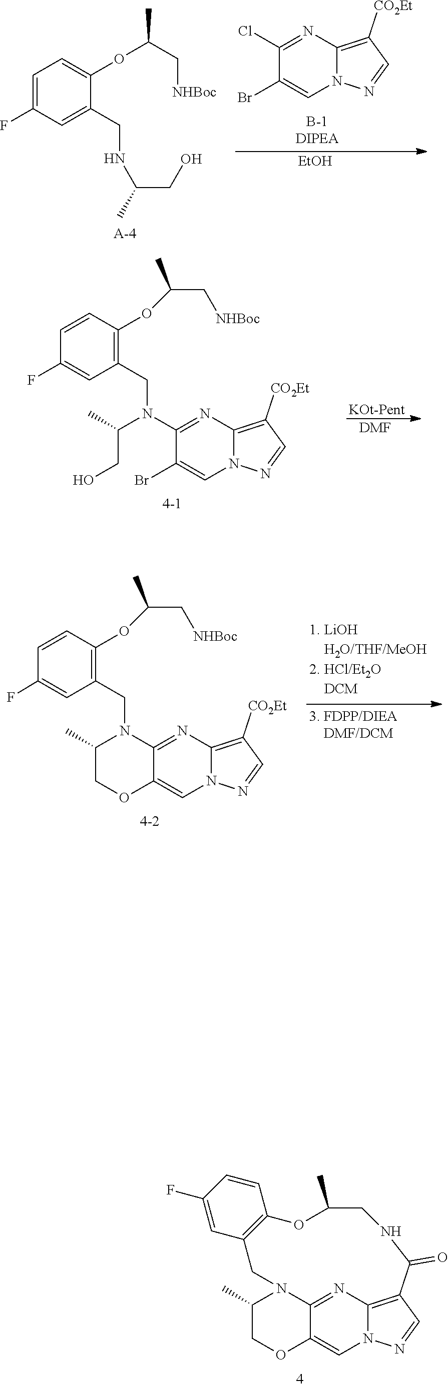

Step 1. To a solution of B-1 (70 mg, 230 μmol) and A-4 (65 mg, 326 μmol) in EtOH (1.6 mL) was added Hunig's base (148 mg, 1.15 mmol, 200 μL). The mixture was heated to 70° C. for 30 hr. The reaction cooled and concentrated under reduced pressure. Flash chromatography (ISCO system, silica (12 g), 10-60% ethyl acetate in hexane) provided 4-1 (75.5 mg, 121 μmol, 52% yield).

Step 2. To a solution of 4-1 (75.5 mg, 121 μmol) in DMF (4.8 mL) was added KOt-Pent (1.7 M, 178 μL) in toluene. The reaction stirred at room temperature for 1.5 hours. The reaction was cooled to −20° C. and quenched with saturated NH₄Cl sol (5 mL) then extracted with DCM (3×10 mL). Combined extracts were dried with Na₂SO₄ and concentrated under reduced pressure. Flash chromatography (ISCO system, silica (12 g), 20-50% ethyl acetate in hexane) provided 4-2 (21.8 mg, 40 μmol, 33% yield).

Step 3. This step was performed in a manner similar to that of step 5 in General Method E to give compound 4 in 77% yield.

Compounds 5 through 6 were prepared according to General Method G using A-5 through A-6 in step 1 respectively.

### General Method H

### Preparation of (4R,12S)-8,10-difluoro-4,12-dimethyl-3,4,13,14-tetrahydro-6H-18,1-(metheno)[1,4]oxazino[3,4-i]pyrazolo[4,3-f][1,4,8,10]benzoxatriazacyclotridecin-15(12H)-one (7)

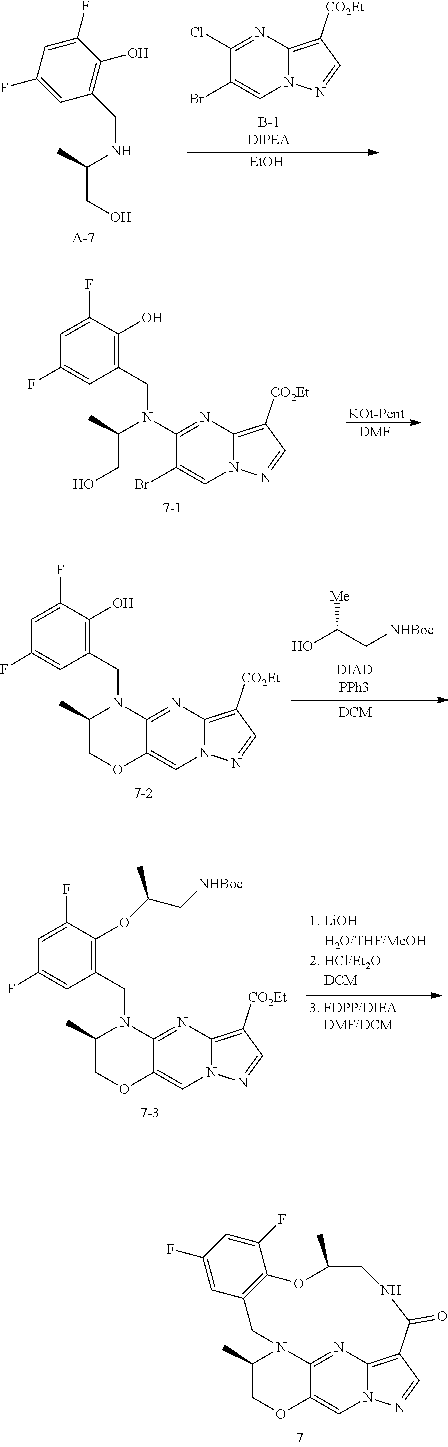

Step 1. To a solution of B-1 (250 mg, 821 μmol) and A-7 (196 mg, 903 μmol) in EtOH (4.1 mL) was added Hunig's base (1.06 g, 8.2 mmol, 1.43 mL). The mixture was heated to 50° C. for 3 hr. The reaction cooled and concentrated under reduced pressure. Flash chromatography (ISCO system, silica (12 g), 20-30% ethyl acetate in hexane) provided 7-1 (56.5 mg, 116 umol, 14% yield).

Step 2. To a solution of 7-1 (56.5 mg, 116 μmol) in DMF (6.0 mL) was added KOt-Pent (1.7 M, 205 μL) in toluene. The reaction stirred at room temperature for 1 hr. The reaction was quenched with saturated NH₄Cl sol (5 mL) then extracted with DCM (3×10 mL). Combined extracts were dried with Na₂SO₄ and concentrated under reduced pressure. Flash chromatography (ISCO system, silica (12 g), 20-60% ethyl acetate in hexane) provided 7-2 (11.2 mg, 27.7 μmol, 23% yield).

Step 3. To a mixture of 7-2 (18 mg, 44 μmol) and (R)-tert-butyl (2-hydroxypropyl)carbamate (9.4 mg, 53 μmol) and PPh₃ (14.6 mg, 55.6 μmol) dissolved in DCM (200 μL) was added DIAD (11.7 mg, 57.8 μmol, 11.3 μL) very slowly with mixing. The reaction was warmed to 35° C. and stirred for 3 hr. Flash chromatography (ISCO system, silica (12 g), 10-50% ethyl acetate in hexane) provided 7-3 (10.5 mg, 18.7 μmol, 42% yield).

Step 4. This step was performed in a manner similar to that of step 5 in General Method E to give compound 7 in 79% yield.

### General Method I

### Preparation of (4R,12S)-8-fluoro-4,12-dimethyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (8)

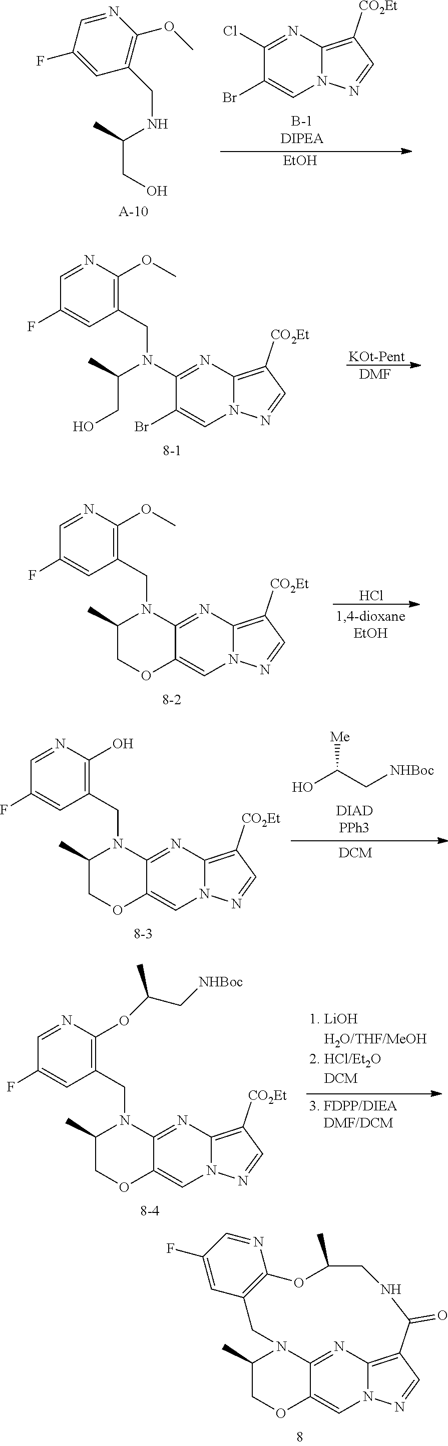

Step 1. To a solution of B-1 (315 mg, 1.04 mmol) and A-8 (222 mg, 1.04 mmol) in EtOH (4.1 mL) was added Hunig's base (1.34 g, 10.3 mmol, 1.8 mL). The mixture was heated to 85° C. for 22 hours. The reaction cooled and concentrated under reduced pressure. Flash chromatography (ISCO system, silica (24 g), 5-50% ethyl acetate in hexane) provided 8-1 (136.7 mg, 283 umol, 27% yield).

Step 2. To a solution of 8-1 (136.7 mg, 283 μmol) in DMF (12 mL) was added KOt-Pent (1.7 M, 500 μL) in toluene. The reaction stirred at room temperature for 45 min. The reaction was quenched with saturated NH₄Cl sol (5 mL) then extracted with DCM (3×10 mL). Combined extracts were dried with Na₂SO₄ and concentrated under reduced pressure. Flash chromatography (ISCO system, silica (12 g), 10-50% ethyl acetate in hexane) provided 8-2 (34.6 mg, 86 μmol, 30% yield).

Step 3. To a solution of 8-2 (34.6 mg, 86 μmol) in EtOH (3.0 mL) was added HCl (4M, 3 mL). The mixture was heated to 75° C. for 3.5 hr. The reaction cooled and concentrated under reduced pressure. Flash chromatography (ISCO system, silica (12 g), 0-10% methanol in dichloromethane) provided 8-3 (20.5 mg, 53 μmol, 61% yield).

Step 4. To a mixture of 8-3 (20.5 mg, 53 μmol) and (R)-tert-butyl (2-hydroxypropyl)carbamate (11.1 mg, 63 μmol) and PPh₃ (17.3 mg, 66 μmol) dissolved in DCM (150 μL) and cooled to −20° C. was added DIAD (13.9 mg, 68.8 μmol, 13.5 μL) very slowly with mixing. The reaction was warmed to room temperature and stirred for 20 hours. Flash chromatography (ISCO system, silica (12 g), 20-60% ethyl acetate in hexane) provided 10-4 (12.3 mg, 22.5 μmol, 42% yield).

Step 5. This step was performed in a manner similar to that of step 5 in General Method E to give compound 8 in 84% yield.

Compound 9 was prepared according to General Method G using A-9 in step 1.

Compound 10 was prepared according to General Method I using A-10 in step 1.

Compound 11 was prepared according to General Method G using A-11 in step 1.

### General Method J

### Preparation of (4R,12S)-8-fluoro-4-(hydroxymethyl)-12-methyl-3,4,13,14-tetrahydro-6H-18,1-(metheno)[1,4]oxazino[3,4-i]pyrazolo[4,3-f][1,4,8,10]benzoxatriazacyclotridecin-15(12H)-one (12)

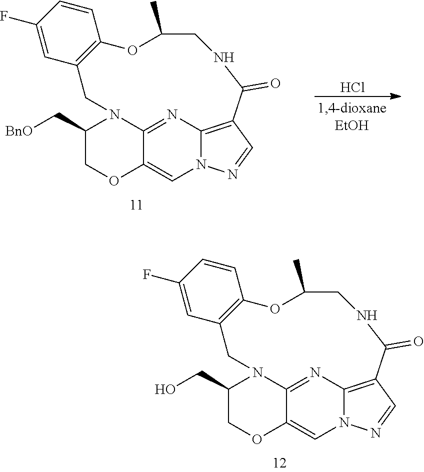 [SMILES: C[C@H]1CNC(=O)c2cnn3cc4c(nc23)N(Cc2cc(F)ccc2O1)[C@H](COCc1ccccc1)CO4 ; Cl ; C[C@H]1CNC(=O)c2cnn3cc4c(nc23)N(Cc2cc(F)ccc2O1)[C@H](CO)CO4]

Step 1. To a solution of 11 (8.3 mg, 16 μmol) in EtOH (4.0 mL) was added HCl (4M, 4 mL) in dioxane. The mixture was heated to 80° C. for 6 days. The reaction cooled and concentrated under reduced pressure. Flash chromatography (ISCO system, silica (12 g), 0-7.5% methanol in dichloromethane) provided 12 (1.24 mg, 3 μmol, 18% yield).

Compound 13 was prepared according to General Method G using A-12 in step 1.

### General Method K

### Preparation of (4R,13R)-8-fluoro-4,13-dimethyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (14)

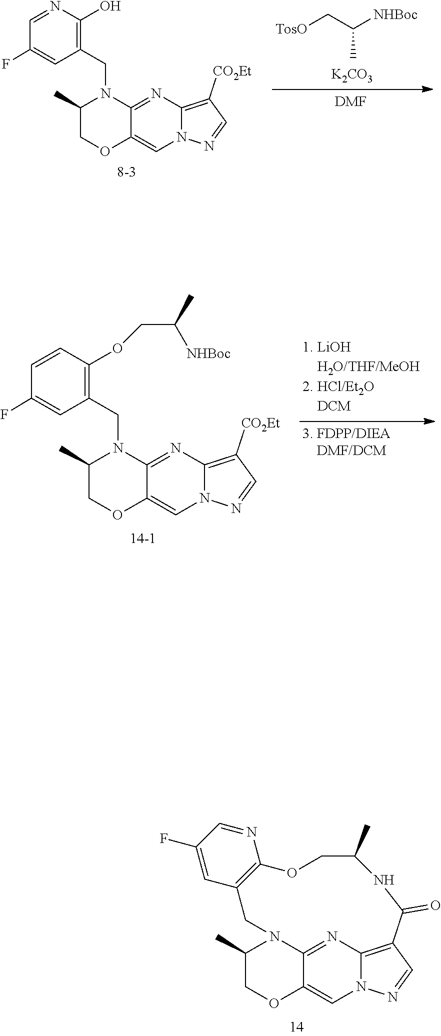

Step 1. To a solution of to 8-3 (17.1 mg, 44 μmol) and (2R)-2-[(tert-butoxycarbonyl)amino]propyl 4-methylbenzene-1-sulfonate (72.7 mg, 220 μmol) in DMF (1 mL) was added K₂CO₃ (36.3 mg, 264 μmol). The mixture was heated to 80° C. with stirring for 15 hours. The reaction was cooled and quenched with water (5 mL) then extracted with DCM (3×10 mL). Combined extracts were dried with Na₂SO₄ and concentrated under reduced pressure. Flash chromatography (ISCO system, silica (12 g), 20-100% ethyl acetate in hexane) provided 14-1 (13.6 mg, 25 μmol, 56% yield).

Step 2. This step was performed in a manner similar to that of step 5 in General Method E to give compound 14 in 86% yield.

Compound 15 was prepared using the procedures of General Method I and General Method K starting with A-10 in step 1.

Compound 16 through 20 were prepared according to General Method G using A-13 through A-17 respectively.

### General Method L

### Preparation (5R,13S)-9-fluoro-5,13-dimethyl-4,5,14,15-tetrahydro-3H,7H-19,1-(metheno)[1,4]oxazepino[3,4-i]pyrazolo[4,3-f]pyrido[3,2-l][1,4,8,10]oxatriazacyclotridecin-16(13H)-one (21)

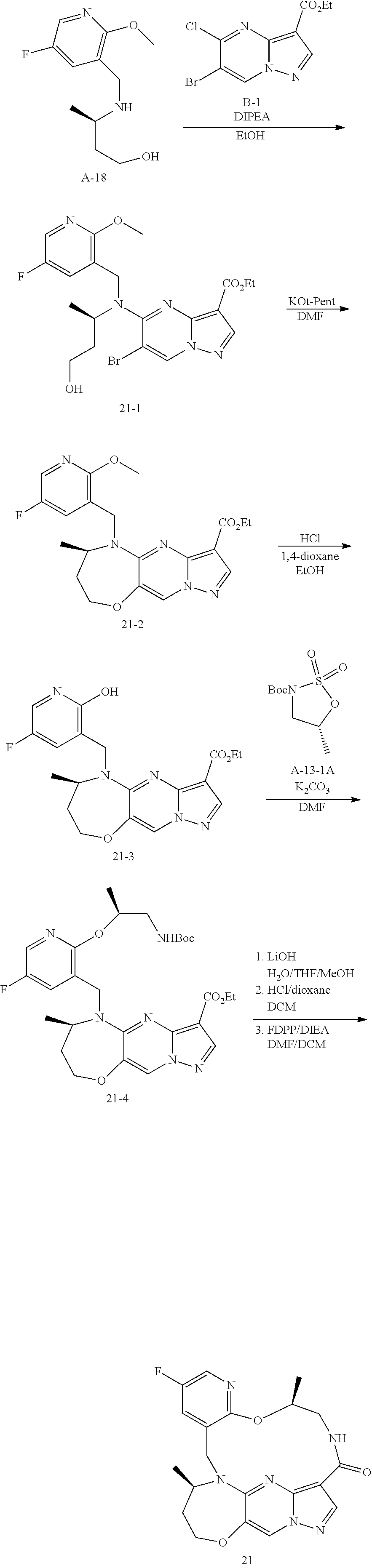

Steps 1 through 3 were performed according to General Method I.

Step 4. Added K₂CO₃ (50 mg, 361 μmol) to 21-3 (14.5 mg, 36.1 μmol) and A-3-1A (43 mg, 180 μmol) in DMF (750 μL) and stirred for 15 minutes. The reaction was quenched reaction with 1M citric acid sol (3 mL) and stirred for 15 minutes. The mixture was extracted with DCM (3×3 mL). Combined extracts were dried with Na₂SO₄ and concentrated under reduced pressure. Flash chromatography (ISCO system, silica (12 g), 0-100% ethyl acetate in hexane) provide 21-4 (16.9 mg, 84% yield).

Step 5. This step was performed in a manner similar to that of step 5 in General Method E to give compound 21 in 79% yield.

Compound 22 was prepared according to General Method L using (R)-3-Boc-4-methyl-2,2-dioxo-[1,2,3]oxathiazolidine in step 4.

Compound 23 was prepared according to General Method G using A-19 in step 1.

Compound 24 was prepared according to General Method I.

### General Method M

### Preparation of Ethyl 5-chloro-6-hydroxy-pyrazolo[1,5-a]pyrimidine-3-carboxylate (C-1)

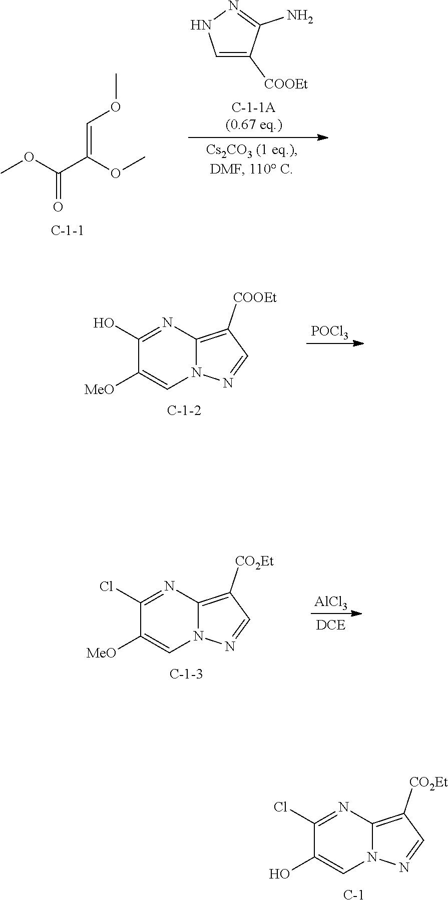

Step 1. To a solution of ethyl 3-amino-1H-pyrazole-4-carboxylate (15.0 g, 96.7 mmol, 1.00 eq.) in dimethyl formamide (250 mL) was added cesium carbonate (47.3 g, 145 mmol, 1.50 eq.) and methyl (E)-2,3-dimethoxyprop-2-enoate (21.2 g, 145 mmol, 1.50 eq.). The mixture was stirred at 110° C. for 12 hours. The reaction mixture was diluted with water (1.00 L). Hydrochloric acid (5.00 M, 50.0 mL) was added to the mixture slowly at 20° C., and yellow solid precipitate out. The mixture was filtered and filter cake washed with methanol (50.0 mL). The filter cake was concentrated under reduced pressure to give crude product C-1-2 (13.5 g, crude) as a yellow solid.

Step 2. C-1-2 (9.50 g, 28.8 mmol, 1.00 eq.) was added to phosphorus oxychloride (140 mL). The mixture was stirred at 110° C. for 12 hours. The reaction mixture was concentrated under reduced pressure to remove solvent and precipitate out. The residue was diluted with ice water (80.0 mL), filtered to remove the solvent. Then the filter cake was added to the solution of dichloromethane (300 mL) and water (200 mL). The mixture was stirred at 20° C. for 10 mins then partitioned between water and dichloromethane. The organic phase was separated, washed with brine (300 mL), dried over anhydrous sodium sulfate, filtered and the filtrate was concentrated under reduced pressure to give a residue. The residue was purified by column chromatography (SiO₂, Petroleum ether/Ethyl acetate=10/1 to 1:1) to give compound C-1-3 (5.10 g, 19.6 mmol, 67.9% yield, 98.2% purity) as a white solid. ¹H NMR (400 MHz, CDCl₃) δ=8.47 (s, 1H), 8.27 (s, 1H), 4.42 (q, J=7.2 Hz, 2H), 3.99 (s, 3H), 1.42 (t, J=7.2 Hz, 3H). Step 3. Aluminium trichloride (33.4 g, 250 mmol, 13.7 mL, 8.00 eq.) was added in one portion to anhydrous dichloroethane (120 mL) and the mixture was stirred under nitrogen at 20° C. for 10 min, then C-1-3 (8.00 g, 31.3 mmol, 1.00 eq.) was added to the mixture in five equal portions. The mixture was stirred at 20° C. for 24 hours. The reaction mixture was quenched by addition hydrochloric acid (100 mL, 3.00 M) at 0° C. Then the mixture was diluted with water (50.0 mL) and extracted with ethyl acetate (300 mL×2). The combined organic layers were washed with brine (50.0 mL×2), dried over anhydrous sodium sulfate, filtered and concentrated under reduced pressure to give a residue. The residue was purified by column chromatography (SiO₂, Petroleum ether/Ethyl acetate=20/1 to 2:1) to give compound C-1 (5.90 g, 21.7 mmol, 69.5% yield, 89.0% purity) as a gray solid. ¹H NMR (400 MHz, CDCl₃) δ=8.50 (s, 1H), 8.45 (s, 1H), 4.41 (q, J=7.2 Hz, 2H), 1.42 (t, J=7.2 Hz, 3H).

### General Method N

### Preparation of Ethyl 5-chloro-6-fluoropyrazolo[1,5-a]pyrimidine-3-carboxylate (C-2)

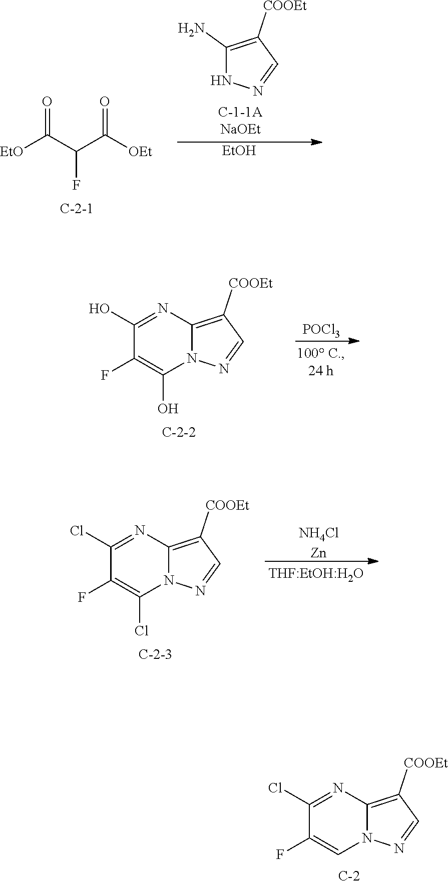

Step 1. To a solution of C-1-1A (5.0 g, 28.1 mmol, 1 eq.) and C-2-1 (6.1 g, 39.3 mmol, 1.4 eq.) in EtOH (56 mL) at 90° C. was added NaOEt (2.68 M, 26.2 mL, 2.5 eq.) and was stirred for 6 hours. The reaction mixture cooled and diluted with Toluene (60 mL) and concentrated to dryness under reduced pressure. The material was resuspended in Toluene (60 mL) and again concentrated to dryness and placed on a high vac overnight to provide crude C-2-2. Crude material was used as is in next step.

Step 2. The crude C-2-2 from step 1 was suspended in POCl₃ (99 g, 60 mL, 646 mmol, 23.00 eq.) and heated to 100° C. for 24 hours. The reaction was cooled to room temperature and concentrated to dryness under reduced pressure. The crude material was suspended in DCM (100 mL) and water (100 mL) was added. The mixture was stirred for 30 min then extracted with DCM (3×100 mL). The combined organic extracts were washed by brine (100 mL), dried over anhydrous sodium sulfate, filtered and concentrated under reduced pressure. Purification through a Silica plug (60 g Si), eluted with DCM (˜1.5 L) gave C-2-3 (5.68 g, 72% yield, purity=86% by LC/MS) as a yellow solid.

Step 3. To a solution of C-2-3 (5.68 g, 20.4 mmol) and NH₄Cl (5.46 g, 102 mmol) in THF (68 mL), EtOH (204 mL) and water (136 mL) at 0° C. was added Zn powder (5.34 g, 81.7 mmol). The mixture was stirred at 0° C. for 3 hours. The reaction mixture was filtered through a celite pad and the celite pad was rinsed with DCM (100 mL). The filtrate was concentrated to dryness under reduced pressure then resuspended in DCM (500 mL) dried with Na₂SO₄ and concentrated under reduced pressure. Purification using a silica plug (50 g Si) and elution with DCM provided C-2 (3.17 g, 63.8% yield) as a white solid.

### General Method O

### Preparation of Ethyl 5-chloro-6-hydroxypyrazolo[1,5-a]pyrimidine-3-carboxylate (D-1)

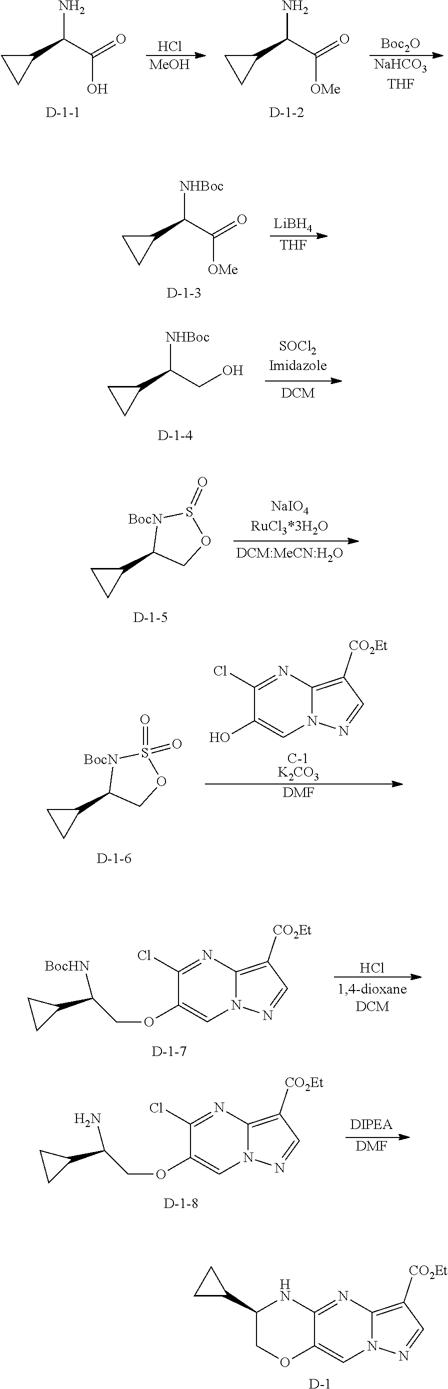

Step 1. To a solution of D-1-1 (1.0 g, 8.69 mmol) in dry MeOH (87 mL) was added HCl (4.0 M, 4.3 mL, 2.0 eq.) in dioxane. The mixture was heated to 70° C. and stirred for 40 hours. The reaction mixture cooled and concentrated to dryness under reduced pressure to provide crude D-1-2. The material was used as is in next step.

Step 2. To a solution of crude D-1-2 from step 1 in THF (60 mL) was added Boc₂O (2.08 g, 9.54 mmol) and NaHCO₃ solution (1 M, 34.69 mL). The reaction was stirred for 4 hours then diluted with water (50 mL) and then extracted with ethyl acetate (3×50 mL). The combined organic extracts were washed by brine (50 mL), dried over anhydrous sodium sulfate, filtered and concentrated under reduced pressure. Flash chromatography (ISCO system, silica (40 g), 0-50% ethyl acetate in hexane) provided D-1-3 (1.66 g, 83% yield).

Step 3. To a solution of D-1-3 (1.66 g, 7.24 mmol) in THF (36 mL) at 0° C. was added LiBH₄ (789 mg, 36 mmol). The mixture was slowly warmed to room temperature and stirred for 20 hours. The reaction mixture was quenched by addition of water (20 mL) and aqueous saturated NH₄Cl (25 mL) then extracted with ethyl acetate (3×50 mL). Combined extracts were dried with brine (50 mL), Na₂SO₄ and concentrated under reduced pressure. Flash chromatography (ISCO system, silica (40 g), 10-40% ethyl acetate in hexane) provide D-1-4 (1.23 g, 84% yield).

Step 4. To a solution of Imidazole (1.0 g, 14.9 mmol) in DCM (16 mL) at −5° C. was added SOCl₂ (532 mg, 4.47 mmol, 324 μL) in DCM (5 mL) dropwise. The mixture was stirred at −5° C. for 1 hour. The mixture was cooled to −10° C. and D-1-4 (0.5 g, 2.48 mmol) in DCM (4 mL) was added dropwise. The mixture was slowly warmed to 10° C. and stirred at this temperature for 2 hr. The reaction was quenched with water (10 mL) and stirred at 10° C. for 10 min. The organic layer was removed and washed with 10% citric acid solution (10 mL) then dried with brine (5 mL) and Na₂SO₄ and concentrated under reduced pressure. Flash chromatography (ISCO system, silica (24 g), 0-20% ethyl acetate in hexane) provide D-1-5 (294 mg, 48% yield).

Step 5. To a solution of D-1-5 (294 mg, 1.19 mmol) in DCM (5.66 mL) and NaIO4 (610.25 mg, 2.85 mmol) in H2O (5.66 mL) at 0° C. was added RuCl₃*3H₂O (6.2 mg, 24 μmol). The reaction was warmed to room temperature and stirred for 1 hour. The reaction was quenched with water (15 mL) then extracted with DCM (3×15 mL). Combined extracts were dried with brine (5 mL), Na₂SO₄ and concentrated under reduced pressure. Flash chromatography (ISCO system, silica (12 g), 0-30% ethyl acetate in hexane) provide D-1-6 (308 mg, 98% yield).

Step 6. To a solution of C-1 (75 mg, 310 μmol) and D-1-6 (102 mg, 388 μmol) in DMF (1.6 mL) was added K₂CO₃ (107 mg, 776 μmol). The mixture was stirred for 1 hour then quenched with 1M citric acid sol (10 mL), MeOH (5 mL) and THF (5 mL) and the mixture stirred for 3 hours. The mixture was extracted with DCM (3×15 mL). Combined extracts were dried with Na₂SO₄ and concentrated under reduced pressure to provided crude D-1-7.

Step 7. The crude D-1-7 material from the previous step was dissolved in DCM (5 mL) followed by addition of HCl in 1,4-dioxane (4 M, 6 mL). The mixture was stirred ambient temperature for 30 min, concentrated under reduced pressure, and dried under high vacuum to provide crude D-1-8.

Step 8. To a solution of crude D-1-8 in DMF (10 mL) was added Hunig's base (1.34 g, 10.3 mmol, 1.8 mL). The mixture was stirred for 15 minutes then concentrated under reduced pressure. Flash chromatography (ISCO system, silica (12 g), 0-5% methanol in dichloromethane) provided D-1 (121 mg, compound used as is).

Compound D-2 was prepared according to General Method O using tert-butyl [1-(hydroxymethyl)cyclopropyl]carbamate in step 4.

### General Method P

### Preparation of Ethyl (7S)-7-methyl-5,6,7,8-tetrahydropyrazolo[1′,5′:1,2]pyrimido[5,4-b][1,4]oxazepine-3-carboxylate (D-3)

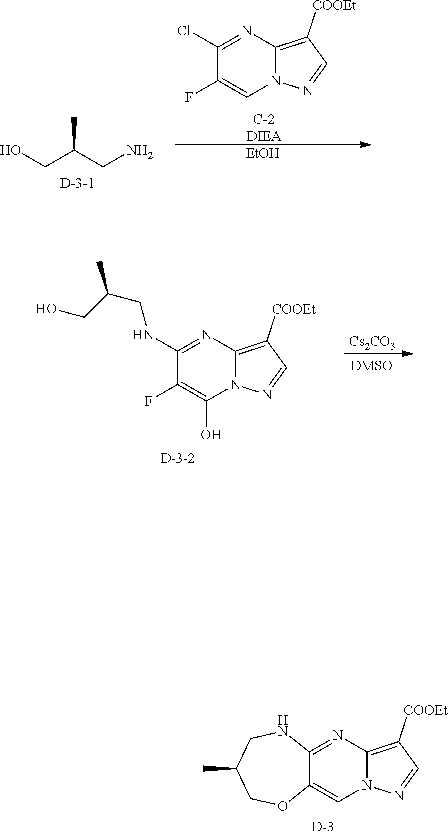

Step 1. To a solution of C-2 (200 mg, 821 μmol) and D-3-1 (77 mg, 862 μmol) in EtOH (4 mL) was added DIEA (106 mg, 143 μL 821 μmol). The mixture was heated to 80° C. for 30 minutes. The reaction cooled and concentrated under reduced pressure. Flash chromatography (ISCO system, silica (24 g), 0-80% ethyl acetate in hexane) provided D-3-2 (213 mg, 87% yield).

Step 2. To a solution of D-3-2 (202 mg, 682 μmol) in DMSO (34 mL) was added Cs₂CO₃ (2.22 g, 6.8 mmol). The mixture was heated to 150° C. and stirred for 12 hours. The reaction mixture was cooled and quenched with 30% brine solution (600 mL) then extracted with ethyl acetate (3×150 mL). Organic extracts were combined and dried Na₂SO₄ and concentrated under reduced pressure. Flash chromatography (ISCO system, silica (12 g), 0-5% methanol in dichloromethane) provided D-3 (17.6 mg, 9% yield).

Compounds D-4 and D-5 were prepared according to General Method P.

### General Method Q

### Preparation of Ethyl (3R)-3-(hydroxymethyl)-3,4-dihydro-2H-pyrazolo[1′,5′:1,2]pyrimido[5,4-b][1,4]oxazine-6-carboxylate (D-6)

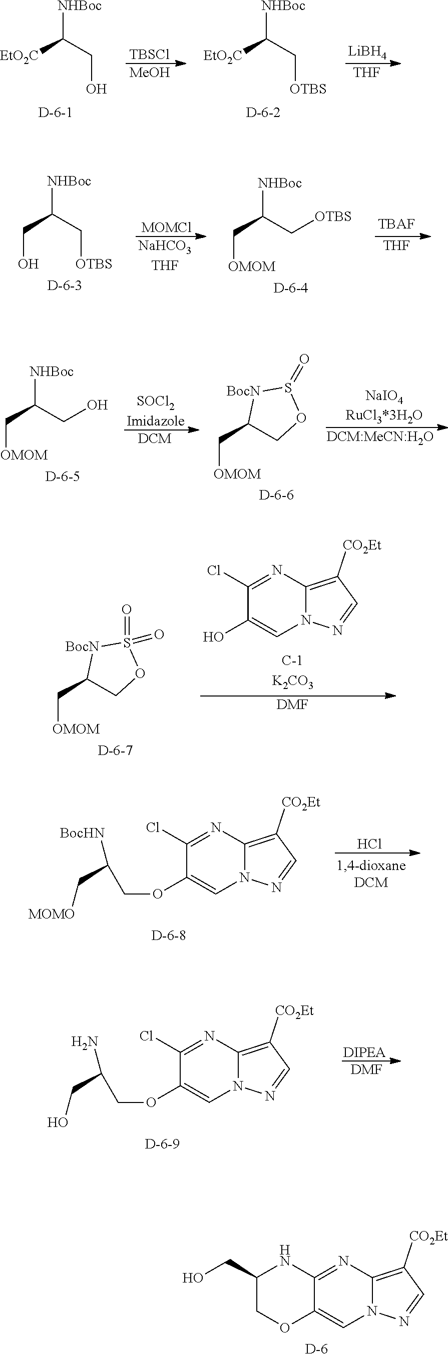

Step 1. To a solution of D-6-1 (5.03 g, 21.6 mmol) and imidazole (3.67 g, 54 mmol) in DMF (43 mL) was added TBSCl (3.9 g, 26 mmol), the reaction was stirred under room temperature for 18 hours, reaction was quenched by water (100 mL) then extracted by DCM (3×50 mL). Combined extracts were washed with water (50 mL), brine (50 mL), dried with Na2SO4 and concentrated under reduced pressure. Flash column chromatography (ISCO system, silica (120 g), 0-35% ethyl acetate in hexanes) afforded D-6-2 (6.1 g, 81% yield).

Step 2. To a solution of D-6-2 (1.02 g, 2.94 mmol) in THF (14.39 mL) under argon at 0° C. was added LiBH₄ (192.29 mg, 8.83 mmol) and the mixture was stirred warming to ambient temperature over 72 hr. Reaction was cooled in an ice bath and quenched carefully with 2N NaOH (4 mL) followed by water (10 mL), then again with 2N NaOH (6 mL), while stirring vigorously. DCM (20 mL) was added and layers were separated. The aqueous layer was extracted twice more with DCM (2×10 mL) and the combined organic layer was washed with brine and dried over sodium sulfate. Flash column chromatography (ISCO, 24 g silica, 0-50% EtOAc in hexanes) afforded D-6-3 (797.3 mg, 2.61 mmol, 88.70% yield).

Step 3. Compound D-6-3 (797.3 mg, 2.61 mmol) was dissolved in DCM (13.05 mL) and MOM chloride (315.20 mg, 3.91 mmol, 297.36 uL) was added followed by DIEA (1.01 g, 7.83 mmol, 1.36 mL). The mixture was stirred at 22° C. for 18 hr. Water (20 mL) was added and the layers were partitioned. The aqueous layer was extracted twice more with DCM (2×10 mL). The combined organic layer was washed with brine and dried over sodium sulfate. Salts were filtered and volatiles were carefully removed under reduced pressure and the resulting crude was purified by flash column chromatography (ISCO, 24 g silica, 0-50% EtOAc in Hexanes) provided D-6-4 (645.8 mg, 1.85 mmol, 70.79% yield). 1H NMR (500 MHz, DMSO-d6) δ ppm 6.60 (br d, J=8.02 Hz, 1H) 4.53 (s, 2H) 3.36-3.66 (m, 5H) 3.24 (s, 3H) 1.37 (s, 9H) 0.86 (s, 9H) 0.03 (s, 6H).

Step 4. Compound D-6-4 (645 mg, 1.85 mmol) was dissolved in THF (9.23 mL) and cooled to 0° C. TBAF (964.95 mg, 3.69 mmol) was added and the mixture was stirred for 2 hr, warming to room temperature. Quenched with water (20 mL) and extracted with DCM (3×20 mL). Flash column chromatography (ISCO, 12 g, silica, EtOAc in Hexanes) afforded D-6-5 (425.3 mg, 1.81 mmol, 97.96% yield). 1H NMR (500 MHz, DMSO-d6) δ ppm 6.52-6.56 (m, 1H) 4.64 (br t, J=5.44 Hz, 1H) 4.53 (s, 2H) 3.51-3.57 (m, 1H) 3.47 (br dd, J=9.45, 6.01 Hz, 1H) 3.36-3.40 (m, 2H) 3.24 (s, 3H) 1.37 (s, 9H).

Steps 5 through 8 were performed according to General Method O.

Compounds D-7 and D-8 were prepared according to General Method P.

### General Method R

### Preparation of Ethyl (7S)-7-hydroxy-5,6,7,8-tetrahydropyrazolo[1′,5′:1,2]pyrimido[5,4-b][1,4]oxazepine-3-carboxylate (D-9)

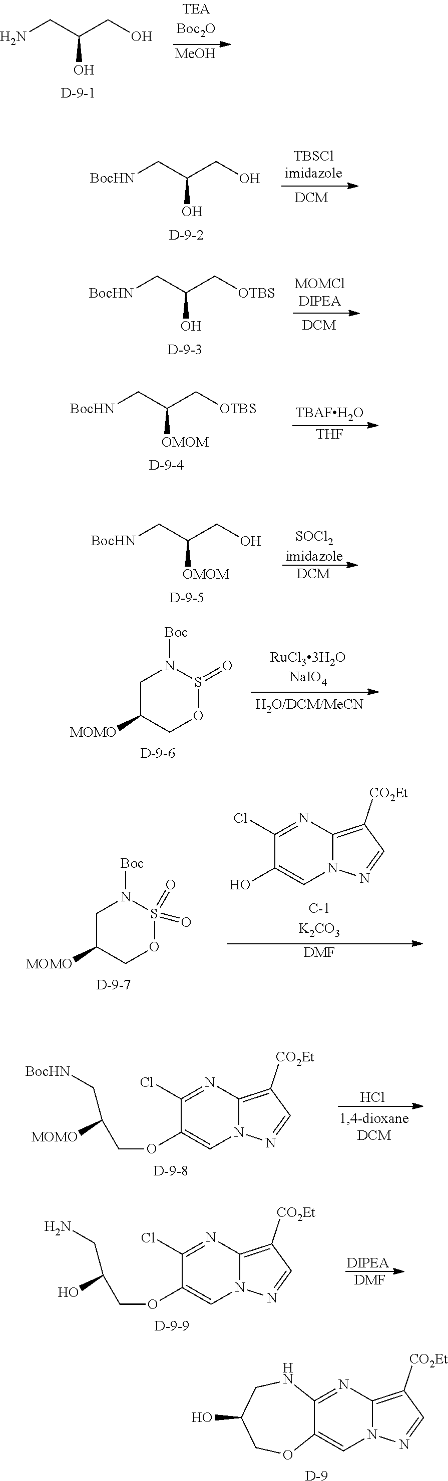

Step 1. To a solution of D-9-1 (3.1 g, 34.03 mmol) and Boc₂O (7.43 g, 34.03 mmol) in MeOH (68.05 mL) was added triethylamine (6.89 g, 68.05 mmol, 9.48 mL), the reaction was stirred under room temperature for 16 hours, reaction was concentrated under reduced pressure. Flash column chromatography (ISCO system, silica (80 g), methanol in DCM) afforded D-9-2 (6.36 g, 33.26 mmol, 97.75% yield).

Step 2. To a solution of D-9-2 (6.36 g, 33.26 mmol) and imidazole (4.53 g, 66.52 mmol) in THF (110.86 mL) was added TBSCl (6.02 g, 39.91 mmol), the reaction was stirred under room temperature for 2 hours, reaction was quenched by water (200 mL) then extracted by DCM (3×200 mL). Combined extracts were dried with Na2SO4 and concentrated under reduced pressure. Flash column chromatography (ISCO system, silica (80 g), ethyl acetate in hexanes) afforded D-9-3 (8.75 g, 28.64 mmol, 86.12% yield).

Step 3. To a solution of D-9-3 (8.75 g, 28.64 mmol) and DIPEA (11.11 g, 85.93 mmol, 14.97 mL) in DCM (95.48 mL) under 0° C. was slowly added MOMCl (3.46 g, 42.96 mmol, 3.26 mL), the reaction was allowed to slowly warm up to room temperature while stirring overnight, reaction was quenched by water (100 mL) then extracted by DCM (3×100 mL). Combined extracts were dried with Na₂SO₄ and concentrated under reduced pressure. Flash column chromatography (ISCO system, silica (80 g), ethyl acetate in hexanes) afforded D-9-4 (7.44 g, 21.29 mmol, 74.31% yield).

Step 4. To a solution of D-9-4 (7.44 g, 21.29 mmol) in THF (106.43 mL) was added TBAF monohydrate (11.90 g, 42.57 mmol), the reaction was stirred under room temperature for 1 hour, reaction was quenched by saturated NH₄Cl solution (100 mL) then extracted by DCM (3×200 mL). Combined extracts were dried with Na₂SO₄ and concentrated under reduced pressure. Flash column chromatography (ISCO system, silica (80 g), ethyl acetate in hexanes) afforded D-9-5 (4.67 g, 19.85 mmol, 93% yield).

Steps 5 through 9 were performed according to General Method O.

Compounds D-10 through D-12 were prepared according to General Method P.

Compounds D-13 was prepared according to General Method O.

Compounds D-14 was prepared according to General Method R.

### General Method S

### Preparation of Ethyl (7R)-7-fluoro-5,6,7,8-tetrahydropyrazolo[1′,5′:1,2]pyrimido[5,4-b][1,4]oxazepine-3-carboxylate (D-15)

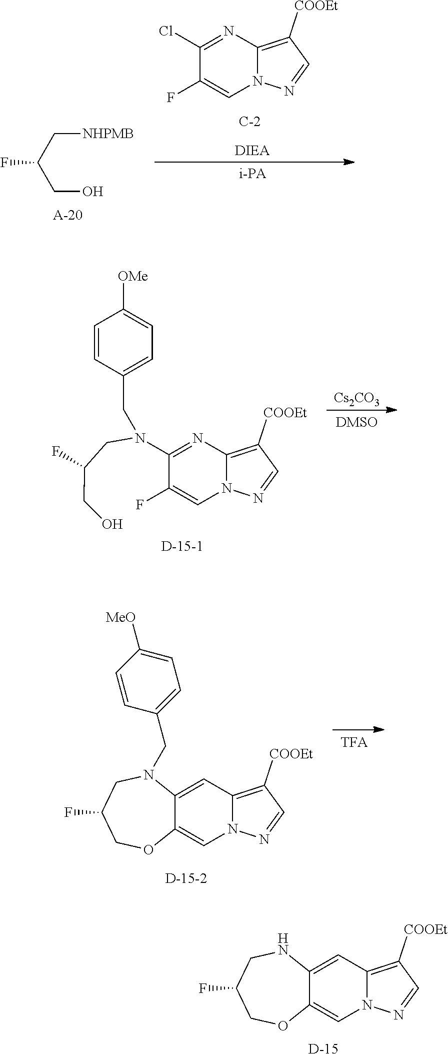

Step 1: To a solution of A-20 (166.3 mg, 780 μmol) and C-2 (190 mg, 780 μmol) in isopropyl alcohol (3.9 mL) was added DIPEA (403 mg, 545 μL). The mixture was stirred at 90° C. for 3 hours. The reaction mixture was concentrated under reduced pressure. Flash chromatography (ISCO system, silica (24 g), 10-50% EtOAc in hexanes) to afford D-15-1 (211.5 mg, 65% yield).

Step 3. To a solution of D-15-1 (211.5 mg, 503 μmol) in DMSO (25 mL) was added Cs₂CO₃ (983 mg, 3.0 mmol) and the mixture stirred at room temperature for 1 hour. The reaction mixture was quenched with 30% brine (150 mL) and extracted with EtOAc (150 mL). The organic layer was washed with 30% brine solution (3×75 mL), dried over Na₂SO₄ and concentrated under reduced pressure. Flash chromatography (ISCO system, silica (24 g), 0-50% EtOAc in hexanes) provided D-15-2 (163.5 mg, 81% yield).

Step 4. D-15-2 (163.5 mg, 408 μmol) was dissolved in TFA (27 mL) and stirred at 70° C. for 3 hours, the reaction was cooled to room temperature and TFA was removed under reduced pressure. The residue was loaded onto a column with DCM and triethylamine and purified by flash column chromatography (ISCO system, silica (24 g), 0-50% EtOAc in Hexanes) to afford D-15 (112.2 mg, 98% yield).

| | | MS |
|---|---|---|
| | | [M + H] |
| Compd# | Structure | m/z |
| D-1 | 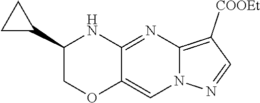 [SMILES: CCOC(=O)c1cnn2cc3c(nc12)N[C@H](C1CC1)CO3] | 289.0 |
| D-2 | 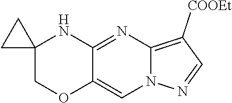 [SMILES: CCOC(=O)c1cnn2cc3c(nc12)NC1(CC1)CO3] | 275.1 |
| D-3 | 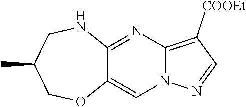 [SMILES: CCOC(=O)c1cnn2cc3c(nc12)NC[C@H](C)CO3] | 277.0 |
| D-4 | 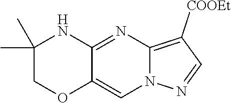 [SMILES: CCOC(=O)c1cnn2cc3c(nc12)NC(C)(C)CO3] | 277.0 |
| D-5 | 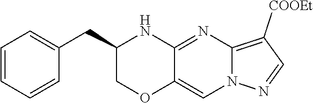 [SMILES: CCOC(=O)c1cnn2cc3c(nc12)N[C@H](Cc1ccccc1)CO3] | 339.1 |
| D-6 | 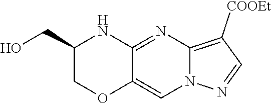 [SMILES: CCOC(=O)c1cnn2cc3c(nc12)N[C@H](CO)CO3] | 278.9 |
| D-7 | 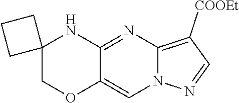 [SMILES: CCOC(=O)c1cnn2cc3c(nc12)NC1(CCC1)CO3] | 289.0 |
| D-8 | 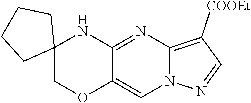 [SMILES: CCOC(=O)c1cnn2cc3c(nc12)NC1(CCCC1)CO3] | 303.1 |
| D-9 | 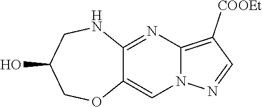 [SMILES: CCOC(=O)c1cnn2cc3c(nc12)NC[C@H](O)CO3] | 279.0 |
| D-10 | 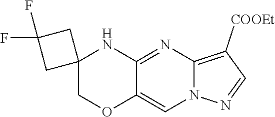 [SMILES: CCOC(=O)c1cnn2cc3c(nc12)NC1(CO3)CC(F)(F)C1] | 325.0 |
| D-11 | 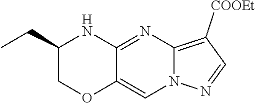 [SMILES: CCOC(=O)c1cnn2cc3c(nc12)N[C@H](CC)CO3] | 276.9 |
| D-12 | 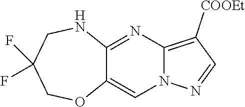 [SMILES: CCOC(=O)c1cnn2cc3c(nc12)NCC(F)(F)CO3] | 299.0 |
| D-13 | 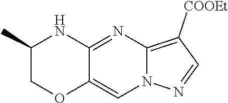 [SMILES: CCOC(=O)c1cnn2cc3c(nc12)N[C@H](C)CO3] | |
| D-14 | 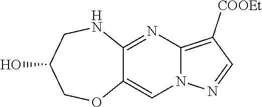 [SMILES: CCOC(=O)c1cnn2cc3c(nc12)NC[C@@H](O)CO3] | 279.1 |
| D-15 | 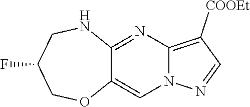 [SMILES: CCOC(=O)c1cnn2cc3c(nc12)NC[C@@H](F)CO3] | 281.1 |

### General Method T

### Preparation of (S)-(2-((1-((tert-butoxycarbonyl)amino)propan-2-yl)oxy)-5-fluoropyridin-3-yl)methyl methanesulfonate (E-1)

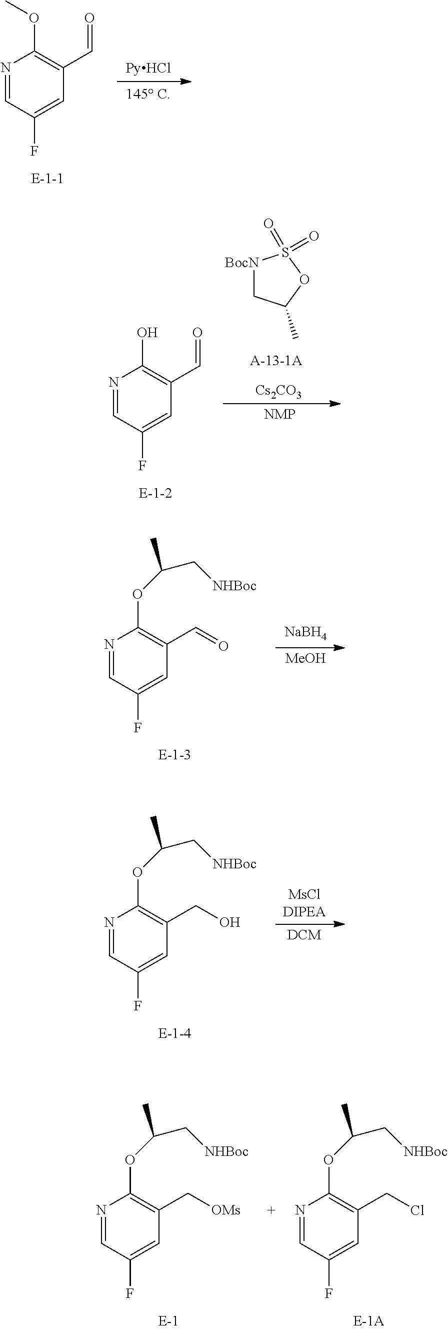

Step 1. E-1-1 (7 g, 45.12 mmol) and pyridine hydrochloride (20.86 g, 180.5 mmol) were mixed in a round bottom flask and heated up to 145° C. and the molten mixture was stirred at 145° C. for 30 min then cooled down. The mixture was diluted with H₂O (200 mL) and ethyl acetate (200 mL), partitioned and the aqueous layer was extracted with EA (5×100 mL), organic phases were combined and dried over Na₂SO₄, the solution was then concentrated under reduced pressure to afford desired product E-1-2 (5.19 g, 36.78 mmol, 81.51% yield) as yellow solid.

Step 2. To an ice-bathed mixture of compound E-1-2 (2.37 mg, 16.79 mmol) and Cs₂CO₃ (21.88 g, 67.15 mmol in NMP (33.57 mL) was added compound A-13-1A (4 g, 16.79 mmol), the reaction was stirred at 0° C. for 2 hours. The reaction was diluted with dichloromethane (200 mL) and H₂O (100 mL). Citric acid solution (1 M in H₂O, 100 mL) was added and the mixture was vigorously stirred for 10 minutes, layers were separated, organic layer was collected and dried over Na₂SO₄, filtered and concentrated under reduced pressure. Purification by flash chromatography (ISCO system, silica (80 g), 0-30% ethyl acetate in hexanes) afforded desired product E-1-3 (4.2 g, 14.06 mmol, 83.79% yield) as white solid.

Step 3. To an ice-bathed solution of compound E-1-3 (4.2 g, 14.06 mmol) in MeOH (46.88 mL) was added NaBH₄ (798.17 mg, 21.10 mmol). The reaction was stirred under 0° C. for 1 hour. The reaction was quenched with H₂O (100 mL) and was extracted with dichloromethane (3×100 mL). The organic phases were combined and dried over Na₂SO₄, filtered and concentrated under reduced pressure. Purification by flash chromatography (ISCO system, silica (80 g), 0-50% ethyl acetate in hexanes) afforded desired product E-1-4 (3.46 g, 11.53 mmol, 81.96% yield) as colorless oil.

Step 4. To a solution of E-1-4 (2.41 g, 8.02 mmol) and the DIPEA (4.15 g, 5.6 mL, 32.1 mmol) in DCM (14 mL) at 0° C. was added MsCl (1.10 g, 0.74 mL 9.62 mmol) dropwise. The mixture stirred at 0° C. for 2 hours. The was quenched with 1% HCl solution (100 mL) and extracted with DCM (3×100 mL). The organic phases were combined, dried over Na2SO4, and concentrated under reduced pressure. Flash chromatography (ISCO system, silica (80 g), 0-40% ethyl acetate in hexane) provided E-1(2.0 g, 66% yield) as a white solid and E-1A (627 mg, 24% yield) as an oil.

Compound E-2 was prepared according to General Method T using (R)-3-boc-4-methyl-2,2-dioxo-[1,2,3]oxathiazolidine in step 2.

Compound E-3 and E-4 were prepared according to General Method T.

### General Method U

### Preparation of 3-(1-chloroethyl)-5-fluoro-2-methoxypyridine (E-5)

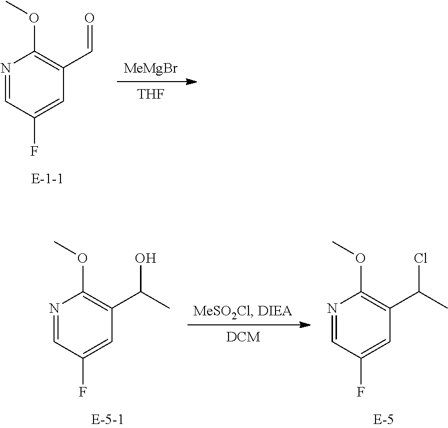

Step 1. To a solution of E-1-1 (1 g, 6.45 mmol) in THF (32.23 mL) was added MeMgBr (3 M, 6.45 mL) in Et₂O at −78° C. Let slowly warm to 5° C. over 3 hr. Cooled down to −78° C. and quenched by addition of saturated aqueous NH₄Cl solution (20 mL). Warmed to room temperature and extracted with DCM (3×10 mL). Combined extracts were dried with Na₂SO₄ and concentrated under reduced pressure. Flash chromatography (ISCO system, silica 24 g, 0-50% ethyl acetate in hexane) provide E-5-1 (980.2 mg, 5.73 mmol, 88.83% yield).

Step 2. To a solution of E-5-1 (239.3 mg, 1.40 mmol) in DCM (6.99 mL) was added mesyl chloride (208.19 mg, 1.82 mmol, 140.67 uL). Cooled to 0° C. and DIEA (722.73 mg, 5.59 mmol, 974.03 uL) was added. Stirred as temperature increase from 0-22° C. over 1 hr and then quenched with 2M HCl(aq) (5 mL) at 0° C. The solution was diluted with water and DCM (10 mL each) and the layers partitioned. The aqueous layer was extracted 2× with DCM (5 mL). Combined organic layers was washed with brine and dried over sodium sulfate. Flash column chromatography (ISCO, 12 g, EtOAc in Hexanes) afforded E-5 (205.1 mg, 1.08 mmol, 77.25% yield).

| | | MS |
|---|---|---|
| | | [M + Na] |
| Compd# | Structure | m/z |
| E-1 | 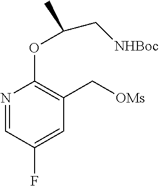 [SMILES: C[C@@H](CNC(=O)OC(C)(C)C)Oc1ncc(F)cc1COS(C)(=O)=O] | 401.1 |
| E-2 | 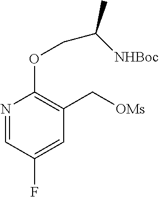 [SMILES: C[C@H](COc1ncc(F)cc1COS(C)(=O)=O)NC(=O)OC(C)(C)C] | 401.1 |
| E-3 | 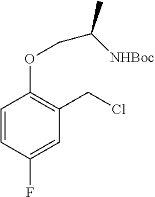 [SMILES: C[C@H](COc1ccc(F)cc1CCl)NC(=O)OC(C)(C)C] | 317.9 |
| E-4 | 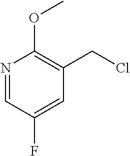 | |
| E-5 | 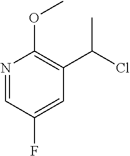 | 190.0 |

### General Method V

### Preparation of (4R,13R)-4-cyclopropyl-8-fluoro-13-methyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (7)

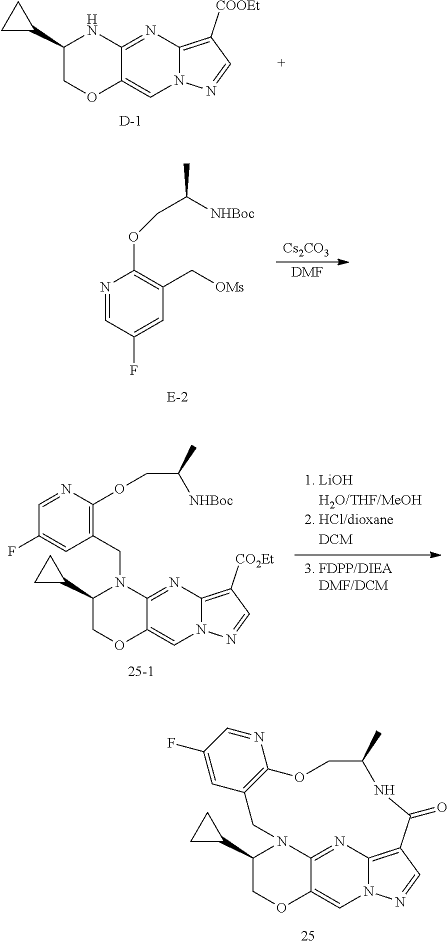

Step 1. To a solution of D-1 (44.5 mg, 154 μmol) and E-2 (60 mg, 159 μmol) in DMF (750 μL) was added Cs₂CO₃ (151 mg, 463 μmol). The reaction was stirred at room temperature for 1 hour. The reaction was cooled, quenched with water (3 mL), extracted with DCM (3×3 mL) dried with sodium sulfate and concentrated under reduced pressure. Flash chromatography (ISCO system, silica (12 g), 10-40% ethyl acetate in hexane) provided 25-1 (82 mg, 93% yield).

Step 2. Compound 25-1 was converted to 25 following the procedure used in General Method E.

Compound 26 was prepared according to General Method V using E-1 in step 1.

Compound 27 and 28 were prepared according to General Method V using D-2 in combination with E-1 and E-2 respectively in step 1.

Compound 29 and 30 were prepared according to General Method V using D-3 in combination with E-2 and E-1 respectively in step 1.

Compound 31 was prepared according to General Method V using D-3 and E-3 in step 1.

Compound 32 and 33 were prepared according to General Method I using appropriate boc-protected aminopropanols in step 4.

### General Method W

### Preparation of (4′R)-8′-fluoro-4′-methyl-3′H,4′H,6′H,12′H,14′H,15′H-spiro[cyclopropane-1,13′-[2,11]dioxa[5,10,14,17,18,19]hexaaza[18,1](metheno)[1,4]oxazino[3,4-i]pyrazolo[4,3-f]pyrido[3,2-l][1,4,8,10]oxatriazacyclotridecin]-15′-one (34)

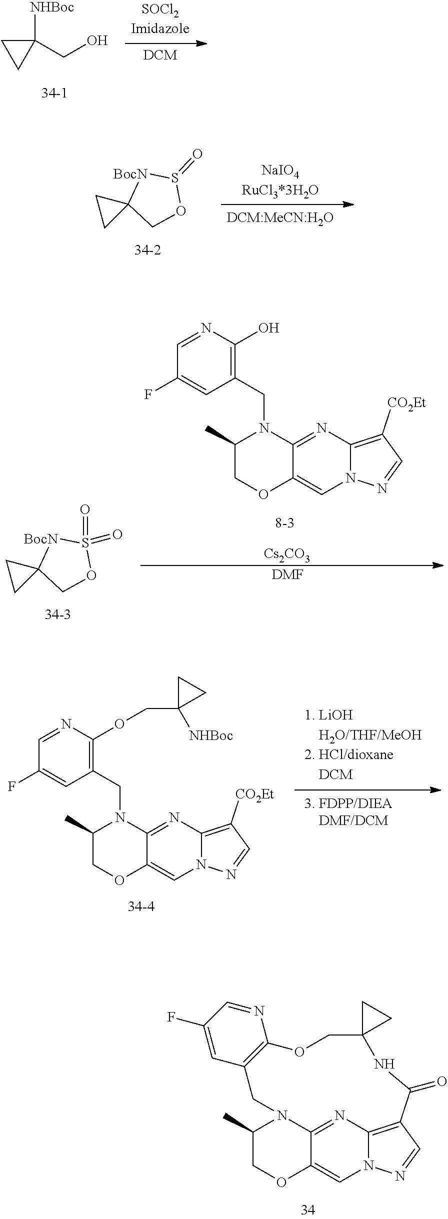

Steps 1 and 2 were performed according to General Method O using 34-1 in step 4.

Steps 3 and 4 were performed according to General Method L.

Compounds 35 and 36 was prepared according to General Method I using appropriate boc-protected aminopropanols in step 4.

Compound 37 was prepared according to General Method V using D-4 and E-1 in step 1.

Compounds 38 and 39 were prepared according to General Method V using D-5 in combination with E-1 and E-2 respectively in step 1.

Compound 40 was prepared according to General Method W.

### General Method X

### Preparation of [(4R,13R)-8-fluoro-13-methyl-15-oxo-3,4,12,13,14,15-hexahydro-6H-18,1-(metheno)[1,4]oxazino[3,4-i]pyrazolo[4,3-f]pyrido[3,2-][1,4,8,10]oxatriazacyclotridecin-4-yl]acetonitrile (41)

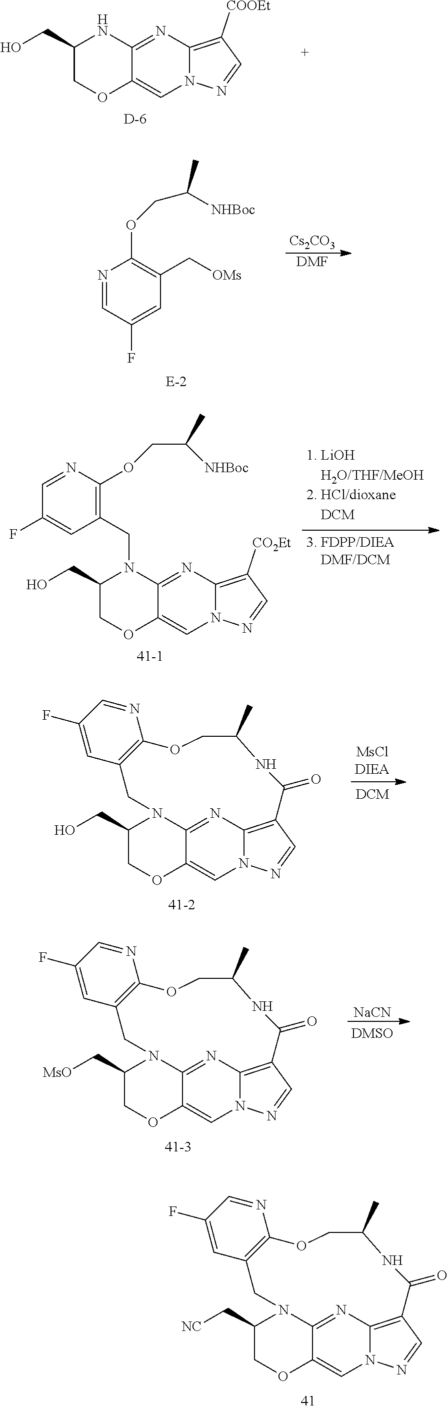

Step 1 was performed using the procedure used in General Method V.

Step 2. Compound 41-1 was converted to 41-2 following the procedure used in General Method E.

Step 3. To a solution of 41-2 (30 mg, 72 μmol) in DCM (360 μL) at 0° C. was added mesyl chloride (10.4 mg, 90 μmol, 7.0 μL) and DIEA (47 mg, 362 μmol, 63 μL). The reaction was stirred as temperature increase from 0-22° C. over 2.5 hours. Crude 41-3 in solution was transferred directly into next reactions.

Step 4. To a solution of 41-3 (12.5 mg (theoretical), 25 μmol) in DMSO (500 μL) was added NaCN (62 mg, 1.3 mmol). The reaction was heated to 60° C. and stirred for 6 hours. The reaction was cooled, diluted with ethyl acetate (15 mL) and water (20 mL). The mixture was mixed vigorously then layers separated and aqueous layer further extracted with ethyl acetate (2×15 mL), Combined organic layers were dried with sodium sulfate and concentrated under reduced pressure. Flash chromatography (ISCO system, silica (12 g), 1.25-5% methanol in dichloromethane) provided 41(2.26 mg, 21% yield).

### General Method Y

### Preparation of (13R)-8-fluoro-13-methyl-4-methylidene-3,4,13,14-tetrahydro-6H-18,1-(metheno)[1,4]oxazino[3,4-i]pyrazolo[4,3-f]pyrido[3,2-1][1,4,8,10]oxatriazacyclotridecin-15(12H)-one (42)

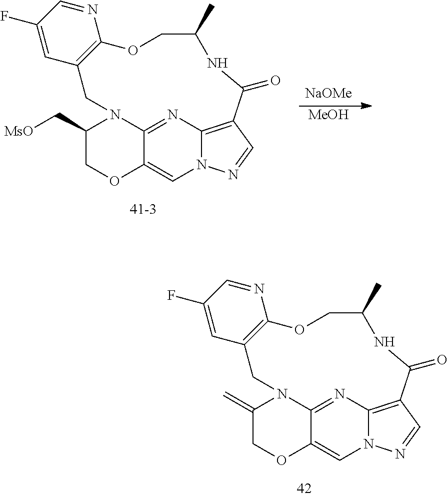 [SMILES: "MeOH" ; C[C@@H]1COc2ncc(F)cc2CN2c3nc4c(cnn4cc3OC[C@H]2COS(C)(=O)=O)C(=O)N1 ; C=C1COc2cn3ncc4c3nc2N1Cc1cc(F)cnc1OC[C@@H](C)NC4=O ; "NaOMe"]

To a solution of 41-3 (12.5 mg (theoretical), 25 μmol) in MeOH (250 μL) was added NaOMe solution (4.6 M, 270 μL). The reaction was stirred for 16 hours, diluted with DCM (10 mL) and water (20 mL). The mixture was mixed vigorously then layers separated and aqueous layer further extracted with DCM (2×5 mL), Combined organic layers were dried with sodium sulfate and concentrated under reduced pressure. Flash chromatography (ISCO system, silica (12 g), 0-5% methanol in dichloromethane) provided 42 (2.26 mg, 21% yield).

Compound 43 was prepared according to General Method Y using NaN₃ in step 4.

### General Method Z

### Preparation of (4R,13R)-8-fluoro-4-(methoxymethyl)-13-methyl-3,4,13,14-tetrahydro-6H-18,1-(metheno)[1,4]oxazino[3,4-i]pyrazolo[4,3-f]pyrido[3,2-1][1,4,8,10]oxatriazacyclotridecin-15(12H)-one (44)

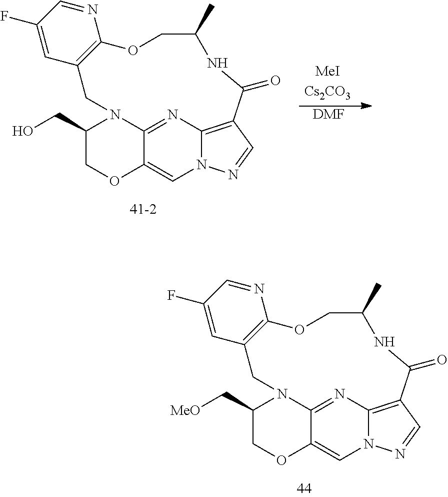

To a solution of 41-2 (10 mg, 24 μmol) and Mel (10.3 mg, 72 μmol, 4.5 μL) in DMF (250 μL) was added Cs₂CO₃ (39 mg, 121 μmol). The reaction was stirred for 2 days then diluted with DCM (3 mL), filtered through a syringe filter and concentrated under reduced pressure. Flash chromatography (ISCO system, silica (12 g), 0-5% methanol in dichloromethane) provided 44 (3.08 mg, 29% yield).

Compound 45 was prepared according to General Method W.

### General Method AA

### Preparation of (4R,13R)-8-fluoro-13-methyl-4-phenyl-3,4,13,14-tetrahydro-6H-18,1-(metheno)[1,4]oxazino[3,4-i]pyrazolo[4,3-f]pyrido[3,2-1][1,4,8,10]oxatriazacyclotridecin-15(12H)-one (46)

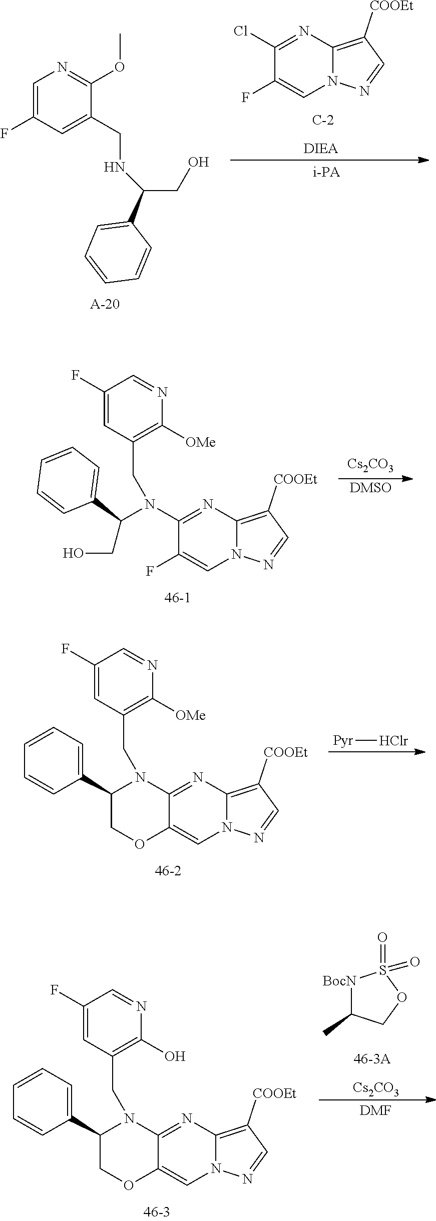

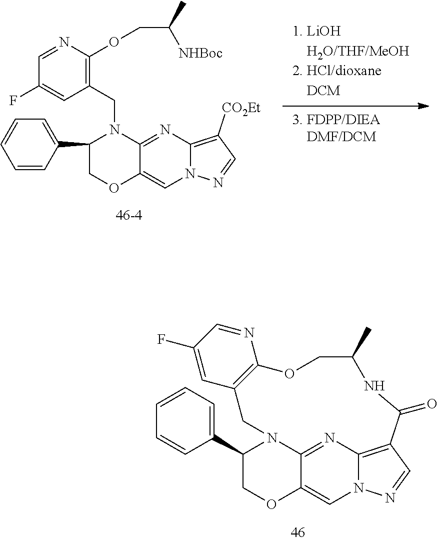 [SMILES: CCOC(=O)c1cnn2cc3c(nc12)N(Cc1cc(F)cnc1OC[C@@H](C)NC(=O)OC(C)(C)C)[C@H](c1ccccc1)CO3 ; C[C@@H]1COc2ncc(F)cc2CN2c3nc4c(cnn4cc3OC[C@H]2c2ccccc2)C(=O)N1]

Step 1: Compound A-20 (228.9 mg, 828.43 umol) was dissolved in i-PA (4.14 mL) at room temperature and DIEA (107.07 mg, 828.43 umol, 144.30 uL) was added followed by C-2 (201.82 mg, 828.43 umol). The mixture stirred at 80° C. for 18 hr and then concentrated under reduced pressure. Flash column chromatography (ISCO, 12 g, silica, ethyl acetate in hexanes) gave 46-1 (124.4 mg, 257.31 umol, 31.06% yield).

Step 2. Compound 46-1 (124.4 mg, 257.31 umol) was dissolved in DMSO (1.29 mL) and Cs₂CO₃ (335.34 mg, 1.03 mmol) was added. The mixture was stirred at RT for 2 hr, then diluted with DCM (20 mL) and washed with water (20 mL), the organic layer was washed with brine and dried in sodium sulfate. Flash column chromatography (ISCO, 12 g, silica, Ethyl acetate in hexanes) provided 46-2 (50.9 mg, 109.83 umol, 42.68% yield).

Step 2. Compound 46-2 (47.5 mg, 102.49 umol) and Pyridine HCl (47.37 mg, 409.96 umol) were combined and heated to 145° C. neat for 1 hr. Cooled and diluted with EtOAc (5 mL) and water (5 mL). Layers were partitioned and aqueous layer was extracted twice more with EtOAc (2×5 mL). The combined organic layer was washed with brine and dried over sodium sulfate. Purified by flash column chromatography (12 g, silica, 0-40% EtOAc in Hexanes) to afford 46-3 (20.9 mg, 46.50 umol, 45.37% yield).

Steps 4 and 5 were performed using the procedures of General Method W.

Compound 47 and 48 were prepared according to General Method I using appropriate boc-protected aminopropanols in step 4.

Compound 49 was prepared according to General Method I using appropriate boc-protected aminocyclopentanol in step 4.

Compound 50 was prepared according to General Method W.

Compound 51 was prepared according to General Method I using appropriate boc-protected aminoethanol in step 4.

Compound 52 was prepared according to General Method W.

Compound 53 was prepared according to General Method I using appropriate boc-protected aminocyclopentanol in step 4.

Compound 54 was prepared according to General Method W using D-9-7 in step 3.

Compound 55 was prepared according to General Method I using appropriate boc-protected aminopropanol in step 4.

Compound 56 through 59 were prepared according to General Method V using combinations of D-7 and D-8 with E-1 and E-2 in step 1.

### General Method BB

### Preparation of (4S,13R)-8-fluoro-13-methyl-15-oxo-3,4,12,13,14,15-hexahydro-6H-18,1-(metheno)[1,4]oxazino[3,4-i]pyrazolo[4,3-f]pyrido[3,2-l][1,4,8,10]oxatriazacyclotridecine-4-carboxamide (60)

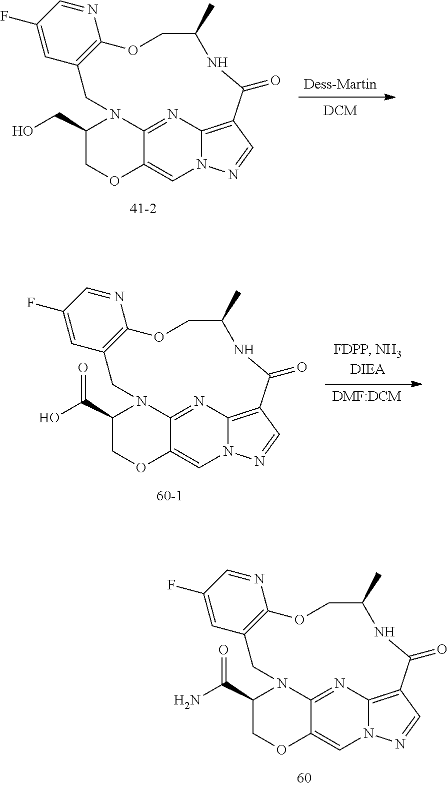 [SMILES: C[C@@H]1COc2ncc(F)cc2CN2c3nc4c(cnn4cc3OC[C@H]2CO)C(=O)N1 ; C[C@@H]1COc2ncc(F)cc2CN2c3nc4c(cnn4cc3OC[C@H]2C(=O)O)C(=O)N1 ; C[C@@H]1COc2ncc(F)cc2CN2c3nc4c(cnn4cc3OC[C@H]2C(N)=O)C(=O)N1]

Step 1. To a solution of 41-2 (59.6 mg, 144 μmol) in DCM (1.45 mL) was added Dess-Martin Periodinane (92 mg, 216 μmol). The reaction was stirred for 3 hours then quenched with saturated NaHCO₃ solution (10 mL) and stirred for 5 minutes then extracted with DCM (3×15 mL). Washed extract with 0.5 M NaOH solution. Combined aqueous portions and adjusted to acidic with 2 M HCl solution then reextracted with DCM (4×35 mL). Combined extracts were dried with Na₂SO₄ and concentrated under reduced pressure. Compound was used as is in next step.

Step 2. Crude 60-1 (61.6 mg (theoretical), 144 μmol) was dissolved in in DMF (2 mL) and DCM (8 mL) and DIEA (465 mg, 3.6 mmol, 625 μL) then NH₃ solution (0.5 M in dioxane, 7.2 mL) and FDPP (220 mg, 575 μmol) were added. The reaction was stirred for 20 hours then quenched with 2 M Na₂CO₃ solution (5 mL). The mixture was stirred for 5 min then extracted with DCM (3×10 mL). Combined extracts were dried with Na₂SO₄ and concentrated under reduced pressure. Flash chromatography (ISCO system, silica (12 g), 0-10% methanol in dichloromethane) provided 60 (14.2 mg, 18% yield).

Compound 61 through 64 were prepared according to General Method V using combinations of D-9 and D-10 with E-1 and E-2 in step 1.

### General Method CC

### Preparation of (4R)-4-ethyl-8-fluoro-13,13-dimethyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (65)

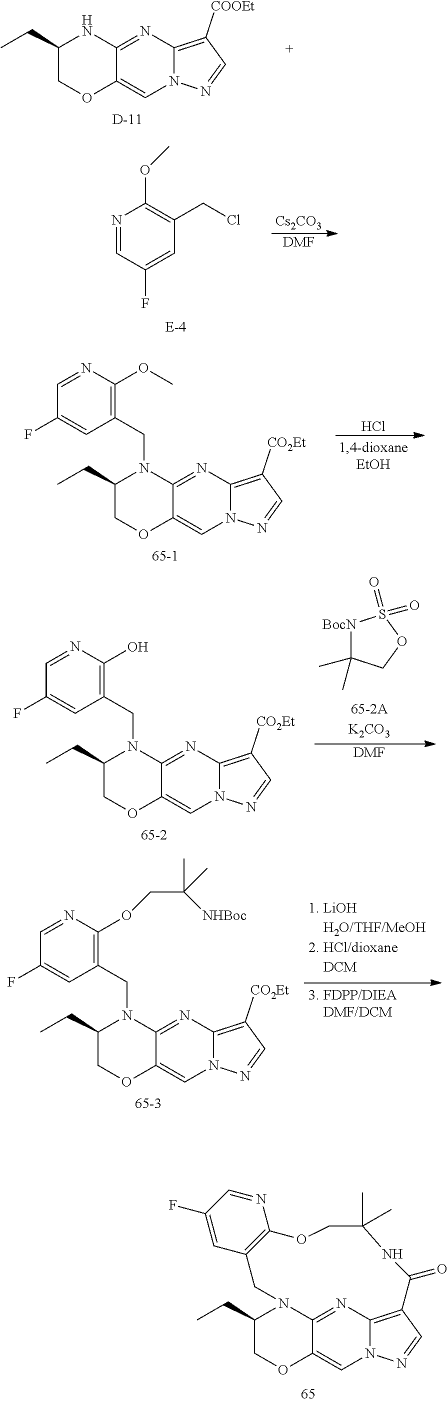

Step 1. To a solution of D-11 (145 mg, 525 μmol) and E-4 (106 mg, 03 μmol) in DMF (4 mL) was added Cs₂CO₃ (684 mg, 2.1 mmol). The reaction was stirred at room temperature for 1 hour. The reaction was cooled, diluted with DCM (3 mL), filtered through a syringe filter and concentrated under reduced pressure. Flash chromatography (ISCO system, silica (12 g), 20-100% ethyl acetate in hexane) provided 65-1 (210.9 mg, 97% yield).

Step 2. To a solution of 65-1 (210.9 mg, 508 μmol) in EtOH (6.0 mL) was added HCl (4M, 10 mL). The mixture was heated to 70° C. for 8 hours. The reaction cooled and concentrated under reduced pressure. Flash chromatography (ISCO system, silica (12 g), 0-10% methanol in dichloromethane) provided 65-2 (169.2 mg, 83% yield).

Steps 3 and 4 were performed using the procedures of General Method W.

### General Method DD

### Preparation of (4R,13R)-8-fluoro-13-methyl-15-oxo-3,4,12,13,14,15-hexahydro-6H-18,1-(metheno)[1,4]oxazino[3,4-i]pyrazolo[4,3-f]pyrido[3,2-1][1,4,8,10]oxatriazacyclotridecine-4-carbonitrile (66)

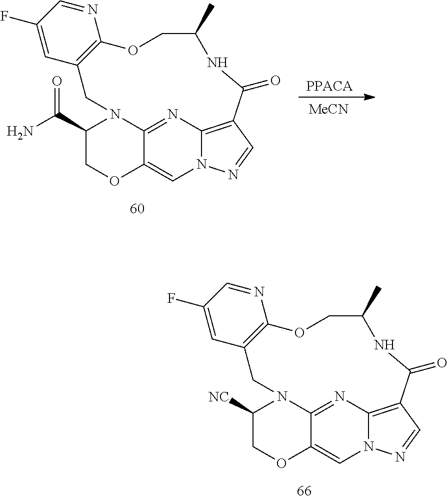 [SMILES: "MeCN" ; C[C@@H]1COc2ncc(F)cc2CN2c3nc4c(cnn4cc3OC[C@H]2C(N)=O)C(=O)N1 ; C[C@@H]1COc2ncc(F)cc2CN2c3nc4c(cnn4cc3OC[C@H]2C#N)C(=O)N1]

To a solution of 60 (4.2 mg, 9.8 μmol) in acetonitrile (1 mL) was added propylphosphonic anhydride (PPACA) solution (500 mg, 1.57 mmol, 1 mL) in DMF. The reaction was heated to 70° C. and stirred for 16 hours. The reaction was cooled and quenched with 2 M Na₂CO₃ solution (20 mL) then extracted with DCM (3×10 mL). Combined extracts were dried with Na₂SO₄ and concentrated under reduced pressure. Flash chromatography (ISCO system, silica (12 g), 0-7.5% methanol in dichloromethane) provided 66 (1.80 mg, 45% yield).

Compound 67 was prepared according to General Method I using appropriate boc-protected aminopropanol in step 4.

Compound 68 was prepared according to General Method W.

Compounds 69 through 71 were prepared according to General Method I using appropriate boc-protected aminopropanols in step 4.

Compounds 72 and 73 were prepared according to General Method CC.

Compound 74 was prepared according to General Method V using D-12 and E-2 in step 1.

Compounds 75 and 76 were prepared according to General Method I using 65-2 and appropriate boc-protected aminopropanols in step 4.

Compound 77 was prepared according to General Method V using D-12 and E-1 in step 1.

Compounds 78 through 80 were prepared according to General Method I using appropriate boc-protected aminopropanols in step 4.

### General Method EE

### Preparation of (16R)-12-fluoro-7,7-dihydroxy-16-methyl-5,6,7,8,16,17-hexahydro-4H,14H-1,19-(metheno)[1,4]oxazino[4,3-e]pyrazolo[3,4-h]pyrido[2,3-b][1,5,7,11]oxatriazacyclotetradecin-4-one

(66)

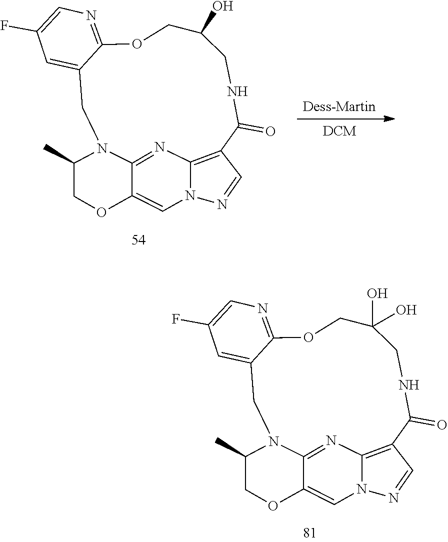 [SMILES: C[C@@H]1COc2cn3ncc4c3nc2N1Cc1cc(F)cnc1OC[C@@H](O)CNC4=O ; C[C@@H]1COc2cn3ncc4c3nc2N1Cc1cc(F)cnc1OCC(O)(O)CNC4=O]

To a solution of 54 (47 mg, 113 μmol) in DCM (1.45 mL) was added Dess-Martin Periodinane (96 mg, 227 μmol). The reaction was stirred for 30 minutes then quenched with saturated NaHCO₃ solution (10 mL) and stirred for 5 minutes then extracted with DCM (3×5 mL). Combined extracts were dried with Na₂SO₄ and concentrated under reduced pressure. Flash chromatography (ISCO system, silica (12 g), 0-100% ethyl acetate in hexane) provided 81 (30.9 mg, 63% yield).

Compound 82 was prepared according to General Method I using commercially available 3-Oxazolidinecarboxylic acid, 4-(hydroxymethyl)-2,2-dimethyl-, 1,1-dimethylethyl ester, (4R)— in step 4.

### General Method FF

### Preparation of (4R,6R,13R)-8-fluoro-4,6,13-trimethyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (83) and (4R,6S,13R)-8-fluoro-4,6,13-trimethyl-3,4,13,14-tetrahydro-6H-18,1-(metheno)[1,4]oxazino[3,4-i]pyrazolo[4,3-f]pyrido[3,2-l][1,4,8,10]oxatriazacyclotridecin-15(12H)-one (84)

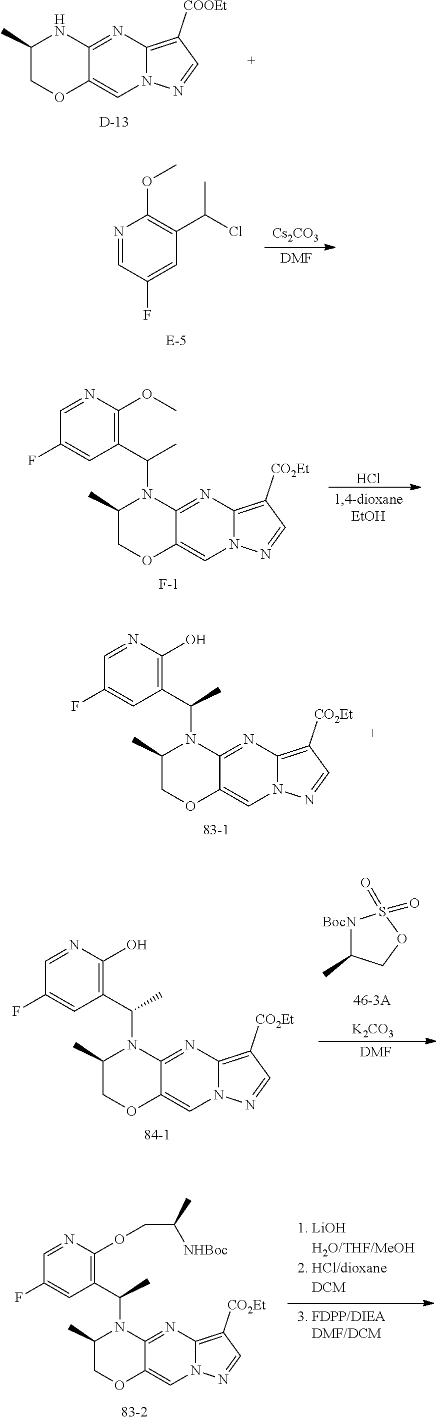

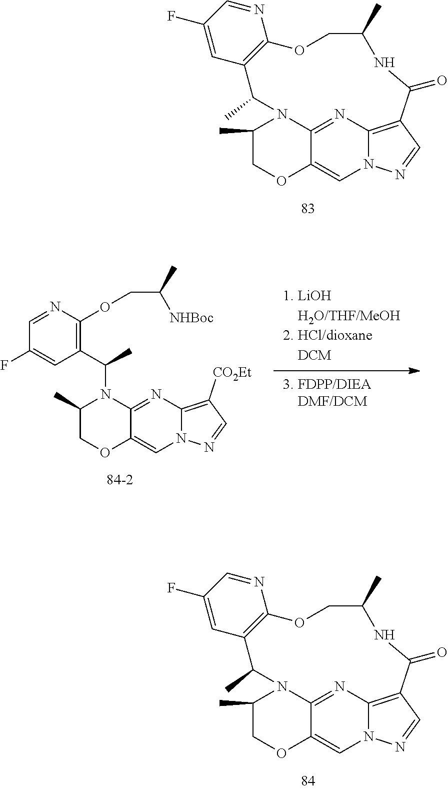 [SMILES: C[C@@H]1COc2ncc(F)cc2[C@@H](C)N2c3nc4c(cnn4cc3OC[C@H]2C)C(=O)N1 ; CCOC(=O)c1cnn2cc3c(nc12)N([C@H](C)c1cc(F)cnc1OC[C@@H](C)NC(=O)OC(C)(C)C)[C@H](C)CO3 ; C[C@@H]1COc2ncc(F)cc2[C@H](C)N2c3nc4c(cnn4cc3OC[C@H]2C)C(=O)N1]

Step 1. Compound D-13 (39.76 mg, 209.71 umol) was dissolved in DMF (381.30 uL) at room temperature and E-5 (50 mg, 190.65 umol) was added followed by Cs₂CO₃ (186.35 mg, 571.94 umol). The mixture stirred at 22° C. for 16 hr and then diluted with DCM (10 mL). The solution was filtered and the filtrate was concentrated under reduced pressure. Flash column chromatography (ISCO, 12 g, ethyl acetate in hexanes) afforded F-1 (41.4 mg, 99.66 umol, 52.27% yield).

Step 2. Compound F-1 was dissolved in anhydrous ethanol (2 mL) followed by the addition of HCl in dioxane (4M, 2 mL). The mixture was stirred at ambient temperature for 18 hr and then concentrated under reduced pressure. The crude was purified by flash column chromatography (ISCO, 12 g, EtOAc in Hexanes) after adding 0.5 mL of TEA to afford enantiomers 83-1 (9.9 mg, 24.66 umol, 24.93% yield) and 84-1 (16.6 mg, 41.36 umol, 41.80% yield).

Steps 3 and 4 were performed independently on 83-1 and 84-1 using procedures similar to that of General Method W to give 83 and 84.

Compounds 85 through 87 were prepared according to General Method I using appropriate boc-protected aminoalcohols in step 4.

Compound 88 was prepared according to General Method V using D-14 and E-2 in step 1.

Compound 89 was prepared according to General Method W using ent-D-9-7 in step 3.

Compound 90 was prepared according to General Method V using D-14 and E-1 in step 1.

### General Method GG

### Preparation of (4S,13S)-9-fluoro-4-methoxy-13-methyl-4,5,14,15-tetrahydro-3H,7H-19,1-(metheno)[1,4]oxazepino[3,4-i]pyrazolo[4,3-f]pyrido[3,2-1][1,4,8,10]oxatriazacyclotridecin-16(13H)-one (91)

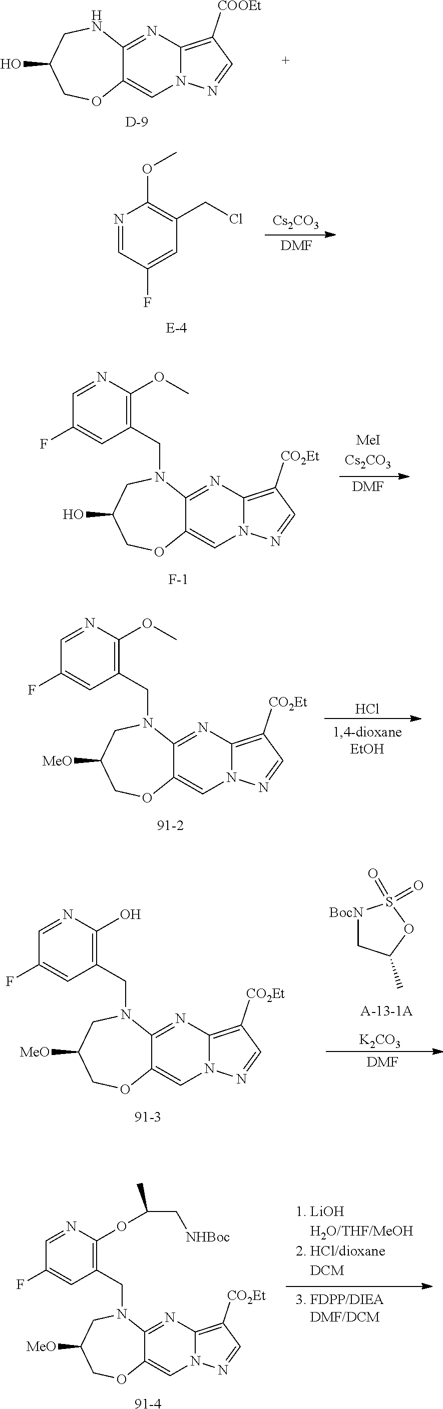

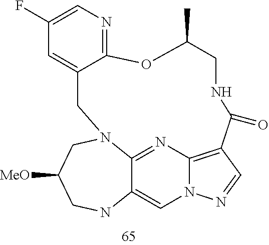 [SMILES: CO[C@@H]1CNc2cn3ncc4c3nc2N(Cc2cc(F)cnc2O[C@@H](C)CNC4=O)C1]

Step 1 was performed using the procedure in General Method V step 1.

Step 2. was performed using the procedure in General Method Z.

Steps 3 through 5 were performed using the procedures of General Method FF.

Compounds 92 was made from 82 according to General Method Z.

Compounds 93 and 94 were prepared according to General Method V using D-15 in combination with E-1 and E-2 respectively in step 1.

Compounds 95 through 97 were prepared according to General Method I using appropriate boc-protected aminoalcohols in step 4.

### General Method HH

### Preparation of (4R,12S)-8-fluoro-12-(hydroxymethyl)-4-methyl-3,4,13,14-tetrahydro-6H-18,1-(metheno)[1,4]oxazino[3,4-i]pyrazolo[4,3-f]pyrido[3,2-1][1,4,8,10]oxatriazacyclotridecin-15(12H)-one (98)

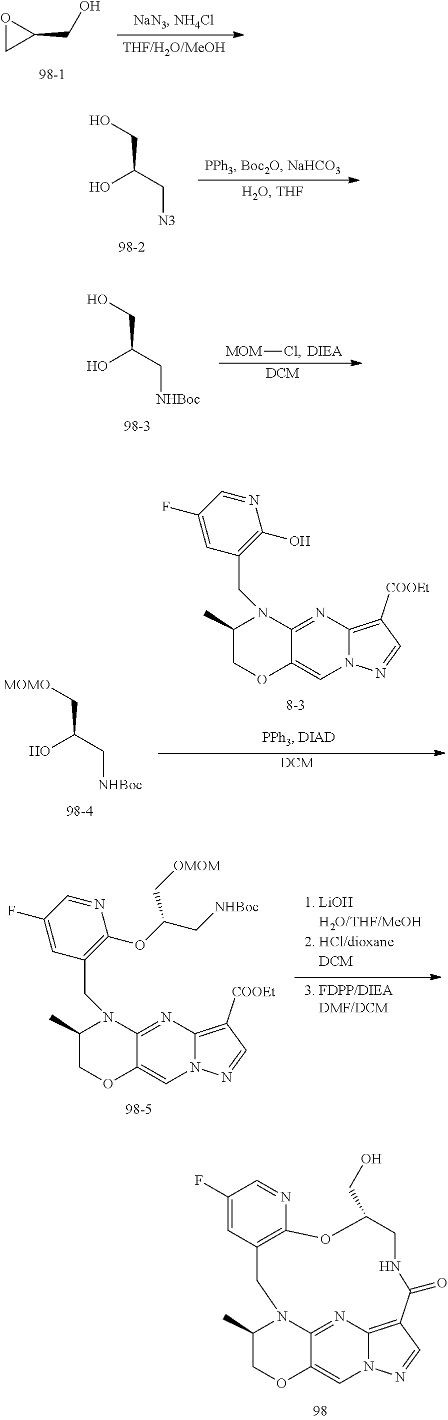

Step 198-1 (1.00 g, 13.50 mmol) was dissolved in THF (2.70 mL), water (2.70 mL) and methanol (21.60 mL). Ammonium chloride (1.66 g, 31.05 mmol) was added followed by sodium azide (4.39 g, 67.50 mmol). The mixture was stirred at 75° C. for 3 hr and then cooled to ambient temperature. Volume was carefully reduced under reduced pressure to a third and then diluted with DCM (50 mL) and water (50 mL). The layers were partitioned and the aqueous layer was extracted 2× with DCM (2×20 mL). The combined organic layer was washed with brine and dried over sodium sulfate. Flash column chromatography (ISCO, 24 g, silica, ethyl acetate in hexanes) gave 98-2 (450.00 mg, 3.84 mmol, 28.46% yield).

Step 2. 98-2 (450.00 mg, 3.84 mmol) was dissolved in THF (19.20 mL) and PPh₃ (2.32 g, 8.83 mmol) was added. Stirred for 4 hr and water (1.59 g, 88.32 mmol, 1.59 mL) was added and continued to stir for 16 hr when boc anhydride (1.09 g, 4.99 mmol) was added followed by sodium bicarbonate (32.26 mg, 384.00 umol). The mixture was stirred at RT for 4 hr and ethyl acetate and water were added (30 mL each). The layers were partitioned and the aqueous layer was extracted 2× with ethyl acetate (2×20 mL). The combined organic layer was washed with brine and then dried over sodium sulfate. Purified by flash column chromatography (ISCO, 24 g, silica, EtOAc in Hexanes) to provide 98-3 (525.30 mg, 2.75 mmol, 71.54% yield).

Step 3. 98-3 (525.86 mg, 2.75 mmol) was dissolved in DCM (4.58 mL) and MOM-C₁ (332.10 mg, 4.13 mmol, 313.30 uL) was added followed by DIEA (710.82 mg, 5.50 mmol, 960.57 uL) at 0° C. Stirred for 18 hr slowly warming to RT. Water (5 mL) was added and the layers were partitioned. The aqueous layer was extracted 2× with DCM (5 mL). The combined organic layer was washed with brine and dried over sodium sulfate. Salts were filtered and volatiles were carefully removed via rotary evaporation at temperatures<30° C. to afford 98-4 (132.2 mg, 0.561 mmol, 20% yield). Used directly without further purification.

Step 4 and 5 were performed using the procedure of according to General Method E.

### General Method II

### Preparation of (17S)-12-fluoro-6,6,17-trimethyl-5,6,7,8,17,18-hexahydro-4H,14H,16H-1,20-(metheno)[1,4]oxazepino[4,3-e]pyrazolo[3,4-h]pyrido[2,3-b][1,5,7,11]oxatriazacyclotetradecin-4-one (99)

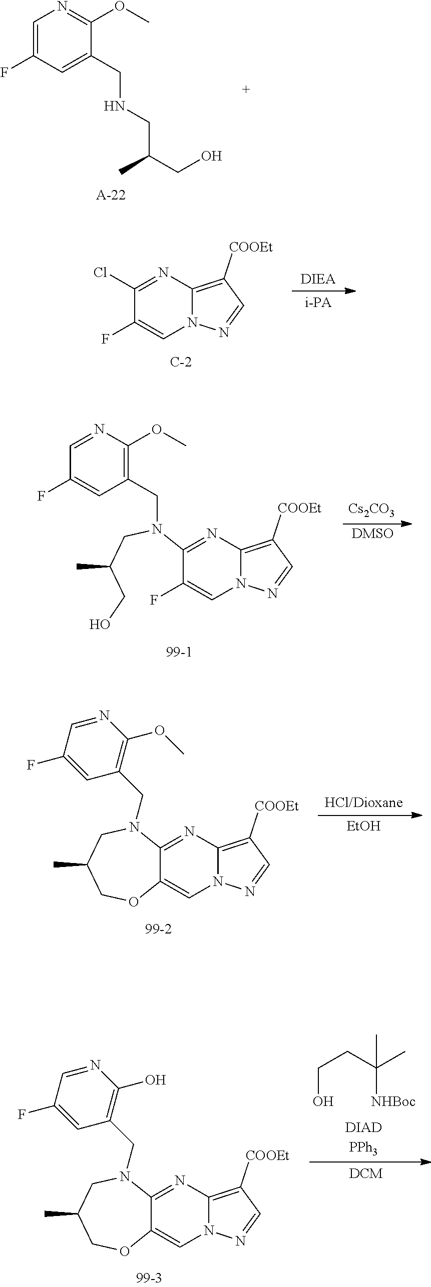

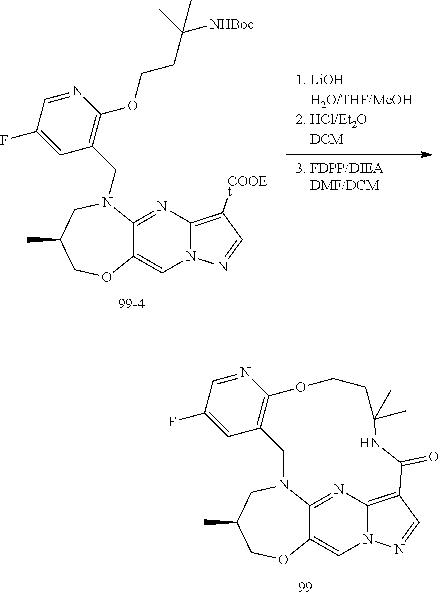 [SMILES: CCOC(=O)c1cnn2cc3c(nc12)N(Cc1cc(F)cnc1OCCC(C)(C)NC(=O)OC(C)(C)C)C[C@H](C)CO3 ; C[C@@H]1COc2cn3ncc4c3nc2N(Cc2cc(F)cnc2OCCC(C)(C)NC4=O)C1]

Step 1. A-22 (612.3 mg, 2.68 mmol) and C-22 (784.20 mg, 3.22 mmol) were combined in i-PA (13.41 mL) and DIEA (1.04 g, 8.05 mmol, 1.40 mL) was added. Mixture was stirred and heated to 80° C. for 18 hr. Concentrated under reduced pressure and the crude was purified by flash column chromatography (ISCO, 24 g, silica, EtOAc in Hexanes) to afford 99-1 (1.05 g, 2.41 mmol, 89.90% yield).

Step 2. 99-1 (1.05 g, 2.41 mmol) was dissolved in DMSO (50 mL) and CS₂CO₃ (3.1 g, 9.51 mmol) was added. The mixture was stirred at 22° C. for 16 hr then diluted with DCM (150 mL) and washed with 20% brine (200 mL). Aqueous layer extracted with DCM twice more (50 mL each). The combined organic layer was washed with brine and dried over sodium sulfate. The crude was purified by flash column chromatography (ISCO, 24, silica, ethyl acetate in hexanes) to afford 99-2 (480 mg, 1.16 mmol, 47.92% yield).

Step 3. To a solution of 99-2(480 mg, 1.16 mmol) in Ethanol (10 mL), HCl in Dioxane (4 M, 5 mL) was added. Stirred and heat to 75° C. for 16 hr. Concentrated under reduced pressure and the crude was purified by flash column chromatography (ISCO, 24 g, silica, 30 to 100% EtOAc in Hexanes) to afford 99-3(371.6 mg, 925.78 umol, 80.12% yield).

Step 4 and 5 were performed using the procedure of according to General Method E using appropriate boc-protected aminoalcohols in step 4.

Compound 100 was prepared according to General Method II using appropriate boc-protected aminoalcohols in step 4.

| | | MS | |
|---|---|---|---|
| | | [M + H] | |
| Cpd | Structure | m/z | ¹H NMR (DMSO-d₆) δ ppm |
| 1 | 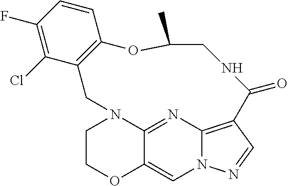 [SMILES: C[C@H]1CNC(=O)c2cnn3cc4c(nc23)N(CCO4)Cc2c(ccc(F)c2Cl)O1] | 418.2 | (500 MHz) 9.08 (dd, J = 6.30, 2.86 Hz, 1 H) 8.56 (s, 1 H) 7.99 (s, 1 H) 7.25-7.33 (m, 1 H) 7.17-7.23 (m, 1 H) 5.55 (dd, J = 14.89, 1.72 Hz, 1 H) 4.92-5.01 (m, 1 H) 4.45 (dt, J = 11.17, 3.01 Hz, 1 H) 4.24 (ddd, J = 11.31, 9.02, 2.58 Hz, 1 H) 4.06- 4.15 (m, 2 H) 3.90 (ddd, J = 12.75, 9.31, 3.15 Hz, 1 H) 3.71-3.80 (m, 1 H) 3.35-3.42 (m, 1 H) 1.44 (d, J = 5.73 Hz, 3 H) |
| 2 | 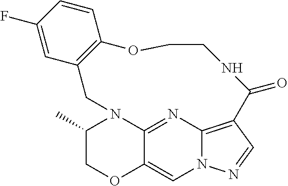 [SMILES: C[C@H]1COc2cn3ncc4c3nc2N1Cc1cc(F)ccc1OCCNC4=O] | 384.2 | (300 MHz) 9.43 (dd, J = 6.28, 3.26 Hz, 1 H) 8.56 (s, 1 H) 8.00 (s, 1 H) 7.26-7.35 (m, 1 H) 7.03-7.11 (m, 2 H) 5.36 (dd, J = 14.67, 1.19 Hz, 1 H) 4.41-4.53 (m, 2 H) 4.30 (s, 2 H) 4.20 (d, J = 14.95 Hz, 1 H) 4.06- 4.17 (m, 1 H) 3.73-3.88 (m, 1 H) 3.43 (dq, J = 13.56, 3.86 Hz, 1 H) 1.38 (d, J = 6.42 Hz, 3 H) |
| 3 | 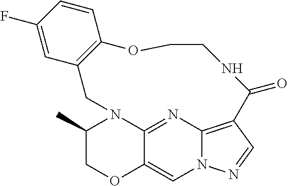 [SMILES: C[C@@H]1COc2cn3ncc4c3nc2N1Cc1cc(F)ccc1OCCNC4=O] | 384.2 | (300 MHz) 9.43 (dd, J = 6.19, 3.44 Hz, 1 H) 8.56 (s, 1 H) 8.00 (s, 1 H) 7.27-7.35 (m, 1 H) 7.03-7.09 (m, 2 H) 5.36 (dd, J = 14.53, 1.24 Hz, 1 H) 4.43-4.53 (m, 2 H) 4.30 (s, 2 H) 4.20 (d, J = 14.86 Hz, 1 H) 4.09- 4.17 (m, 1 H) 3.74-3.87 (m, 1 H) 3.36-3.50 (m, 1 H) 1.38 (d, J = 6.42 Hz, 3 H) |
| 4 | 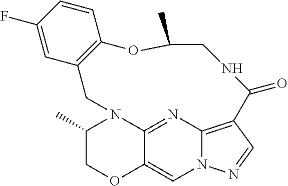 [SMILES: C[C@H]1CNC(=O)c2cnn3cc4c(nc23)N(Cc2cc(F)ccc2O1)[C@@H](C)CO4] | 398.2 | (300 MHz) 9.03 (dd, J = 6.56, 2.89 Hz, 1 H) 8.60 (s, 1 H) 7.99 (s, 1 H) 7.17 (dd, J = 9.22, 4.72 Hz, 1 H) 6.97- 7.12 (m, 2 H) 5.28-5.39 (m, 1 H) 4.87-4.99 (m, 1 H) 4.18-4.29 (m, 3 H) 3.97-4.09 (m, 1 H) 3.64-3.75 (m, 1 H) 3.38 (dd, J = 6.42, 3.30 Hz, 1 H) 1.58 (d, J = 6.60 Hz, 3 H) 1.44 (d, J = 6.24 Hz, 3 H) |
| 5 | 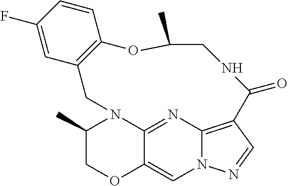 [SMILES: C[C@@H]1COc2cn3ncc4c3nc2N1Cc1cc(F)ccc1O[C@@H](C)CNC4=O] | 398.2 | (300 MHz) 9.47 (dd, J = 6.24, 3.48 Hz, 1 H) 8.56 (s, 1 H) 7.98 (s, 1 H) 7.26 (dd, J = 9.35, 2.93 Hz, 1 H) 6.95- 7.14 (m, 2 H) 5.28 (d, J = 15.59 Hz, 1 H) 4.57-4.72 (m, 1 H) 4.41-4.54 (m, 1 H) 4.31 (s, 2 H) 4.20 (d, J = 14.76 Hz, 1 H) 3.77-3.89 (m, 1 H) 3.13-3.26 (m, 1 H) 1.45 (d, J = 6.05 Hz, 3 H) 1.37 (d, J = 6.42 Hz, 3 H) |
| 6 | 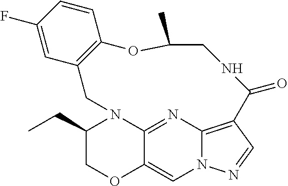 [SMILES: CC[C@@H]1COc2cn3ncc4c3nc2N1Cc1cc(F)ccc1O[C@@H](C)CNC4=O] | 412.2 | (500 MHz) 9.51 (dd, J = 6.87, 3.44 Hz, 1 H) 8.56 (s, 1 H) 7.98 (s, 1 H) 7.26 (dd, J = 9.45, 3.15 Hz, 1 H) 7.05- 7.10 (m, 1 H) 6.98-7.05 (m, 1 H) 5.32 (dd, J = 14.61, 1.43 Hz, 1 H) 4.56-4.65 (m, 1 H) 4.49 (d, J = 10.88 Hz, 1 H) 4.19-4.30 (m, 3 H) 3.85 (ddd, J = 13.46, 7.16, 4.58 Hz, 1 H) 3.17 (ddd, J = 13.32, 7.30, 3.44 Hz, 1 H) 1.78-1.90 (m, 1 H) 1.61-1.72 (m, 1 H) 1.45 (d, J = 6.30 Hz, 3 H) 1.02 (t, J = 7.45 Hz, 3 H) |
| 7 | 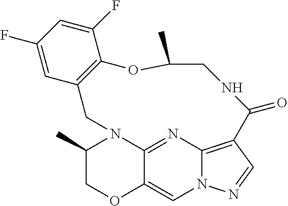 [SMILES: C[C@@H]1COc2cn3ncc4c3nc2N1Cc1cc(F)cc(F)c1O[C@@H](C)CNC4=O] | 416.1 | (500 MHz) 9.43 (t, J = 5.16 Hz, 1 H) 8.61 (s, 1 H) 8.00 (s, 1 H) 7.20 (ddd, J = 13.75, 8.59, 2.86 Hz, 1 H) 7.09 (br d, J = 8.59 Hz, 1 H) 5.22 (d, J = 14.89 Hz, 1 H) 4.99 (dt, J = 6.30, 3.15 Hz, 1 H) 4.41-4.48 (m, 1 H) 4.35-4.41 (m, 1 H) 4.33 (d, J = 11.46 Hz, 1 H) 4.26 (d, J = 14.89 Hz, 1 H) 3.42-3.57 (m, 2 H) 1.46 (d, J = 5.73 Hz, 3 H) 1.37 (d, J = 6.30 Hz, 3 H) |
| 8 | 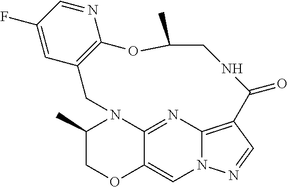 [SMILES: C[C@@H]1COc2cn3ncc4c3nc2N1Cc1cc(F)cnc1O[C@@H](C)CNC4=O] | 399.2 | (500 MHz) 9.35-9.44 (m, 1 H) 8.57 (s, 1 H) 8.07 (d, J = 2.86 Hz, 1 H) 7.99 (s, 1 H) 7.88 (dd, J = 8.59, 2.86 Hz, 1 H) 5.09-5.20 (m, 2 H) 4.50 (q, J = 6.30 Hz, 1 H) 4.25-4.36 (m, 3 H) 3.93 (ddd, J = 13.03, 8.16, 4.58 Hz, 1 H) 3.14 (ddd, J = 13.17, 9.17, 2.29 Hz, 1 H) 1.45 (d, J = 6.30 Hz, 3 H) 1.37 (d, J = 6.30 Hz, 3 H) |
| 9 | 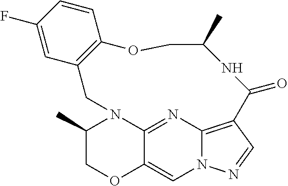 [SMILES: C[C@@H]1COc2ccc(F)cc2CN2c3nc4c(cnn4cc3OC[C@H]2C)C(=O)N1] | 398.2 | (500 MHz) 9.74 (d, J = 8.59 Hz, 1 H) 8.56 (s, 1 H) 7.99 (s, 1 H) 7.34 (dd, J = 9.17, 2.86 Hz, 1 H) 6.95-7.12 (m, 2 H) 5.41 (d, J = 14.89 Hz, 1 H) 4.46-4.59 (m, 1 H) 4.33 (br d, J = 10.31 Hz, 1 H) 4.20-4.30 (m, 4 H) 3.96 (dd, J = 9.45, 3.72 Hz, 1 H) 1.38 (d, J = 6.30 Hz, 3 H) 1.35 (d, J = 6.87 Hz, 3 H) |
| 10 | 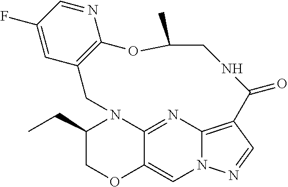 [SMILES: CC[C@@H]1COc2cn3ncc4c3nc2N1Cc1cc(F)cnc1O[C@@H](C)CNC4=O] | 413.2 | (500 MHz) 9.41 (br d, J = 7.45 Hz, 1 H) 8.56 (s, 1 H) 8.06 (d, J = 2.86 Hz, 1 H) 7.99 (s, 1 H) 7.87 (dd, J = 8.59, 2.86 Hz, 1 H) 5.20 (d, J = 14.89 Hz, 1 H) 5.08-5.17 (m, 1 H) 4.47 (d, J = 11.46 Hz, 1 H) 4.35 (d, J = 14.89 Hz, 1 H) 4.28 (br d, J = 6.87 Hz, 1 H) 4.24 (br d, J = 10.88 Hz, 1 H) 3.95 (ddd, J = 13.03, 8.45, 4.30 Hz, 1 H) 3.13 (ddd, J = 12.89, 9.45, 1.72 Hz, 1 H) 1.79-1.90 (m, 1 H) 1.61-1.72 (m, 1 H) 1.45 (d, J = 6.30 Hz, 3 H) |
| | | | 1.02 (t, J = 7.45 Hz, 3 H) |
| 11 | 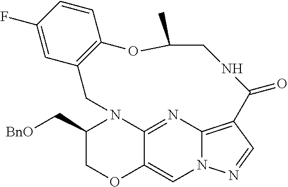 [SMILES: C[C@H]1CNC(=O)c2cnn3cc4c(nc23)N(Cc2cc(F)ccc2O1)[C@H](COCc1ccccc1)CO4] | 504.2 | (500 MHz) 9.45 (dd, J = 6.87, 3.44 Hz, 1 H) 8.55 (s, 1 H) 7.99 (s, 1 H) 7.31 (s, 2 H) 7.28-7.31 (m, 3 H) 7.21-7.28 (m, 1 H) 7.04-7.10 (m, 1 H) 6.96-7.04 (m, 1 H) 5.32 (d, J = 15.47 Hz, 1 H) 4.60-4.66 (m, 2 H) 4.58 (d, J = 13.75 Hz, 2 H) 4.50- 4.56 (m, 1 H) 4.32 (dd, J = 11.46, 2.29 Hz, 1 H) 4.26 (d, J = 14.89 Hz, 1 H) 3.85 (ddd, J = 13.46, 6.87, 4.30 Hz, 1 H) 3.71-3.82 (m, 2 H) 3.19 (ddd, J = 13.60, 7.02, 3.44 Hz, 1 H) |
| | | | 1.45 (d, J = 6.30 Hz, 3 H) |
| 12 | 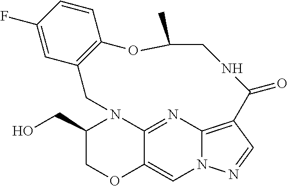 [SMILES: C[C@H]1CNC(=O)c2cnn3cc4c(nc23)N(Cc2cc(F)ccc2O1)[C@H](CO)CO4] | 414.1 | (500 MHz) 9.47 (dd, J = 6.30, 3.44 Hz, 1 H) 8.54 (s, 1 H) 7.98 (s, 1 H) 7.29 (dd, J = 9.74, 2.86 Hz, 1 H) 7.04- 7.11 (m, 1 H) 6.97-7.04 (m, 1 H) 5.37 (d, J = 14.32 Hz, 1 H) 5.24 (t, J = 5.44 Hz, 1 H) 4.58-4.69 (m, 1 H) 4.54 (d, J = 11.46 Hz, 1 H) 4.22- 4.36 (m, 3 H) 3.84 (ddd, J = 13.46, 6.59, 4.01 Hz, 1 H) 3.68-3.78 (m, 1 H) 3.64 (dq, J = 7.45, 5.54 Hz, 1 H) 3.18 (ddd, J = 13.60, 7.02, 3.44 Hz, 1 H) 1.45 (d, J = 5.73 Hz, 3 H) |
| 13 | 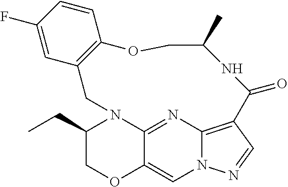 [SMILES: CC[C@@H]1COc2cn3ncc4c3nc2N1Cc1cc(F)ccc1OC[C@@H](C)NC4=O] | 412.2 | (500 MHz) 9.71 (d, J = 8.59 Hz, 1 H) 8.55 (s, 1 H) 7.99 (s, 1 H) 7.34 (dd, J = 9.17, 2.86 Hz, 1 H) 7.07 (td, J = 8.59, 2.86 Hz, 1 H) 6.94-7.03 (m, 1 H) 5.44 (d, J = 14.32 Hz, 1 H) 4.48 (d, J = 11.46 Hz, 1 H) 4.21- 4.37 (m, 4 H) 4.18 (br d, J = 11.46 Hz, 1 H) 3.96 (dd, J = 9.74, 3.44 Hz, 1 H) 1.83-1.94 (m, 1 H) 1.60-1.72 (m, 1 H) 1.36 (d, J = 6.87 Hz, 3 H) 1.03 (t, J = 7.16 Hz, 3 H) |
| 14 | 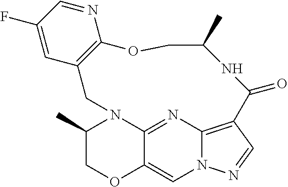 [SMILES: C[C@@H]1COc2ncc(F)cc2CN2c3nc4c(cnn4cc3OC[C@H]2C)C(=O)N1] | 399.2 | (500 MHz) 9.49 (d, J = 8.02 Hz, 1 H) 8.57 (s, 1 H) 8.08 (d, J = 2.86 Hz, 1 H) 7.99 (s, 1 H) 7.93 (dd, J = 8.59, 2.86 Hz, 1 H) 5.23 (d, J = 14.89 Hz, 1 H) 4.72 (dd, J = 10.88, 4.01 Hz, 1 H) 4.47-4.57 (m, 1 H) 4.35 (d, J = 14.89 Hz, 1 H) 4.29 (s, 2 H) 4.20- 4.28 (m, 1 H) 4.15 (d, J = 10.88 Hz, 1 H) 1.38 (d, J = 6.30 Hz, 3 H) 1.37 (d, J = 6.87 Hz, 3 H) |
| 15 | 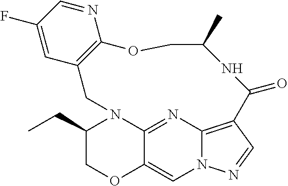 [SMILES: CC[C@@H]1COc2cn3ncc4c3nc2N1Cc1cc(F)cnc1OC[C@@H](C)NC4=O] | 413.2 | (500 MHz) 9.47 (d, J = 8.59 Hz, 1 H) 8.55 (s, 1 H) 8.07 (d, J = 2.86 Hz, 1 H) 7.98 (s, 1 H) 7.92 (dd, J = 8.59, 2.86 Hz, 1 H) 5.27 (d, J = 14.89 Hz, 1 H) 4.73 (dd, J = 10.88, 4.01 Hz, 1 H) 4.47 (d, J = 11.46 Hz, 1 H) 4.37 (d, J = 14.89 Hz, 1 H) 4.24-4.33 (m, 2 H) 4.19-4.24 (m, 1 H) 4.14 (dd, J = 10.88, 1.72 Hz, 1 H) 1.82-1.92 (m, 1 H) 1.62-1.73 (m, 1 H) 1.37 (d, J = 6.87 Hz, 3 H) 1.03 (t, J = 7.45 Hz, 3 H) |
| 16 | 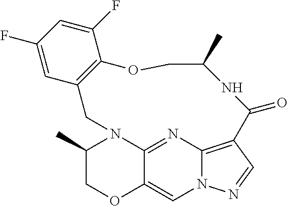 [SMILES: C[C@@H]1COc2c(F)cc(F)cc2CN2c3nc4c(cnn4cc3OC[C@H]2C)C(=O)N1] | 416.2 | (500 MHz) 9.88 (br d, J = 9.17 Hz, 1 H) 8.60 (s, 1 H) 8.01 (s, 1 H) 7.18- 7.31 (m, 1 H) 7.14 (br d, J = 8.59 Hz, 1 H) 5.38 (br d, J = 14.89 Hz, 1 H) 4.75 (br dd, J = 9.45, 6.59 Hz, 1 H) 4.49 (br d, J = 6.87 Hz, 1 H) 4.34- 4.40 (m, 1 H) 4.29-4.34 (m, 1 H) 4.26 (br d, J = 14.89 Hz, 1 H) 4.21 (br t, J = 7.45 Hz, 1 H) 3.98 (br d, J = 4.01 Hz, 1 H) 1.37 (br d, J = 6.30 Hz, 3 H) 1.34 (d, J = 6.87 Hz, 3 H) |
| 17 | 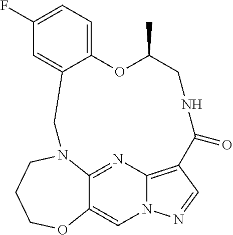 [SMILES: C[C@H]1CNC(=O)c2cnn3cc4c(nc23)N(CCCO4)Cc2cc(F)ccc2O1] | 398.2 | (300 MHz) 9.24 (dd, J = 2.7, 7.2 Hz, 1H), 8.57 (s, 1H), 8.02 (s, 1H), 7.15 (dd, J = 3.0, 9.5 Hz, 1H), 7.11-6.96 (m, 2H), 5.56 (d, J = 14.8 Hz, 1H), 4.61-4.49 (m, 1H), 4.41-4.21 (m, 3H), 4.15-4.04 (m, 2H), 3.92-3.80 (m, 1H), 3.15 (ddd, J = 3.2, 7.8, 13.5 Hz, 1H), 2.44-2.32 (m, 2H), 1.45 (d, J = 6.1 Hz, 3H) |
| 18 | 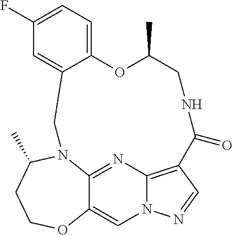 [SMILES: C[C@H]1CNC(=O)c2cnn3cc4c(nc23)N(Cc2cc(F)ccc2O1)[C@@H](C)CCO4] | 412.3 | 9.28 (dd, J = 7.73, 2.58 Hz, 1 H) 8.65 (s, 1 H) 8.05 (s, 1 H) 7.05 (dd, J = 9.17, 4.58 Hz, 1 H) 6.92-7.01 (m, 2H) 5.52 (d, J = 14.89 Hz, 1 H) 4.46-4.58 (m, 1 H) 4.33-4.46 (m, 1 H) 4.06 (dd, J = 10.88, 6.87 Hz, 1 H) 4.02 (d, J = 14.89 Hz, 1 H) 3.86- 3.97 (m, 2H) 3.06-3.15 (m, 1 H) 2.53-2.60 (m, 1 H) 2.01 (dt, J = 15.90, 5.23 Hz, 1 H) 1.73 (d, J = 6.87 Hz, 3 H) 1.45 (d, J = 5.73 Hz, 3 H) |
| 19 | 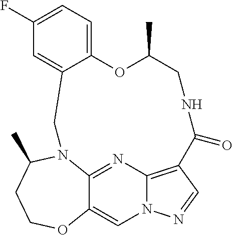 [SMILES: C[C@@H]1CCOc2cn3ncc4c3nc2N1Cc1cc(F)ccc1O[C@@H](C)CNC4=O] | 412.2 | 9.00-8.96 (m, 1H), 8.61 (s, 1H), 8.01 (s, 1H), 7.06 (dd, J = 4.0, 9.2 Hz, 2H), 7.00-6.94 (m, 1H), 5.44 (d, J = 14.9 Hz, 1H), 4.65-4.56 (m, 1H), 4.41-4.28 (m, 2H), 4.23-4.15 (m, 1H), 4.11 (d, J = 14.9 Hz, 1H), 3.79 (ddd, J = 4.0, 6.4, 13.6 Hz, 1H), 3.20-3.14 (m, 1H), 2.63-2.55 (m, 1H), 2.19 (qd, J = 5.0, 14.9 Hz, 1H), 1.44 (d, J = 6.9 Hz, 3H), 1.41 (d, J = 5.7 Hz, 3H) |
| 20 | 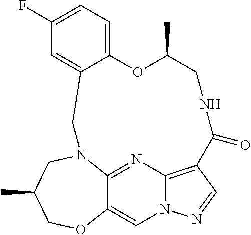 [SMILES: C[C@@H]1COc2cn3ncc4c3nc2N(Cc2cc(F)ccc2O[C@@H](C)CNC4=O)C1] | 412.2 | 9.17 (dd, J = 6.59, 3.72 Hz, 1 H) 8.57 (s, 1 H) 8.02 (s, 1 H) 7.13 (dd, J = 9.45, 3.15 Hz, 1 H) 7.05-7.11 (m, 1H) 6.95-7.05 (m, 1 H) 5.54 (d, J = 14.89 Hz, 1 H) 4.56-4.66 (m, 1 H) 4.32 (dd, J = 12.03, 6.30 Hz, 1 H) 4.15 (dd, J = 14.32, 4.58 Hz, 1 H) 4.10 (d, J = 14.89 Hz, 1 H) 3.89- 3.98 (m, 2 H) 3.84 (ddd, J = 13.60, 6.73, 4.30 Hz, 1 H) 3.18 (ddd, J = 13.75, 6.87, 4.01 Hz, 1 H) 2.64- 2.73 (m, 1 H) 1.45 (d, J = 6.30 Hz, 3 H) 1.07 (d, J = 6.87 Hz, 3 H) |
| 21 | 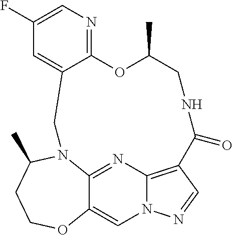 [SMILES: C[C@@H]1CCOc2cn3ncc4c3nc2N1Cc1cc(F)cnc1O[C@@H](C)CNC4=O] | 413.1 | 8.92 (dd, J = 7.73, 2.58 Hz, 1 H) 8.65 (s, 1 H) 8.06 (s, 1 H) 8.03 (d, J = 2.86 Hz, 1 H) 7.69 (dd, J = 9.16, 2.86 Hz, 1 H) 5.33 (dd, J = 14.89, 1.15 Hz, 1 H) 5.09-5.20 (m, 1 H) 4.37-4.46 (m, 1 H) 4.33 (ddd, J = 11.74, 8.88, 5.16 Hz, 1 H) 4.18-4.28 (m, 2 H) 3.95 (ddd, J = 13.03, 8.16, 4.58 Hz, 1 H) 3.15 (ddd, J = 13.17, 8.59, 2.86 Hz, 1 H) 2.64 (qd, J = 9.64, 5.44 Hz, 1 H) 2.18 (dq, J = 15.04, 4.73 Hz, 1 H) 1.46 (d, J = 6.87 Hz, 3 H) 1.44 (d, J = 6.30 Hz, 3 H) |
| 22 | 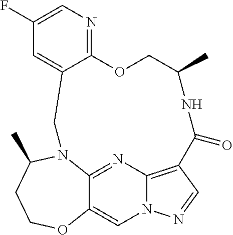 [SMILES: C[C@@H]1COc2ncc(F)cc2CN2c3nc4c(cnn4cc3OCC[C@H]2C)C(=O)N1] | 413.1 | 8.93 (d, J = 8.02 Hz, 1 H) 8.64 (s, 1 H) 8.00-8.08 (m, 2 H) 7.72 (dd, J = 8.88, 2.58 Hz, 1 H) 5.38 (d, J = 14.89 Hz, 1 H) 4.86 (dd, J = 11.17, 4.30 Hz, 1 H) 4.41-4.50 (m, 1 H) 4.34 (ddd, J = 11.60, 8.74, 5.44 Hz, 1 H) 4.23-4.31 (m, 2 H) 4.20 (dt, J = 11.46, 5.73 Hz, 1 H) 4.11 (dd, J = 10.88, 2.29 Hz, 1 H) 2.57-2.67 (m, 1 H) 2.19 (dq, J = 14.89, 4.96 Hz, 1 H) 1.49 (d, J = 6.87 Hz, 3 H) 1.37 (d, J = 6.87 Hz, 3 H) |
| 23 | 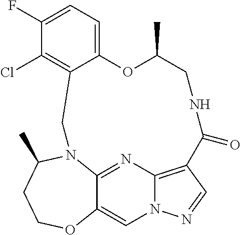 [SMILES: C[C@@H]1CCOc2cn3ncc4c3nc2N1Cc1c(ccc(F)c1Cl)O[C@@H](C)CNC4=O] | 446.2 | 9.16 (dd, J = 8.59, 2.29 Hz, 1 H) 8.68 (s, 1 H) 8.12 (s, 1 H) 7.20 (t, J = 9.17 Hz, 1 H) 7.03 (dd, J = 9.17, 4.58 Hz, 1 H) 5.79 (dd, J = 14.61, 1.43 Hz, 1 H) 4.49-4.56 (m, 1 H) 4.28-4.37 (m, 2 H) 4.08-4.19 (m, 2 H) 4.02 (ddd, J = 13.46, 8.88, 4.01 Hz, 1 H) 3.13 (ddd, J = 13.75, 8.59, 2.29 Hz, 1 H) 2.55-2.67 (m, 1 H) 2.00-2.09 (m, 1 H) 1.40 (d, J = 6.30 Hz, 3 H) 1.28 (d, J = 6.87 Hz, 3 H) |
| 24 | 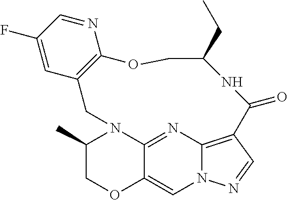 [SMILES: CC[C@@H]1COc2ncc(F)cc2CN2c3nc4c(cnn4cc3OC[C@H]2C)C(=O)N1] | 413.2 | 9.54 (d, J = 9.17 Hz, 1 H) 8.57 (s, 1 H) 8.08 (d, J = 2.86 Hz, 1 H) 7.99 (s, 1 H) 7.93 (dd, J = 9.17, 2.86 Hz, 1 H) 5.21 (dd, J = 14.89, 1.72 Hz, 1 H) 4.62 (dd, J = 10.60, 4.30 Hz, 1 H) 4.53 (q, J = 6.68 Hz, 1 H) 4.34 (d, J = 14.89 Hz, 1 H) 4.23-4.31 (m, 3 H) 4.03-4.11 (m, 1 H) 1.70-1.81 (m, 2 H) 1.38 (d, J = 6.30 Hz, 3 H) 0.97 (t, J = 7.16 Hz, 3 H) |
| 25 | 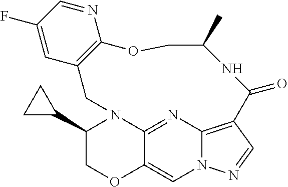 [SMILES: C[C@@H]1COc2ncc(F)cc2CN2c3nc4c(cnn4cc3OC[C@H]2C2CC2)C(=O)N1] | 425.1 | 9.45 (d, J = 8.59 Hz, 1 H) 8.59 (s, 1 H) 8.07 (d, J = 2.86 Hz, 1 H) 8.00 (s, 1 H) 7.85 (dd, J = 9.16, 2.86 Hz, 1 H) 5.31 (d, J = 14.89 Hz, 1 H) 4.75 (dd, J = 10.88, 4.01 Hz, 1 H) 4.50 (d, J = 14.89 Hz, 1 H) 4.36-4.44 (m, 1 H) 4.31 (dd, J = 11.46, 2.29 Hz, 1 H) 4.27 (ddd, J = 10.60, 6.30, 2.00 Hz, 1 H) 4.14 (dd, J = 10.31, 1.72 Hz, 1 H) 3.77 (br d, J = 9.74 Hz, 1 H) 1.38 (d, J = 6.87 Hz, 3 H) 1.08-1.17 (m, 1 H) 0.89 (dq, J = 9.52, 4.85 Hz, 1 H) 0.65- |
| | | | 0.73 (m, 1 H) 0.50-0.59 (m, 1 H) |
| | | | 0.33 (dq, J = 9.52, 4.85 Hz, 1 H) |
| 26 | 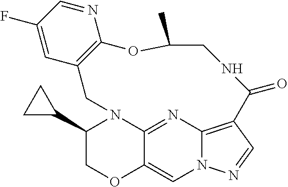 [SMILES: C[C@H]1CNC(=O)c2cnn3cc4c(nc23)N(Cc2cc(F)cnc2O1)[C@H](C1CC1)CO4] | 425.1 | 9.33-9.43 (m, 1 H) 8.60 (s, 1 H) 8.05 (d, J = 2.86 Hz, 1 H) 8.00 (s, 1 H) 7.80 (dd, J = 8.59, 2.86 Hz, 1 H) 5.19-5.29 (m, 1 H) 5.15 (ddd, J = 8.88, 6.01, 4.58 Hz, 1 H) 4.48 (d, J = 14.89 Hz, 1 H) 4.37-4.43 (m, 1 H) 4.31-4.37 (m, 1 H) 3.95 (ddd, J = 12.89, 8.31, 4.01 Hz, 1 H) 3.75 (br d, J = 9.17 Hz, 1 H) 3.15 (ddd, J = 12.75, 9.02, 2.29 Hz, 1 H) 1.45 (d, J = 5.73 Hz, 3 H) 1.06-1.17 (m, 1 H) 0.85 (dq, J = 9.67, 4.80 Hz, 1 H) |
| | | | 0.63-0.71 (m, 1 H) 0.54 (tt, J = 8.88, |
| | | | 4.58 Hz, 1 H) 0.33 (dq, J = 9.59, 5.01 |
| | | | Hz, 1 H) |
| 27 | 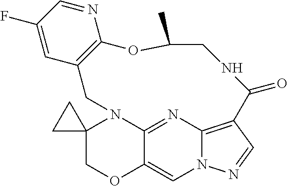 [SMILES: C[C@H]1CNC(=O)c2cnn3cc4c(nc23)N(Cc2cc(F)cnc2O1)C1(CC1)CO4] | 411.1 | 9.33 (br d, J = 6.30 Hz, 1 H) 8.63 (s, 1 H) 8.08 (d, J = 2.86 Hz, 1 H) 8.02 (s, 1 H) 7.56 (dd, J = 8.59, 2.86 Hz, 1H) 5.08-5.17 (m, 1 H) 5.00 (d, J = 16.04 Hz, 1 H) 4.61 (dd, J = 11.46, 1.72 Hz, 1 H) 3.97 (br d, J = 15.47 Hz, 1 H) 3.87-3.95 (m, 2 H) 3.14 (ddd, J = 13.17, 9.45, 2.00 Hz, 1 H) 1.79 (dt, J = 9.31, 6.23 Hz, 1 H) 1.45 (d, J = 5.73 Hz, 3 H) 1.12-1.24 (m, 2 H) 1.05 (dt, J = 10.74, 5.23 Hz, 1 H) |
| 28 | 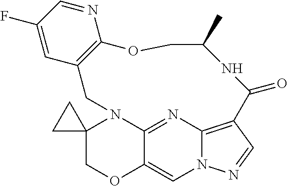 [SMILES: C[C@@H]1COc2ncc(F)cc2CN2c3nc4c(cnn4cc3OCC23CC3)C(=O)N1] | 411.1 | 9.41 (d, J = 8.59 Hz, 1 H) 8.63 (s, 1 H) 8.09 (d, J = 2.86 Hz, 1 H) 8.02 (s, 1 H) 7.58 (dd, J = 8.02, 2.86 Hz, 1 H) 5.04-5.14 (m, 1 H) 4.74 (dd, J = 10.60, 4.30 Hz, 1 H) 4.60 (dd, J = 11.17, 2.00 Hz, 1 H) 4.21-4.30 (m, 1 H) 4.17 (dd, J = 10.60, 1.43 Hz, 1 H) 3.99 (d, J = 16.04 Hz, 1 H) 3.91 (d, J = 11.46 Hz, 1 H) 1.82 (dt, J = 9.74, 6.30 Hz, 1 H) 1.36 (d, J = 6.87 Hz, 3 H) 1.12-1.25 (m, 2 H) 1.05 (dt, J = 10.74, 5.23 Hz, 1 H) |
| 29 | 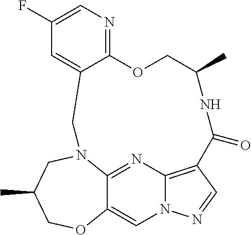 [SMILES: C[C@@H]1COc2cn3ncc4c3nc2N(Cc2cc(F)cnc2OC[C@@H](C)NC4=O)C1] | 413.1 | 9.14 (br d, J = 8.59 Hz, 1 H) 8.57 (s, 1 H) 8.06 (d, J = 2.86 Hz, 1 H) 8.02 (s, 1 H) 7.73 (dd, J = 8.59, 2.29 Hz, 1 H) 5.47 (br d, J = 14.89 Hz, 1 H) 4.79 (dd, J = 10.88, 4.01 Hz, 1 H) 4.21- 4.36 (m, 3 H) 4.09-4.17 (m, 2 H) 3.86-4.00 (m, 2 H) 2.70 (br d, J = 5.73 Hz, 1 H) 1.36 (d, J = 6.87 Hz, 3 H) 1.07 (d, J = 6.87 Hz, 3 H) |
| 30 | 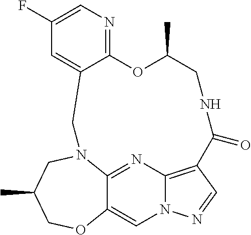 [SMILES: C[C@@H]1COc2cn3ncc4c3nc2N(Cc2cc(F)cnc2O[C@@H](C)CNC4=O)C1] | 413.1 | 9.09 (br d, J = 6.30 Hz, 1 H) 8.57 (s, 1 H) 8.05 (d, J = 2.86 Hz, 1 H) 8.03 (s, 1 H) 7.69 (dd, J = 8.59, 2.86 Hz, 1 H) 5.36-5.43 (m, 1 H) 5.08-5.17 (m, 1 H) 4.33 (dd, J = 12.32, 6.59 Hz, 1 H) 4.21 (d, J = 14.89 Hz, 1 H) 4.14 (dd, J = 14.03, 4.87 Hz, 1 H) 3.86- 4.00 (m, 3 H) 3.13 (ddd, J = 13.17, 9.16, 2.29 Hz, 1 H) 2.70 (br dd, J = 11.74, 6.01 Hz, 1 H) 1.45 (d, J = 6.30 Hz, 3 H) 1.07 (d, J = 6.87 Hz, 3 H) |
| 31 | 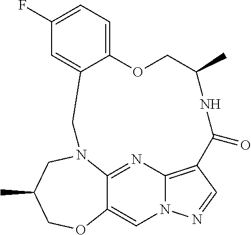 [SMILES: C[C@@H]1COc2cn3ncc4c3nc2N(Cc2cc(F)ccc2OC[C@@H](C)NC4=O)C1] | 412.1 | 9.39 (br d, J = 8.59 Hz, 1 H) 8.56 (s, 1 H) 8.03 (s, 1 H) 7.20 (dd, J = 9.17, 2.29 Hz, 1 H) 7.03-7.09 (m, 1 H) 6.95-7.02 (m, 1 H) 5.67 (br d, J = 14.89 Hz, 1 H) 4.33 (br d, J = 9.74 Hz, 1 H) 4.25-4.31 (m, 2 H) 4.10 (br d, J = 14.89 Hz, 2 H) 3.90-4.05 (m, 3 H) 2.69 (br d, J = 4.58 Hz, 1 H) 1.35 (d, J = 6.87 Hz, 3 H) 1.08 (d, J = 6.30 Hz, 3 H) |
| 32 | 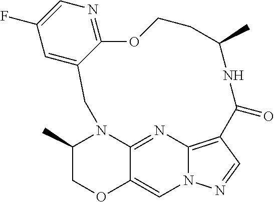 [SMILES: C[C@@H]1CCOc2ncc(F)cc2CN2c3nc4c(cnn4cc3OC[C@H]2C)C(=O)N1] | 413.1 | 8.53 (s, 1 H) 8.07 (br d, J = 7.45 Hz, 1 H) 8.03 (d, J = 2.29 Hz, 1 H) 7.99 (s, 1 H) 7.74-7.81 (m, 1 H) 5.25 (br d, J = 14.89 Hz, 1 H) 4.75 (br dd, J = 10.88, 5.73 Hz, 1 H) 4.45 (br d, J = 5.73 Hz, 1 H) 4.20-4.38 (m, 5 H) 2.22-2.32 (m, 1 H) 1.85-1.94 (m, 1 H) 1.37 (d, J = 6.30 Hz, 3 H) 1.25 (d, J = 6.30 Hz, 3 H) |
| 33 | 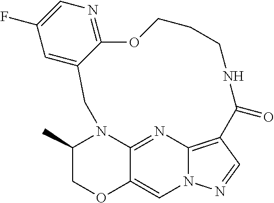 [SMILES: C[C@@H]1COc2cn3ncc4c3nc2N1Cc1cc(F)cnc1OCCCNC4=O] | 399.1 | 8.54 (s, 1 H) 8.09 (br s, 1 H) 8.05 (d, J = 2.29 Hz, 1 H) 8.00 (s, 1 H) 7.80 (br d, J = 8.59 Hz, 1 H) 5.25 (br d, J = 15.47 Hz, 1 H) 4.97-5.04 (m, 1 H) 4.45 (br d, J = 6.30 Hz, 1 H) 4.34- 4.41 (m, 1 H) 4.25-4.32 (m, 2 H) 4.20 (br t, J = 9.17 Hz, 1 H) 3.65- 3.74 (m, 1 H) 3.36-3.42 (m, 1 H) 1.95- 2.18 (m, 2 H) 1.36 (br d, J = 6.30 Hz, 3 H) |
| 34 | 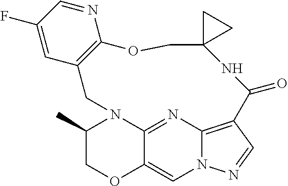 [SMILES: C[C@@H]1COc2cn3ncc4c3nc2N1Cc1cc(F)cnc1OCC1(CC1)NC4=O] | 411.1 | 9.08 (s, 1 H) 8.54 (s, 1 H) 8.06 (d, J = 2.29 Hz, 1 H) 7.90-7.96 (m, 2 H) 5.30 (br d, J = 14.89 Hz, 1 H) 4.84 (d, J = 10.88 Hz, 1 H) 4.51 (br d, J = 6.87 Hz, 1 H) 4.32 (br d, J = 14.89 Hz, 1 H) 4.28 (s, 2 H) 3.76 (d, J = 10.88 Hz, 1 H) 1.95-2.05 (m, 1 H) 1.39 (d, J = 6.87 Hz, 3 H) 0.97-1.04 (m, 1 H) 0.90-0.97 (m, 1 H) 0.78 (br t, J = 10.02 Hz, 1 H) |
| 35 | 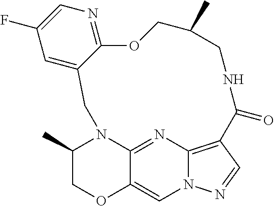 [SMILES: C[C@H]1CNC(=O)c2cnn3cc4c(nc23)N(Cc2cc(F)cnc2OC1)[C@H](C)CO4] | 413.1 | 8.54 (s, 1 H) 8.17 (br d, J = 5.73 Hz, 1 H) 8.05 (d, J = 2.86 Hz, 1 H) 7.99 (s, 1 H) 7.78 (dd, J = 8.31, 2.58 Hz, 1 H) 5.20 (br d, J = 15.47 Hz, 1 H) 4.95 (br d, J = 10.31 Hz, 1 H) 4.41-4.49 (m, 1 H) 4.34-4.40 (m, 1 H) 4.25- 4.32 (m, 2 H) 3.85-3.97 (m, 2 H) 3.01-3.09 (m, 1 H) 2.29-2.36 (m, 1 H) 1.36 (d, J = 6.87 Hz, 3 H) 1.02 (d, J = 6.87 Hz, 3 H) |
| 36 | 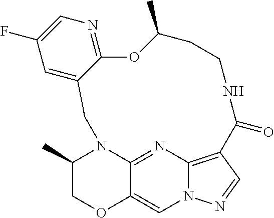 [SMILES: C[C@@H]1COc2cn3ncc4c3nc2N1Cc1cc(F)cnc1O[C@@H](C)CCNC4=O] | | 8.54 (s, 1 H) 8.09 (t, J = 4.30 Hz, 1 H) 8.04 (d, J = 2.86 Hz, 1 H) 7.99 (s, 1 H) 7.77 (dd, J = 8.59, 2.86 Hz, 1 H) 5.54 (br t, J = 6.30 Hz, 1 H) 5.21 (d, J = 14.89 Hz, 1 H) 4.35-4.46 (m, 2 H) 4.25-4.31 (m, 2 H) 3.61-3.68 (m, 1 H) 3.33-3.37 (m, 1 H) 2.12 (br dd, J = 15.18, 6.59 Hz, 1 H) 1.89- 2.00 (m, 1 H) 1.37 (d, J = 6.87 Hz, 3 H) 1.31 (d, J = 6.30 Hz, 3 H) |
| 37 | 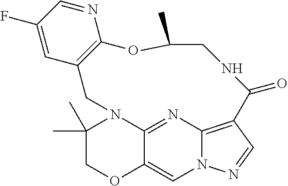 [SMILES: C[C@H]1CNC(=O)c2cnn3cc4c(nc23)N(Cc2cc(F)cnc2O1)C(C)(C)CO4] | 413.0 | 8.87 (t, J = 5.16 Hz, 1 H) 8.63 (s, 1 H) 8.05 (d, J = 2.86 Hz, 1 H) 8.01 (s, 1 H) 7.61 (dd, J = 8.88, 2.58 Hz, 1 H) 5.30-5.38 (m, 1 H) 4.93 (dd, J = 15.18, 1.43 Hz, 1 H) 4.53 (d, J = 15.47 Hz, 1 H) 4.20-4.33 (m, 2 H) 3.81 (dt, J = 13.17, 4.87 Hz, 1 H) 3.22-3.31 (m, 1 H) 1.61 (s, 3 H) 1.45 (d, J = 6.30 Hz, 3 H) 1.36 (s, 3 H) |
| 38 | 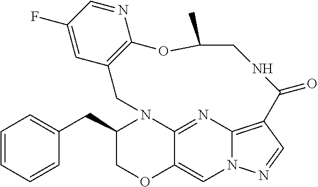 [SMILES: C[C@H]1CNC(=O)c2cnn3cc4c(nc23)N(Cc2cc(F)cnc2O1)[C@H](Cc1ccccc1)CO4] | 475.1 | 9.40 (dd, J = 8.02, 1.72 Hz, 1 H) 8.60 (s, 1 H) 8.06 (d, J = 2.86 Hz, 1 H) 8.01 (s, 1 H) 7.93 (dd, J = 9.17, 2.86 Hz, 1 H) 7.34-7.41 (m, 4 H) 7.25- 7.32 (m, 1 H) 5.06-5.19 (m, 2 H) 4.66 (br t, J = 7.45 Hz, 1 H) 4.13- 4.26 (m, 3 H) 3.95 (ddd, J = 13.03, 8.45, 4.30 Hz, 1 H) 3.10-3.23 (m, 2 H) 2.92 (dd, J = 13.75, 9.16 Hz, 1 H) 1.45 (d, J = 6.30 Hz, 3 H) |
| 39 | 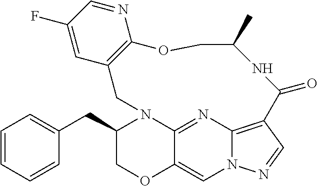 [SMILES: C[C@@H]1COc2ncc(F)cc2CN2c3nc4c(cnn4cc3OC[C@H]2Cc2ccccc2)C(=O)N1] | 475.1 | 9.40 (dd, J = 8.02, 1.72 Hz, 1 H) 8.60 (s, 1 H) 8.06 (d, J = 2.86 Hz, 1 H) 8.01 (s, 1 H) 7.93 (dd, J = 9.17, 2.86 Hz, 1 H) 7.34-7.41 (m, 4 H) 7.25- 7.32 (m, 1 H) 5.06-5.19 (m, 2 H) 4.66 (br t, J = 7.45 Hz, 1 H) 4.13- 4.26 (m, 3 H) 3.95 (ddd, J = 13.03, 8.45, 4.30 Hz, 1 H) 3.10-3.23 (m, 2 H) 2.92 (dd, J = 13.75, 9.16 Hz, 1 H) 1.45 (d, J = 6.30 Hz, 3 H) |
| 40 | 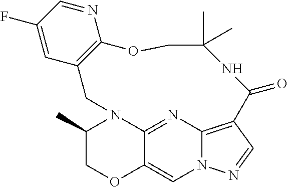 [SMILES: C[C@@H]1COc2cn3ncc4c3nc2N1Cc1cc(F)cnc1OCC(C)(C)NC4=O] | 413.1 | (300 MHz) 9.01 (s, 1 H) 8.54 (s, 1 H) 8.10 (d, J = 2.93 Hz, 1 H) 7.96- 8.01 (m, 1 H) 7.95 (s, 1 H) 5.24 (dd, J = 14.63, 1.60 Hz, 1 H) 4.65 (d, J = 10.73 Hz, 1 H) 4.53 (q, J = 6.39 Hz, 1 H) 4.33 (d, J = 14.86 Hz, 1 H) 4.28 (s, 2 H) 3.93 (d, J = 10.73 Hz, 1 H) 1.61 (s, 3 H) 1.49 (s, 3 H) 1.37 (d, J = 6.51 Hz, 3 H) |
| 41 | 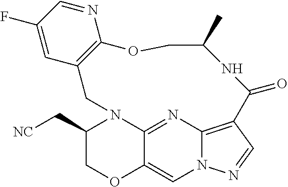 [SMILES: C[C@@H]1COc2ncc(F)cc2CN2c3nc4c(cnn4cc3OC[C@H]2CC#N)C(=O)N1] | 424.1 | (300 MHz) 9.41 (d, J = 8.16 Hz, 1 H) 8.67 (s, 1 H) 8.08 (d, J = 2.93 Hz, 1 H) 8.01-8.06 (m, 1 H) 7.97 (dd, J = 8.89, 2.84 Hz, 1 H) 5.27 (dd, J = 14.53, 1.51 Hz, 1 H) 4.90 (br t, J = 6.24 Hz, 1 H) 4.74 (dd, J = 10.73, 4.22 Hz, 1 H) 4.44-4.55 (m, 2 H) 4.34-4.41 (m, 1 H) 4.27 (ddt, J = 8.26, 4.14, 2.19, 2.19 Hz, 1 H) 4.14 (dd, J = 10.73, 2.02 Hz, 1 H) 3.19 (d, J = 6.60 Hz, 2 H) 1.37 (d, J = 6.69 Hz, 3 H) |
| 42 | 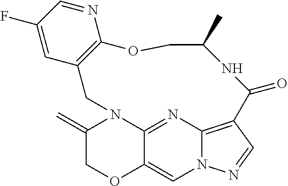 [SMILES: C=C1COc2cn3ncc4c3nc2N1Cc1cc(F)cnc1OC[C@@H](C)NC4=O] | 397.1 | (300 MHz) 9.31 (d, J = 8.53 Hz, 1 H) 8.81 (s, 1 H) 8.12 (s, 1 H) 8.11 (d, J = 3.03 Hz, 1 H) 7.53 (dd, J = 8.53, 2.93 Hz, 1 H) 5.53 (dd, J = 15.63, 1.24 Hz, 1 H) 5.34 (d, J = 2.11 Hz, 1 H) 5.01 (d, J = 15.68 Hz, 1 H) 4.86- 4.95 (m, 1 H) 4.70-4.86 (m, 3 H) 4.24-4.39 (m, 1 H) 4.20 (dd, J = 10.77, 1.60 Hz, 1 H) 1.40 (d, J = 6.79 Hz, 3 H) |
| 43 | 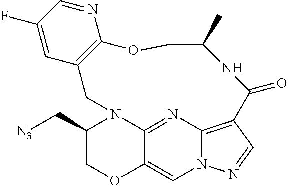 [SMILES: C[C@@H]1COc2ncc(F)cc2CN2c3nc4c(cnn4cc3OC[C@H]2CN=[N+]=[N-])C(=O)N1] | 440.2 | (300 MHz) 9.42 (d, J = 8.25 Hz, 1 H) 8.62 (s, 1 H) 8.07 (d, J = 2.93 Hz, 1 H) 8.01 (s, 1 H) 7.97 (dd, J = 8.76, 2.89 Hz, 1 H) 5.26 (dd, J = 14.81, 1.60 Hz, 1 H) 4.76 (dd, J = 10.82, 4.22 Hz, 1 H) 4.63 (br t, J = 6.24 Hz, 1 H) 4.50 (d, J = 2.84 Hz, 1 H) 4.46 (d, J = 6.97 Hz, 1 H) 4.21-4.36 (m, 2 H) 4.14 (dd, J = 10.73, 1.93 Hz, 1 H) 3.92 (dd, J = 12.56, 5.23 Hz, 1 H) 3.70 (dd, J = 12.56, 7.61 Hz, 1 H) 1.37 (d, J = 6.69 Hz, 3 H) |
| 44 | 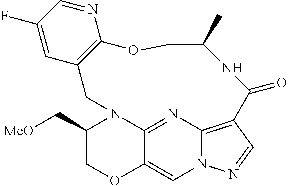 [SMILES: COC[C@@H]1COc2cn3ncc4c3nc2N1Cc1cc(F)cnc1OC[C@@H](C)NC4=O] | 429.1 | 9.42 (d, J = 8.02 Hz, 1 H) 8.57 (s, 1 H) 8.07 (d, J = 2.86 Hz, 1 H) 7.94- 8.02 (m, 2 H) 5.27 (d, J = 14.89 Hz, 1 H) 4.76 (dd, J = 10.60, 4.30 Hz, 1 H) 4.60 (br t, J = 6.30 Hz, 1 H) 4.46 (d, J = 11.46 Hz, 1 H) 4.41 (d, J = 14.89 Hz, 1 H) 4.23-4.32 (m, 2 H) 4.13 (d, J = 10.88 Hz, 1 H) 3.64-3.72 (m, 1 H) 3.56-3.64 (m, 1 H) 3.36 (s, 3 H) 1.37 (d, J = 6.87 Hz, 3 H) |
| 45 | 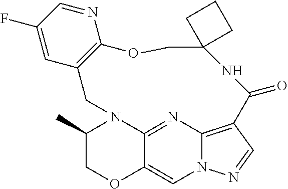 [SMILES: C[C@@H]1COc2cn3ncc4c3nc2N1Cc1cc(F)cnc1OCC1(CCC1)NC4=O] | 425.1 | 8.99 (s, 1 H) 8.55 (s, 1 H) 8.09- 8.13 (m, 1 H) 7.98 (s, 1 H) 7.96 (dd, J = 8.59, 2.86 Hz, 1 H) 5.16 (d, J = 14.89 Hz, 1 H) 4.81 (d, J = 10.88 Hz, 1 H) 4.50 (q, J = 6.30 Hz, 1 H) 4.37 (d, J = 10.88 Hz, 1 H) 4.22- 4.33 (m, 3 H) 3.55 (q, J = 10.31 Hz, 1 H) 2.65-2.89 (m, 1 H) 2.16-2.27 (m, 1 H) 2.02-2.11 (m, 1 H) 1.76- 1.93 (m, 2 H) 1.36 (d, J = 6.87 Hz, 3 H) |
| 46 | 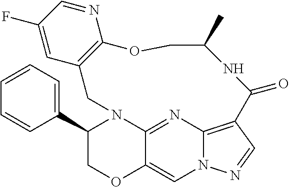 [SMILES: C[C@@H]1COc2ncc(F)cc2CN2c3nc4c(cnn4cc3OC[C@H]2c2ccccc2)C(=O)N1] | 461.2 | 9.49 (d, J = 8.02 Hz, 1 H) 8.63 (s, 1 H) 8.06-8.13 (m, 2 H) 8.05 (s, 1 H) 7.41-7.47 (m, 2 H) 7.38 (d, J = 7.45 Hz, 1 H) 7.35 (d, J = 8.02 Hz, 2 H) 5.77 (s, 1 H) 5.35 (d, J = 14.32 Hz, 1 H) 4.70 (dd, J = 10.60, 4.30 Hz, 1 H) 4.53-4.60 (m, 1 H) 4.44-4.51 (m, 1 H) 4.26-4.32 (m, 1 H) 4.14 (dd, J = 10.88, 1.72 Hz, 1 H) 4.09 (d, J = 14.89 Hz, 1 H) 1.38 (d, J = 6.87 Hz, 3 H) |
| 47 | 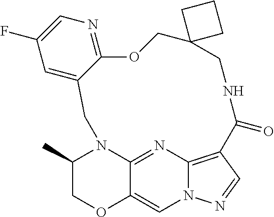 [SMILES: C[C@@H]1COc2cn3ncc4c3nc2N1Cc1cc(F)cnc1OCC1(CCC1)CNC4=O] | 439.2 | 8.54 (s, 1 H) 8.04-8.10 (m, 2 H) 8.01 (s, 1 H) 7.84 (dd, J = 8.88, 2.58 Hz, 1 H) 5.22 (d, J = 14.89 Hz, 1 H) 5.11 (d, J = 10.88 Hz, 1 H) 4.43- 4.51 (m, 1 H) 4.24-4.35 (m, 3 H) 4.05-4.14 (m, 1 H) 3.71 (dd, J = 13.46, 6.01 Hz, 1 H) 3.17 (d, J = 5.16 Hz, 1 H) 2.11-2.19 (m, 1 H) 1.87-2.06 (m, 4 H) 1.76-1.83 (m, 1 H) 1.36 (d, J = 6.30 Hz, 3 H) |
| 48 | 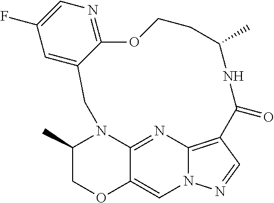 [SMILES: C[C@@H]1COc2cn3ncc4c3nc2N1Cc1cc(F)cnc1OCC[C@H](C)NC4=O] | 413.1 | 8.53-8.58 (m, 1 H) 8.14 (d, J = 7.45 Hz, 1 H) 8.09 (d, J = 2.86 Hz, 1 H) 7.98-8.05 (m, 1 H) 7.85 (dd, J = 8.31, 2.58 Hz, 1 H) 5.56 (t, J = 12.03 Hz, 1 H) 5.29 (br d, J = 14.89 Hz, 1 H) 4.43- 4.50 (m, 1 H) 4.20-4.41 (m, 4 H) 3.95-4.00 (m, 1 H) 2.23-2.33 (m, 1 H) 1.95 (br d, J = 14.89 Hz, 1 H) 1.34-1.39 (m, 3 H) 1.16-1.24 (m, 3 H) |
| 49 | 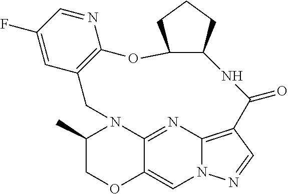 [SMILES: C[C@@H]1COc2cn3ncc4c3nc2N1Cc1cc(F)cnc1O[C@H]1CCC[C@H]1NC4=O] | 425.1 | 9.27 (d, J = 6.87 Hz, 1 H) 8.56 (s, 1 H) 8.03 (d, J = 2.86 Hz, 1 H) 7.97 (s, 1 H) 7.87 (dd, J = 9.17, 2.86 Hz, 1 H) 5.73 (td, J = 6.30, 3.44 Hz, 1 H) 5.14 (d, J = 14.89 Hz, 1 H) 4.47 (q, J = 6.30 Hz, 1 H) 4.27-4.36 (m, 3 H) 4.20- 4.27 (m, 1 H) 2.12-2.22 (m, 1 H) 2.02-2.12 (m, 1 H) 1.79-1.90 (m, 2 H) 1.68-1.79 (m, 1 H) 1.54-1.67 (m, 1 H) 1.38 (d, J = 6.30 Hz, 3 H) |
| 50 | 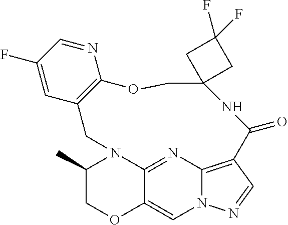 [SMILES: C[C@@H]1COc2cn3ncc4c3nc2N1Cc1cc(F)cnc1OCC1(CC(F)(F)C1)NC4=O] | 461.1 | 9.01 (s, 1 H) 8.58 (s, 1 H) 8.07- 8.14 (m, 1 H) 7.99-8.05 (m, 1 H) 7.96 (dd, J = 8.59, 2.86 Hz, 1 H) 5.11- 5.29 (m, 1 H) 5.04 (d, J = 11.46 Hz, 1 H) 4.49 (q, J = 6.30 Hz, 1 H) 4.22- 4.37 (m, 4 H) 3.89-4.02 (m, 1 H) 3.56-3.71 (m, 1 H) 2.99-3.10 (m, 1 H) 2.77-2.88 (m, 1 H) 1.37 (d, J = 6.30 Hz, 3 H) |
| 51 | 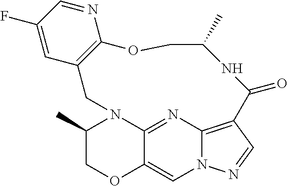 [SMILES: C[C@@H]1COc2cn3ncc4c3nc2N1Cc1cc(F)cnc1OC[C@H](C)NC4=O] | 399.1 | 8.95-9.21 (m, 1 H) 8.54-8.65 (m, 1 H) 8.08 (dd, J = 7.45, 2.86 Hz, 1 H) 7.98 (d, J = 5.73 Hz, 1 H) 7.65-7.97 (m, 1 H) 5.10-5.38 (m, 1 H) 4.77- 4.92 (m, 1 H) 4.00-4.57 (m, 6 H) 1.38-1.56 (m, 3 H) 1.33-1.38 (m, 3 H) |
| 52 | 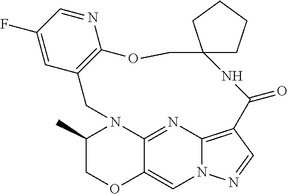 [SMILES: C[C@@H]1COc2cn3ncc4c3nc2N1Cc1cc(F)cnc1OCC1(CCCC1)NC4=O] | 439.2 | 9.11 (s, 1 H) 8.54 (s, 1 H) 8.10 (d, J = 2.86 Hz, 1 H) 7.89-8.01 (m, 2 H) 5.23 (d, J = 14.89 Hz, 1 H) 4.63 (d, J = 10.88 Hz, 1 H) 4.49-4.57 (m, 1 H) 4.23-4.36 (m, 3 H) 4.01 (d, J = 10.31 Hz, 1 H) 2.87 (ddd, J = 13.17, 9.16, 4.01 Hz, 1 H) 1.93- 2.07 (m, 2 H) 1.89 (dt, J = 12.17, 8.23 Hz, 1 H) 1.72-1.84 (m, 1 H) 1.67 (qd, J = 7.64, 4.58 Hz, 1 H) 1.50- 1.63 (m, 1 H) 1.44 (dt, J = 13.03, 8.09 Hz, 1 H) 1.37 (d, J = 6.30 Hz, 3 H) |
| 53 | 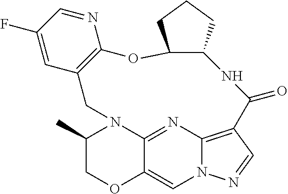 [SMILES: C[C@@H]1COc2cn3ncc4c3nc2N1Cc1cc(F)cnc1O[C@H]1CCC[C@@H]1NC4=O] | 425.2 | 9.08 (s, 1 H) 8.57 (s, 1 H) 8.14 (d, J = 2.86 Hz, 1 H) 7.96 (s, 1 H) 7.93 (dd, J = 8.59, 2.86 Hz, 1 H) 5.23 (br d, J = 14.32 Hz, 1 H) 4.61-4.68 (m, 1 H) 4.54 (q, J = 5.73 Hz, 1 H) 4.26- 4.36 (m, 3 H) 3.60-3.71 (m, 1 H) 2.39-2.48 (m, 1 H) 2.19-2.29 (m, 1 H) 1.79-1.90 (m, 2 H) 1.70-1.79 (m, 1 H) 1.59-1.70 (m, 1 H) 1.36 (d, J = 6.30 Hz, 3 H) |
| 54 | 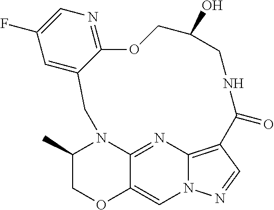 [SMILES: C[C@@H]1COc2cn3ncc4c3nc2N1Cc1cc(F)cnc1OC[C@@H](O)CNC4=O] | 415.1 | m 8.55 (s, 1 H) 8.08-8.13 (m, 1 H) 8.04 (d, J = 2.86 Hz, 1 H) 8.00 (s, 1 H) 7.77 (dd, J = 8.59, 2.86 Hz, 1 H) 5.38 (d, J = 4.58 Hz, 1 H) 5.21 (d, J = 14.89 Hz, 1 H) 5.01 (br d, J = 9.74 Hz, 1 H) 4.35-4.46 (m, 2 H) 4.25- 4.33 (m, 2 H) 3.98-4.10 (m, 2 H) 3.91-3.98 (m, 1 H) 3.08-3.16 (m, 1H) 1.36 (d, J = 6.87 Hz, 3 H) |
| 55 | 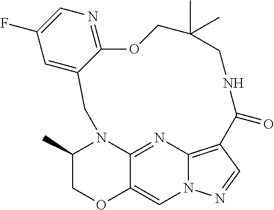 [SMILES: C[C@@H]1COc2cn3ncc4c3nc2N1Cc1cc(F)cnc1OCC(C)(C)CNC4=O] | 427.2 | 8.54 (s, 1 H) 8.20 (dd, J = 6.30, 2.29 Hz, 1 H) 8.05 (d, J = 2.86 Hz, 1 H) 8.00 (s, 1 H) 7.86 (dd, J = 8.59, 2.86 Hz, 1 H) 5.28 (d, J = 16.04 Hz, 1 H) 5.02 (d, J = 10.88 Hz, 1 H) 4.44- 4.51 (m, 1 H) 4.23-4.37 (m, 3 H) 3.68 (d, J = 10.31 Hz, 1 H) 3.46 (dd, J = 13.17, 6.30 Hz, 1 H) 3.13 (dd, J = 13.17, 2.29 Hz, 1 H) 1.37 (d, J = 6.87 Hz, 3 H) 1.21 (s, 3 H) 1.01 (s, 3H) |
| 56 | 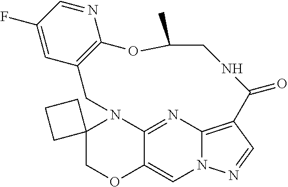 [SMILES: C[C@H]1CNC(=O)c2cnn3cc4c(nc23)N(Cc2cc(F)cnc2O1)C1(CCC1)CO4] | 425.0 | 9.13 (dd, J = 6.87, 3.44 Hz, 1 H) 8.63 (s, 1 H) 8.07 (d, J = 2.86 Hz, 1 H) 8.01 (s, 1 H) 7.28 (dd, J = 8.59, 2.86 Hz, 1 H) 5.19-5.29 (m, 1 H) 5.15 (d, J = 16.04 Hz, 1 H) 4.78 (d, J = 15.47 Hz, 1 H) 4.68 (d, J = 11.46 Hz, 1 H) 4.27 (dd, J = 11.46, 2.29 Hz, 1 H) 3.87 (ddd, J = 13.17, 6.87, 4.58 Hz, 1 H) 3.22 (ddd, J = 13.17, 7.45, 3.44 Hz, 1 H) 2.90-2.99 (m, 1 H) 2.33-2.42 (m, 1 H) 2.21-2.29 (m, 1 H) 2.12-2.19 (m, 1 H) 1.89-2.00 |
| | | | (m, 1 H) 1.78-1.89 (m, 1 H) 1.47 |
| | | | (d, J = 6.30 Hz, 3 H) |
| 57 | 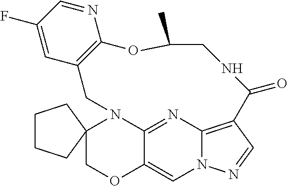 [SMILES: C[C@H]1CNC(=O)c2cnn3cc4c(nc23)N(Cc2cc(F)cnc2O1)C1(CCCC1)CO4] | 439.0 | 8.96 (t, J = 5.16 Hz, 1 H) 8.63 (s, 1 H) 8.06 (d, J = 2.86 Hz, 1 H) 8.01 (s, 1 H) 7.52 (dd, J = 8.59, 2.86 Hz, 1 H) 5.26-5.35 (m, 1 H) 5.02-5.11 (m, 1 H) 4.38 (d, J = 15.47 Hz, 1 H) 4.22- 4.32 (m, 2 H) 3.79-3.87 (m, 1 H) 3.20-3.28 (m, 1 H) 2.21-2.29 (m, 1 H) 1.95-2.03 (m, 1 H) 1.84-1.94 (m, 2 H) 1.74-1.84 (m, 2 H) 1.63- 1.74 (m, 2 H) 1.45 (d, J = 6.30 Hz, 3 H) |
| 58 | 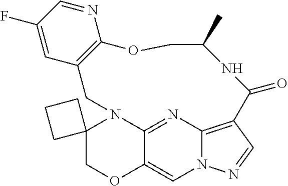 [SMILES: C[C@@H]1COc2ncc(F)cc2CN2c3nc4c(cnn4cc3OCC23CCC3)C(=O)N1] | 425.0 | 9.20 (d, J = 7.45 Hz, 1 H) 8.62 (s, 1 H) 8.07 (d, J = 2.86 Hz, 1 H) 8.00 (s, 1 H) 7.28 (dd, J = 8.59, 2.86 Hz, 1 H) 5.16-5.24 (m, 1 H) 4.91 (dd, J = 11.17, 4.30 Hz, 1 H) 4.79 (d, J = 15.47 Hz, 1 H) 4.67 (d, J = 11.46 Hz, 1 H) 4.26 (dd, J = 11.46, 1.72 Hz, 1 H) 4.18-4.24 (m, 1 H) 4.15 (dd, J = 10.88, 2.29 Hz, 1 H) 2.93-3.02 (m, 1 H) 2.34-2.44 (m, 1 H) 2.26 (ddd, J = 11.89, 8.16, 4.01 Hz, 1 H) 2.12-2.20 (m, 1 H) 1.90-2.01 (m, |
| | | | 1 H) 1.81-1.90 (m, 1 H) 1.37 (d, |
| | | | J = 6.87 Hz, 3 H) |
| 59 | 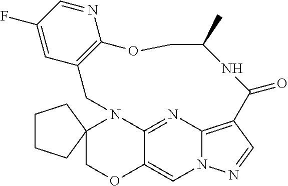 [SMILES: C[C@@H]1COc2ncc(F)cc2CN2c3nc4c(cnn4cc3OCC23CCCC3)C(=O)N1] | 439.0 | 8.96 (d, J = 6.87 Hz, 1 H) 8.61 (s, 1 H) 8.05 (d, J = 2.86 Hz, 1 H) 7.99 (s, 1 H) 7.52 (dd, J = 8.59, 2.86 Hz, 1 H) 5.11 (d, J = 14.32 Hz, 1 H) 5.03 (dd, J = 10.88, 4.58 Hz, 1 H) 4.39 (d, J = 14.89 Hz, 1 H) 4.22-4.31 (m, 2 H) 4.19 (ddd, J = 10.74, 6.73, 4.30 Hz, 1 H) 4.08 (dd, J = 11.17, 3.72 Hz, 1 H) 2.24-2.32 (m, 1 H) 1.95-2.04 (m, 1 H) 1.84-1.95 (m, 2 H) 1.75- 1.84 (m, 2 H) 1.62-1.75 (m, 2 H) 1.36 (d, J = 6.87 Hz, 3 H) |
| 60 | 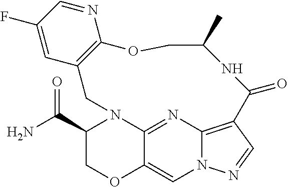 [SMILES: C[C@@H]1COc2ncc(F)cc2CN2c3nc4c(cnn4cc3OC[C@H]2C(N)=O)C(=O)N1] | 428.1 | 9.45 (d, J = 8.02 Hz, 1 H) 8.58 (s, 1 H) 8.08 (d, J = 3.44 Hz, 1 H) 8.03 (s, 1 H) 7.93 (dd, J = 8.59, 2.86 Hz, 1 H) 7.77 (s, 1 H) 7.59 (s, 1 H) 5.37 (dd, J = 14.89, 1.15 Hz, 1 H) 5.07 (s, 1 H) 4.74 (dd, J = 10.60, 4.30 Hz, 1 H) 4.70 (d, J = 10.88 Hz, 1 H) 4.45 (dd, J = 11.46, 2.86 Hz, 1 H) 4.27 (dddd, J = 8.31, 6.30, 4.30, 1.72 Hz, 2 H) 4.12-4.20 (m, 4 H) 1.39 (d, J = 6.87 Hz, 3 H) |
| 61 | 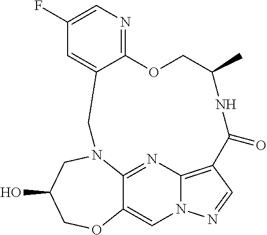 [SMILES: C[C@@H]1COc2ncc(F)cc2CN2C[C@H](O)COc3cn4ncc(c4nc32)C(=O)N1] | 415.1 | 9.16 (br d, J = 8.59 Hz, 1 H) 8.59 (d, J = 1.72 Hz, 1 H) 8.01-8.07 (m, 2 H) 7.75 (br d, J = 8.02 Hz, 1 H) 5.44- 5.54 (m, 2 H) 4.78 (br dd, J = 10.60, 3.72 Hz, 1 H) 4.49 (br s, 1 H) 4.39 (br dd, J = 12.60, 5.16 Hz, 1 H) 4.18- 4.30 (m, 3 H) 4.00-4.16 (m, 3 H) 1.36 (br d, J = 6.87 Hz, 3 H) |
| 62 | 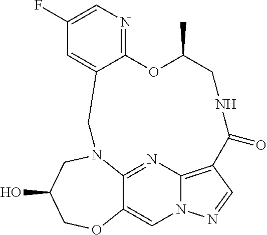 [SMILES: C[C@H]1CNC(=O)c2cnn3cc4c(nc23)N(Cc2cc(F)cnc2O1)C[C@H](O)CO4] | 415.1 | 9.09 (dd, J = 8.02, 1.72 Hz, 1 H) 8.60 (s, 1 H) 8.02-8.06 (m, 2 H) 7.71 (dd, J = 8.59, 2.86 Hz, 1 H) 5.50 (d, J = 4.58 Hz, 1 H) 5.41 (dd, J = 15.18, 1.43 Hz, 1 H) 5.06-5.17 (m, 1 H) 4.46-4.54 (m, 1 H) 4.39 (dd, J = 12.60, 5.16 Hz, 1 H) 4.16-4.29 (m, 2 H) 4.01-4.10 (m, 2 H) 3.96 (ddd, J = 13.17, 8.59, 4.58 Hz, 1 H) 3.10-3.19 (m, 1 H) 1.45 (d, J = 5.73 Hz, 3 H) |
| 63 | 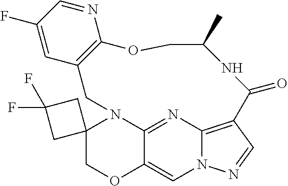 [SMILES: C[C@@H]1COc2ncc(F)cc2CN2c3nc4c(cnn4cc3OCC23CC(F)(F)C3)C(=O)N1] | 461.1 | 9.12 (br d, J = 8.02 Hz, 1 H) 8.72 (s, 1 H) 8.07 (br s, 1 H) 8.04 (br s, 1 H) 7.38 (br d, J = 8.02 Hz, 1 H) 5.29 (br d, J = 16.04 Hz, 1 H) 4.95 (br dd, J = 10.88, 4.01 Hz, 1 H) 4.56 (br d, J = 16.61 Hz, 1 H) 4.52 (s, 1 H) 4.40 (br d, J = 12.03 Hz, 1 H) 4.23 (br d, J = 1.15 Hz, 1 H) 4.13 (br d, J = 10.88 Hz, 1 H) 3.76 (q, J = 13.75 Hz, 1 H) 3.09- 3.28 (m, 2 H) 2.95 (br t, J = 13.46 Hz, 1 H) 1.37 (br d, J = 6.30 Hz, 3 H) |
| 64 | 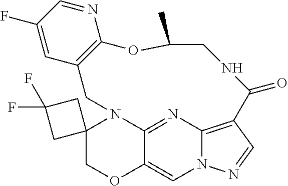 [SMILES: C[C@H]1CNC(=O)c2cnn3cc4c(nc23)N(Cc2cc(F)cnc2O1)C1(CO4)CC(F)(F)C1] | 461.1 | 9.05 (br s, 1 H) 8.73 (s, 1 H) 8.07 (br s, 1 H) 8.05 (br s, 1 H) 7.39 (br d, J = 8.59 Hz, 1 H) 5.24-5.32 (m, 1 H) 5.22 (br d, J = 16.04 Hz, 1 H) 4.55 (br s, 1 H) 4.52 (s, 1 H) 4.41 (br d, J = 12.03 Hz, 1 H) 3.82-3.92 (m, 1 H) 3.71 (q, J = 14.70 Hz, 1 H) 3.09- 3.27 (m, 3 H) 2.90-3.01 (m, 1 H) 1.47 (d, J = 6.30 Hz, 3 H) |
| 65 | 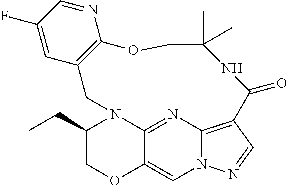 [SMILES: CC[C@@H]1COc2cn3ncc4c3nc2N1Cc1cc(F)cnc1OCC(C)(C)NC4=O] | 427.2 | 8.98 (s, 1 H) 8.53 (s, 1 H) 8.10 (d, J = 2.29 Hz, 1 H) 7.98 (br d, J = 7.45 Hz, 1 H) 7.94 (s, 1 H) 5.27 (br d, J = 14.89 Hz, 1 H) 4.69 (d, J = 10.88 Hz, 1 H) 4.47 (br d, J = 10.88 Hz, 1 H) 4.36 (br d, J = 14.89 Hz, 1 H) 4.27- 4.33 (m, 1 H) 4.21 (br d, J = 10.88 Hz, 1 H) 3.92 (d, J = 10.88 Hz, 1 H) 1.82-1.94 (m, 1 H) 1.63-1.70 (m, 1 H) 1.61 (s, 3 H) 1.50 (s, 3 H) 1.02 (t, J = 7.45 Hz, 3 H) |
| 66 | 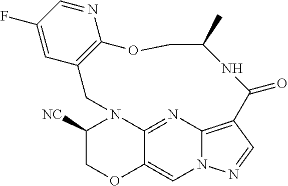 [SMILES: C[C@@H]1COc2ncc(F)cc2CN2c3nc4c(cnn4cc3OC[C@H]2C#N)C(=O)N1] | 410.1 | 9.30 (br d, J = 8.02 Hz, 1 H) 8.87 (s, 1 H) 8.11 (s, 1 H) 8.10 (d, J = 2.29 Hz, 1 H) 8.03 (br d, J = 8.59 Hz, 1 H) 5.90 (s, 1 H) 5.37 (br d, J = 14.32 Hz, 1 H) 4.86 (br d, J = 12.03 Hz, 1 H) 4.68 (br dd, J = 10.60, 3.72 Hz, 1 H) 4.51-4.61 (m, 2 H) 4.29 (br d, J = 1.72 Hz, 1 H) 4.16 (br d, J = 10.88 Hz, 1 H) 1.39 (br d, J = 6.30 Hz, 3 H) |
| 67 | 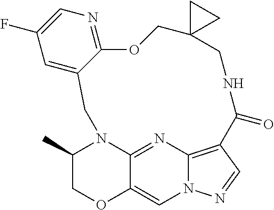 [SMILES: C[C@@H]1COc2cn3ncc4c3nc2N1Cc1cc(F)cnc1OCC1(CC1)CNC4=O] | | 8.55 (s, 1 H) 8.26 (t, J = 4.58 Hz, 1 H) 8.03 (d, J = 3.44 Hz, 1 H) 8.00 (s, 1 H) 7.81 (dd, J = 9.16, 2.86 Hz, 1 H) 5.32 (dd, J = 14.89, 1.15 Hz, 1 H) 5.24 (d, J = 11.46 Hz, 1 H) 4.46 (q, J = 6.30 Hz, 1 H) 4.24-4.38 (m, 3 H) 3.70 (d, J = 11.46 Hz, 1 H) 3.63 (dd, J = 13.75, 4.58 Hz, 1 H) 3.07 (dd, J = 13.75, 4.58 Hz, 1 H) 1.38 (d, J = 6.87 Hz, 3 H) 0.73-0.87 (m, 2 H) 0.52-0.69 (m, 2 H) |
| 68 | 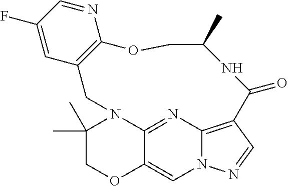 [SMILES: C[C@@H]1COc2ncc(F)cc2CN2c3nc4c(cnn4cc3OCC2(C)C)C(=O)N1] | | 8.83 (d, J = 6.30 Hz, 1 H) 8.61 (s, 1 H) 8.04 (d, J = 2.86 Hz, 1 H) 7.98 (s, 1 H) 7.60 (dd, J = 8.59, 2.86 Hz, 1 H) 5.06 (dd, J = 11.17, 4.87 Hz, 1 H) 4.97 (d, J = 15.47 Hz, 1 H) 4.53 (d, J = 14.89 Hz, 1 H) 4.28-4.32 (m, 1 H) 4.16-4.23 (m, 2 H) 4.04 (dd, J = 10.88, 4.58 Hz, 1 H) 1.63 (s, 3 H) 1.34- 1.39 (m, 6 H) |
| 69 | 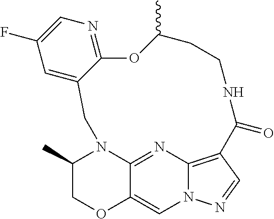 [SMILES: CC1CCNC(=O)c2cnn3cc4c(nc23)N(Cc2cc(F)cnc2O1)[C@H](C)CO4] | 413.1 | 8.56 (d, J = 17.18 Hz, 1 H) 7.92- 8.11 (m, 3 H) 7.46-7.80 (m, 1 H) 5.51-5.72 (m, 1 H) 5.17-5.28 (m, 1 H) 4.16-4.45 (m, 3 H) 3.99-4.06 (m, 1 H) 3.61-3.70 (m, 1 H) 2.08-2.22 (m, 1 H) 1.81-1.99 (m, 1 H) 1.29- 1.60 (m, 6H) |
| 70 | 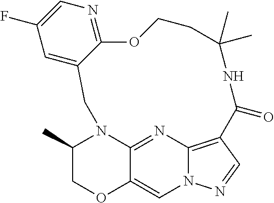 [SMILES: C[C@@H]1COc2cn3ncc4c3nc2N1Cc1cc(F)cnc1OCCC(C)(C)NC4=O] | 427.2 | 8.50 (s, 1 H) 8.07 (d, J = 2.86 Hz, 1 H) 7.99 (s, 1 H) 7.87 (dd, J = 8.59, 2.86 Hz, 1 H) 7.81 (s, 1 H) 5.35 (d, J = 14.32 Hz, 1 H) 4.94 (t, J = 10.88 Hz, 1 H) 4.49 (br d, J = 6.30 Hz, 1 H) 4.21-4.27 (m, 3 H) 4.06 (dd, J = 11.17, 5.44 Hz, 1 H) 2.24 (br dd, J = 15.47, 10.31 Hz, 1 H) 1.88 (dd, J = 15.47, 5.73 Hz, 1 H) 1.54 (s, 3 H) 1.52 (s, 3 H) 1.36 (d, J = 6.30 Hz, 3 H) |
| 71 | 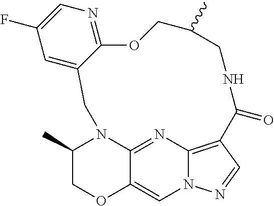 [SMILES: CC1CNC(=O)c2cnn3cc4c(nc23)N(Cc2cc(F)cnc2OC1)[C@H](C)CO4] | | 8.54 (d, J = 2.29 Hz, 1 H) 8.15-8.25 (m, 1 H) 8.05 (d, J = 2.86 Hz, 1 H) 8.00 (d, J = 7.45 Hz, 1 H) 7.76-7.88 (m, 1 H) 5.17-5.33 (m, 1 H) 4.79- 4.97 (m, 1 H) 4.24-4.51 (m, 4 H) 3.58-3.99 (m, 2 H) 2.98-3.11 (m, 1 H) 2.30-2.39 (m, 1 H) 1.36 (d, J = 6.30 Hz, 3 H) 1.03 (dd, J = 7.16, 3.72 Hz, 3 H) |
| 72 | 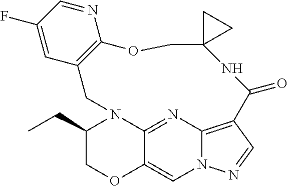 [SMILES: CC[C@@H]1COc2cn3ncc4c3nc2N1Cc1cc(F)cnc1OCC1(CC1)NC4=O] | 425.1 | 9.07 (s, 1 H) 8.53 (s, 1 H) 8.06 (d, J = 2.86 Hz, 1 H) 7.88-7.95 (m, 2 H) 5.34 (d, J = 14.32 Hz, 1 H) 4.86 (d, J = 10.31 Hz, 1 H) 4.46 (d, J = 11.46 Hz, 1 H) 4.34 (d, J = 14.89 Hz, 1 H) 4.30 (br dd, J = 8.02, 5.73 Hz, 1 H) 4.16-4.23 (m, 1 H) 3.74 (d, J = 10.88 Hz, 1 H) 1.95-2.02 (m, 1 H) 1.82-1.92 (m, 1 H) 1.63-1.73 (m, 1 H) 1.03 (t, J = 7.45 Hz, 3 H) 0.92-1.00 (m, 2 H) 0.75-0.82 (m, 1H) |
| 73 | 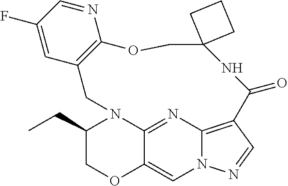 [SMILES: CC[C@@H]1COc2cn3ncc4c3nc2N1Cc1cc(F)cnc1OCC1(CCC1)NC4=O] | 439.2 | 8.96 (s, 1 H) 8.53 (s, 1 H) 8.09 (d, J = 2.86 Hz, 1 H) 7.98 (s, 1 H) 7.95 (dd, J = 8.59, 2.86 Hz, 1 H) 5.19 (d, J = 14.89 Hz, 1 H) 4.84 (d, J = 10.88 Hz, 1 H) 4.46 (d, J = 11.46 Hz, 1 H) 4.30-4.38 (m, 2 H) 4.28 (br d, J = 6.30 Hz, 1 H) 4.21 (br d, J = 11.46 Hz, 1 H) 3.49-3.58 (m, 1 H) 2.68- 2.76 (m, 1 H) 2.18-2.27 (m, 1 H) 2.03-2.13 (m, 1 H) 1.78-1.91 (m, 3 H) 1.59-1.71 (m, 1 H) 1.01 (t, J = 7.45 Hz, 3 H) |
| 74 | 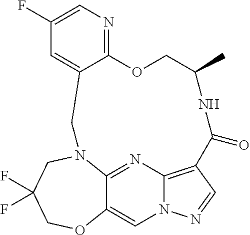 [SMILES: C[C@@H]1COc2ncc(F)cc2CN2CC(F)(F)COc3cn4ncc(c4nc32)C(=O)N1] | 435.1 | 9.07 (d, J = 8.59 Hz, 1 H) 8.86 (s, 1 H) 8.13 (s, 1 H) 8.08 (d, J = 2.86 Hz, 1 H) 7.69 (dd, J = 8.88, 2.58 Hz, 1 H) 5.48 (d, J = 14.89 Hz, 1 H) 4.80 (dd, J = 10.88, 4.01 Hz, 1 H) 4.59-4.78 (m, 4 H) 4.38 (d, J = 15.47 Hz, 1 H) 4.24-4.33 (m, 1 H) 4.14 (dd, J = 10.88, 1.15 Hz, 1 H) 1.37 (d, J = 6.30 Hz, 3 H) |
| 75 | 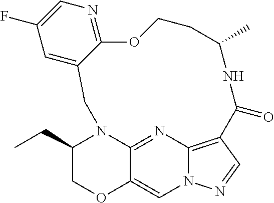 [SMILES: CC[C@@H]1COc2cn3ncc4c3nc2N1Cc1cc(F)cnc1OCC[C@H](C)NC4=O] | 427.2 | 8.52 (s, 1 H) 8.13 (d, J = 6.87 Hz, 1 H) 8.08 (d, J = 2.86 Hz, 1 H) 7.99- 8.01 (m, 1 H) 7.84 (dd, J = 8.88, 3.15 Hz, 1 H) 5.57 (t, J = 11.74 Hz, 1 H) 5.33 (br d, J = 14.32 Hz, 1 H) 4.45 (d, J = 11.46 Hz, 1 H) 4.22-4.32 (m, 4 H) 3.95-4.00 (m, 1 H) 2.25-2.34 (m, 1 H) 1.95 (br d, J = 16.04 Hz, 1 H) 1.79- 1.89 (m, 1 H) 1.61-1.69 (m, 1 H) 1.20 (d, J = 6.30 Hz, 3 H) 1.01 (t, J = 7.45 Hz, 3 H) |
| 76 | 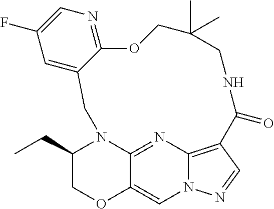 [SMILES: CC[C@@H]1COc2cn3ncc4c3nc2N1Cc1cc(F)cnc1OCC(C)(C)CNC4=O] | 441.2 | 8.53 (s, 1 H) 8.21 (dd, J = 6.01, 2.58 Hz, 1 H) 8.05 (d, J = 3.44 Hz, 1 H) 8.00 (s, 1 H) 7.85 (dd, J = 8.59, 2.86 Hz, 1 H) 5.32 (d, J = 14.89 Hz, 1 H) 5.02 (d, J = 10.88 Hz, 1 H) 4.45 (d, J = 10.88 Hz, 1 H) 4.23-4.36 (m, 3 H) 3.65-3.72 (m, 1 H) 3.40-3.48 (m, 1 H) 3.15 (dd, J = 13.46, 2.58 Hz, 1 H) 1.80-1.90 (m, 1 H) 1.67 (ddd, J = 14.03, 8.88, 7.45 Hz, 1 H) 1.21 (s, 3 H) 0.99-1.04 (m, 6 H) |
| 77 | 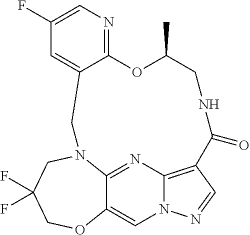 [SMILES: C[C@H]1CNC(=O)c2cnn3cc4c(nc23)N(Cc2cc(F)cnc2O1)CC(F)(F)CO4] | 435.1 | 9.00 (dd, J = 7.73, 2.00 Hz, 1 H) 8.87 (s, 1 H) 8.14 (s, 1 H) 8.07 (d, J = 2.86 Hz, 1 H) 7.65 (dd, J = 8.88, 2.58 Hz, 1 H) 5.41 (d, J = 14.89 Hz, 1 H) 5.09- 5.17 (m, 1 H) 4.59-4.77 (m, 4 H) 4.35 (d, J = 15.47 Hz, 1 H) 3.96 (ddd, J = 13.03, 8.45, 4.30 Hz, 1 H) 3.16 (ddd, J = 13.32, 8.74, 2.58 Hz, 1 H) 1.45 (d, J = 6.30 Hz, 3 H) |
| 78 | 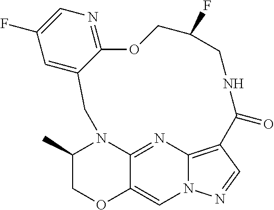 [SMILES: C[C@@H]1COc2cn3ncc4c3nc2N1Cc1cc(F)cnc1OC[C@@H](F)CNC4=O] | 417.1 | 8.56 (s, 1 H) 8.16 (t, J = 4.87 Hz, 1 H) 8.08 (d, J = 2.86 Hz, 1 H) 8.02 (s, 1 H) 7.86 (dd, J = 8.88, 2.58 Hz, 1 H) 5.07-5.40 (m, 3 H) 4.26-4.47 (m, 5 H) 3.93-4.01 (m, 1 H) 3.56-3.69 (m, 1 H) 1.37 (d, J = 6.30 Hz, 3 H) |
| 79 | 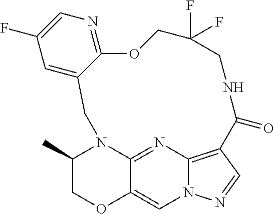 [SMILES: C[C@@H]1COc2cn3ncc4c3nc2N1Cc1cc(F)cnc1OCC(F)(F)CNC4=O] | 435.1 | 8.59 (s, 1 H) 8.10-8.18 (m, 2 H) 8.05 (s, 1 H) 7.90 (dd, J = 8.88, 2.58 Hz, 1 H) 5.59-5.71 (m, 1 H) 5.19 (d, J = 14.89 Hz, 1 H) 4.43-4.49 (m, 1 H) 4.35-4.41 (m, 3 H) 4.29 (d, J = 11.46 Hz, 1 H) 3.87-4.10 (m, 2 H) 1.55 (d, J = 6.87 Hz, 1 H) 1.37 (d, J = 6.30 Hz, 3 H) |
| 80 | 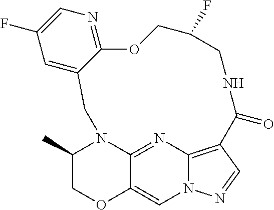 [SMILES: C[C@@H]1COc2cn3ncc4c3nc2N1Cc1cc(F)cnc1OC[C@H](F)CNC4=O] | 417.1 | 8.55-8.62 (m, 1 H) 8.22 (dd, J = 6.87, 3.44 Hz, 1 H) 8.05-8.09 (m, 1 H) 7.99-8.04 (m, 1 H) 7.54- 7.87 (m, 1 H) 5.45-5.58 (m, 1 H) 5.20-5.32 (m, 1 H) 4.98-5.18 (m, 1 H) 4.43- 4.51 (m, 1 H) 4.03-4.38 (m, 5 H) 3.44-3.63 (m, 1 H) 1.33-1.59 (m, 3 H) |
| 81 | 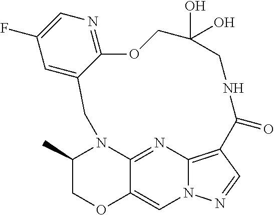 [SMILES: C[C@@H]1COc2cn3ncc4c3nc2N1Cc1cc(F)cnc1OCC(O)(O)CNC4=O] | 431.1 | |
| 82 | 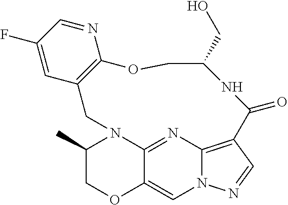 [SMILES: C[C@@H]1COc2cn3ncc4c3nc2N1Cc1cc(F)cnc1OC[C@H](CO)NC4=O] | 415.1 | (300 MHz) 9.07-9.29 (m, 1 H) 8.53- 8.65 (m, 1 H) 8.05-8.12 (m, 1 H) 7.99-8.04 (m, 1 H) 7.69-7.98 (m, 1 H) 5.24-5.37 (m, 1 H) 5.01-5.21 (m, 1 H) 4.88 (dd, J = 11.00, 4.86 Hz, 1 H) 4.22-4.56 (m, 4 H) 4.03-4.22 (m, 2 H) 3.89-4.03 (m, 1 H) 3.75 (dt, J = 10.52, 5.05 Hz, 1 H) 3.49- 3.65 (m, 2 H) 1.32-1.61 (m, 3 H) |
| 83 | 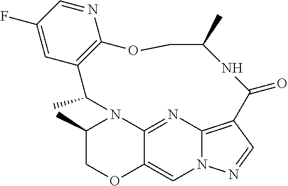 [SMILES: C[C@@H]1COc2ncc(F)cc2[C@@H](C)N2c3nc4c(cnn4cc3OC[C@H]2C)C(=O)N1] | 413.2 | 9.33 (d, J = 8.59 Hz, 1 H) 8.59 (s, 1 H) 7.97-8.03 (m, 2 H) 7.83 (dd, J = 9.17, 2.86 Hz, 1 H) 5.84-5.92 (m, 1 H) 4.85 (dd, J = 10.88, 4.01 Hz, 1 H) 4.75-4.81 (m, 1 H) 4.36 (d, J = 10.88 Hz, 1 H) 4.22-4.30 (m, 1 H) 4.09-4.19 (m, 2 H) 1.66 (d, J = 7.45 Hz, 3 H) 1.38 (dd, J = 9.17, 6.87 Hz, 6 H) |
| 84 | 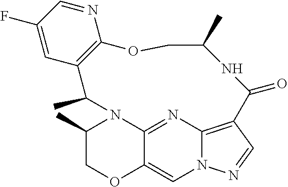 [SMILES: C[C@@H]1COc2ncc(F)cc2[C@H](C)N2c3nc4c(cnn4cc3OC[C@H]2C)C(=O)N1] | 413.2 | (300 MHz) 9.31 (br d, J = 6.79 Hz, 1 H) 9.26 (d, J = 1.83 Hz, 2 H) 9.14 (d, J = 8.90 Hz, 1 H) 8.65 (s, 2 H) 8.55 (s, 1 H) 8.17-8.21 (m, 1 H) 8.15 (t, J = 2.75 Hz, 3 H) 8.04 (d, J = 2.93 Hz, 1 H) 8.02 (s, 2 H) 7.82 (dd, J = 9.26, 3.03 Hz, 2 H) 6.24 (dd, J = 7.24, 1.65 Hz, 2 H) 5.00-5.11 (m, 1 H) 4.92 (dd, J = 10.91, 4.22 Hz, 1 H) 4.60 (q, J = 7.00 Hz, 2 H) 4.45-4.54 (m, 1 H) 4.27-4.41 (m, 7 H) 3.99-4.10 (m, 6 H) 2.04 (d, J = 7.34 Hz, 4 H) 1.59 |
| | | | (d, J = 7.24 Hz, 7 H) 1.46 (d, J = 6.42 |
| | | | Hz, 7 H) 1.31-1.43 (m, 12 H) 1.20- |
| | | | 1.27 (m, 16 H) |
| 85 | 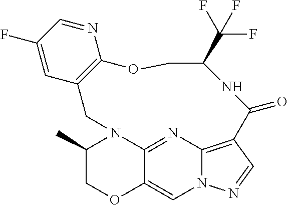 [SMILES: C[C@@H]1COc2cn3ncc4c3nc2N1Cc1cc(F)cnc1OC[C@@H](C(F)(F)F)NC4=O] | 453.1 | (300 MHz) 10.02 (d, J = 8.44 Hz, 1 H) 8.62 (s, 1 H) 8.12 (d, J = 2.93 Hz, 1 H) 8.08 (s, 1 H) 7.97 (dd, J = 8.80, 2.93 Hz, 1 H) 5.09-5.20 (m, 1 H) 4.92-5.09 (m, 2 H) 4.50-4.62 (m, 1 H) 4.34-4.50 (m, 2 H) 4.29 (s, 2 H) 1.39 (d, J = 6.60 Hz, 3 H) |
| 86 | 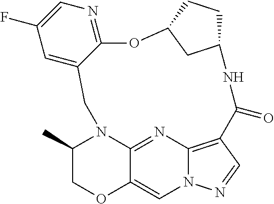 [SMILES: C[C@@H]1COc2cn3ncc4c3nc2N1Cc1cc(F)cnc1O[C@@H]1CC[C@@H](C1)NC4=O] | 425.1 | (300 MHz) 8.51-8.62 (m, 1 H) 8.27- 8.50 (m, 1 H) 7.50-8.13 (m, 3 H) 5.15-5.51 (m, 2 H) 4.48-4.82 (m, 1 H) 4.00- 4.46 (m, 4 H) 2.27-2.42 (m, 1 H) 2.08-2.19 (m, 2 H) 1.89-2.05 (m, 1 H) 1.67-1.84 (m, 2 H) 1.35-1.60 (m, 3 H) |
| 87 | 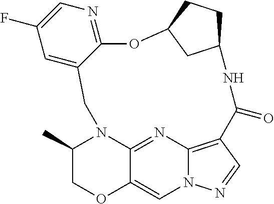 [SMILES: C[C@@H]1COc2cn3ncc4c3nc2N1Cc1cc(F)cnc1O[C@H]1CC[C@H](C1)NC4=O] | 425.1 | (300 MHz) 8.55 (s, 1 H) 8.38 (d, J = 10.36 Hz, 1 H) 8.06 (d, J = 2.84 Hz, 1 H) 7.98 (s, 1 H) 7.75 (dd, J = 8.76, 2.80 Hz, 1 H) 5.38 (br d, J = 1.38 Hz, 1 H) 5.20 (br d, J = 14.95 Hz, 1 H) 4.67-4.84 (m, 1 H) 4.24- 4.51 (m, 4 H) 2.22-2.37 (m, 1 H) 2.10-2.19 (m, 1 H) 1.91-2.04 (m, 3 H) 1.73-1.86 (m, 1 H) 1.37 (d, J = 6.42 Hz, 3 H) |
| 88 | 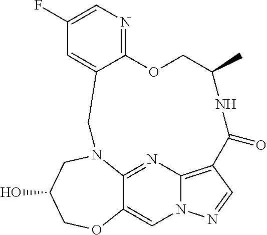 [SMILES: C[C@@H]1COc2ncc(F)cc2CN2C[C@@H](O)COc3cn4ncc(c4nc32)C(=O)N1] | 415.0 | (300 MHz) 9.20 (d, J = 8.80 Hz, 1 H) 8.59 (s, 1 H) 8.51 (dd, J = 9.26, 2.93 Hz, 1 H) 8.01-8.07 (m, 2 H) 5.94 (d, J = 3.67 Hz, 1 H) 5.46 (dd, J = 14.95, 1.65 Hz, 1 H) 4.81 (dd, J = 10.87, 4.08 Hz, 1 H) 4.36-4.51 (m, 3 H) 4.11-4.30 (m, 3 H) 3.76- 3.94 (m, 2 H) 1.36 (d, J = 6.69 Hz, 3 H) |
| 89 | 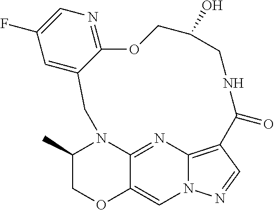 [SMILES: C[C@@H]1COc2cn3ncc4c3nc2N1Cc1cc(F)cnc1OC[C@H](O)CNC4=O] | 415.2 | (300 MHz) 8.55 (s, 1 H) 8.07 (d, J = 2.84 Hz, 1 H) 8.01 (s, 1 H) 7.95 (dd, J = 6.24, 2.20 Hz, 1 H) 7.85 (dd, J = 8.67, 2.80 Hz, 1 H) 5.45 (d, J = 4.95 Hz, 1 H) 5.24 (dd, J = 14.95, 1.28 Hz, 1 H) 4.87-4.99 (m, 1 H) 4.46 (br d, J = 6.69 Hz, 1 H) 4.23- 4.36 (m, 3 H) 4.04 (br d, J = 10.64 Hz, 2 H) 3.80 (ddd, J = 9.42, 6.12, 3.26 Hz, 1 H) 3.02-3.15 (m, 1 H) 1.36 (d, J = 6.51 Hz, 3 H) |
| 90 | 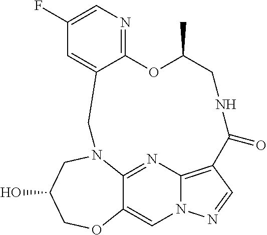 [SMILES: C[C@H]1CNC(=O)c2cnn3cc4c(nc23)N(Cc2cc(F)cnc2O1)C[C@@H](O)CO4] | 415.2 | (300 MHz) 9.14 (br d, J = 7.15 Hz, 1 H) 8.59 (s, 1 H) 8.49 (dd, J = 9.22, 2.98 Hz, 1 H) 8.00-8.06 (m, 2 H) 5.95 (d, J = 3.67 Hz, 1 H) 5.40 (dd, J = 15.08, 1.42 Hz, 1 H) 5.03-5.16 (m, 1 H) 4.32-4.52 (m, 3 H) 4.18 (br d, J = 15.50 Hz, 1 H) 3.76-4.03 (m, 3 H) 3.11 (ddd, J = 13.32, 9.65, 1.79 Hz, 1 H) 1.45 (d, J = 6.14 Hz, 3 H) |
| 91 | 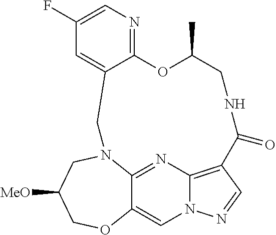 [SMILES: CO[C@@H]1COc2cn3ncc4c3nc2N(Cc2cc(F)cnc2O[C@@H](C)CNC4=O)C1] | 429.1 | (300 MHz) 9.10 (br d, J = 6.79 Hz, 1 H) 8.61 (s, 1 H) 7.98-8.11 (m, 2 H) 7.75 (dd, J = 8.57, 2.80 Hz, 1 H) 5.43 (dd, J = 14.95, 1.47 Hz, 1 H) 5.05- 5.17 (m, 1 H) 4.33-4.45 (m, 2 H) 4.11-4.28 (m, 4 H) 3.92-4.03 (m, 1 H) 3.38 (s, 3 H) 3.07-3.17 (m, 1 H) 1.45 (d, J = 6.14 Hz, 3 H) |
| 92 | 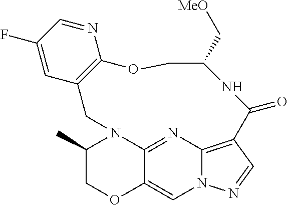 [SMILES: COC[C@H]1COc2ncc(F)cc2CN2c3nc4c(cnn4cc3OC[C@H]2C)C(=O)N1] | 429.1 | (300 MHz) 8.94-9.36 (m, 1 H) 8.53- 8.68 (m, 1 H) 8.06-8.13 (m, 1 H) 7.98-8.04 (m, 1 H) 7.71-7.97 (m, 1 H) 4.83-5.40 (m, 2 H) 4.01-4.55 (m, 7 H) 3.50-3.71 (m, 2 H) 3.32 (s, 2 H) 1.33-1.57 (m, 4 H) |
| 93 | 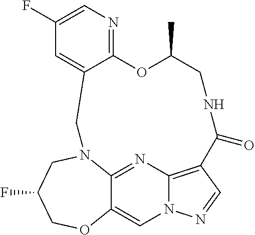 [SMILES: C[C@H]1CNC(=O)c2cnn3cc4c(nc23)N(Cc2cc(F)cnc2O1)C[C@@H](F)CO4] | 417.1 | (300 MHz) 9.02 (dd, J = 7.61, 2.29 Hz, 1 H) 8.71 (s, 1 H) 8.08 (s, 1 H) 8.06 (d, J = 2.93 Hz, 1 H) 7.82 (br d, J = 8.99 Hz, 1 H) 5.27-5.57 (m, 2 H) 5.04-5.21 (m, 1 H) 4.40-4.69 (m, 3 H) 4.23-4.40 (m, 2 H) 3.88-4.01 (m, 1 H) 3.15 (ddd, J = 13.20, 8.80, 2.66 Hz, 1 H) 1.45 (d, J = 6.24 Hz, 3 H) |
| 94 | 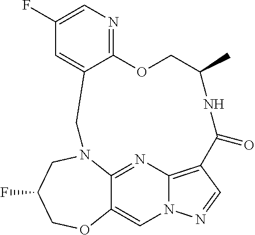 [SMILES: C[C@@H]1COc2ncc(F)cc2CN2C[C@@H](F)COc3cn4ncc(c4nc32)C(=O)N1] | 417.1 | (300 MHz) 9.11 (d, J = 8.34 Hz, 1 H) 8.70 (s, 1 H) 8.02-8.14 (m, 2 H) 7.86 (br d, J = 9.08 Hz, 1 H) 5.29- 5.57 (m, 2 H) 4.81 (dd, J = 10.87, 4.26 Hz, 1 H) 4.40-4.69 (m, 3 H) 4.19-4.40 (m, 3 H) 4.13 (dd, J = 10.82, 1.93 Hz, 1 H) 1.36 (d, J = 6.79 Hz, 3 H) |
| 95 | 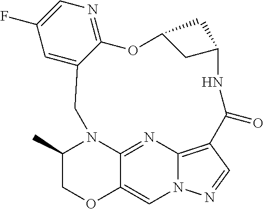 [SMILES: C[C@@H]1COc2cn3ncc4c3nc2N1Cc1cc(F)cnc1O[C@H]1C[C@H](C1)NC4=O] | 411.1 | (300 MHz) 9.02 (br d, J = 10.18 Hz, 1 H) 8.58 (d, J = 1.10 Hz, 1 H) 7.98- 8.10 (m, 2 H) 7.89 (br d, J = 8.80 Hz, 1 H) 5.42 (br d, J = 15.31 Hz, 1 H) 5.12 (br d, J = 2.84 Hz, 1 H) 4.67 (br dd, J = 5.18, 1.60 Hz, 1 H) 4.44- 4.57 (m, 1 H) 4.24-4.42 (m, 3 H) 2.98-3.12 (m, 1 H) 2.82-2.96 (m, 1 H) 2.11 (br dd, J = 12.70, 8.02 Hz, 1 H) 1.66 (br dd, J = 13.89, 7.57 Hz, 1 H) 1.39 (br d, J = 5.96 Hz, 3 H) |
| 96 | 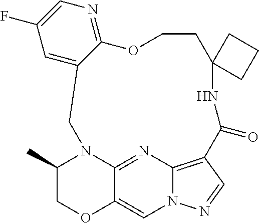 [SMILES: C[C@@H]1COc2cn3ncc4c3nc2N1Cc1cc(F)cnc1OCCC1(CCC1)NC4=O] | 439.2 | (300 MHz) 8.51 (d, J = 1.74 Hz, 1 H) 8.04 (br d, J = 6.33 Hz, 2 H) 7.83- 7.92 (m, 2 H) 5.33 (br d, J = 15.04 Hz, 1 H) 4.62-4.77 (m, 1 H) 4.41- 4.55 (m, 1 H) 4.20-4.32 (m, 3 H) 4.08-4.17 (m, 1 H) 3.13-3.28 (m, 2 H) 2.15-2.31 (m, 2 H) 1.81-1.94 (m, 2 H) 1.65-1.77 (m, 2 H) 1.35 (br d, J = 5.50 Hz, 3 H) |
| 97 | 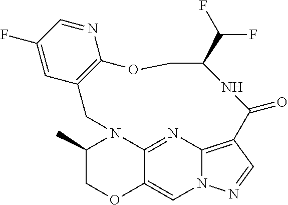 [SMILES: C[C@@H]1COc2cn3ncc4c3nc2N1Cc1cc(F)cnc1OC[C@@H](C(F)F)NC4=O] | 435.1 | (300 MHz) 9.79 (d, J = 8.99 Hz, 1 H) 8.61 (s, 1 H) 8.10 (d, J = 2.93 Hz, 1 H) 8.05 (s, 1 H) 7.95 (dd, J = 8.71, 3.03 Hz, 1 H) 6.10-6.55 (m, 1 H) 5.23 (dd, J = 14.81, 1.42 Hz, 1 H) 4.88 (br dd, J = 11.78, 4.81 Hz, 1 H) 4.47-4.63 (m, 2 H) 4.43 (d, J = 11.74 Hz, 1 H) 4.31-4.40 (m, 1 H) 4.29 (s, 2 H) 1.39 (d, J = 6.42 Hz, 3 H) |
| 98 | 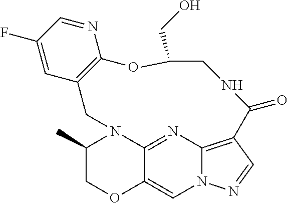 [SMILES: C[C@@H]1COc2cn3ncc4c3nc2N1Cc1cc(F)cnc1O[C@H](CO)CNC4=O] | 415.1 | (300 MHz, DMSO-d₆) δ ppm 8.82- 8.93 (m, 1 H) 8.57-8.62 (m, 1 H) 8.06 (d, J = 2.84 Hz, 1 H) 7.96-8.01 (m, 1 H) 7.60 (dd, J = 9.03, 2.71 Hz, 1 H) 5.15-5.25 (m, 2 H) 5.05-5.11 (m, 1 H) 4.18-4.41 (m, 3 H) 4.06 (dt, J = 6.76, 3.54 Hz, 1 H) 3.72- 3.83 (m, 2 H) 3.62-3.71 (m, 1 H) 3.48 (dt, J = 13.16, 5.02 Hz, 1 H) 1.59 (d, J = 6.60 Hz, 3 H) |
| 99 | 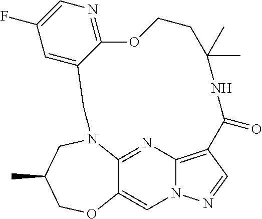 [SMILES: C[C@@H]1COc2cn3ncc4c3nc2N(Cc2cc(F)cnc2OCCC(C)(C)NC4=O)C1] | 441.2 | (300 MHz, DMSO-d₆) δ ppm 8.48- 8.54 (m, 1 H) 8.01-8.07 (m, 2 H) 7.63-7.82 (m, 2 H) 5.54-5.67 (m, 1 H) 4.95-5.08 (m, 1 H) 4.24-4.44 (m, 1 H) 4.01-4.20 (m, 3 H) 3.72- 3.93 (m, 2 H) 2.63-2.76 (m, 1 H) 2.17-2.31 (m, 1 H) 1.87 (br dd, J = 15.63, 5.09 Hz, 1 H) 1.53 (d, J = 10.00 Hz, 6 H) 1.04 (dd, J = 11.00, 6.88 Hz, 3 H) |
| 100 | 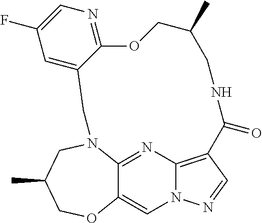 [SMILES: C[C@@H]1COc2cn3ncc4c3nc2N(Cc2cc(F)cnc2OC[C@@H](C)CNC4=O)C1] | 427.2 | (300 MHz, DMSO-d₆) δ ppm 8.47- 8.65 (m, 1 H) 7.94-8.09 (m, 3 H) 7.52-7.81 (m, 1 H) 5.34-5.63 (m, 1 H) 4.77-5.04 (m, 1 H) 4.30-4.47 (m, 1 H) 4.07-4.25 (m, 2 H) 3.79- 3.99 (m, 4 H) 2.93-3.07 (m, 1 H) 2.61-2.77 (m, 1 H) 2.27-2.38 (m, 1H) 0.97-1.10 (m, 6 H) |

Biologic Assays

In-Vitro Assays

Materials and Methods

Biochemical Kinase Assay Method

The biochemical kinase assay was performed at Reaction Biology Corporation (www.reactionbiology.com, Malvern, Pa.) following the procedures described in the reference (Anastassiadis T, et al Nat Biotechnol. 2011, 29, 1039). Specific kinase/substrate pairs along with required cofactors were prepared in reaction buffer; 20 mM Hepes pH 7.5, 10 mM MgC₂, 1 mM EGTA, 0.02% Brij35, 0.02 mg/ml BSA, 0.1 mM Na₃VO₄, 2 mM DTT, 1% DMSO. Compounds were delivered into the reaction, followed ˜20 min later by addition of a mixture of ATP (Sigma, St. Louis Mo.) and ³³P ATP (Perkin Elmer, Waltham Mass.) to a final concentration of 10 μM. Reactions were carried out at room temperature for 120 min, followed by spotting of the reactions onto P81 ion exchange filter paper (Whatman Inc., Piscataway, N.J.). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

Cell Lines and Cell Culture:

Colorectal cell line KM 12 (harboring endogenous TPM3-TRKA fusion gene) was obtained from NCI. Acute myelogenous cell line KG-1 (harboring endogenous OP2-FGFR1 fusion gene) were purchased from ATCC.

Cloning and Ba/F3 or NIH3T3 Stable Cell Line Creation

The EML4-ALK gene (variant 1) was synthesized at GenScript and cloned into pCDH-CMV-MCS-EF1-Puro plasmid (System Biosciences, Inc). Ba/F3-EML4-ALK wild type were generated by transducing Ba/F3 cells with lentivirus containing EML4-ALK wide type. Stable cell lines were selected by puromycin treatment, followed by IL-3 withdrawal. Briefly, 5×10⁶ Ba/F3 cells were transduced with lentivirus supernatant in the presence of 8 μg/mL protamine sulfate. The transduced cells were subsequently selected with 1 μg/mL puromycin in the presence of IL3-containing medium RPMI1640, plus 10% FBS. After 10-12 days of selection, the surviving cells were further selected for IL3 independent growth.

Cell Proliferation Assays:

Two thousand cells per well were seeded in 384 well white plate for 24 hrs, and then treated with compounds for 72 hours (37° C., 5% CO₂). Cell proliferation was measured using CellTiter-Glo luciferase-based ATP detection assay (Promega) following the manufactures's protocol. IC₅₀ determinations were performed using GraphPad Prism software (GraphPad, Inc., San Diego, Calif.).

Data and Results:

Enzymatic Kinase Activities of Compound 7.

| | Compound 7 |
|---|---|
| Kinase | IC₅₀ (nM) at 10 μM ATP |
| ALK | 0.853 |
| ALK (G1202R) | 1.25 |
| ALK (L1196M) | 0.358 |
| FGFR1 | 53.0 |
| FGFR1(V561M) | 137 |
| FGFR2 | 14.7 |
| FGFR3 | 30.5 |
| FGFR4 | 88.4 |
| ROS1 | 0.111 |
| ROS1 (G2032R) | 0.678 |

### Anti-Cell Proliferation Activity

| | BaF3 | KM12 | KG-1 cell |
|---|---|---|---|
| | EML4-ALK | (TPM3-TRKA) | (OP2-FGFR1) |
| Cpd | IC₅₀ (nM) | IC₅₀ (nM) | IC₅₀ (nM) |
| 1 | 968.7 | 0.2 | 1334 |
| 2 | >10000 | 26.7 | >10000 |
| 3 | 94.2 | 0.2 | 271 |
| 4 | >10000 | 0.2 | >10000 |
| 5 | 37.7 | 0.2 | 194.5 |
| 6 | 20.6 | 0.2 | 233.7 |
| 7 | 18.6 | 0.2 | 145.2 |
| 8 | 101.2 | 0.2 | 77.5 |
| 9 | 11.7 | 0.2 | 111.4 |
| 10 | 71.2 | 0.2 | 63.1 |
| 11 | 3174 | 91.1 | 4000 |
| 12 | 1457 | 0.8 | 2000 |
| 13 | 28 | 0.2 | 145.4 |
| 14 | 13.9 | <0.2 | 33.6 |
| 15 | 14 | <0.2 | 25.2 |
| 16 | 90.2 | <0.2 | 827.2 |
| 17 | 2645 | <0.2 | 5000 |
| 18 | >10000 | <0.2 | >10000 |
| 19 | 6215 | <0.2 | 9951 |
| 20 | 4906 | 5.1 | 2425 |
| 21 | 3000 | 0.6 | 1823 |
| 22 | 730.7 | <0.2 | 783.7 |
| 23 | 1934 | <0.2 | 9507 |
| 24 | 103.6 | — | 50 |
| 25 | 89.1 | <0.2 | 130.9 |
| 26 | 692.4 | <0.2 | 693.9 |
| 27 | 603.6 | <0.2 | 3568 |
| 28 | 179.9 | <0.2 | 786.5 |
| 29 | 826.4 | 9.6 | 129.6 |
| 30 | 7382 | 42 | 536.1 |
| 31 | 1330 | <0.2 | 779 |
| 32 | 74.6 | <0.2 | 137 |
| 33 | 58.4 | <0.2 | 37.1 |
| 34 | 23.7 | <0.2 | 12.5 |
| 35 | 48.2 | <0.2 | 33.5 |
| 36 | 60.9 | <0.2 | 25.3 |
| 37 | 1553 | — | 5000 |
| 38 | 7242 | — | 357.4 |
| 39 | 1609 | — | 73.7 |
| 40 | <0.2 | <0.2 | 0.2 |
| 41 | 1171 | 0.5 | 610.6 |
| 42 | 1200 | <0.2 | 1230 |
| 43 | 85 | 0.2 | 30.6 |
| 44 | 548.5 | <0.2 | 168.2 |
| 45 | 2.7 | <0.2 | 5 |
| 46 | 1056 | <0.2 | 0.2 |
| 47 | 68 | <0.2 | 24.2 |
| 48 | 22.1 | <0.2 | 12.9 |
| 49 | 55.1 | <0.2 | 9.5 |
| 50 | 582.6 | <0.2 | 412 |
| 51 | 233.1 | <0.2 | 100.7 |
| 52 | 276.5 | <0.2 | 358.8 |
| 53 | 113 | <0.2 | 182.9 |
| 54 | 661.1 | <0.2 | 677.7 |
| 55 | 14.8 | <0.2 | 4.2 |
| 56 | 919.4 | <0.2 | 3000 |
| 57 | 2208 | <0.2 | 4000 |
| 58 | 203.3 | <0.2 | 1622 |
| 59 | 1015 | <0.2 | 2000 |
| 60 | >10000 | 51.2 | >10000 |
| 61 | 4000 | 482 | 482.2 |
| 62 | 10000 | 94.8 | 681.3 |
| 63 | 576.2 | <0.2 | 10000 |
| 64 | 1229 | <0.2 | 10000 |
| 65 | 164 | <0.2 | 42.1 |
| 66 | 10000 | 462.9 | 10000 |
| 67 | 32.4 | <0.2 | 8.8 |
| 68 | 339.2 | <0.2 | 5690 |
| 69 | 87.9 | <0.2 | 23.7 |
| 70 | 35.7 | <0.2 | 51.7 |
| 71 | 19.9 | <0.2 | 11.9 |
| 72 | 18.8 | <0.2 | 3.9 |
| 73 | 93.1 | <0.2 | 5 |
| 74 | >10000 | 84 | 3000 |
| 75 | 19.8 | <0.2 | 9.7 |
| 76 | 35.3 | <0.2 | 0.8 |
| 77 | >10000 | 445.9 | 10000 |
| 78 | 33 | <0.2 | 10.2 |
| 79 | 31.7 | <0.2 | 9.9 |
| 80 | 133.7 | <0.2 | 44.4 |
| 81 | 10000 | <0.2 | 165.8 |
| 82 | 657.1 | <0.2 | 37.3 |
| 83 | 139 | <0.2 | 30.2 |
| 84 | 366.1 | <0.2 | 129.7 |
| 85 | 12.7 | <0.2 | 17.3 |
| 86 | 7.3 | <0.2 | 2.7 |
| 87 | 15 | <0.2 | 17.5 |
| 88 | 5000 | 3.1 | 3000 |
| 89 | 1893 | <0.2 | 394.3 |
| 90 | 10000 | <0.2 | 359.4 |
| 91 | 233.5 | 64.2 | 989.6 |
| 92 | 2000 | 5.7 | 328.5 |
| 93 | >10000 | 188.8 | >10000 |
| 94 | 10000 | 40.3 | 6114 |
| 95 | 16.6 | <0.2 | 14.1 |
| 96 | 348.7 | <0.2 | 499.9 |
| 97 | 20.2 | <0.2 | 19.9 |
| 98 | 4000 | 13.3 | 3000 |
| 99 | 2000 | 63.7 | 1366 |
| 100 | 2500 | 72.8 | 2798 |

1. A compound of the formula I 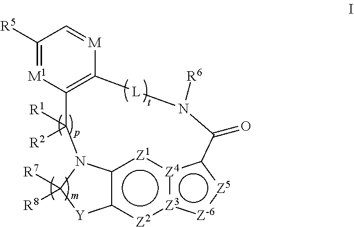
wherein
L is independently —C(R¹)(R²)— or X, with the proviso that when t is 1, then L is —C(R¹)(R²)_;
X is —O—, —S—, —S(O)—, or —S(O)₂—;
each R¹ and R² is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, —OR^{a}, —OC(O)R^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂R^{a}, —CN, or —NO₂, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
M is CR³ or N;
M¹ is CR⁴;
each R³, R⁴, and R⁵ is independently hydrogen, deuterium, halogen, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{C}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or R⁴ and R⁵ taken together with the ring atoms to which they are attached form a C₅-C₈ cycloalkyl, or a 5- to 8-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, mono- or bicyclic heteroaryl, C₅—C cycloalkyl, or 5- to 8-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁶ is H, deuterium, C₁-C₆ alkyl, C₂-C₆ alkenyl, C₂-C₆ alkynyl, C₃-C₆ cycloalkyl, 3- to 7-membered heterocycloalkyl, C₆₋₁₀ aryl, or mono- or bicyclic heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by deuterium, halogen, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
Y is O, S, NR⁸, or CR⁷R⁸;
each R⁷ and R⁸ is independently H, deuterium, halogen, —CN, —OR^{e}, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₂-C₆ alkenyl, C₂-C₆ alkynyl, C₃-C₆ cycloalkyl, 4- to 6-membered heterocycloalkyl, 3- to 7-membered heterocycloalkyl, C₆-C₁₀ aryl, exocyclic ethylene group, or mono- or bicyclic heteroaryl is optionally substituted by a —N₃, —CN, —OR^{e}, —OC(O)R^{e}, —OC(O)NR^{e}R^{f}, —OC(═N)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, or —P(O)₂OR^{e};
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl;
each of Z¹, Z², Z³, Z⁴, Z⁵, and Z⁶ is independently N, NH, C or CH;
m is 0, 1, 2, or 3;
p is 1, 2, 3, or 4; and
t is 1, 2, 3, 4, or 5;
or a pharmaceutically acceptable salt thereof.

2. The compound of claim 1, or a pharmaceutically acceptable salt thereof, wherein p is 1.

3. The compound of claim 1, or a pharmaceutically acceptable salt thereof, wherein t is 3 or 4.

4. The compound of claim 1, or a pharmaceutically acceptable salt thereof, having the formula II 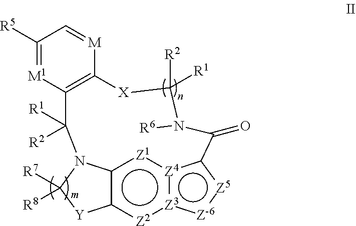
wherein
M is CR³ or N;
M¹ is CR⁴;
X is O, S, S(O), or S(O)₂;
each R¹ and R² is independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}, or R¹ and R² taken together with the carbon to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;
R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;
R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;
Y is O, S, NR⁸, or CR⁷R⁸;
each R⁷ and R⁸ is independently H, deuterium, halogen, —CN, —OR^{c}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group is optionally substituted by a —N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl;
each of Z¹, Z², Z³, Z⁴, Z⁵, and Z⁶ is independently N, NH, C or CH;
m is 0, 1, 2, or 3; and
n is 2, 3, or 4.

5. The compound of 4, having the formula III 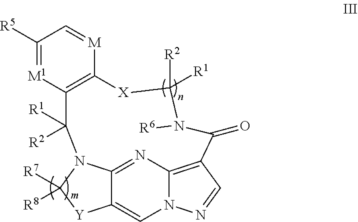
or a pharmaceutically acceptable salt thereof.

6. The compound of claim 5, or a pharmaceutically acceptable salt thereof, wherein n is 2 or 3.

7. The compound of claim 6, or a pharmaceutically acceptable salt thereof, wherein m is 2 or 3.

8. The compound of claim 1, having the formula 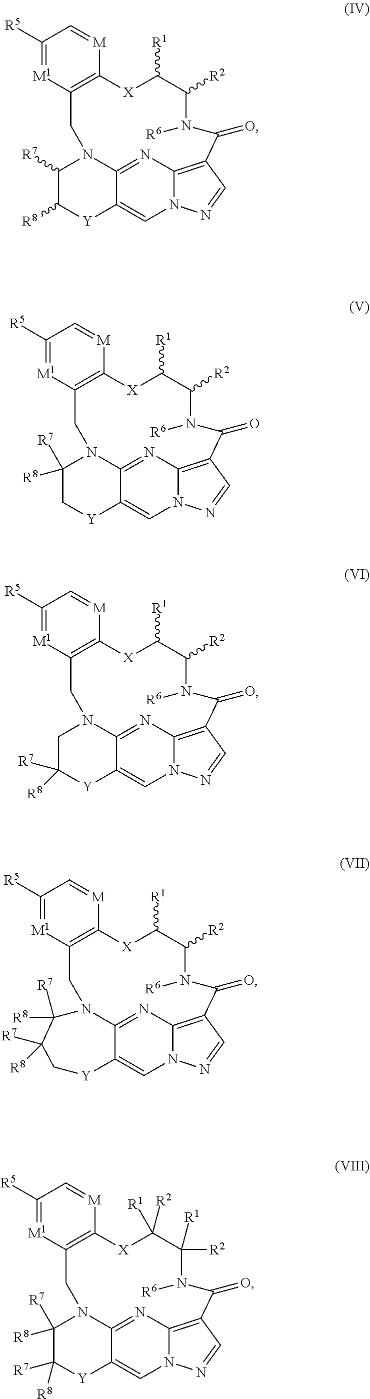 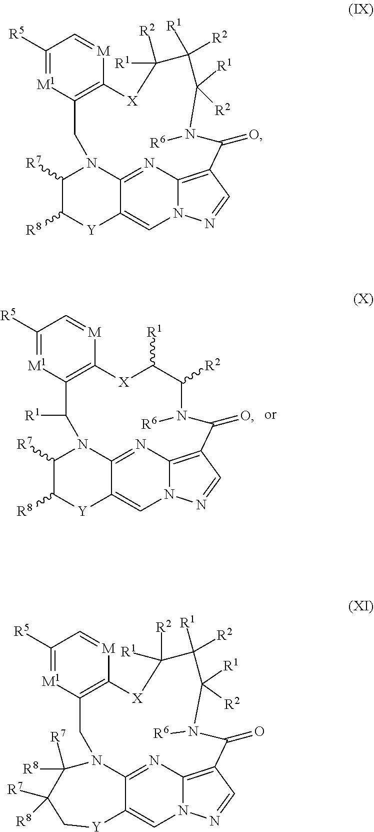
or a pharmaceutically acceptable salt thereof.

9. The compound of claim 1, or a pharmaceutically acceptable salt thereof, wherein Y is O.

10. The compound of claim 9, or a pharmaceutically acceptable salt thereof, wherein M is CR³.

11. The compound of claim 10, or a pharmaceutically acceptable salt thereof, wherein R³ is H, deuterium, C₁-C₆ alkyl or halogen.

12. (canceled)

13. The compound of claim 1, or a pharmaceutically acceptable salt thereof, wherein M is N.

14. (canceled)

15. The compound of claim 11, or a pharmaceutically acceptable salt thereof, wherein R⁴ is H, deuterium, —CN, C₁-C₆ alkyl or halogen.

16. (canceled)

17. The compound of claim 15, or a pharmaceutically acceptable salt thereof, wherein R⁵ is F.

18.-23. (canceled)

24. The compound of claim 17 wherein R⁷ is H or C₁-C₆ alkyl, wherein each hydrogen atom in C₁-C₆ alkyl is independently optionally substituted by deuterium, —OH, or —OC₁-C₆ alkyl.

25. The compound of claim 1, selected from the group consisting of 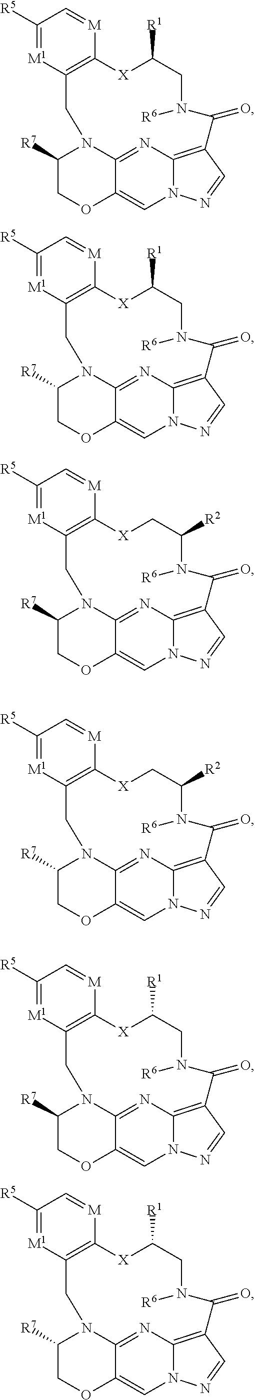 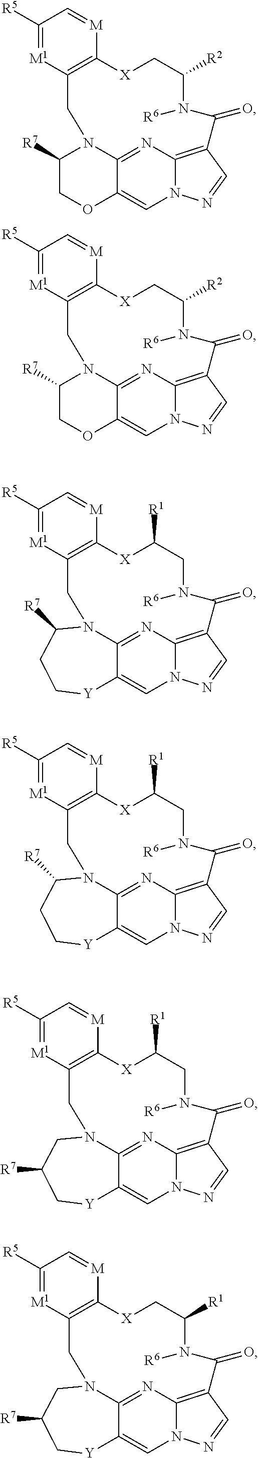 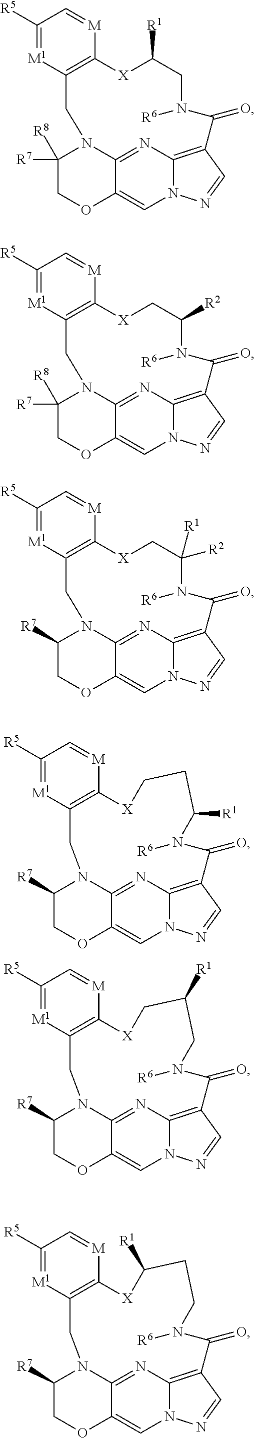 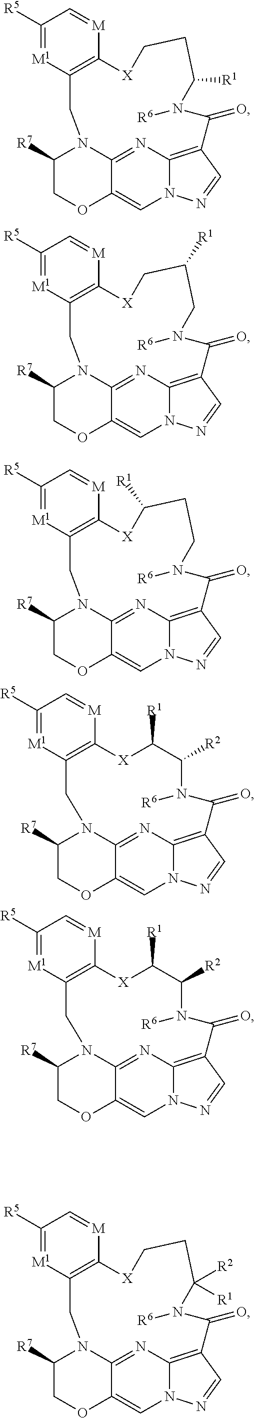 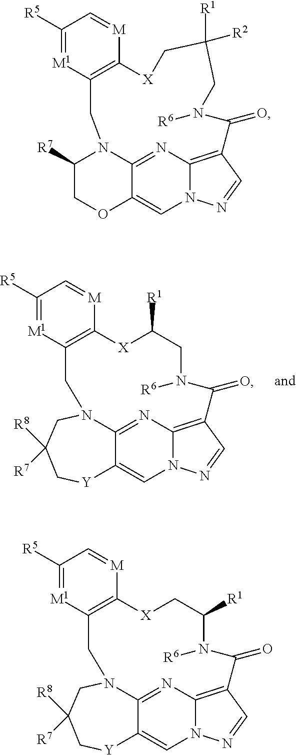
wherein
M is CR³ or N;
M¹ is CR⁴;
X is O, S, S(O), or S(O)₂;
R¹ and R² are each independently H, deuterium, C₁-C₆ alkyl, C₂-C₆ alkenyl, C₂-C₆ alkynyl, C₃-C₆ cycloalkyl, C₆-C₁₀ aryl, —OR^{a}, —SR^{a}, —NR^{a}R^{b}, —C(O)OR^{a}, —C(O)NR^{a}R^{b}, or R¹ and R² taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl and C₆-C₁₀ aryl is independently optionally substituted by deuterium, halogen, —OH, —CN, —OC₁-C₆ alkyl, —OC₁-C₆ alkyl(C₆-C₁₀ aryl), —NH₂, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NH(C₁-C₆ alkyl), —N(C₁-C₆ alkyl)C(O)NH₂, —N(C₁-C₆ alkyl)C(O)NH(C₁-C₆ alkyl), —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHC(O)OH, —N(C₁-C₆ alkyl)C(O)OH, —NHS(O)C₁-C₆ alkyl, —NHS(O)₂C₁-C₆ alkyl, —N(C₁-C₆ alkyl)S(O)C₁-C₆ alkyl, —N(C₁-C₆ alkyl)S(O)₂C₁-C₆ alkyl, —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —C(O)C₁-C₆ alkyl, —CO₂H, —C(O)OC₁-C₆ alkyl, —C(O)NH₂, —C(O)NH(C₁-C₆ alkyl), —C(O)N(C₁-C₆ alkyl)₂, —SC₁-C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —S(O)NH₂, —S(O)₂NH₂, —OS(O)N(C₁-C₆ alkyl)₂, —OS(O)₂N(C₁-C₆ alkyl)₂, —OS(O)NH(C₁-C₆ alkyl), —OS(O)₂NH(C₁-C₆ alkyl), —OS(O)NH₂, —OS(O)₂NH₂, —P(C₁-C₆ alkyl)₂, —P(O)(C₁-C₆ alkyl)₂, C₃-C₆ cycloalkyl, or 3- to 7-membered heterocycloalkyl;
R³, R⁴, and R⁵ are each independently H, fluoro, chloro, bromo, C₁-C₆ alkyl, —OH, —CN, —OC₁-C₆ alkyl, —NHC₁-C₆ alkyl, —N(C₁-C₆ alkyl)₂ or —CF₃;
R⁶ is H, C₁-C₆ alkyl or 3- to 7-membered heterocycloalkyl, wherein each hydrogen atom in C₁-C₆ alkyl or 3- to 7-membered heterocycloalkyl is independently optionally substituted by halogen, —OH, —CN, —OC₁-C₆ alkyl, —NH₂, —NH(C₁-C₆ alkyl), —N(C₁-C₆ alkyl)₂, —CO₂H, —C(O)OC₁-C₆ alkyl, —C(O)NH₂, —C(O)NH(C₁-C₆ alkyl), —C(O)N(C₁-C₆ alkyl)₂, C₃-C₆ cycloalkyl, or monocyclic 5- to 7-membered heterocycloalkyl;
Y is O, S, NR⁸, or CR⁷R⁸; and
each R⁷ and R⁸ is independently H, deuterium, halogen, —CN, —OR^{e}, or C₁-C₆ alkyl, or alternatively, R⁷ and R⁸ taken together with the carbon to which they are attached form a C₃-C₆ cycloalkyl or a 4- to 6-membered heterocycloalkyl, or alternatively, R⁷ and R⁸ taken together with the carbon to which they are attached form an exocyclic ethylene group, wherein each hydrogen atom in C₁-C₆ alkyl, C₃-C₆ cycloalkyl, 4- to 6-membered heterocycloalkyl, or exocyclic ethylene group, or mono- or bicyclic heteroaryl wherein each hydrogen atom in C₁-C₆ alkyl is optionally substituted by a —N₃, —CN, —OH, —OC₁-C₆ alkyl, —OC(O)C₁-C₆ alkyl, —OC(O)N(C₁-C₆ alkyl)₂, —OC(O)NH(C₁-C₆ alkyl), —OC(O)NH₂, —OC(═N)N(C₁-C₆ alkyl)₂, —OC(═N)NH(C₁-C₆ alkyl), —OC(═N)NH₂, —OS(O)C₁-C₆ alkyl, —OS(O)₂C₁-C₆ alkyl, —OS(O)N(C₁-C₆ alkyl)₂, —OS(O)NH(C₁-C₆ alkyl), —OS(O)NH₂, —OS(O)₂N(C₁-C₆ alkyl)₂, —OS(O)₂NH(C₁-C₆ alkyl), —OS(O)₂NH₂, —SH, —SC₁-C₆ alkyl, —S(O)C₁-C₆ alkyl, —S(O)₂C₁-C₆ alkyl, —S(O)N(C₁-C₆ alkyl)₂, —S(O)NH(C₁-C₆ alkyl), —S(O)NH₂, —S(O)₂N(C₁-C₆ alkyl)₂, —S(O)₂NH(C₁-C₆ alkyl), —S(O)₂NH₂, —N(C₁-C₆ alkyl)₂, —NH(C₁-C₆ alkyl), —NH₂, —N(C₁-C₆ alkyl)C(O)C₁-C₆ alkyl, —NHC(O)C₁-C₆ alkyl, —N(C₁-C₆ alkyl)C(O)OC₁-C₆ alkyl, —N(C₁-C₆ alkyl)C(O)OH, —NHC(O)OC₁-C₆ alkyl, —NHC(O)OH, —N(C₁-C₆ alkyl)C(O)N(C₁-C₆ alkyl)₂, —N(C₁-C₆ alkyl)C(O)NH(C₁-C₆ alkyl), —N(C₁-C₆ alkyl)C(O)NH₂, —NHC(O)N(C₁-C₆ alkyl)₂, —NHC(O)NH(C₁-C₆ alkyl), —NHC(O)NH₂, —N(C₁-C₆ alkyl)S(O)C₁-C₆ alkyl, —NHS(O)C₁-C₆ alkyl, —N(C₁-C₆ alkyl)S(O)₂C₁-C₆ alkyl, —NHS(O)₂C₁-C₆ alkyl, —N(C₁-C₆ alkyl)S(O)N(C₁-C₆ alkyl)₂, —N(C₁-C₆ alkyl)S(O)NH(C₁-C₆ alkyl), —N(C₁-C₆ alkyl)S(O)NH₂, —NHS(O)N(C₁-C₆ alkyl)₂, —NHS(O)NH(C₁-C₆ alkyl), —NHS(O)NH₂, —N(C₁-C₆ alkyl)S(O)₂N(C₁-C₆ alkyl)₂, —N(C₁-C₆ alkyl)S(O)₂NH(C₁-C₆ alkyl), —N(C₁-C₆ alkyl)S(O)₂NH₂, —NHS(O)₂N(C₁-C₆ alkyl)₂, —NHS(O)₂NH(C₁-C₆ alkyl), —NHS(O)₂NH₂, —C(O)C₁-C₆ alkyl, —C(O)OC₁-C₆ alkyl, —C(O)N(C₁-C₆ alkyl)₂, —C(O)NH(C₁-C₆ alkyl), —C(O)NH₂, —P(C₁-C₆ alkyl)₂, —P(O)(C₁-C₆ alkyl)₂, —P(O)₂(C₁-C₆ alkyl)₂, —P(O)N(C₁-C₆ alkyl)₂, —P(O)₂N(C₁-C₆ alkyl)₂, —P(O)OC₁-C₆ alkyl, or —P(O)₂OC₁-C₆ alkyl.

26.-38. (canceled)

39. The compound of claim 1, selected from the group consisting of 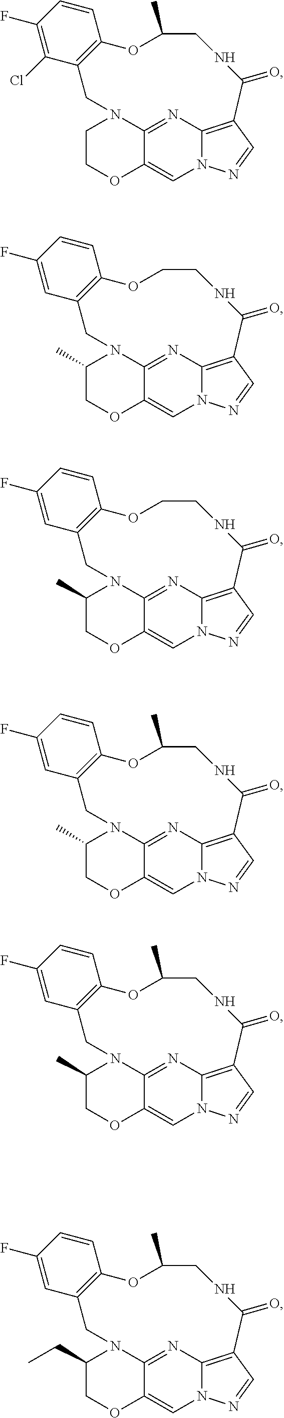 [SMILES: C[C@H]1CNC(=O)c2cnn3cc4c(nc23)N(CCO4)Cc2c(ccc(F)c2Cl)O1 ; C[C@H]1COc2cn3ncc4c3nc2N1Cc1cc(F)ccc1OCCNC4=O ; C[C@@H]1COc2cn3ncc4c3nc2N1Cc1cc(F)ccc1OCCNC4=O ; C[C@H]1CNC(=O)c2cnn3cc4c(nc23)N(Cc2cc(F)ccc2O1)[C@@H](C)CO4 ; C[C@@H]1COc2cn3ncc4c3nc2N1Cc1cc(F)ccc1O[C@@H](C)CNC4=O ; CC[C@@H]1COc2cn3ncc4c3nc2N1Cc1cc(F)ccc1O[C@@H](C)CNC4=O] 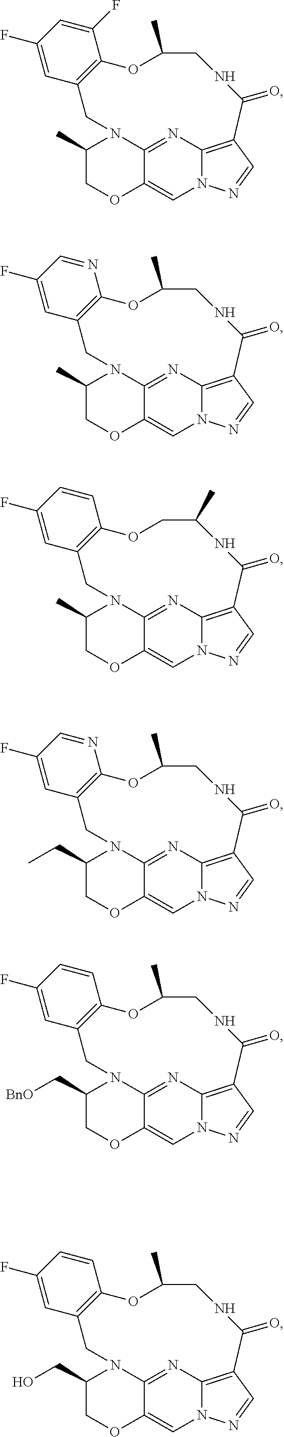 [SMILES: C[C@@H]1COc2cn3ncc4c3nc2N1Cc1cc(F)cc(F)c1O[C@@H](C)CNC4=O ; C[C@@H]1COc2cn3ncc4c3nc2N1Cc1cc(F)cnc1O[C@@H](C)CNC4=O ; C[C@@H]1COc2ccc(F)cc2CN2c3nc4c(cnn4cc3OC[C@H]2C)C(=O)N1 ; CC[C@@H]1COc2cn3ncc4c3nc2N1Cc1cc(F)cnc1O[C@@H](C)CNC4=O ; C[C@H]1CNC(=O)c2cnn3cc4c(nc23)N(Cc2cc(F)ccc2O1)[C@H](COCc1ccccc1)CO4 ; C[C@H]1CNC(=O)c2cnn3cc4c(nc23)N(Cc2cc(F)ccc2O1)[C@H](CO)CO4] 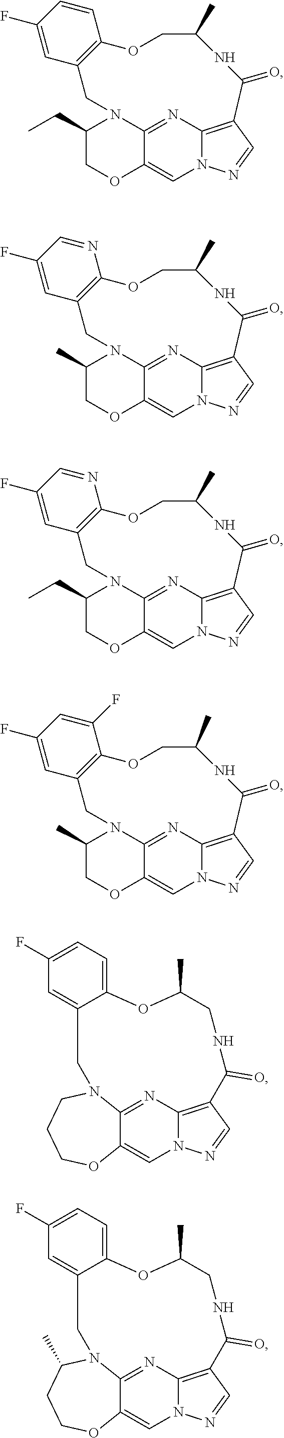 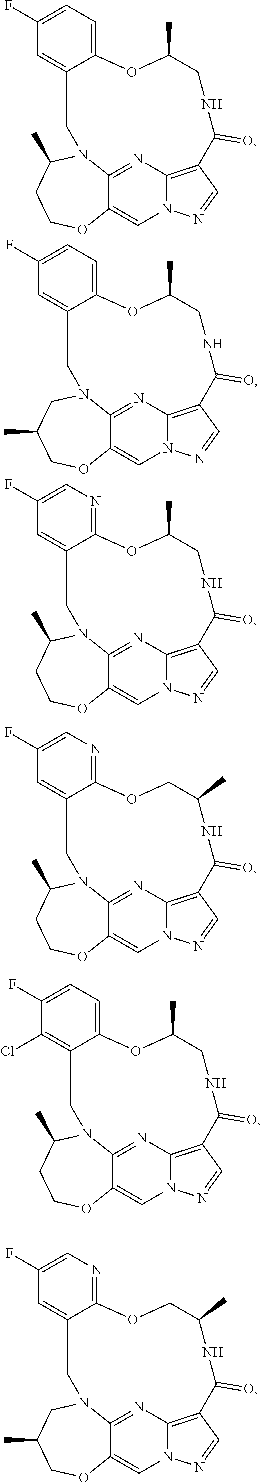 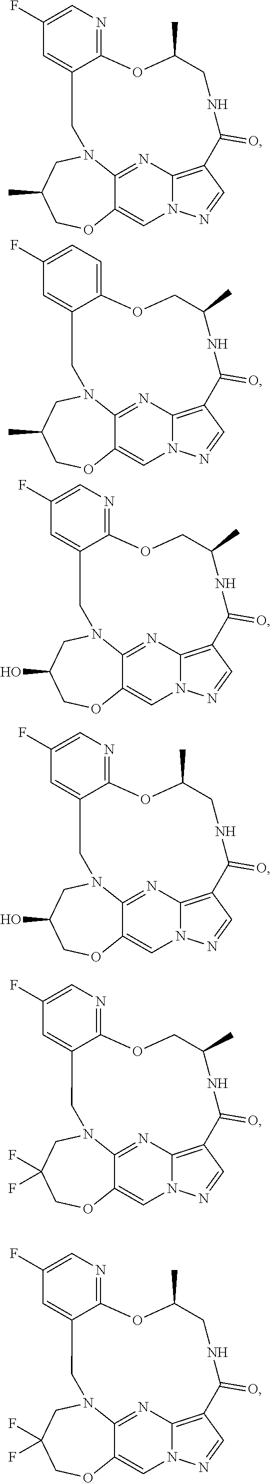 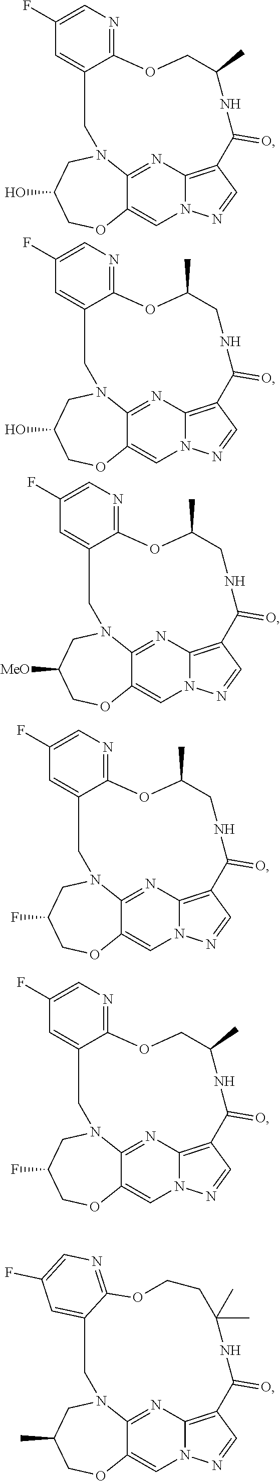 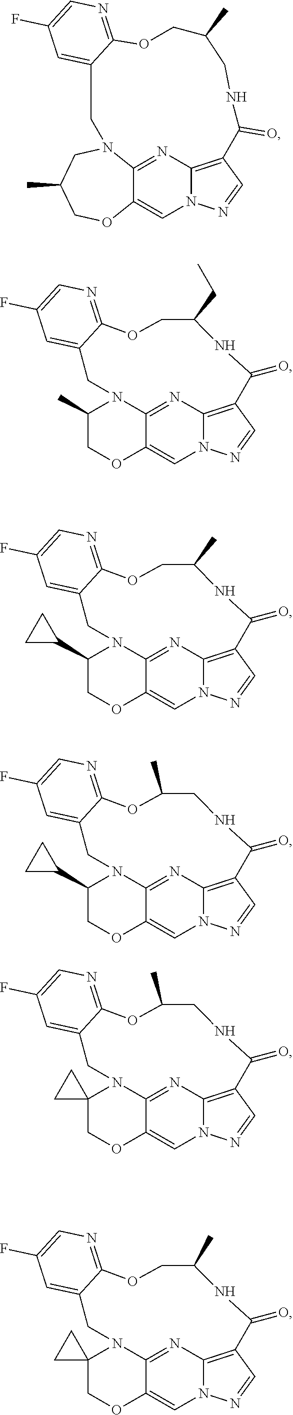 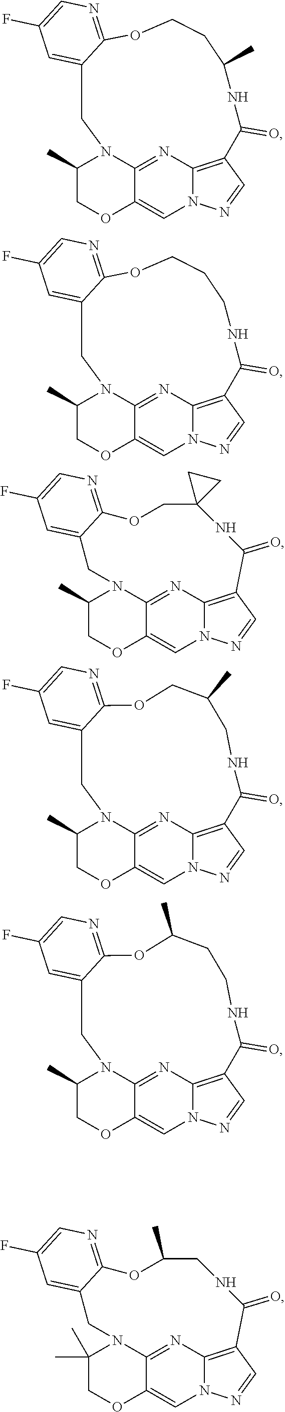 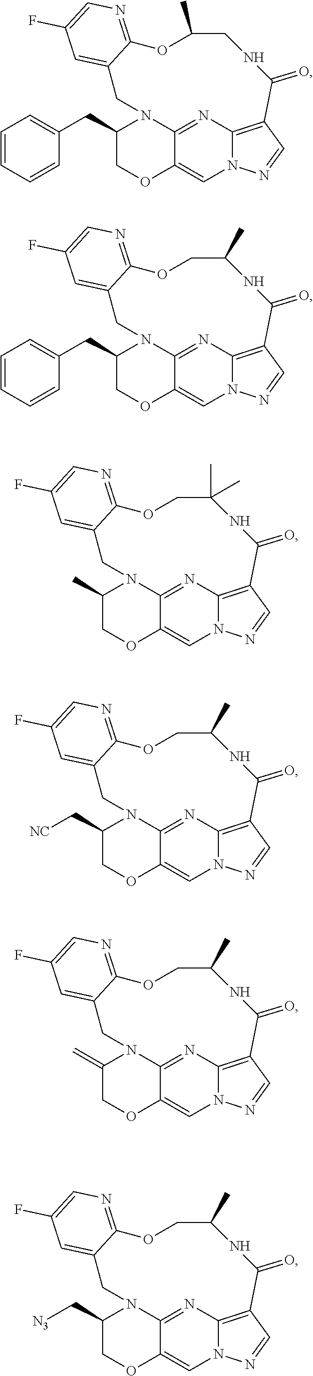 [SMILES: C[C@H]1CNC(=O)c2cnn3cc4c(nc23)N(Cc2cc(F)cnc2O1)[C@H](Cc1ccccc1)CO4 ; C[C@@H]1COc2ncc(F)cc2CN2c3nc4c(cnn4cc3OC[C@H]2Cc2ccccc2)C(=O)N1 ; C[C@@H]1COc2cn3ncc4c3nc2N1Cc1cc(F)cnc1OCC(C)(C)NC4=O ; C[C@@H]1COc2ncc(F)cc2CN2c3nc4c(cnn4cc3OC[C@H]2CC#N)C(=O)N1 ; C=C1COc2cn3ncc4c3nc2N1Cc1cc(F)cnc1OC[C@@H](C)NC4=O ; C[C@@H]1COc2ncc(F)cc2CN2c3nc4c(cnn4cc3OC[C@H]2CN=[N+]=[N-])C(=O)N1] 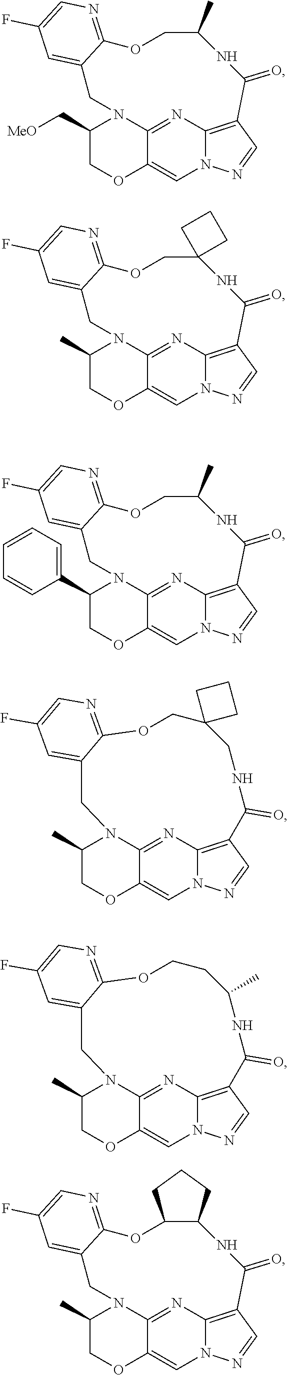 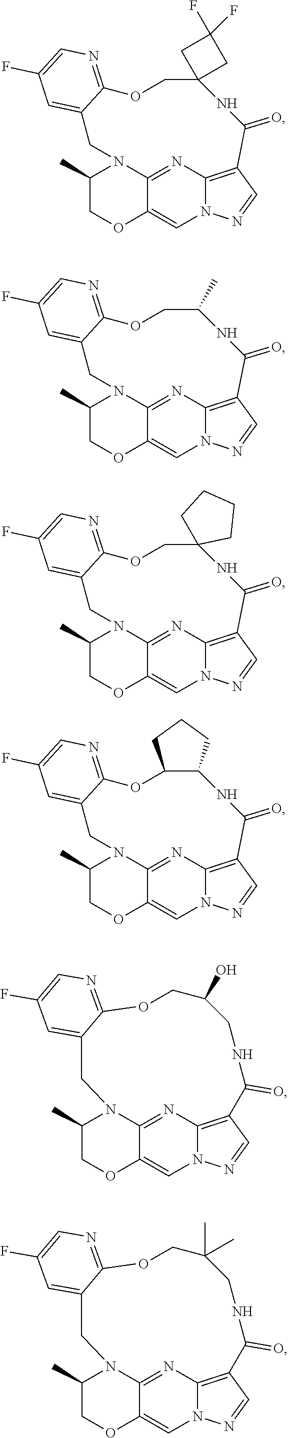 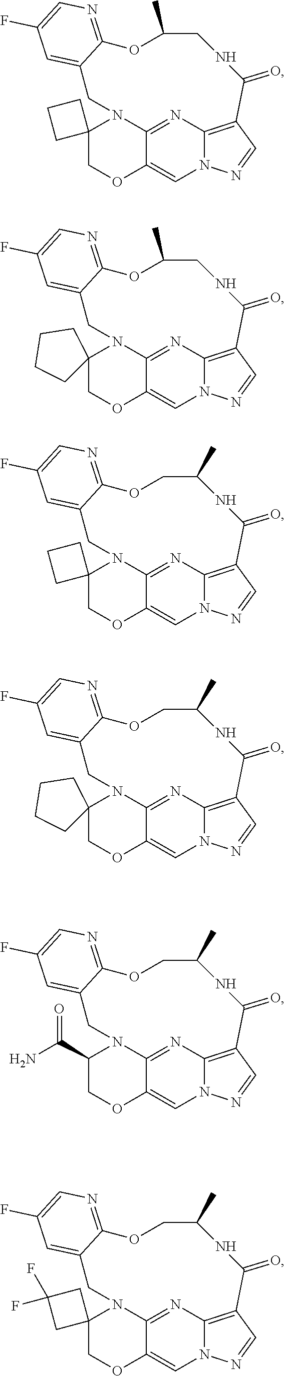 [SMILES: C[C@H]1CNC(=O)c2cnn3cc4c(nc23)N(Cc2cc(F)cnc2O1)C1(CCC1)CO4 ; C[C@H]1CNC(=O)c2cnn3cc4c(nc23)N(Cc2cc(F)cnc2O1)C1(CCCC1)CO4 ; C[C@@H]1COc2ncc(F)cc2CN2c3nc4c(cnn4cc3OCC23CCC3)C(=O)N1 ; C[C@@H]1COc2ncc(F)cc2CN2c3nc4c(cnn4cc3OCC23CCCC3)C(=O)N1 ; C[C@@H]1COc2ncc(F)cc2CN2c3nc4c(cnn4cc3OC[C@H]2C(N)=O)C(=O)N1 ; C[C@@H]1COc2ncc(F)cc2CN2c3nc4c(cnn4cc3OCC23CC(F)(F)C3)C(=O)N1] 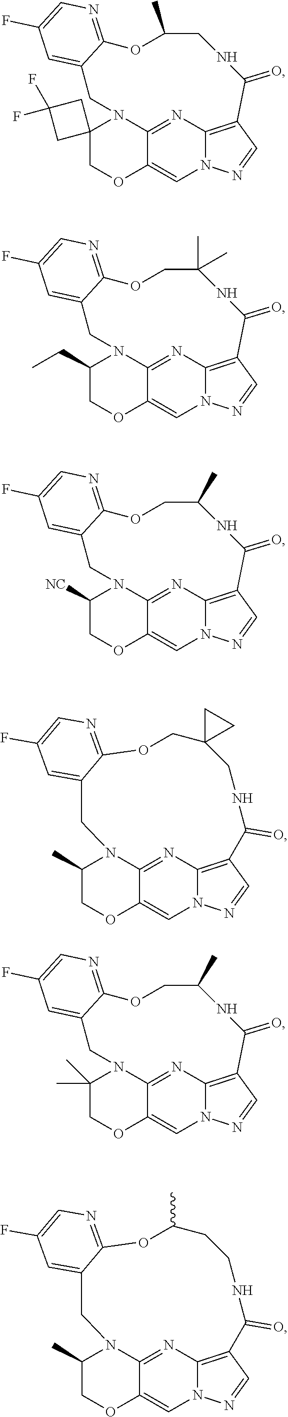 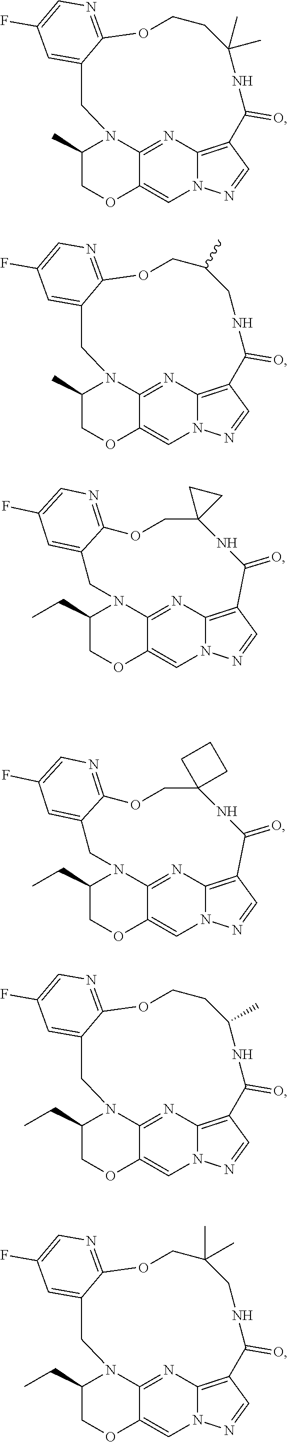 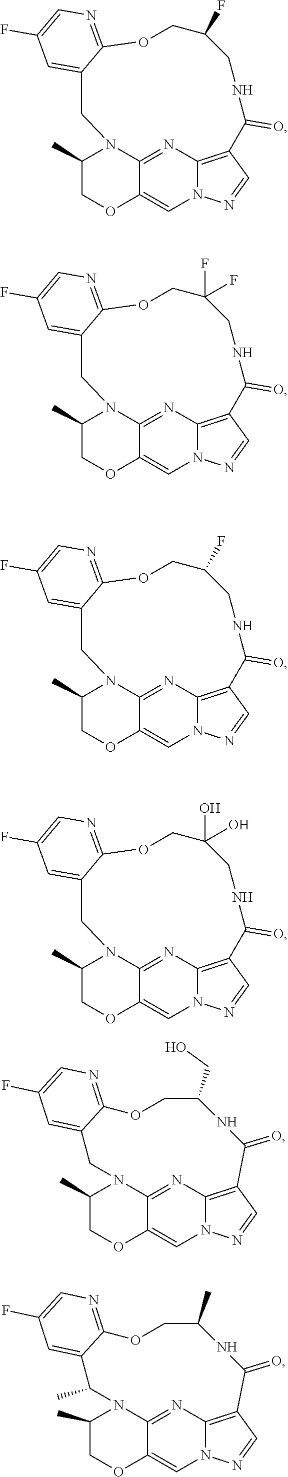 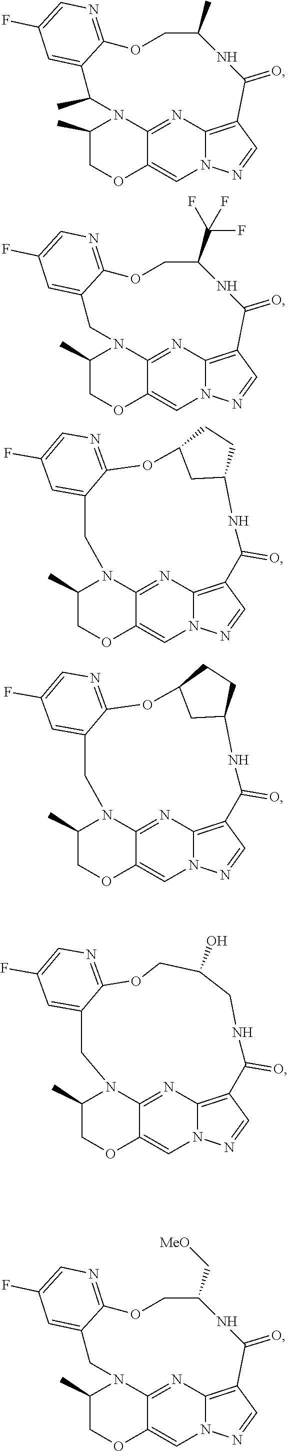 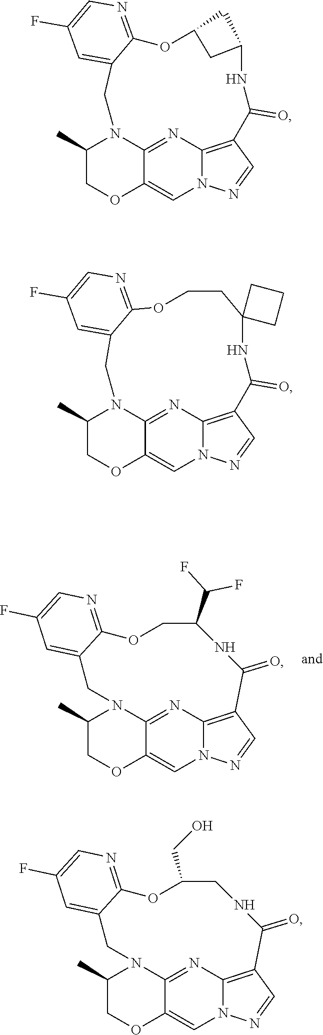 [SMILES: C[C@@H]1COc2cn3ncc4c3nc2N1Cc1cc(F)cnc1O[C@H]1C[C@H](C1)NC4=O ; C[C@@H]1COc2cn3ncc4c3nc2N1Cc1cc(F)cnc1OCCC1(CCC1)NC4=O ; C[C@@H]1COc2cn3ncc4c3nc2N1Cc1cc(F)cnc1OC[C@@H](C(F)F)NC4=O ; C[C@@H]1COc2cn3ncc4c3nc2N1Cc1cc(F)cnc1O[C@H](CO)CNC4=O]
or a pharmaceutically acceptable salt thereof.

40. (canceled)

41. (canceled)

42. A pharmaceutical composition comprising a compound of claim 1, or a pharmaceutically acceptable salt thereof, and optionally at least one diluent, carrier or excipient.

43. A method of treating cancer, pain, neurological diseases, autoimmune diseases, or inflammation comprising administering to a subject in need of such treatment an effective amount of a compound of claim 1, or a pharmaceutically acceptable salt thereof.

44. (canceled)

45. (canceled)

46. A method of inhibiting protein or tyrosine kinases selected from one or more of ALK, ROS1, TRK, JAK, and FGFRs, comprising contacting a cell comprising one or more of such kinases with an effective amount of a compound of claim 1, or a pharmaceutically acceptable salt thereof, wherein the contacting is in vitro, ex vivo, or in vivo.

47. (canceled)
